(19) United States
(12) Patent Application Publication
(10) Pub. No.: US 2024/0299515 A1
(43) Pub. Date: Sep. 12, 2024

(54) MHC CLASS II T-CELL MODULATORY MULTIMERIC POLYPEPTIDES FOR TREATING TYPE 1 DIABETES MELLITUS (T1D) AND METHODS OF USE THEREOF

(71) Applicant: Cue Biopharma, Inc., Boston, MA (US)

(72) Inventors: Ronald D. Seidel, III, Boston, MA (US); Rodolfo J. Chaparro, Cambridge, MA (US); John F. Ross, Boston, MA (US); Chee Meng Low, Boston, MA (US)

(21) Appl. No.: 18/673,179

(22) Filed: May 23, 2024

Related U.S. Application Data
(62) Division of application No. 18/218,943, filed on Jul. 6, 2023, now Pat. No. 12,029,782.
Application No. 18/218,943 is a continuation of application No. 18/107,244, filed on Feb. 8, 2023.
Application No. 18/107,244 is a continuation of application No. PCT/US21/49485, filed on Sep. 8, 2021.
(60) Provisional application No. 63/076,310, filed on Sep. 9, 2020.

(51) Int. Cl. A61K 39/00; A61P 3/10; A61P 37/06; C07K 7/08; C07K 14/495; C07K 14/525; C07K 14/55; C07K 14/62; C07K 14/705; C07K 14/74; C12N 9/88

(52) U.S. Cl. CPC A61K 39/0008; A61P 3/10; A61P 37/06; C07K 7/08; C07K 14/495; C07K 14/525; C07K 14/55; C07K 14/62; C07K 14/70532; C07K 14/70539; C12N 9/88; C12Y 401/01015; A61K 2039/577; A61K 2039/605; A61K 2039/645; C07K 2319/30

(57) ABSTRACT

The present disclosure provides T-cell modulatory multimeric polypeptides (TMMPs) comprising a type 1 diabetes (T1D)-associated peptide epitope, MHC class II polypeptides, and one or more immunomodulatory polypeptides. A TMMP of the present disclosure is useful for modulating activity of a T cell. Thus, the present disclosure provides compositions and methods for modulating the activity of T cells, as well as compositions and methods for treating persons who have T1D.

### CROSS-REFERENCE

This application claims the benefit of U.S. Provisional Patent Application No. 63/076,310, filed Sep. 9, 2020, which application is incorporated herein by reference in its entirety.

### INTRODUCTION

Central to the proper functioning of the mammalian immune system are the coordinated activities and communications between two specialized cell types, antigen-presenting cells (“APCs”) and T cells. APCs serve to capture and break the proteins from foreign organisms, or abnormal proteins (e.g., from genetic mutation in cancer cells), into smaller fragments suitable as signals for scrutiny by the larger immune system, including T cells. In particular, APCs break down proteins into small peptide fragments, which are then paired with proteins of the major histocompatibility complex (“MHC”) and displayed on the cell surface. Cell surface display of an MHC together with a peptide fragment, also known as a T cell epitope, provides the underlying scaffold surveilled by T cells, allowing for specific recognition. The peptide fragments can be pathogen-derived, tumor-derived, or derived from natural host proteins (self-proteins). Moreover, APCs can recognize other foreign components, such as bacterial toxins, viral proteins, viral DNA, viral RNA, etc., whose presence denotes an escalated threat level. The APCs relay this information to T cells through additional costimulatory signals in order to generate a more effective response.

T cells recognize peptide-major histocompatibility complex (“pMHC”) complexes through a specialized cell surface receptor, the T cell receptor (“TCR”). The TCR is unique to each T cell; as a consequence, each T cell is highly specific for a particular pMHC target. In order to adequately address the universe of potential threats, a very large number (˜10,000,000) of distinct T cells with distinct TCRs exist in the human body. Further, any given T cell, specific for a particular T cell peptide, is initially a very small fraction of the total T cell population. Although normally dormant and in limited numbers, T cells bearing specific TCRs can be readily activated and amplified by APCs to generate highly potent T cell responses that involve many millions of T cells. Such activated T cell responses are capable of attacking and clearing viral infections, bacterial infections, and other cellular threats including tumors, as illustrated below. Conversely, the broad, non-specific activation of overly active T cell responses against self or shared antigens can give rise to T cells inappropriately attacking and destroying healthy tissues or cells.

MHC proteins are referred to as human leukocyte antigens (HLA) in humans. HLA class II gene loci include HLA-DM (HLA-DMA and HLA-DMB that encode HLA-DM α chain and HLA-DM β chain, respectively), HLA-DO (HLA-DOA and HLA-DOB that encode HLA-DO α chain and HLA-DO β chain, respectively), HLA-DP (HLA-DPA and HLA-DPB that encode HLA-DP α chain and HLA-DP β chain, respectively), HLA-DQ (HLA-DQA and HLA-DQB that encode HLA-DQ α chain and HLA-DQ β chain, respectively), and HLA-DR (HLA-DRA and HLA-DRB that encode HLA-DR α chain and HLA-DR β chain, respectively).

### SUMMARY

The present disclosure provides T-cell modulatory multimeric polypeptides (TMMPs) comprising a type 1 diabetes (T1D)-associated peptide epitope, MHC class II polypeptides, and one or more immunomodulatory polypeptides. A TMMP of the present disclosure is useful for modulating activity of a T cell. Thus, the present disclosure provides compositions and methods for modulating the activity of T cells, as well as compositions and methods for treating persons who have T1D.

### BRIEF DESCRIPTION OF THE DRAWINGS

FIG. 1A-1E present schematic depictions of TMMPs of the present disclosure, in which the immunomodulatory polypeptide is in Position 1.

FIG. 2A-2D present schematic depictions of TMMPs of the present disclosure, in which the immunomodulatory polypeptide is in Position 2.

FIG. 3A-3D present schematic depictions of TMMPs of the present disclosure, in which the immunomodulatory polypeptide is in Position 3.

FIG. 4A-4D present schematic depictions of TMMPs of the present disclosure, in which the immunomodulatory polypeptide is in Position 5.

FIG. 5 is a schematic depiction of various possible positions of an immunomodulatory polypeptide in a TMMP of the present disclosure.

FIG. 6 provides amino acid sequence of an HLA Class II DRA α chain (SEQ ID NO:130).

FIG. 7A-7D provide amino acid sequences of HLA Class II DRB1 β chains (FIG. 7A), HLA Class II DRB3 β chains (FIG. 7B), an HLA Class II DRB4 β chain (FIG. 7C), and an HLA Class II DRB5 β chain (FIG. 7D).

FIG. 8 provides amino acid sequences of HLA Class II DPA1 α chains.

FIG. 9 provides amino acid sequences of HLA Class II DPB1 β chains.

FIG. 10 provides amino acid sequences of HLA Class II DQA1 α chains.

FIG. 11A-11B provide amino acid sequences of HLA Class II DQB1 β chains.

FIG. 12A-12G provide amino acid sequences of immunoglobulin Fc polypeptides.

FIG. 13A-13N provide amino acid sequences of wild-type (FIG. 13A) and variant (FIG. 13B-3N) DRA*0101 α chains.

FIG. 13O provides an amino acid sequence of a PD-L1 polypeptide.

FIG. 14A-14J provide amino acid sequences of wild-type (FIG. 14A) and variant (FIG. 14B-4J) DRB1*0401 β chains.

FIGS. 15A-15Z and 15AA-15SS provide amino acid sequences of first and second polypeptide chains of TMMPs and APPs of the present disclosure.

FIG. 16A-16C are schematic depictions of disulfide-linked antigen-presenting polypeptides (APPs) of the present disclosure.

FIG. 17A-17C depict various disulfide-linked heterodimers and their characterization. These disulfide-linked heterodimers do not include immunomodulatory polypeptides.

FIG. 18A-18C depict various disulfide-linked heterodimers and their characterization. These disulfide-linked heterodimers include immunomodulatory polypeptides. Linkers: GGSAAAGG (SEQ ID NO:83) and (G4S)3 (SEQ ID NO:74)

FIG. 19A-19D depict production and monomer formation of exemplary TMMPs.

FIG. 20A-20D depict production and monomer formation of exemplary TMMPs. Linkers: (G4S)6 (SEQ ID NO:77), (G4S)3 (SEQ ID NO:74), (G4S)4 (SEQ ID NO:75), GGSAAAGG (SEQ ID NO:83).

FIG. 21A-21E depict the results of stability analysis of TMMP 3005-2639.

FIG. 22A-22B depict the results of stability analysis of disulfide-stabilized TMMPs with the immunomodulatory polypeptide (MOD) at Position 3.

FIG. 23 depict the results of stability analysis of disulfide-stabilized TMMPs with the MOD at Position 1.

FIG. 24 presents a table (Table 6) that provides the expression level (in mg/mL) of various TMMPs. Linkers: GGCGS (SEQ ID NO:234) and GCGGS (SEQ ID NO:235)

FIG. 25 depicts functional response of Proinsulin-specific CD4⁺ T cells following administration with TMMP 3005-2639 or TMMP 3893-2938 as compared to control molecules.

FIG. 26 depicts antigen-specific, PD-L1-dependent suppression of PI-specific CD4⁺ T cells following administration of TMMP.

FIG. 27 depicts suppression of functional response of GAD65-specific CD4⁺ T cells by overnight treatment with TMMP 3005-2580 as compared to control molecules.

FIG. 28 depicts suppression of functional response of Proinsulin-specific CD4⁺ T cells stimulated for 5 days by treatment with TMMP 3005-2639 or TMMP 3893-2938 as compared to control molecules.

FIG. 29 depicts serum pharmacokinetics (PK) analysis of a single 5 mg/kg intravenous dose of TMMP 3893-2938.

FIG. 30 depicts the study design for in vivo analysis of TMMP 3893-2938.

FIG. 31 depicts frequencies of Proins- and HA-reactive T cells following administration of proinsulin (Proins) or hemagglutinin (HA) peptides.

FIG. 32 depicts frequencies of Proins- and HA-reactive T cells following administration of the TMMPs indicated.

FIG. 33 depicts the percent relative change in frequency of proinsulin (PI)- and HA-reactive T cells following treatment with TMMPs, relative to pre-treatment.

FIG. 34 depicts the effect of treatment with TMMPs on cytokine production.

FIG. 35 depicts data showing that cytokine-producing cells are CD4⁺, CD44^{hi}, and PD-1⁺.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

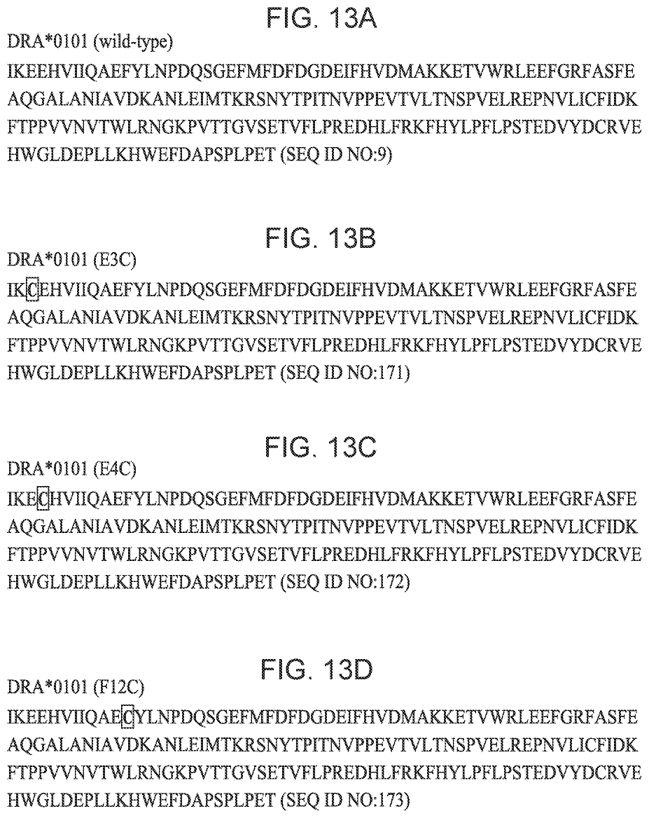

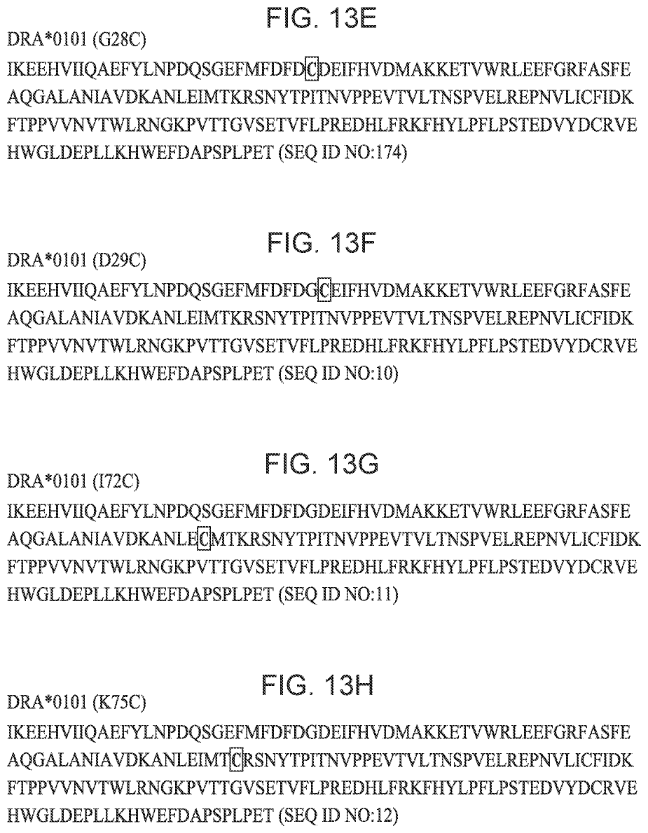

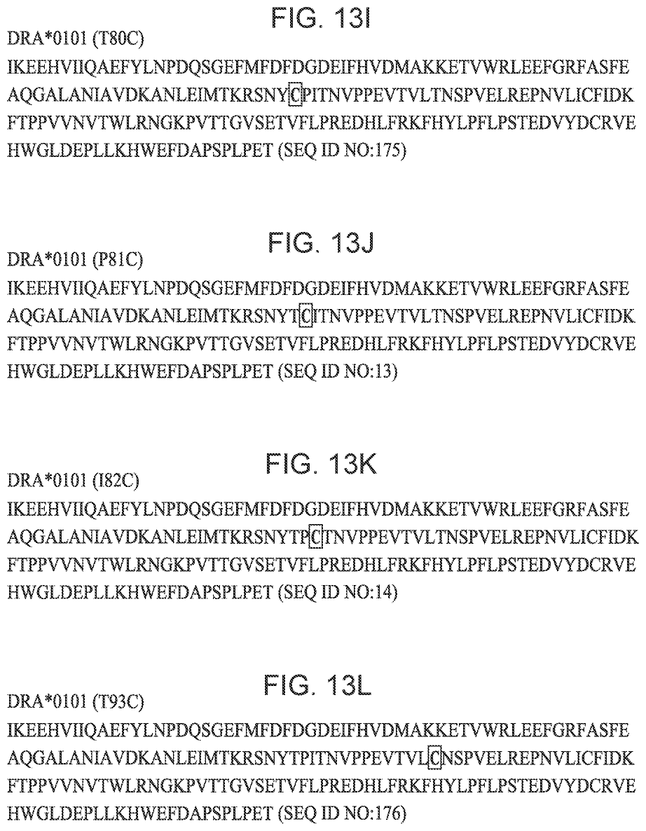

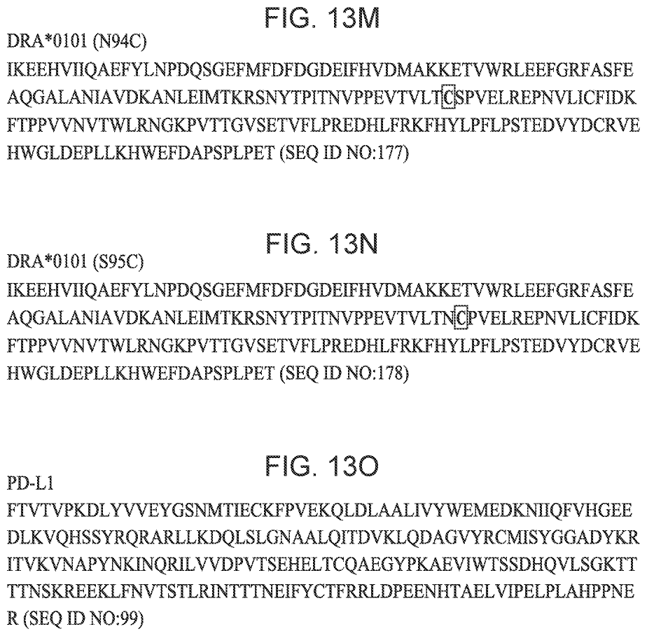

The terms “peptide.” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References herein to a specific residue or residue number in a known polypeptide, e.g., position 72 or 75 of human DRA MHC class II polypeptide, are understood to refer to the amino acid at that position in the wild-type polypeptide (i.e. 172 or K75). To the extent that the sequence of the wild-type polypeptide is altered, either by addition or deletion of one or more amino acids, one of ordinary skill will understand that a reference to the specific residue or residue number will be correspondingly altered so as to refer to the same specific amino acid in the altered polypeptide, which would be understood to reside at an altered position number. For example, if a human DRA MHC class II polypeptide is altered by the addition of one amino acid at the N-terminus, then a reference to position 72 or 75, or to residue 172 or K75, will be understood to indicate the amino acids that are at positions 73 or 76, or to residues 173 and K76. Likewise, a reference herein to substitution of a specific amino acid as a specific position, e.g., 172C, is understood to refer to a substitution of a cysteine for the amino acid at position 72 in the wild-type polypeptide, i.e., isoleucine. If, e.g., the wild-type polypeptide is altered to change the amino acid at position 72 from isoleucine to an alternate amino acid, then the reference to 172C will be understood to refer to the substitution of a cysteine for the alternate amino acid. If in such case the polypeptide is also altered by the addition or deletion of one or more amino acids, then the reference to I72C will be understood to refer to the substitution of a cysteine for the alternate amino acid at the altered position number. A reference to a “non-naturally occurring Cys residue” in a polypeptide, e.g., a DRA MHC class II polypeptide, means that the polypeptide comprises a Cys residue in a location where there is no Cys in the corresponding wild-type polypeptide. This can be accomplished through routine protein engineering in which a cysteine is substituted for the amino acid that occurs in the wild-type sequence, e.g., at position 72 or 75 of the DRA*0101 polypeptide (see FIG. 13G and FIG. 13H) instead of the isoleucine (I) or lysine (K) residues that are present in the wild-type DRA*0101 polypeptide (see FIG. 13A).

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST. ebi.ac.uk/Tools/msa/tcoffee/, ebi.ac.uk/Tools/msa/muscle/, mafft.cbrc.jp/alignment/software/. See, e.g., Altschul et al. (1990) J. Mol. Biol. 215:403-10. Unless otherwise stated, “sequence identity” as referred to herein is determined by BLAST (Basic Local Alignment Search Tool), as described in Altschul et al. (1990) J. Mol. Biol. 215:403.

As used herein the term “in vivo” refers to any process or procedure occurring inside of the body, e.g., of a T1D patient.

As used herein, “in vitro” refers to any process or procedure occurring outside of the body.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The term “binding.” as used herein (e.g. with reference to binding of a T-cell modulatory antigen-presenting polypeptide to a polypeptide (e.g., a T-cell receptor) on a T cell), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. “Covalent binding” or “covalent bond,” as used herein, refers to the formation of one or more covalent chemical bonds between two different molecules.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to as a “co-stimulatory polypeptide”), as used herein, includes a polypeptide on an antigen presenting cell (APC) (e.g., a dendritic cell, a B cell, and the like), or a portion of the polypeptide on an APC, that specifically binds a cognate co-immunomodulatory polypeptide on a T cell, thereby providing a signal which, in addition to the primary signal provided by, for instance, binding of a TCR/CD3 complex with a major histocompatibility complex (MHC) polypeptide loaded with peptide, mediates a T cell response, including, but not limited to, proliferation, activation, differentiation, and the like. An immunomodulatory polypeptide can include, but is not limited to, a cytokine (e.g., IL-2; TGFβ), CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor. 3/TR6, ILT3, ILT4, and HVEM.

As noted above, an “immunomodulatory polypeptide” (also referred to herein as a “MOD”) specifically binds a cognate co-immunomodulatory polypeptide on a T cell (e.g., a target T cell).

“Phosphate buffer saline” or “PBS”, as used herein, means a water-based buffer solution, typically available as a concentrated solution. Unless stated otherwise, the PBS solution used in this disclosure contains sodium chloride (500 mM), sodium phosphate dibasic (10 mM), potassium phosphate monobasic (2 mM), potassium chloride (2.7 mM), and the rest water. The pH of the PBS is 7.5±0.15. The buffer can be prepared in 18.2 megohms DNase, and RNase free water and filtered through 0.22 micron filter.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector.” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (K_{D}). As used herein, the term “avidity” refers to the resistance of a complex of two or more agents to dissociation after dilution.

The term “binding” refers to a direct association between two molecules, due to, for example, covalent, electrostatic, hydrophobic, and ionic and/or hydrogen-bond interactions, including interactions such as salt bridges and water bridges.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual.” “subject.” “host.” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc.

Unless indicated otherwise, the term “substantially” is intended to encompass both “wholly” and “largely but not wholly”. For example, an Ig Fc that “substantially does not induce cell lysis” means an Ig Fc that induces no cell lysis at all or that largely but not wholly induces no cell lysis.

As used herein, the term “about” used in connection with an amount indicates that the amount can vary by 10%. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

It is to be understood that this disclosure is not limited to particular embodiments described, as such may, of course, vary. It is also to be understood that the terminology used herein is for the purpose of describing particular embodiments only, and is not intended to be limiting, since the scope of the inventions described in the appended claims will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an immunomodulatory polypeptide” includes a plurality of such immunomodulatory polypeptides and reference to “the Treg” includes reference to one or more Tregs and equivalents thereof known to those skilled in the art, and so forth. It is further noted that the claims may be drafted to exclude any optional element or any element that is included in a list or other recitation of elements that share a common generic or specific feature, property, or activity. As such, this statement is intended to serve as antecedent basis for use of such exclusive terminology as “solely.” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

### DETAILED DESCRIPTION

The present disclosure provides T-cell modulatory multimeric polypeptides (TMMPs) comprising a type 1 diabetes (T1D)-associated peptide, MHC class II polypeptides, and one or more immunomodulatory polypeptides. A TMMP of the present disclosure is useful for modulating activity of a T cell. Thus, the present disclosure provides compositions and methods for modulating the activity of T cells, as well as compositions and methods for treating individuals who have T1D.

### T-Cell Modulatory Multimeric Polypeptides

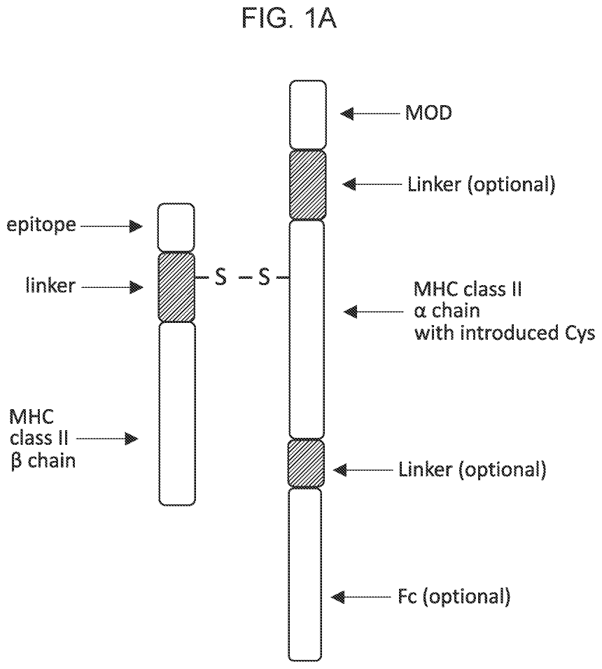

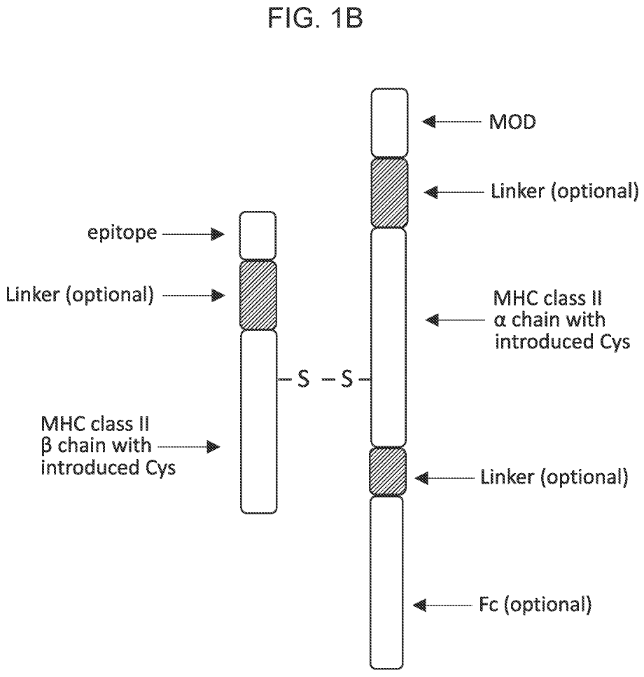

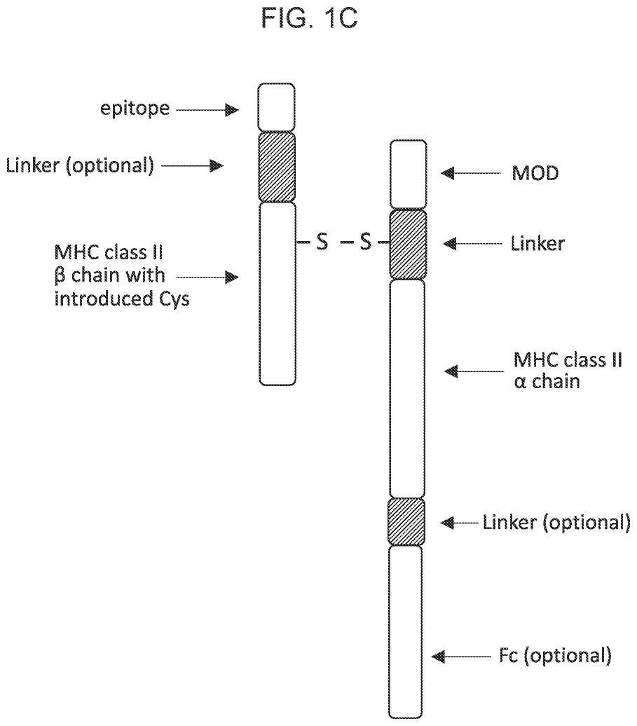

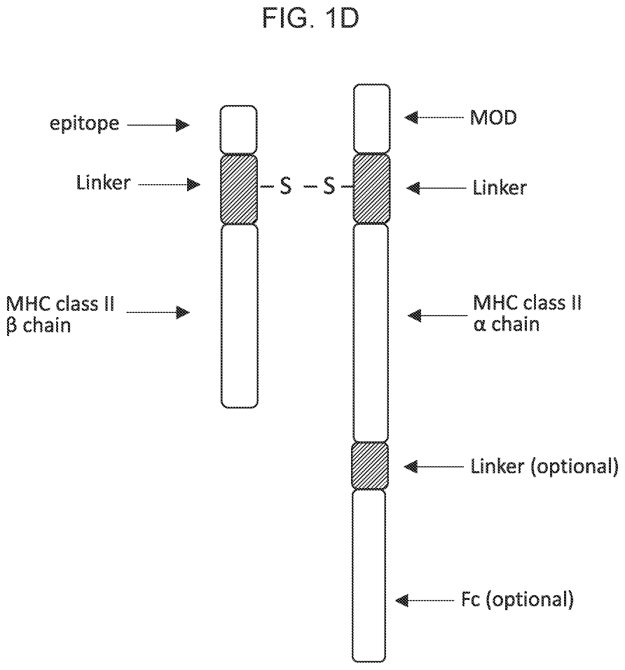

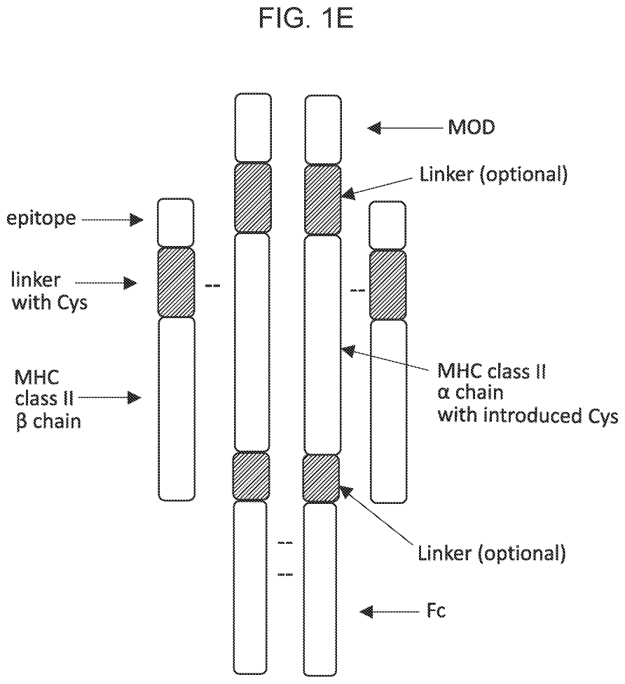

The present disclosure provides TMMPs that comprise a disulfide-linked heterodimer comprising: a) a first polypeptide comprising: i) a peptide that, when present in a TMMP of this disclosure, presents a T1D-associated epitope capable of being bound by a T-cell receptor (TCR) on the surface of a T cell (hereinafter a “T1D peptide”); and ii) a first major histocompatibility complex (MHC) class II polypeptide; and b) a second polypeptide comprising a second MHC class II polypeptide. One or both polypeptides of the heterodimer comprise one or more immunomodulatory polypeptides. The first and the second polypeptides of the heterodimer are covalently linked to one another via at least one disulfide bond. One of the polypeptides of the heterodimer optionally comprises an immunoglobulin (Ig) Fc polypeptide or a non-Ig scaffold. A TMMP of the present disclosure can comprise two disulfide-linked heterodimers. When both heterodimers include Ig Fc polypeptides, disulfide bonds will spontaneously form between the respective Ig Fc polypeptides to covalently link the two heterodimers to one another (depicted schematically in FIG. 1E).

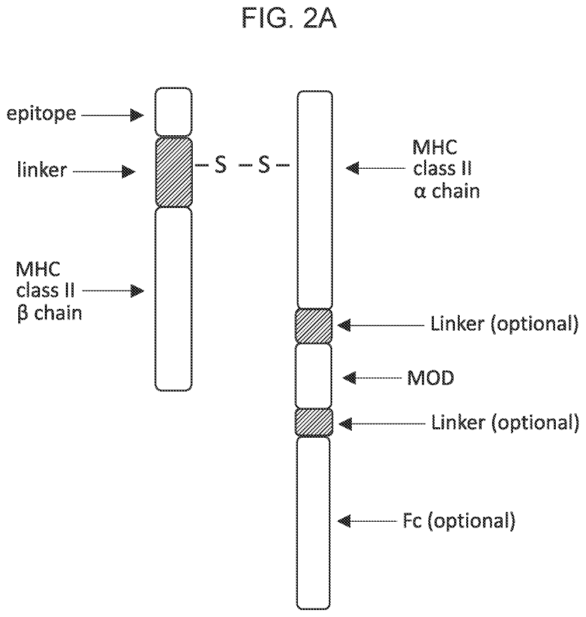

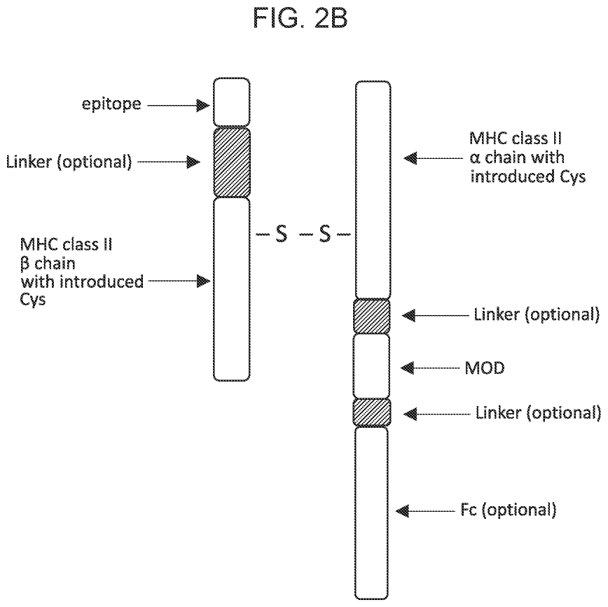

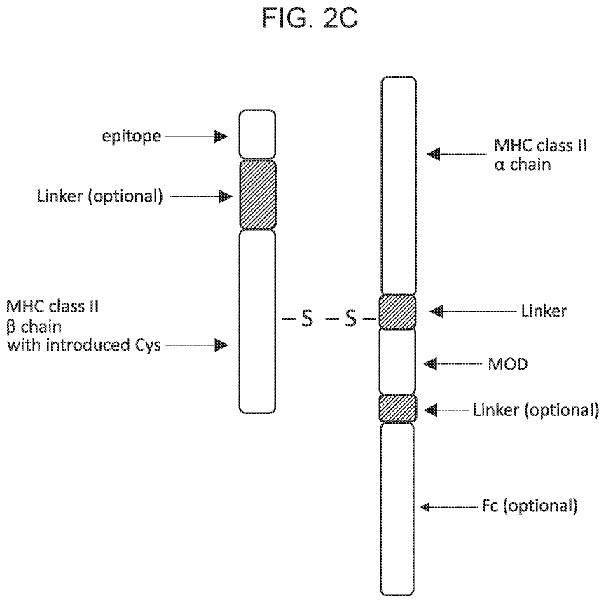

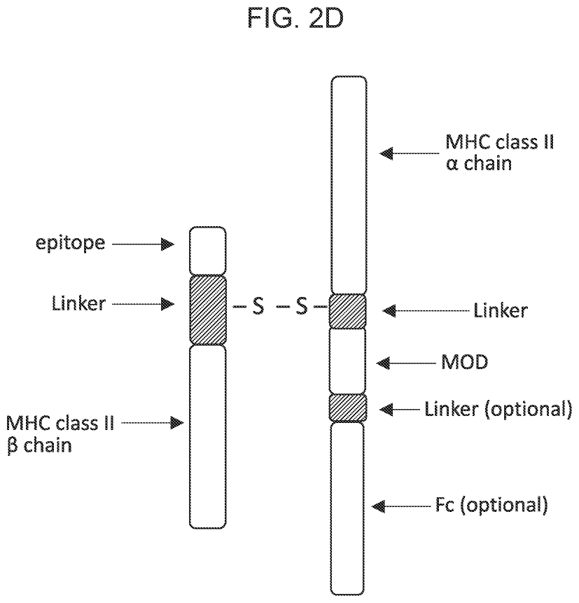

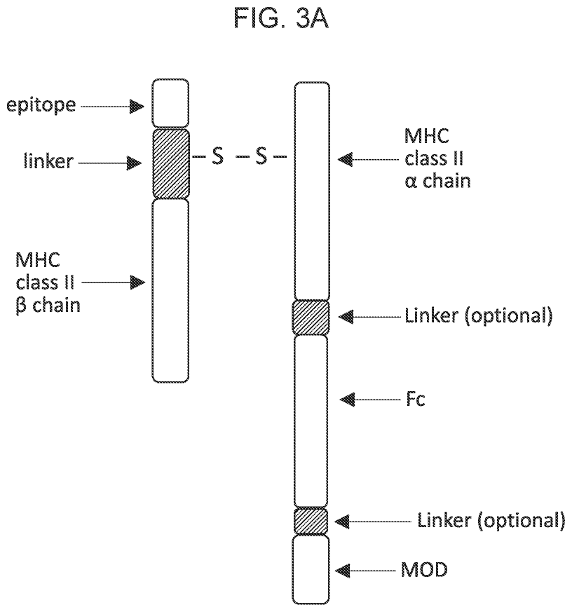

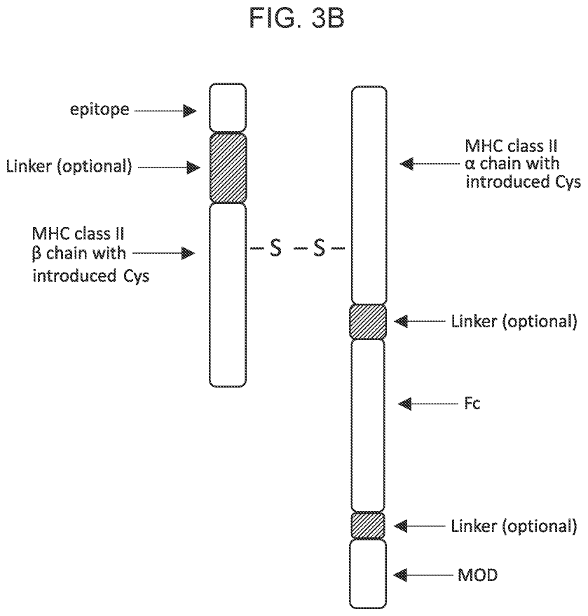

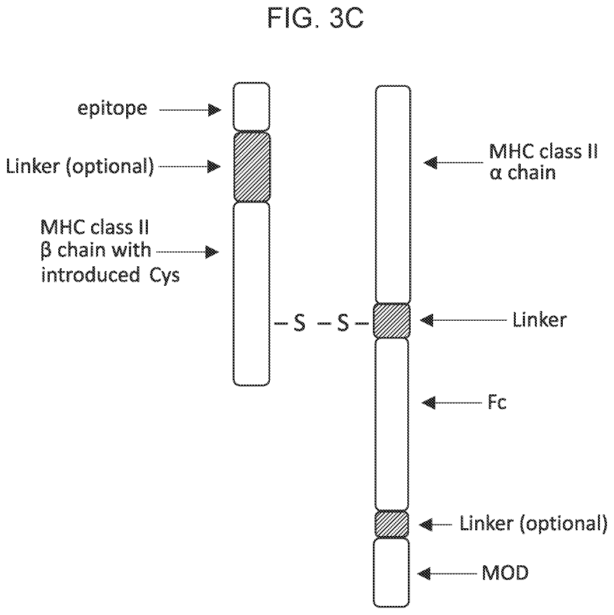

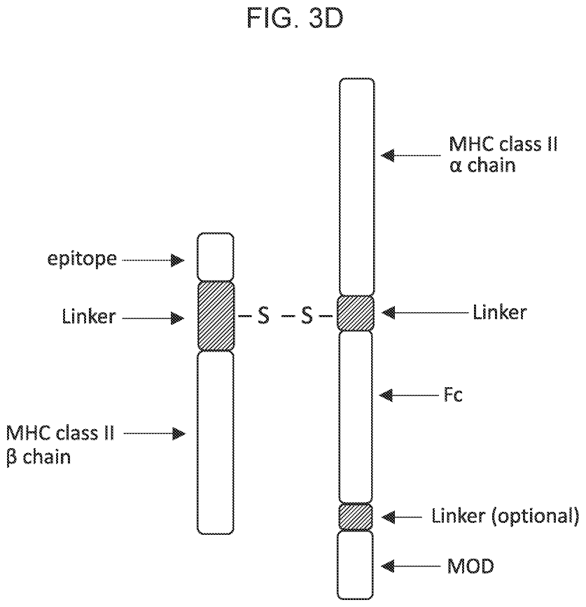

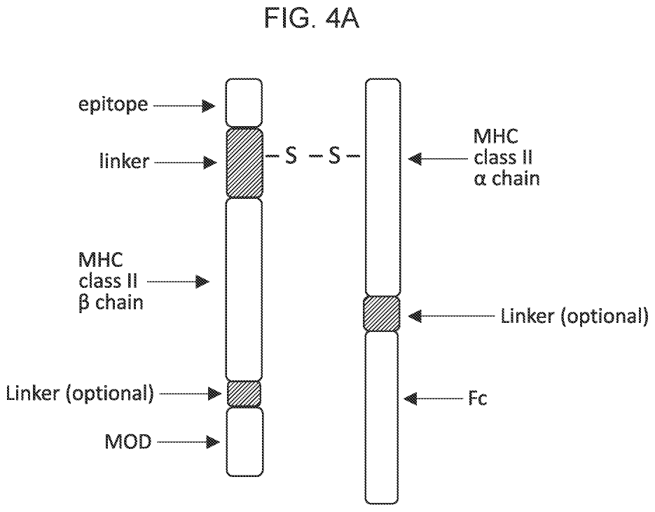

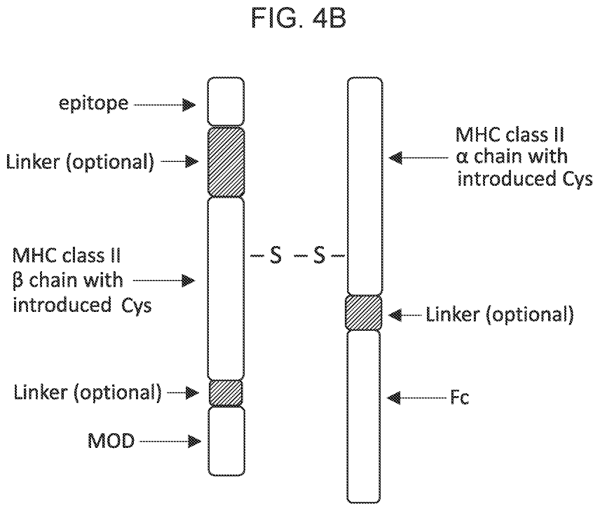

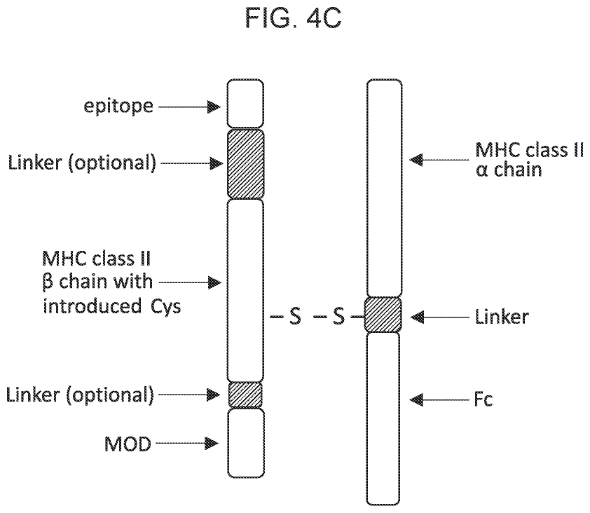

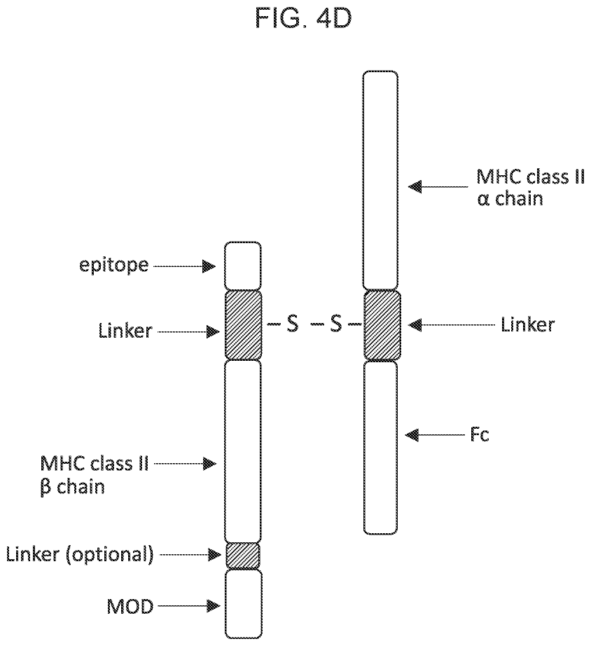

As noted above, the first polypeptide and the second polypeptide of a heterodimer of a TMMP of the present disclosure are covalently linked to one another via at least one disulfide bond. For example, the at least one disulfide bond is present between: i) a Cys present in the first MHC class II polypeptide and a Cys present in the second MHC class II polypeptide; or ii) a Cys present in a peptide linker in the first polypeptide and a Cys present in an MHC class II polypeptide present in the second polypeptide; or iii) a Cys present in a peptide linker in the second polypeptide and a Cys present in an MHC class II polypeptide present in the first polypeptide. These various embodiments are depicted schematically in FIG. 1A-1E, FIG. 2A-2D, FIG. 3A-3D, and FIG. 4A-4D.

The at least one disulfide bond formed between the first polypeptide and the second polypeptide of a heterodimer of a TMMP of the present disclosure provides for increased stability and/or expression of the TMMP, compared to a control TMMP lacking the at least one disulfide bond. For example, in some cases, a TMMP of the present disclosure is at least 10%, at least 20%, at least 30%, at least 40%, at least 50%, at least 60%, at least 70%, at least 80%, at least 90%, at least 2-fold, at least 2.5-fold, at least 3-fold, at least 4-fold, at least 5-fold, at least 10-fold, at least 20-fold, at least 50-fold, at least 100-fold, or more than 100-fold, more stable than a control TMMP lacking the at least one disulfide bond. Stability can be determined by measuring the amount of intact TMMP monomer remaining after incubation of the TMMP for a specific period of time at a specific temperature (e.g., for 1 hour at 37° C.; 1 day at 37° C.; 5 days at 37° C.; 1 hour at 42° C.; 1 day at 42° C.; 5 days at 42° C.; and the like). A disulfide-bonded TMMP of the present disclosure will in some cases exhibit a stability after 3 days at 37° C. at TMMP concentrations of 9.5 mg/mL, 1 mg/mL, or 0.1 mg/mL in the PBS buffer solution containing 500 mM NaCl (described above), as measured by the percent monomer remaining, that is greater than 50%, greater than 60%, greater than 70%, greater than 80%, or greater than 90%. A disulfide-bonded TMMP of the present disclosure will in some cases exhibit a stability after 5 days at 37° C. at TMMP concentrations of 9.5 mg/mL, 1 mg/mL, or 0.1 mg/mL in the PBS buffer solution containing 500 mM NaCl (described above), as measured by the percent monomer remaining, that is greater than 50%, greater than 60%, greater than 70%, or greater than 80%. A disulfide-bonded TMMP of the present disclosure will in some cases exhibit a stability after 3 days at 42° C. at TMMP concentrations of 9.5 mg/mL, 1 mg/mL, or 0.1 mg/mL in the PBS buffer solution containing 500 mM NaCl (described above), as measured by the percent monomer remaining, that is greater than 30%, greater than 40%, greater than 50%, or greater than 60%. A disulfide-bonded TMMP of the present disclosure will in some cases exhibit a stability after 5 days at 42° C. at TMMP concentrations of 9.5 mg/mL, 1 mg/mL, or 0.1 mg/mL in the PBS buffer solution containing 500 mM NaCl (described above), as measured by the percent monomer remaining, that is greater than 30%, greater than 40%, greater than 50%, or greater than 60%. Whether a protein is present in solution as a monomer can be determined using size exclusion chromatography (SEC). Analytical SEC can be performed using a Superdex 200 Increase (3.2×300 mm) column (e.g., from GE Healthcare). For example, for analytical SEC, the running buffer can be PBS containing 500 mM NaCl (described above) and the flow rate can be 0.15 ml/minute.

As another example, in some cases, a TMMP of the present disclosure is expressed (produced) in a Chinese hamster ovary (CHO) cell in vitro at a level that is at least 10%, at least 20%, at least 30%, at least 40%, at least 50%, at least 60%, at least 70%, at least 80%, at least 90%, at least 2-fold, at least 2.5-fold, at least 3-fold, at least 4-fold, at least 5-fold, at least 10-fold, at least 20-fold, at least 50-fold, at least 100-fold, or more than 100-fold, higher than the expression level of a control TMMP lacking the at least one disulfide bond when expressed under the same conditions and in the same CHO cells. Expression levels can be determined by: i) producing the TMMP in a mammalian cell in vitro in a CHO cell in vitro; and ii) determining the amount of TMMP produced by the mammalian cell. For example, where a TMMP comprises an Ig Fc polypeptide, the TMMP can be isolated from the mammalian cells and/or from culture medium in which the mammalian cells are cultured, where isolation of the TMMP can be carried out by affinity chromatography, e.g., on a Protein A column, a Protein G column, or the like. An example of a suitable mammalian cell is a CHO cell; e.g., an Expi-CHO-S™ cell (e.g., ThermoFisher Scientific, Catalog #A29127).

A protein consisting of the desired TMMP is referred to herein as a “monomer.” For example, “monomer” can refer to a single heterodimer that is the desired TMMP when the heterodimer is not covalently bonded to another heterodimer, e.g., when the heterodimer does not include an optional Ig Fc polypeptide. Likewise, a homodimeric TMMP protein comprising 2 heterodimers, each comprising 2 polypeptide chains (for a total of 4 polypeptide chains), where 2 of the four polypeptide chains comprise Ig Fc polypeptides, wherein the Ig Fc polypeptides spontaneously form disulfide bonds that covalently link the heterodimers to each other, also is referred to herein as a “monomer.” The term “monomer” does not include aggregates of monomers. Whether a protein is present in solution as a monomer can be determined using size exclusion chromatography (SEC). Analytical SEC can be performed using a Superdex 200 Increase (3.2×300 mm) column (e.g., from GE Healthcare). For example, for analytical SEC, the running buffer can be PBS containing 500 mM NaCl (described above) and the flow rate can be 0.15 ml/minute.

In some cases, a TMMP of the present disclosure exhibits at least 10%, at least 20%, at least 30%, at least 40%, at least 50%, at least 60%, at least 70%, at least 80%, at least 90%, at least 2-fold, at least 2.5-fold, at least 3-fold, at least 4-fold, at least 5-fold, at least 10-fold, at least 20-fold, at least 50-fold, at least 100-fold, or more than 100-fold, greater stability in the PBS buffer solution containing 500 mM NaCl (described above) in vitro over a specified period of time and at a specified temperature (e.g., in a solution at a temperature of 37° C. to 42° C. for a period of time of from 1 hour to 28 days; e.g., for 1 hour at 37° C.; 1 day at 37° C.; 5 days at 37° C.; 1 hour at 42° C.; 1 day at 42° C.; 5 days at 42° C.; 5 days at 37° C.; 10 days at 37° C.; 14 days at 37° C.; 28 days at 37° C.; and the like), compared to a control TMMP lacking the at least one disulfide bond between the first polypeptide and the second polypeptide of the heterodimer. The TMMP can be present in the PBS buffer solution in a concentration of from 0.1 mg/mL to 10 mg/mL, and the buffer solution can be kept at 37° C. or 42° C. for 1 hour, 5 days, 10 days, 14 days, 21 days, or 28 days. As discussed herein, stability is determined by measuring the percent of monomer (as defined above) remaining in the solution after a specified time in the solution at a specified temperature. Loss of monomer often is characterized by the formation of aggregates, or alternatively by the breakdown of the monomer into one or more separate polypeptide chains or fragments thereof. Thus, for example, “at least 10% greater stability” means that at least 10% more TMMP monomer is present in a test sample than in a control sample comprising the same polypeptide chains but lacking the at least one disulfide bond between the first polypeptide and the second polypeptide of the heterodimer. The percent more monomer in the test sample is calculated based on the amount of monomer in the control sample. Thus, e.g., after a specified period of time at a specified temperature, at least at least 10%, at least 20%, at least 30%, at least 40%, at least 50%, at least 60%, at least 70%, at least 80%, at least 90%, at least 2-fold, at least 2.5-fold, at least 3-fold, at least 4-fold, at least 5-fold, at least 10-fold, at least 20-fold, at least 50-fold, at least 100-fold, or more than 100-fold, more monomer remains of a TMMP of the present disclosure (comprising a disulfide bond between the first and second polypeptide chains) compared to the amount of monomer remaining of a control TMMP lacking a disulfide bond between the first and second polypeptide chains.

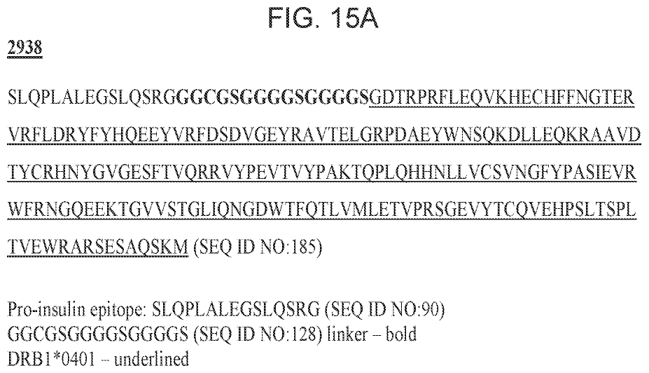

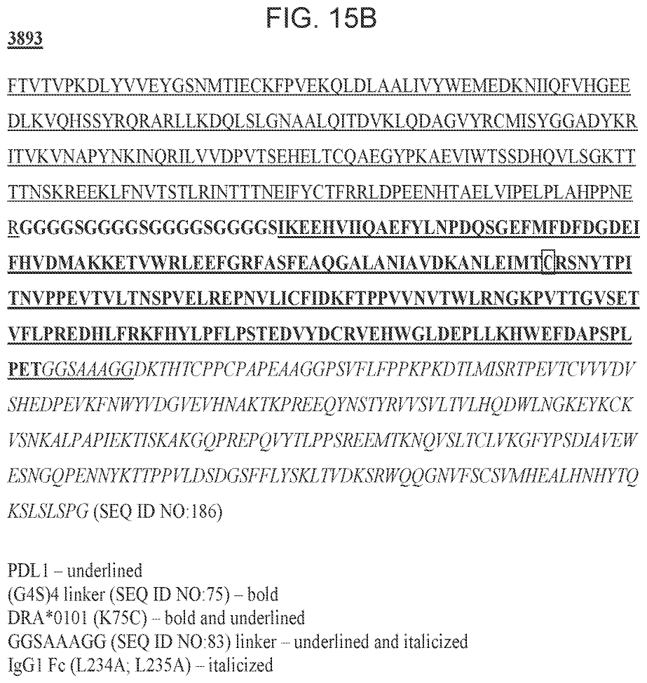

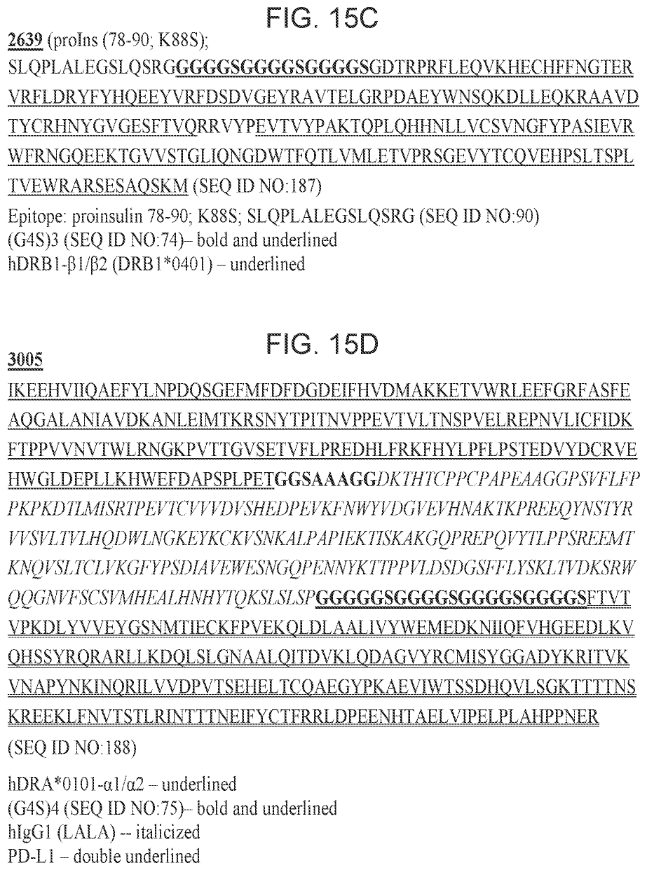

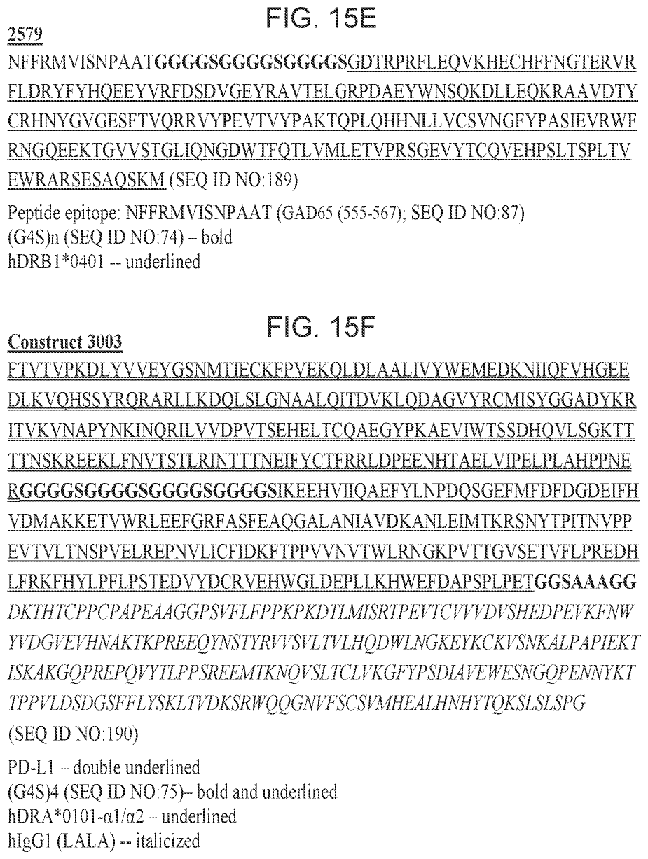

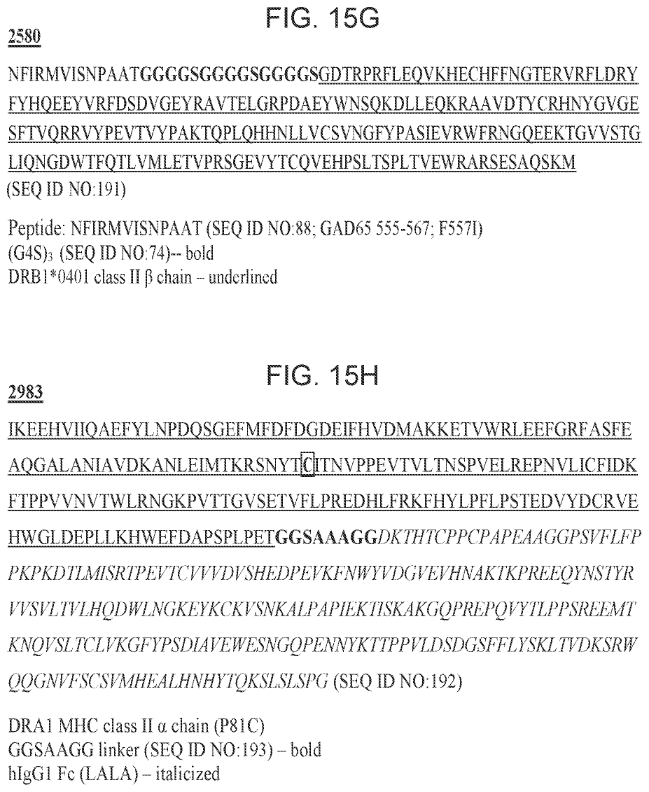

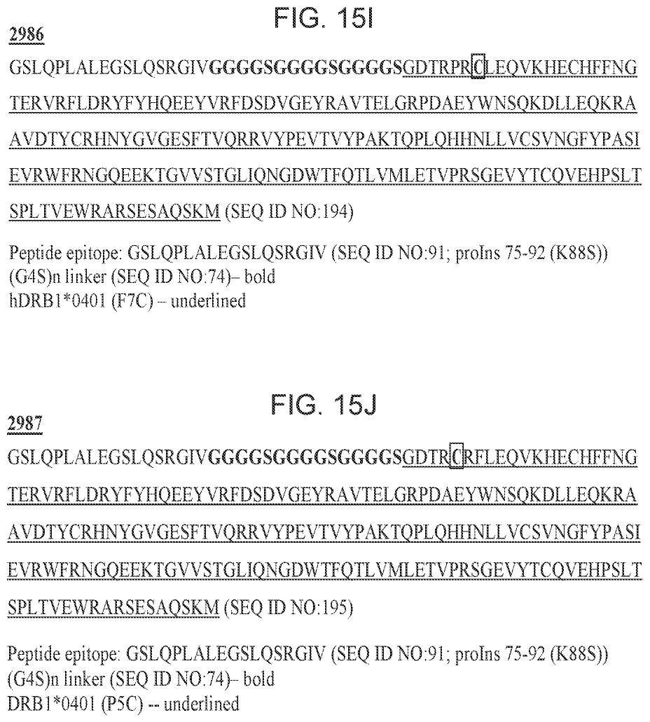

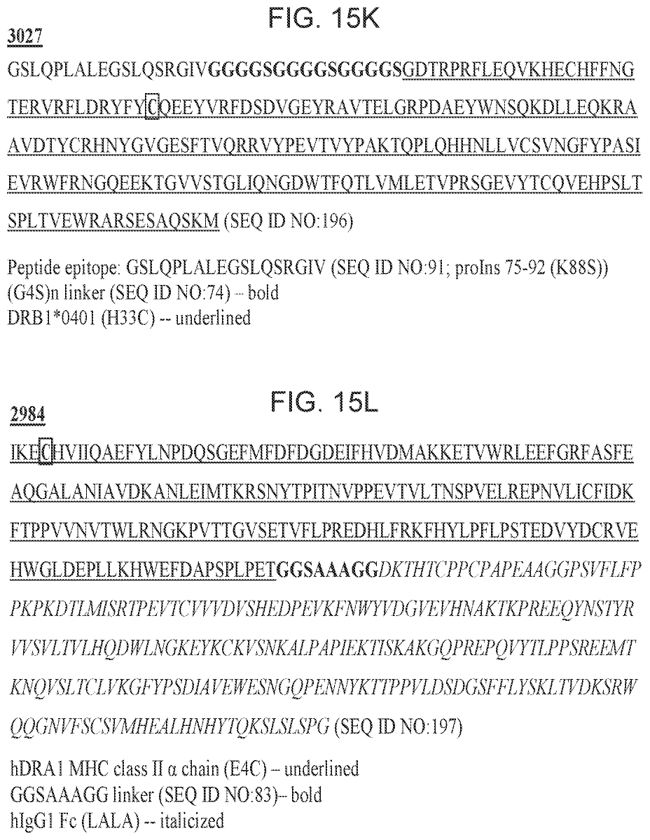

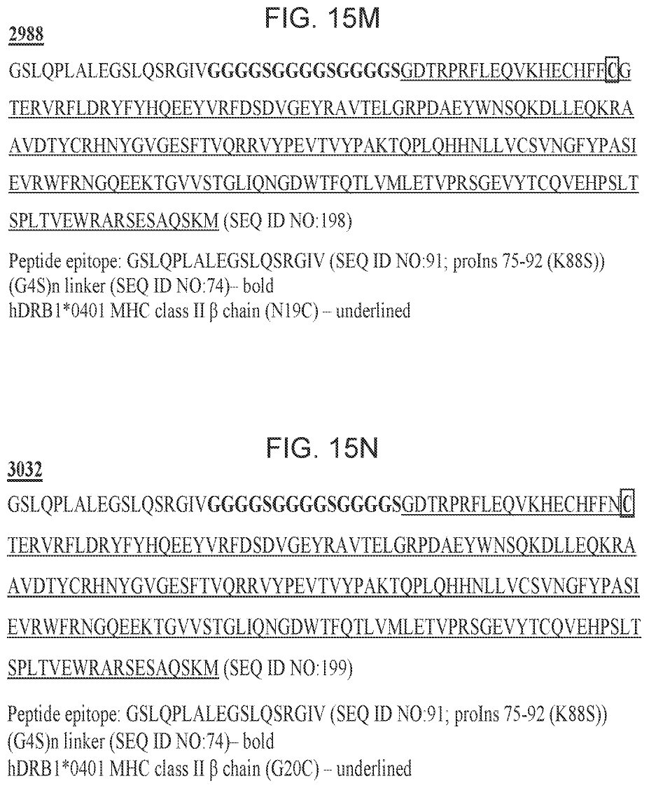

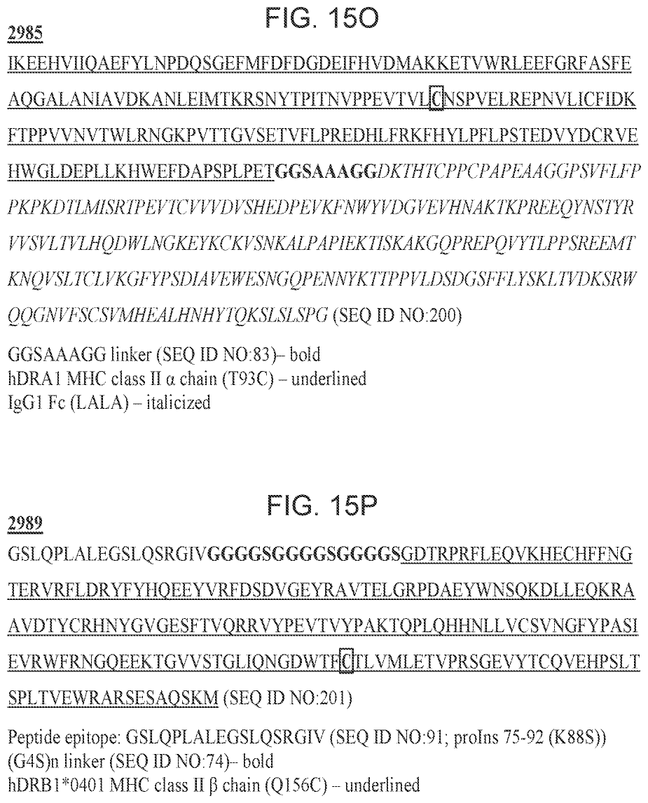

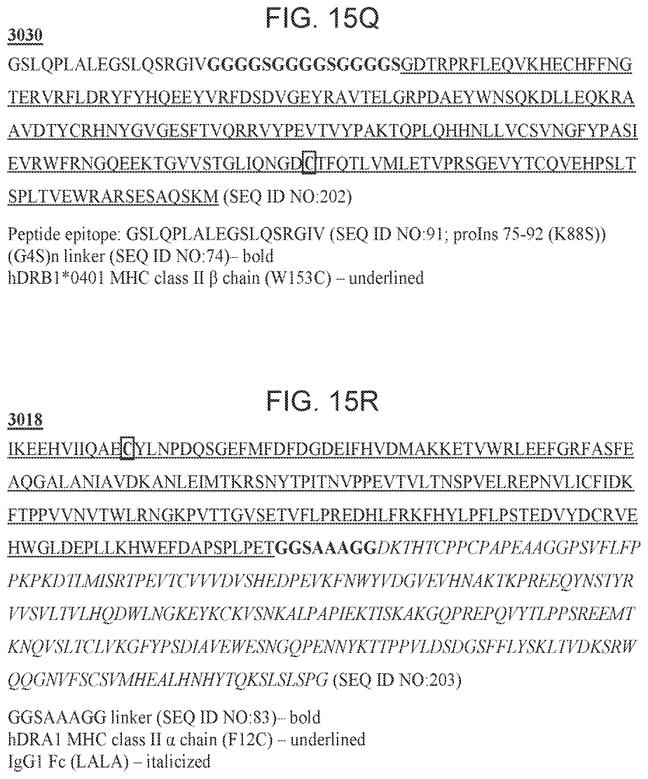

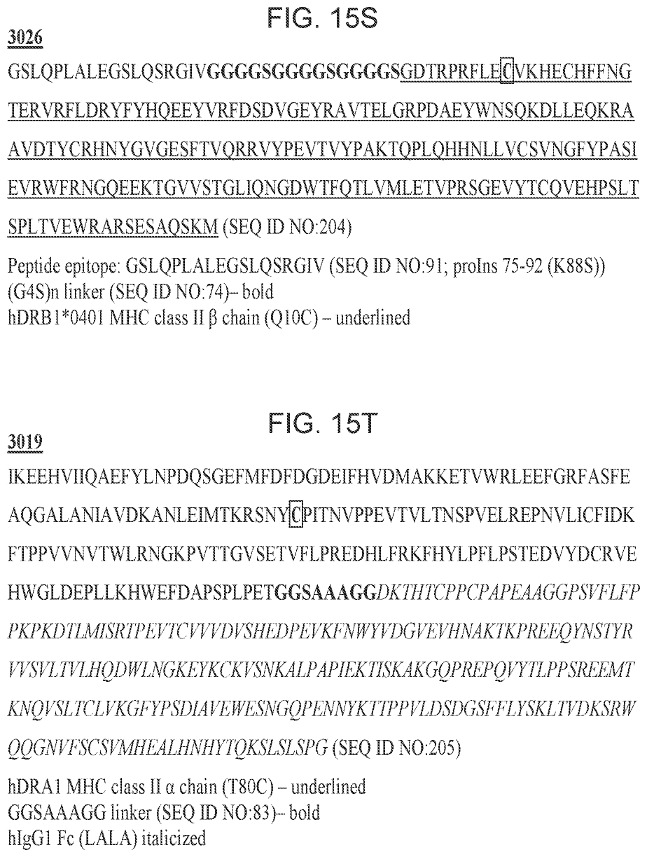

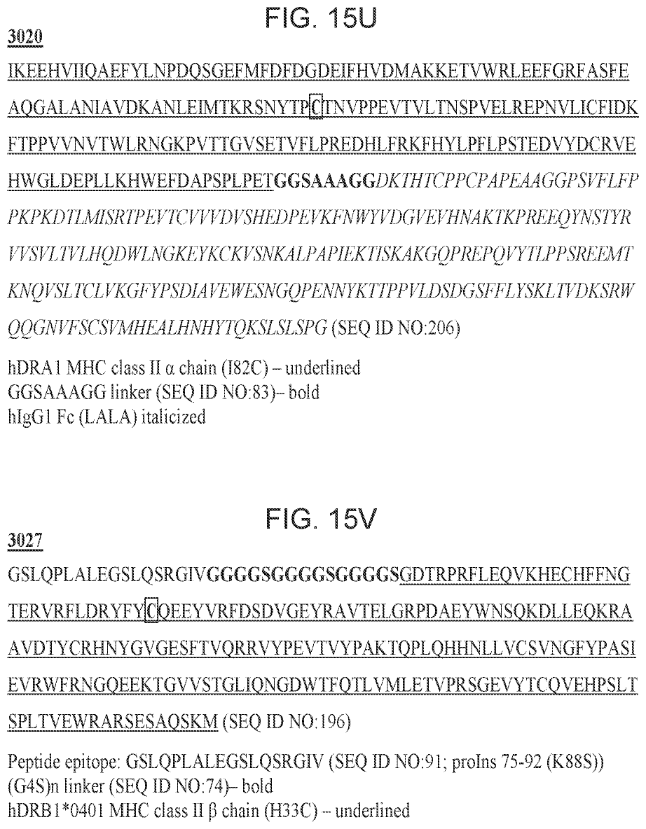

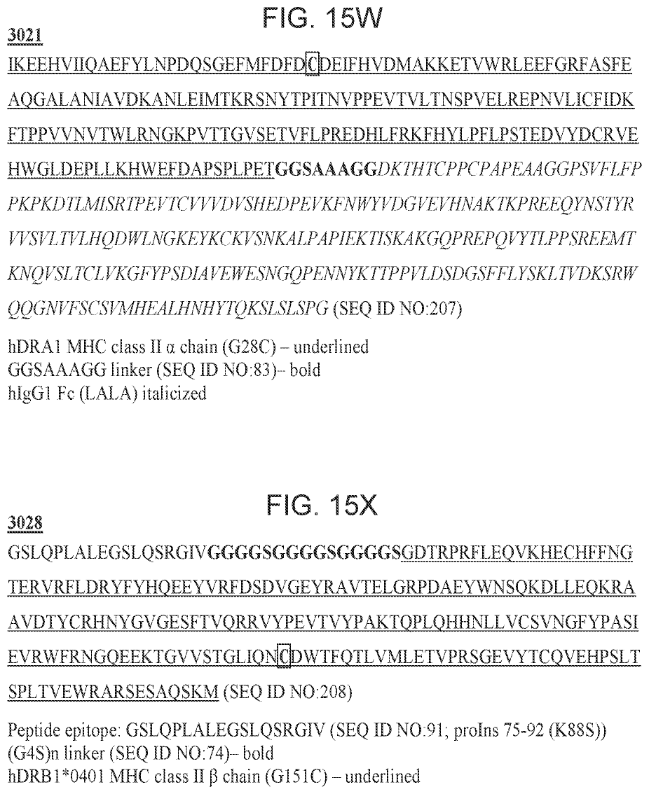

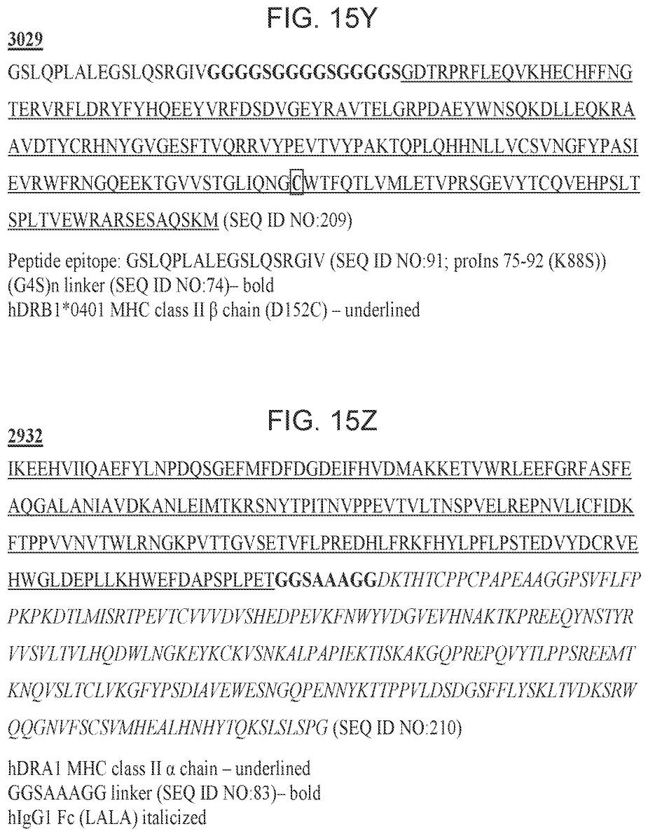

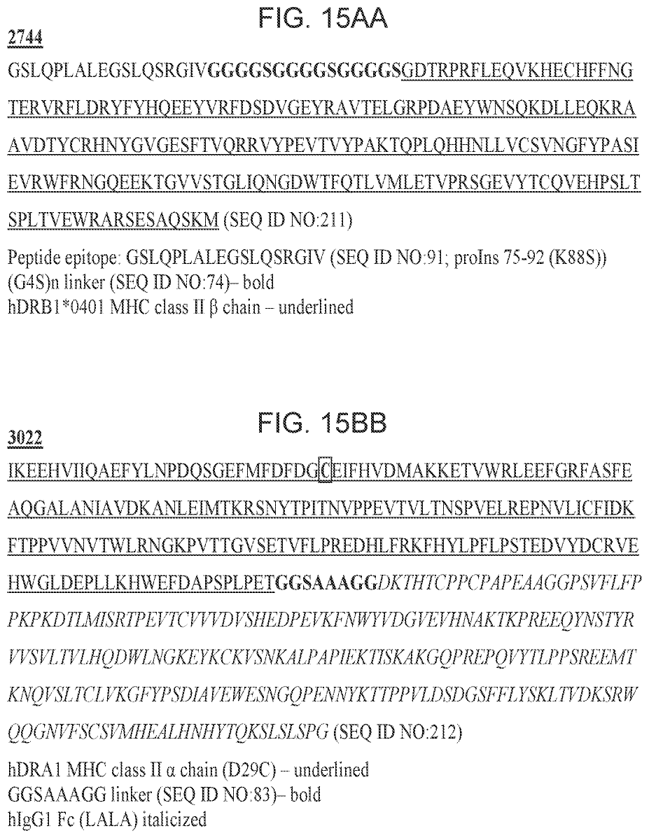

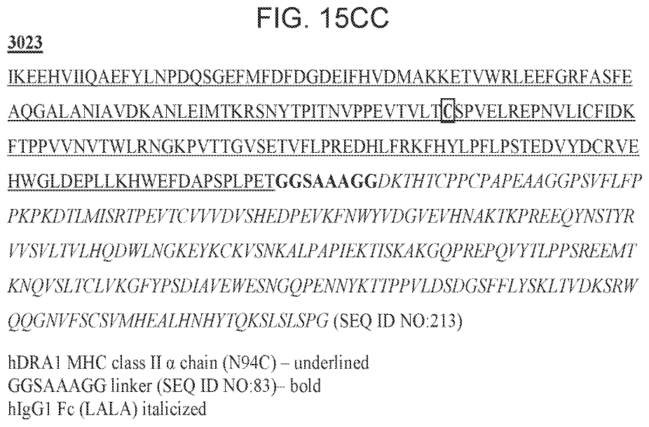

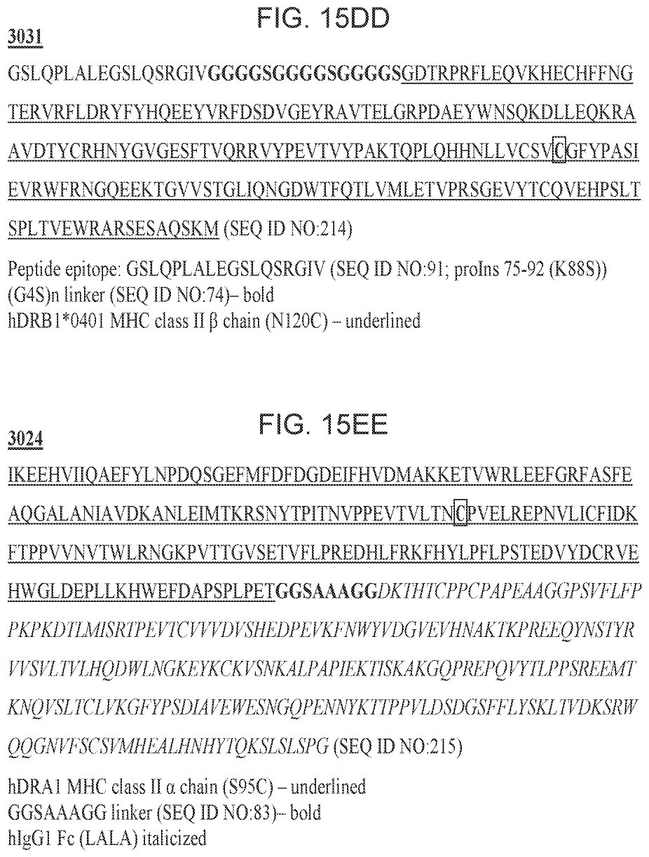

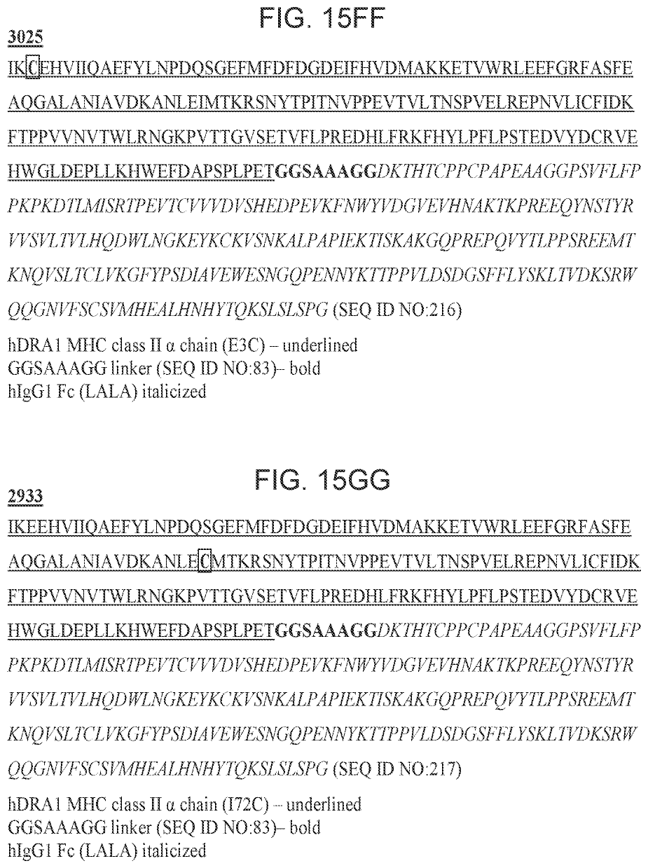

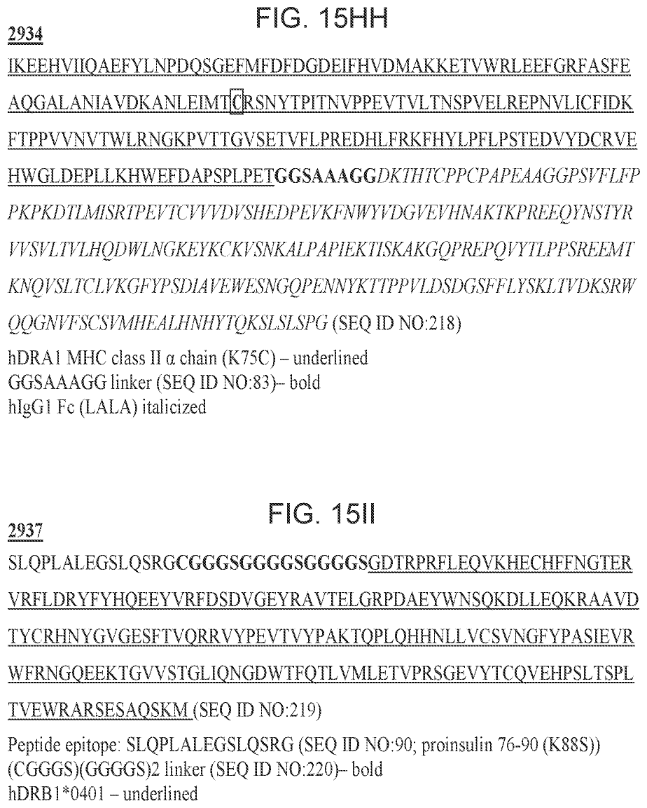

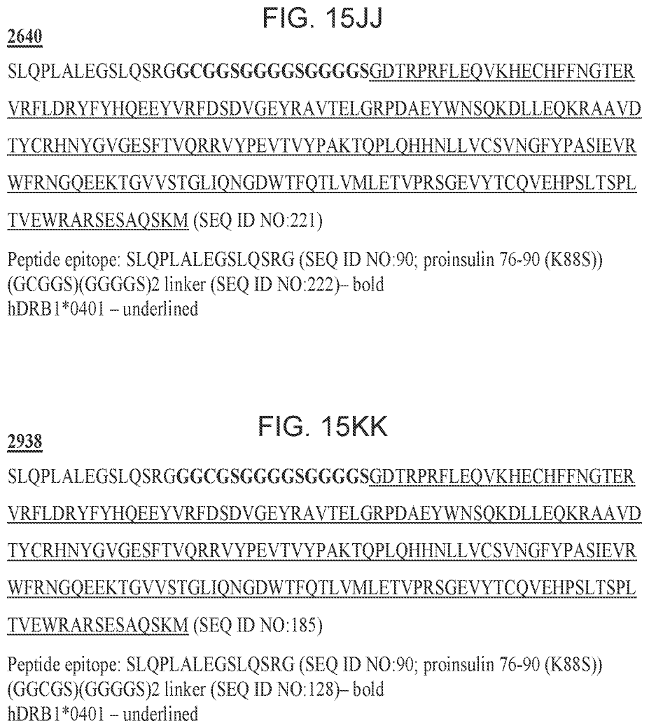

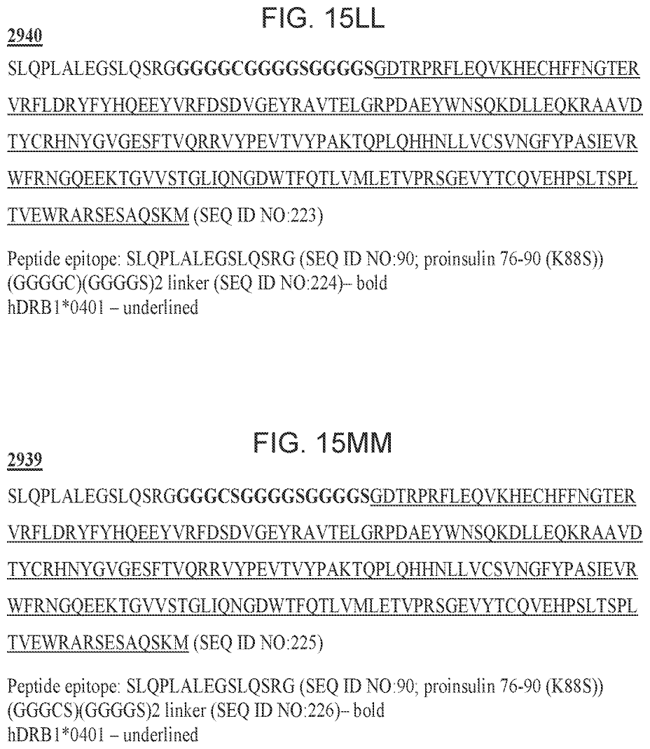

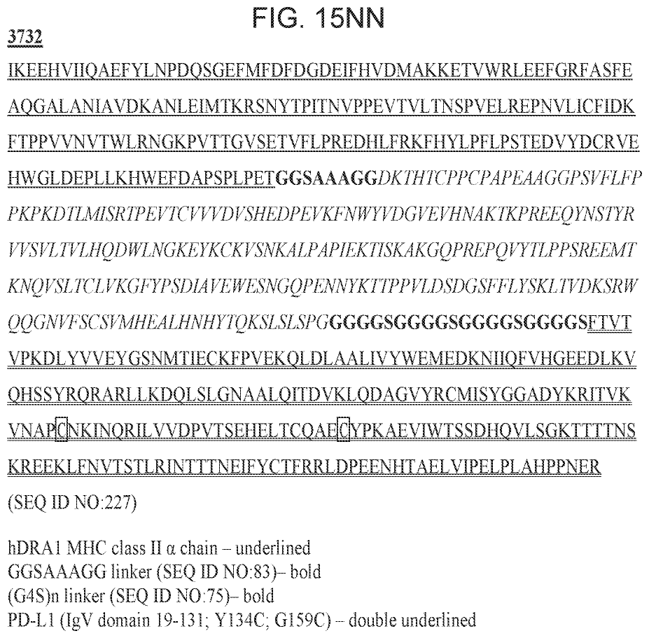

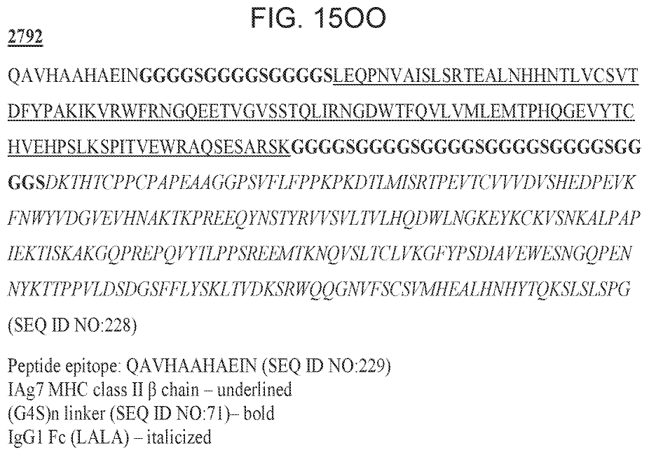

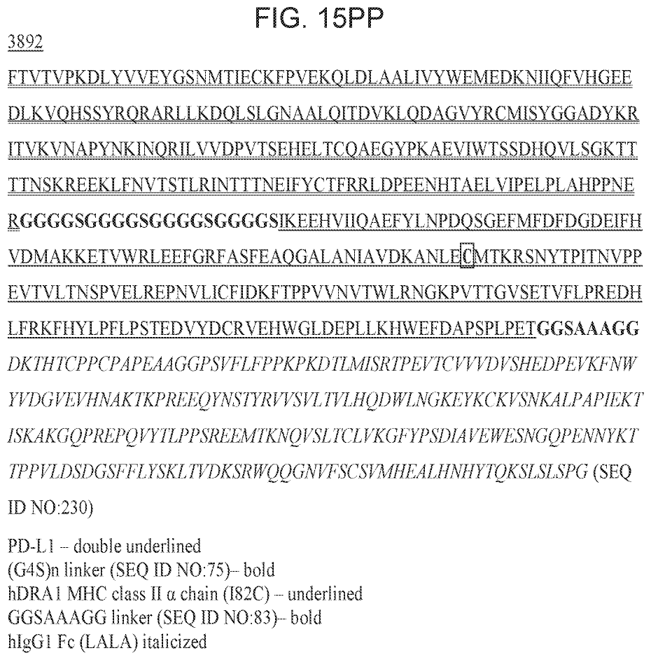

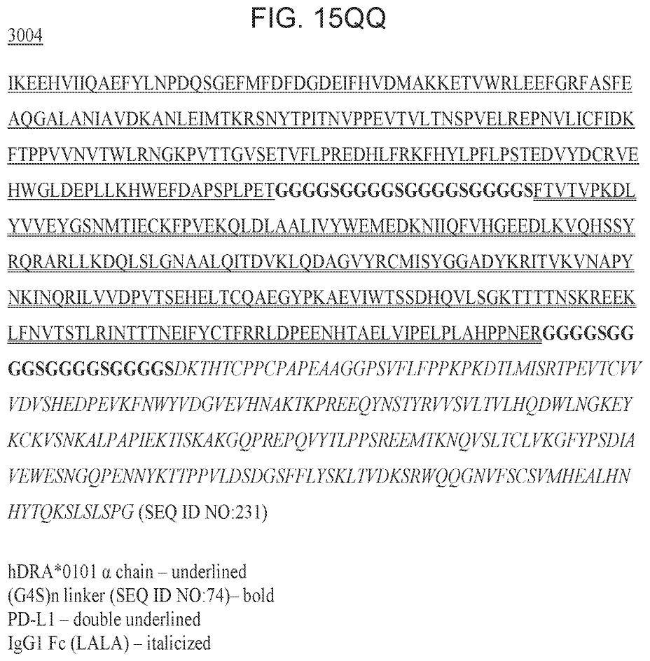

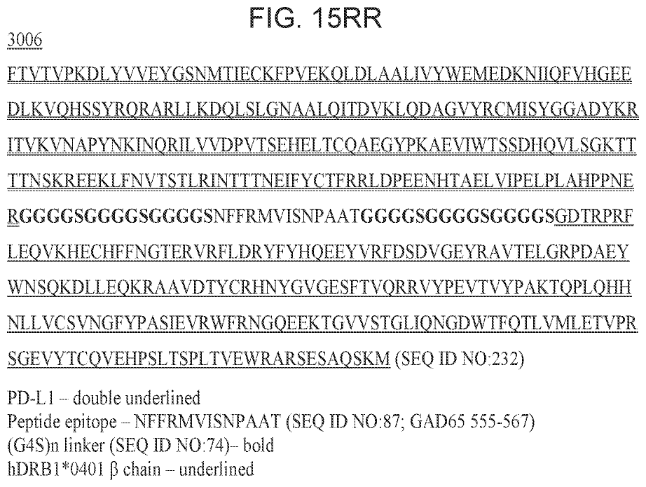

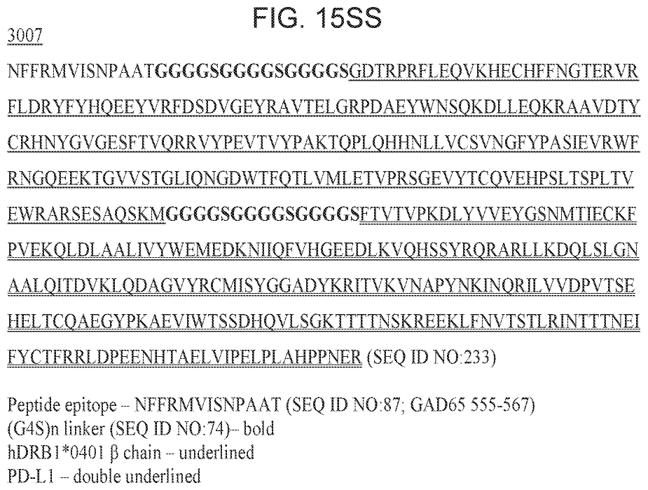

For example, in some cases, a TMMP of the present disclosure exhibits at least at least 10%, at least 20%, at least 30%, at least 40%, at least 50%, at least 60%, at least 70%, at least 80%, at least 90%, at least 2-fold, at least 2.5-fold, at least 3-fold, at least 4-fold, at least 5-fold, at least 10-fold, at least 20-fold, at least 50-fold, at least 100-fold, or more than 100-fold, greater stability in a buffered solution such as the PBS containing 500 mM NaCl (described above) in vitro than the TMMP 3003-2579 (FIG. 15F and FIG. 15E) or than the TMMP 3003-2639 (FIG. 15F and FIG. 15C) when kept for 1 hour, 5 days, or 10 days at 37° C. or at 42° C. As another example, in some cases, a TMMP of the present disclosure exhibits at least at least 10%, at least 20%, at least 30%, at least 40%, at least 50%, at least 60%, at least 70%, at least 80%, at least 90%, at least 2-fold, at least 2.5-fold, at least 3-fold, at least 4-fold, at least 5-fold, at least 10-fold, at least 20-fold, at least 50-fold, at least 100-fold, or more than 100-fold, greater stability in the PBS solution containing 500 mM NaCl (described above) in vitro than the TMMP 3005-2639 (FIG. 15D and FIG. 15C) when kept for 1 hour, 5 days, or 10 days at 37° C. or at 42° C.

As noted above, the presence of the at least one disulfide bond between the first polypeptide and the second polypeptide of the heterodimer can lead to greater expression of the TMMP as compared to a TMMP lacking the at least one disulfide bond. In some cases, a TMMP of the present disclosure is expressed (produced) at a level that is at least 10%, at least 20%, at least 30%, at least 40%, at least 50%, at least 60%, at least 70%, at least 80%, at least 90%, at least 2-fold, at least 2.5-fold, at least 3-fold, at least 4-fold, at least 5-fold, at least 10-fold, at least 20-fold, at least 50-fold, at least 100-fold, or more than 100-fold, higher than the expression level of the TMMP 3003-2579 (FIG. 15F and FIG. 15E) or of the TMMP 3003-2639 (FIG. 15F and FIG. 15C). In some cases, a TMMP of the present disclosure is expressed (produced) at a level that is at least 10%, at least 20%, at least 30%, at least 40%, at least 50%, at least 60%, at least 70%, at least 80%, at least 90%, at least 2-fold, at least 2.5-fold, at least 3-fold, at least 4-fold, at least 5-fold, at least 10-fold, at least 20-fold, at least 50-fold, at least 100-fold, or more than 100-fold, higher than the expression level of the TMMP 3005-2639 (FIG. 15D and FIG. 15C).

As discussed herein and set out in the Examples, stability can be expressed as the “% monomer” (as defined above) remaining after incubation of a TMMP of the present disclosure in the PBS buffer described above at a certain temperature for a certain amount of time. In some cases, the % monomers remaining after incubation of a TMMP of the present disclosure in the PBS buffer for a given time at a given temperature (e.g., 37° C. for 1 hour; 37° C. for 1 day; 37° C. for 5 days; 37° C. for 10 days; 37° C. for 14 days, 37° C. for 28 days; 42° C. for 1 hour; 42° C. for 1 day; 42° C. for 5 days; 42° C. for 10 days; or the like) is at least 10%, at least 20%, at least 30%, at least 40%, at least 50%, at least 60%, at least 70%, at least 80%, at least 90%, at least 2-fold, at least 2.5-fold, at least 3-fold, at least 4-fold, at least 5-fold, at least 10-fold, at least 20-fold, at least 50-fold, at least 100-fold, or more than 100-fold, higher than the % monomers of a control TMMP lacking at least one disulfide bond between the two polypeptides of the heterodimers.

A TMMP of the present disclosure can comprise two heterodimers. In some cases, the two heterodimers each include an Ig Fc polypeptide. In some cases, the TMMP comprises two heterodimers that are disulfide linked to one another via Cys residues present in the Ig Fc polypeptides. See, e.g., FIG. 1E for a schematic depiction of an example of such a TMMP.

### Immunomodulatory Polypeptides

A TMMP of the present disclosure comprises one or more immunomodulatory polypeptides. In some cases, a TMMP of the present disclosure comprises a single immunomodulatory polypeptide. In some cases, where a TMMP of the present disclosure comprises a single immunomodulatory polypeptide, the single immunomodulatory polypeptide is on the first polypeptide. In some cases, where a TMMP of the present disclosure comprises a single immunomodulatory polypeptide, the single immunomodulatory polypeptide is on the second polypeptide. In some cases, where a TMMP of the present disclosure comprises a single immunomodulatory polypeptide, the single immunomodulatory polypeptide is on the second polypeptide, and the second polypeptide comprises an MHC class II alpha polypeptide.

In some cases, a TMMP of the present disclosure comprises two or more immunomodulatory polypeptides (e.g., 2, 3, 4, or 5 immunomodulatory polypeptides). As noted above, a TMMP of the present disclosure comprises a disulfide-linked heterodimer comprising a first polypeptide and a second polypeptide. In some cases, the two or more immunomodulatory polypeptides are present in the first polypeptide chain only. In some cases, the two or more immunomodulatory polypeptides are present in the second polypeptide chain only. In some cases, where a TMPP of the present disclosure comprises a first polypeptide and a second polypeptide, at least one of the two or more immunomodulatory polypeptides are present in the first polypeptide chain; and at least one of the two or more immunomodulatory polypeptides are present in the second polypeptide chain. In some cases, where a TMMP of the present disclosure comprises two or more immunomodulatory polypeptides, the immunomodulatory polypeptides are on the second polypeptide, and the second polypeptide comprises an MHC class II alpha polypeptide. Where a TMPP of the present disclosure comprises 2 or 3 copies of an immunomodulatory polypeptide, in some cases, the 2 or 3 copies are in tandem. Where a TMPP of the present disclosure comprises 2 or 3 copies of an immunomodulatory polypeptide, in some cases, the 2 or 3 copies are separated from one another by a linker.

In some cases, where a TMPP of the present disclosure comprises two immunomodulatory polypeptides, the two immunomodulatory polypeptides have the same amino acid sequence, i.e., the TMPP comprises two copies of an immunomodulatory polypeptide. In some cases, where a TMPP of the present disclosure comprises two immunomodulatory polypeptides, the two immunomodulatory polypeptides do not have the same amino acid sequence; e.g., one of the two immunomodulatory polypeptides comprises a first amino acid sequence and the second of the two immunomodulatory polypeptides comprises a second amino acid sequence, where the first and the second amino acid sequences are not identical. In some cases, the first and the second amino acid sequences differ from one another in amino acid sequence by from 1 amino acid to 10 amino acids, from 10 amino acids to 25 amino acids, or more than 25 amino acids. In some cases, the first and the second amino acid sequences share less than 98%, less than 95%, less than 90%, less than 85%, less than 80%, less than 75%, or less than 70%, amino acid sequence identity with one another.

A TMMP of the present disclosure modulates activity of a T cell. In some cases, a TMMP of the present disclosure reduces activity of an autoreactive T cell and/or an autoreactive B cell, e.g., a T cell reactive with a T1D-associated epitope or a B cell reactive with a T1D-associated epitope (e.g., a B cell that produces antibodies that bind to a T1D-associated epitope). In some cases, a TMMP of the present disclosure increases the number and/or activity of a regulatory T cell (Treg), resulting in reduced activity of an autoreactive T cell and/or an autoreactive B cell. Thus, a TMMP of the present disclosure is useful for treating T1D in an individual.

Immunomodulatory polypeptides that are suitable for inclusion in a TMPP of the present disclosure include, but are not limited to, IL-2, transforming growth factor-beta (TGFβ), JAG1, CD7, B7-1 (CD80), B7-2 (CD86), PD-L1. PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, lymphotoxin beta receptor, 3/TR6, ILT3, ILT4, and HVEM. In some cases, a TMMP of the present disclosure comprises one or more immunomodulatory polypeptides selected from the group consisting of an IL-2 polypeptide, a PD-L1 polypeptide, a FasL polypeptide, a TGF-β polypeptide, and combinations thereof. In some cases, a TMMP of the present disclosure comprises one or more immunomodulatory polypeptides, where at least one of the one or more immunomodulatory polypeptides is a PD-L1 polypeptide. In some cases, a TMMP of the present disclosure comprises one or more immunomodulatory polypeptides, where at least one of the one or more immunomodulatory polypeptides is an IL-2 polypeptide. In some cases, a TMMP of the present disclosure comprises one or more immunomodulatory polypeptides, where at least one of the one or more immunomodulatory polypeptides is a TGF-β polypeptide. In some cases, a TMMP of the present disclosure comprises one or more immunomodulatory polypeptides, where at least one of the one or more immunomodulatory polypeptides is a FasL polypeptide.

In some cases, an immunomodulatory polypeptide suitable for inclusion in a TMPP of the present disclosure comprises a wild-type amino acid sequence. In some cases, an immunomodulatory polypeptide suitable for inclusion in a TMPP of the present disclosure is a variant, e.g., comprising from 1 to 10 amino acid substitutions relative to a wild-type or naturally-occurring immunomodulatory polypeptide. In some cases, an immunomodulatory polypeptide suitable for inclusion in a TMPP of the present disclosure is a variant that exhibits reduced affinity to its cognate co-immunomodulatory polypeptide (e.g., a co-immunomodulatory polypeptide present on the surface of a T cell), compared to the affinity of the wild-type or naturally-occurring immunomodulatory polypeptide for the cognate co-immunomodulatory polypeptide.

### Arrangement of TMMP Components

A TMMP of the present disclosure comprises two disulfide-linked heterodimeric polypeptides comprising the following components: a) a T1D peptide; b) an MHC class II a polypeptide; c) an MHC class II β polypeptide; and d) one or more immunomodulatory polypeptides; and may also one or more peptide linkers; and may also include Ig Fc polypeptide(s). Although the T1D peptide can be on same polypeptide with either the MHC class II a polypeptide or MHC class II β polypeptide, the T1D peptide typically will be on the same polypeptide chain as the MHC class II β polypeptide. These components can be present in a TMMP of the present disclosure in various arrangements. Examples of such arrangements are depicted schematically in FIG. 1A-1E, FIG. 2A-2D, FIG. 3A-3D, and FIG. 4A-4D. In some cases, a TMMP of the present disclosure comprises a heterodimer comprising: a) a first polypeptide comprising, in order from N-terminus to C-terminus: i) a T1D peptide; and ii) an MHC class II β polypeptide, optionally joined by a linker; and b) a second polypeptide comprising: i) an immunomodulatory polypeptide; and ii) an MHC class II a polypeptide, optionally joined by a linker, where the first polypeptide and the second polypeptide are covalently linked to one another via a disulfide bond. In some cases, a TMPP of the present disclosure comprises: a) a first polypeptide comprising, in order from N-terminus to C-terminus: i) a T1D peptide; and ii) an MHC class II β polypeptide, optionally joined by a linker; and b) a second polypeptide comprising: i) one or more immunomodulatory polypeptides (where two or more immunomodulatory polypeptides are present, they optionally may be joined by one or more linkers); ii) an MHC class II a polypeptide, optionally joined by a linker to the immunomodulatory polypeptide(s); and iii) an Ig Fc polypeptide, optionally joined by a linker to the MHC class II a polypeptide; where the first polypeptide and the second polypeptide are covalently linked to one another via a disulfide bond. The arrangement (N-terminus to C-terminus order) of the immunomodulatory polypeptide, the MHC class II a polypeptide, and the Ig Fc polypeptide, can vary.

As one example of a possible arrangement, in some cases, a TMPP of the present disclosure comprises a heterodimer comprising two disulfide-linked polypeptides, wherein the heterodimer comprises: a) a first polypeptide comprising, in order from N-terminus to C-terminus (optionally including linkers): i) a T1D peptide; and ii) an MHC class II β polypeptide; and b) a second polypeptide comprising, in order from N-terminus to C-terminus (optionally including linkers): i) an immunomodulatory polypeptide; ii) an MHC class II a polypeptide; and iii) an Ig Fc polypeptide. (See FIG. 5, MOD position 1.) As another example of a possible arrangement, in some cases, a TMPP of the present disclosure comprises: a) a first polypeptide comprising, in order from N-terminus to C-terminus (optionally including linkers): i) a T1D peptide; and ii) an MHC class II β polypeptide; and b) a second polypeptide comprising, in order from N-terminus to C-terminus (optionally including one or more linkers that do not comprise a Cys): i) an MHC class II a polypeptide; ii) one or more immunomodulatory polypeptides; and iii) an Ig Fc polypeptide. (See FIG. 5, MOD position 2.) As another example of a possible arrangement, in some cases, a TMPP of the present disclosure comprises: a) a first polypeptide comprising, in order from N-terminus to C-terminus (optionally including linkers): i) a T1D peptide; and ii) an MHC class II β polypeptide; and b) a second polypeptide comprising, in order from N-terminus to C-terminus (optionally including linkers): i) an MHC class II a polypeptide; ii) an Ig Fc polypeptide; and iii) one or more immunomodulatory polypeptides. (See FIG. 5, MOD position 3.). As another example of a possible arrangement, in some cases, a TMMP of the present disclosure comprises: a) a first polypeptide comprising, in order from N-terminus to C-terminus (optionally including linkers): i) one or more immunomodulatory polypeptides; ii) a T1D peptide; and iii) an MHC class II B polypeptide; and b) a second polypeptide comprising, in order from N-terminus to C-terminus (optionally including linkers): i) an MHC class II a polypeptide; and ii) an Ig Fc polypeptide. (See FIG. 5. MOD position 4.). As another example of a possible arrangement, in some cases, a TMMP of the present disclosure comprises: a) a first polypeptide comprising, in order from N-terminus to C-terminus (optionally including linkers): i) a T1D peptide; ii) an MHC class II β polypeptide; and iii) one or more immunomodulatory polypeptides; and b) a second polypeptide comprising, in order from N-terminus to C-terminus (optionally including linkers): i) an MHC class II a polypeptide; and ii) an Ig Fc polypeptide. (See FIG. 5, MOD position 5.) The immunomodulatory polypeptide(s) can be on the same polypeptide chain as the MHC class II a polypeptides.

Generally speaking, potential locations in the heterodimer for disulfide bonds are where residues in the first and second polypeptides of the heterodimer are separated by a distance of 5 angstroms or less. Such locations represent potential locations where Cys residues, if not naturally present, can be substituted for the residues that exist in the polypeptides. A first polypeptide and a second polypeptide thus can be linked via a disulfide bond between two Cys residues that are generally no more than about 5 angstroms apart from one another in the heterodimer. In some cases, one or both of the Cys residues are non-naturally-occurring. An amino acid in the alpha chain and an amino acid in the beta chain of MHC class II polypeptides that are no more than 5 angstroms from one another in an MHC class II heterodimer, represent amino acids that, when substituted with a Cys, can form a disulfide bond in a TMMP of the present disclosure. Similarly, a disulfide bond can be formed i) between a Cys residue in a linker and a Cys residue in an MHC class II polypeptide, or ii) between a Cys residue in a linker in one polypeptide of the heterodimer and a Cys residue in a linker in the other polypeptide of the heterodimer, where the two Cys residues are no more than about 5 angstroms apart from each other in the heterodimer. Notably, however, not all pairs of residues separated by about 5 angstroms or less will be suitable for formation of a disulfide bond or provide a disulfide bond that stabilizes the resulting heterodimer.

### Disulfide Bond Between a Peptide Linker and an MHC Class II Polypeptide

In any of the above embodiments, in some cases the TMMP comprises a peptide linker between the T1D peptide and the MHC class II polypeptide. Where desired, the linker can comprise a Cys that can be used to form a disulfide bond between the two polypeptides in the heterodimer.

### Position 1 MOD(s)

In some cases, a TMMP of the present disclosure comprises: a) a first polypeptide comprising, in order from N-terminus to C-terminus: i) a T1D peptide; ii) a peptide linker comprising a Cys; and iii) an MHC class II β polypeptide; and b) a second polypeptide comprising, in order from N-terminus to C-terminus (optionally including one or more linkers that do not comprise a Cys): i) one or more immunomodulatory polypeptides; ii) an MHC class II α chain; and (optionally) iii) an Ig Fc polypeptide. (See FIG. 5, MOD position 1.) The peptide linker comprising a Cys can comprise an amino acid sequence selected from (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where n is 1, 2, 3, 4, 5, 6, 7, 8, 9, or 10). In any of the above embodiments, in some cases the immunomodulatory polypeptide is a PD-L1 polypeptide or variant thereof, in some cases the immunomodulatory polypeptide is a TGF-β polypeptide or variant thereof, in some cases the immunomodulatory polypeptide is an IL-2 polypeptide or variant thereof, and in some cases the immunomodulatory polypeptide is a FasL polypeptide or variant thereof. In some cases, the T1D peptide is a proinsulin peptide. In some cases, the T1D peptide is a GAD peptide. In any of the above embodiments, in some cases, the MHC class II α chain comprises an amino acid substitution of an amino acid (other than a Cys) with a Cys, such that the Cys in the MHC class II α chain forms a disulfide bond with the Cys in the first polypeptide linker.

### Position 2 MOD(s)

In some cases, a TMMP of the present disclosure comprises: a) a first polypeptide comprising, in order from N-terminus to C-terminus: i) a T1D peptide; ii) a peptide linker comprising a Cys; and iii) an MHC class II (polypeptide; and b) a second polypeptide comprising, in order from N-terminus to C-terminus (optionally including one or more linkers that do not comprise a Cys): i) an MHC class II a chain; ii) one or more immunomodulatory polypeptides; and iii) an Ig Fc polypeptide. (See FIG. 5, MOD position 2.) The peptide linker comprising a Cys can comprise, e.g., an amino acid sequence selected from (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where n is 1, 2, 3, 4, 5, 6, 7, 8, 9, or 10). Other peptide linkers comprising a Cys may be used. In any of the above embodiments, in some cases the immunomodulatory polypeptide is a PD-L1 polypeptide or variant thereof, in some cases the immunomodulatory polypeptide is a TGF-β polypeptide or variant thereof, in some cases the immunomodulatory polypeptide is an IL-2 polypeptide or variant thereof, and in some cases, the immunomodulatory polypeptide is a FasL polypeptide or variant thereof. In some cases, the T1D peptide is a proinsulin peptide. In some cases, the T1D peptide is a GAD peptide. In any of the above embodiments, in some cases, the MHC class II α chain comprises an amino acid substitution of an amino acid (other than a Cys) with a Cys, such that the Cys in the MHC class II α chain forms a disulfide bond with the Cys in the linker.

### Position 3 MOD(s)

In some cases, a TMMP of the present disclosure comprises: a) a first polypeptide comprising, in order from N-terminus to C-terminus: i) a T1D peptide; ii) a peptide linker comprising a Cys; and iii) an MHC class II β polypeptide; and b) a second polypeptide comprising, in order from N-terminus to C-terminus (optionally including one or more linkers that do not comprise a Cys): i) an MHC class II a chain; ii) an Ig Fc polypeptide; and iii) one or more immunomodulatory polypeptides. (See FIG. 5, MOD position 3.) The peptide linker comprising a Cys can comprise, e.g., an amino acid sequence selected from (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where n is 1, 2, 3, 4, 5, 6, 7, 8, 9, or 10). Other peptide linkers comprising a Cys may be used. In any of the above embodiments, in some cases, the immunomodulatory polypeptide is a PD-L1 polypeptide or variant thereof. In any of the above embodiments, in some cases the immunomodulatory polypeptide is a TGF-β polypeptide or variant thereof. In any of the above embodiments, in some cases, the immunomodulatory polypeptide is an IL-2 polypeptide or variant thereof. In any of the above embodiments, in some cases, the immunomodulatory polypeptide is a FasL polypeptide or variant thereof. In some cases, the T1D peptide is a proinsulin peptide. In some cases, the T1D peptide is a GAD peptide. In any of the above embodiments, in some cases, the MHC class II α chain comprises an amino acid substitution of an amino acid (other than a Cys) with a Cys, such that the Cys in the MHC class II α chain forms a disulfide bond with the Cys in the linker.

### Position 4 MOD(s)

In some cases, a TMMP of the present disclosure comprises: a) a first polypeptide comprising, in order from N-terminus to C-terminus: i) one or more immunomodulatory polypeptides; ii) a T1D peptide; ii) a peptide linker comprising a Cys; and iii) an MHC class II β polypeptide; and b) a second polypeptide comprising, in order from N-terminus to C-terminus: i) an MHC class II α chain; and (optionally) ii) an Ig Fc polypeptide, optionally joined by a linker that does not comprise a Cys. (See FIG. 5, MOD position 4.) The peptide linker comprising a Cys can comprise, e.g., an amino acid sequence selected from (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where n is 1, 2, 3, 4, 5, 6, 7, 8, 9, or 10). Other peptide linkers comprising a Cys may be used. In any of the above embodiments, in some cases the immunomodulatory polypeptide is a PD-L1 polypeptide or variant thereof, in some cases the immunomodulatory polypeptide is a TGF-β polypeptide or variant thereof, in some cases the immunomodulatory polypeptide is an IL-2 polypeptide or variant thereof, and in some cases the immunomodulatory polypeptide is a FasL polypeptide or variant thereof. In some cases, the T1D peptide is a proinsulin peptide. In some cases, the T1D peptide is a GAD peptide. In any of the above embodiments, in some cases, the MHC class II α chain comprises an amino acid substitution of an amino acid (other than a Cys) with a Cys, such that the Cys in the MHC class II α chain forms a disulfide bond with the Cys in the linker.

### Position 5 MOD(s)

In some cases, a TMMP of the present disclosure comprises: a) a first polypeptide comprising, in order from N-terminus to C-terminus: i) a T1D peptide; ii) a peptide linker comprising a Cys; iii) an MHC class II β polypeptide; and iv) one or more immunomodulatory polypeptides; and b) a second polypeptide comprising, in order from N-terminus to C-terminus: i) an MHC class II α chain; and (optionally) ii) an Ig Fc polypeptide, optionally joined by a linker that does not comprise a Cys. (See FIG. 5, MOD position 4.) The peptide linker comprising a Cys can comprise, e.g., an amino acid sequence selected from (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where n is 1, 2, 3, 4, 5, 6, 7, 8, 9, or 10). Other peptide linkers comprising a Cys may be used. In any of the above embodiments, in some cases the immunomodulatory polypeptide is a PD-L1 polypeptide or variant thereof, in some cases the immunomodulatory polypeptide is a TGF-β polypeptide or variant thereof, in some cases the immunomodulatory polypeptide is an IL-2 polypeptide or variant thereof, and in some cases the immunomodulatory polypeptide is a FasL polypeptide or variant thereof. In some cases, the T1D peptide is a proinsulin peptide. In some cases, the T1D peptide is a GAD peptide. In any of the above embodiments, in some cases, the MHC class II α chain comprises an amino acid substitution of an amino acid (other than a Cys) with a Cys, such that the Cys in the MHC class II α chain forms a disulfide bond with the Cys in the linker.

### Disulfide Bond Between Peptide Linkers in the First and Second Polypeptides

In some cases, the TMMP comprises a first Cys-containing peptide linker between the T1D peptide and the MHC class II polypeptide in the first polypeptide, and a second Cys-containing peptide linker between two of the components in the second polypeptide. In such cases, the Cys residues in the first and second Cys-containing linkers can be used to form a disulfide bond between the two polypeptides in the heterodimer.

### Position 1 MOD(s)

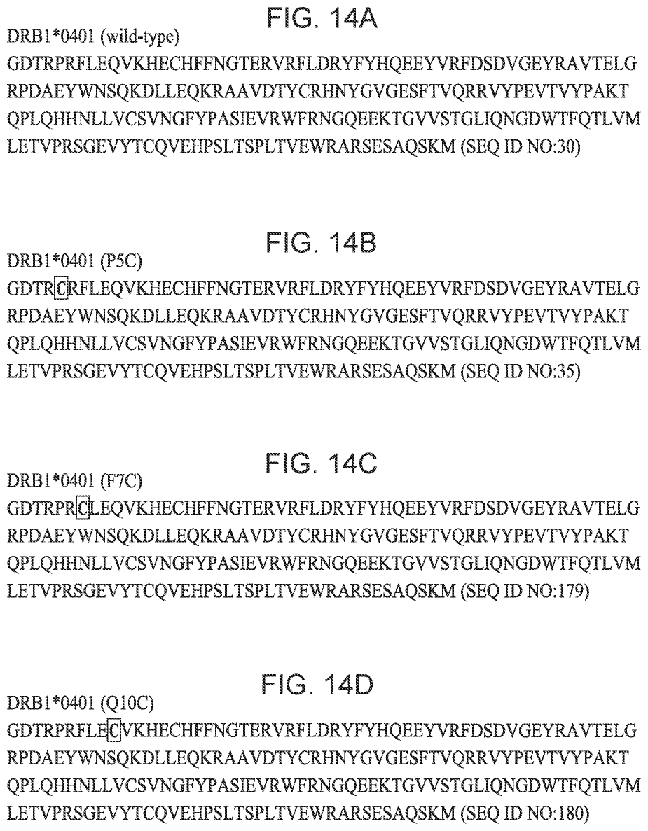

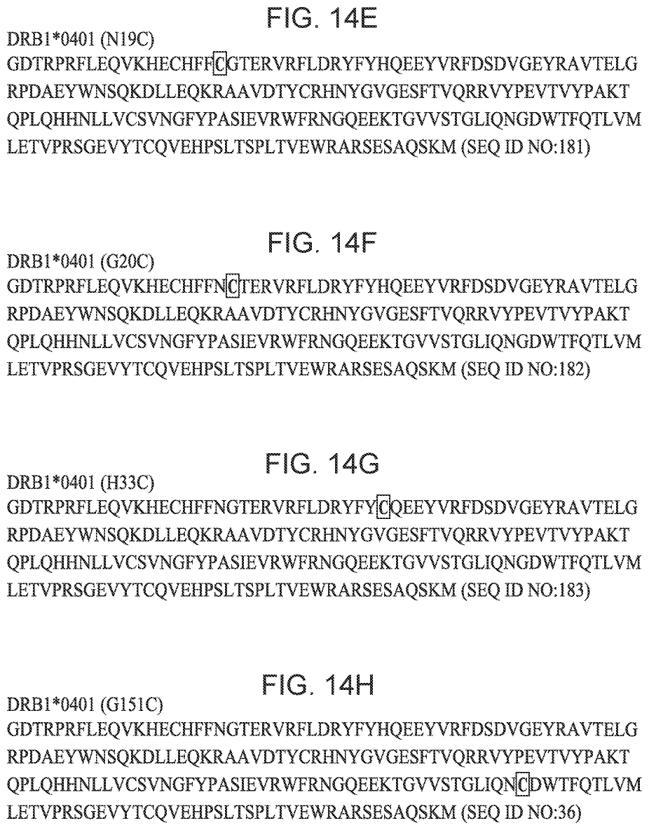

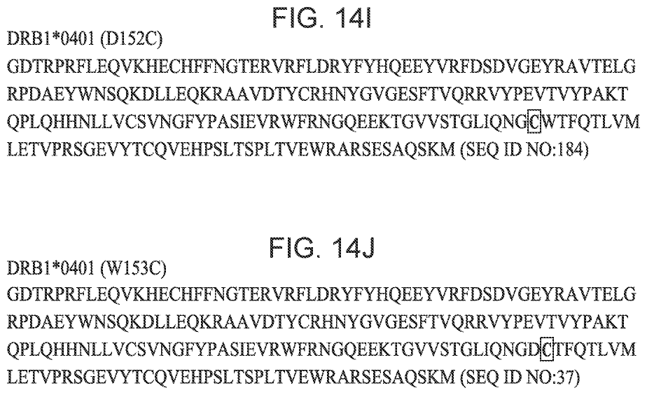

In some cases, a TMMP of the present disclosure comprises: a) a first polypeptide comprising, in order from N-terminus to C-terminus: i) a T1D peptide; ii) a first peptide linker comprising a Cys; and iii) an MHC class II β polypeptide (e.g., a DRB MHC class II polypeptide comprising an amino acid sequence having at least 80%, at least 85%, at least 90%, at least 95%, at least 98%, at least 99%, or 100% amino acid sequence identity to the amino acid sequence depicted in FIG. 14A); and b) a second polypeptide comprising, in order from N-terminus to C-terminus: i) one or more immunomodulatory polypeptides; ii) an MHC class II α chain (e.g., a DRA MHC class II polypeptide comprising an amino acid sequence having at least 80%, at least 85%, at least 90%, at least 95%, at least 98%, at least 99%, or 100% amino acid sequence identity to the amino acid sequence depicted in FIG. 13A); and (optionally) iii) an Ig Fc polypeptide. (See FIG. 5, MOD position 1.) The second polypeptide comprises a second peptide linker that comprises a Cys, where the second peptide linker is positioned either between the immunomodulatory polypeptides and the MHC class II α chain or between the MHC class II α chain and the Ig Fc, if present. The second polypeptide optionally may include one or more linkers that do not comprise a Cys. The peptide linkers comprising a Cys can comprise an amino acid sequence selected from (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where n is 1, 2, 3, 4, 5, 6, 7, 8, 9, or 10). In any of the above embodiments, in some cases the immunomodulatory polypeptide is a PD-L1 polypeptide or variant thereof, in some cases the immunomodulatory polypeptide is a TGF-β polypeptide or variant thereof, in some cases the immunomodulatory polypeptide is an IL-2 polypeptide or variant thereof, and in some cases the immunomodulatory polypeptide is a FasL polypeptide or variant thereof. In some cases, the T1D peptide is a proinsulin peptide. In some cases, the T1D peptide is a GAD peptide.

### Position 3 MOD(s)

In some cases, a TMMP of the present disclosure comprises: a) a first polypeptide comprising, in order from N-terminus to C-terminus: i) a T1D peptide; ii) a first peptide linker comprising a Cys; and iii) an MHC class II β polypeptide (e.g., a DRB MHC class II polypeptide comprising an amino acid sequence having at least 80%, at least 85%, at least 90%, at least 95%, at least 98%, at least 99%, or 100% amino acid sequence identity to the amino acid sequence depicted in FIG. 14A); and b) a second polypeptide comprising, in order from N-terminus to C-terminus: i) an MHC class II α chain (e.g., a DRA MHC class II polypeptide comprising an amino acid sequence having at least 80%, at least 85%, at least 90%, at least 95%, at least 98%, at least 99%, or 100% amino acid sequence identity to the amino acid sequence depicted in FIG. 13A); ii) an Ig Fc polypeptide; and iii) one or more immunomodulatory polypeptides. (See FIG. 5, MOD position 3.) The second polypeptide comprises a second peptide linker that comprises a Cys, where the second peptide linker is positioned either between the MHC class II a chain and the Ig Fc or between the IG Fc and the one or more immunomodulatory polypeptides. The second polypeptide optionally may include one or more linkers that do not comprise a Cys. The peptide linkers comprising a Cys can comprise an amino acid sequence selected from (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where n is 1, 2, 3, 4, 5, 6, 7, 8, 9, or 10). In any of the above embodiments, in some cases the immunomodulatory polypeptide is a PD-L1 polypeptide or variant thereof, in some cases the immunomodulatory polypeptide is a TGF-β polypeptide or variant thereof, in some cases the immunomodulatory polypeptide is an IL-2 polypeptide or variant thereof, and in some cases the immunomodulatory polypeptide is a FasL polypeptide or variant thereof. In some cases, the T1D peptide is a proinsulin peptide. In some cases, the T1D peptide is a GAD peptide. The peptide linkers comprising a Cys can comprise, e.g., an amino acid sequence selected from (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where n is 1, 2, 3, 4, 5, 6, 7, 8, 9, or 10). Other peptide linkers comprising a Cys may be used. In any of the above embodiments, in some cases, the immunomodulatory polypeptide is a PD-L1 polypeptide or variant thereof. In any of the above embodiments, in some cases the immunomodulatory polypeptide is a TGF-β polypeptide or variant thereof. In any of the above embodiments, in some cases, the immunomodulatory polypeptide is an IL-2 polypeptide or variant thereof. In any of the above embodiments, in some cases, the immunomodulatory polypeptide is a FasL polypeptide or variant thereof. In some cases, the T1D peptide is a proinsulin peptide. In some cases, the T1D peptide is a GAD peptide.

Alternatively, following the same design of including a Cys-containing linker in the first polypeptide between the T1D peptide and the MHC class II β polypeptide, and a Cys-containing linker between two of the components in the second polypeptide, a disulfide-linked TMMP can be constructed in which the one or more immunomodulatory polypeptides are at Position 2, 4 or 5 as those positions are described above and depicted schematically in FIG. 5.

### Disulfide Bond Between the Two MHC Class II Polypeptides

As noted above, in some cases, the first polypeptide of a heterodimer of a TMMP of the present disclosure comprises a first MHC class II polypeptide comprising an amino acid substitution that results in a Cys (a “first Cys”); and the second polypeptide comprises a second MHC class II polypeptide comprising an amino acid substitution that results in a Cys (a “second Cys”); where the heterodimer comprises a disulfide bond formed between the first Cys and the second Cys. For example, in some cases, the first polypeptide comprises an MHC class II β polypeptide comprising an amino acid substitution that results in a Cys (a “first Cys”); and the second polypeptide comprises an MHC class II a polypeptide comprising an amino acid substitution that results in a Cys (a “second Cys”); where the heterodimer comprises a disulfide bond formed between the first Cys and the second Cys.

### Position 1 MOD(s)

In some cases, for example, a TMMP of the present disclosure comprises: a) a first polypeptide comprising, in order from N-terminus to C-terminus: i) a T1D peptide; and ii) a first MHC class II polypeptide comprising a substitution of an amino acid (other than a Cys) with a Cys (a “first Cys”), the components of the first polypeptide optionally being joined by a linker that does not comprise a Cys; and b) a second polypeptide comprising, in order from N-terminus to C-terminus: i) one or more immunomodulatory polypeptides; ii) a second MHC class II polypeptide comprising a substitution of an amino acid (other than a Cys) with a Cys (a “second Cys”); and iii) optionally an Ig Fc polypeptide, the components of the second polypeptide optionally being joined by one or more linkers that do not comprise a Cys; where the first polypeptide and the second polypeptide are linked via a disulfide bond between the first Cys and the second Cys. (See FIG. 5, MOD position 1.) In any of the above embodiments, in some cases, the one or more immunomodulatory polypeptides is a PD-L1 polypeptide or variant thereof, in some cases the one or more immunomodulatory polypeptides is a TGF-β polypeptide or variant thereof, in some cases the one or more immunomodulatory polypeptides is an IL-2 polypeptide or variant thereof, and in some cases the one or more immunomodulatory polypeptides is a FasL polypeptide or variant thereof. In some cases, the T1D peptide is a proinsulin peptide. In some cases, the T1D peptide is a GAD peptide. In any of the above embodiments, in some cases, the first MHC class II polypeptide is an MHC class II β chain polypeptide and the second MHC class II polypeptide is an MHC class II α chain polypeptide.

### Position 2 MOD(s)

As another example, in some cases, a TMMP of the present disclosure comprises: a) a first polypeptide comprising, in order from N-terminus to C-terminus: i) a T1D peptide; and ii) a first MHC class II polypeptide comprising a substitution of an amino acid (other than a Cys) with a Cys (a “first Cys”), the components of the first polypeptide optionally being joined by a linker that does not comprise a Cys; and b) a second polypeptide comprising, in order from N-terminus to C-terminus: i) a second MHC class II polypeptide comprising a substitution of an amino acid (other than a Cys) with a Cys (a “second Cys”); ii) one or more immunomodulatory polypeptides; and iii) optionally an Ig Fc polypeptide, the components of the second polypeptide optionally being joined by one or more linkers that do not comprise a Cys; where the first polypeptide and the second polypeptide are linked via a disulfide bond between the first Cys and the second Cys. (See FIG. 5, MOD position 2.) In any of the above embodiments, in some cases the one or more immunomodulatory polypeptides is a PD-L1 polypeptide or variant thereof, in some cases the one or more immunomodulatory polypeptides is a TGF-β polypeptide or variant thereof, in some cases the one or more immunomodulatory polypeptides is an IL-2 polypeptide or variant thereof, and in some cases the one or more immunomodulatory polypeptides is a FasL polypeptide or variant thereof. In some cases, the T1D peptide is a proinsulin peptide. In some cases, the T1D peptide is a GAD peptide. In any of the above embodiments, in some cases, the first MHC class II polypeptide is an MHC class II β chain polypeptide and the second MHC class II polypeptide is an MHC class II α chain polypeptide.

### Position 3 MOD(s)

As another example, in some cases, a TMMP of the present disclosure comprises: a) a first polypeptide comprising, in order from N-terminus to C-terminus: i) a T1D peptide; and ii) a first MHC class II polypeptide comprising a substitution of an amino acid (other than a Cys) with a Cys (a “first Cys”), the components of the first polypeptide optionally being joined by a linker that does not comprise a Cys; and b) a second polypeptide comprising, in order from N-terminus to C-terminus: i) a second MHC class II polypeptide comprising a substitution of an amino acid (other than a Cys) with a Cys (a “second Cys”); ii) an Ig Fc polypeptide; and iii) one or more immunomodulatory polypeptides, the components of the second polypeptide optionally being joined by a linker that does not comprise a Cys; where the first polypeptide and the second polypeptide are linked via a disulfide bond between the first Cys and the second Cys. (See FIG. 5, MOD position 3.) In any of the above embodiments, in some cases the one or more immunomodulatory polypeptides is a PD-L1 polypeptide or variant thereof, in some cases the one or more immunomodulatory polypeptides is a TGF-β polypeptide or variant thereof, in some cases the one or more immunomodulatory polypeptides is an IL-2 polypeptide or variant thereof, and in some cases the one or more immunomodulatory polypeptides is a FasL polypeptide or variant thereof. In some cases, the T1D peptide is a proinsulin peptide. In some cases, the T1D peptide is a GAD peptide. In any of the above embodiments, in some cases, the first MHC class II polypeptide is an MHC class II β chain polypeptide and the second MHC class II polypeptide is an MHC class II α chain polypeptide.

### Position 4 MOD(s)

As another example, in some cases, a TMMP of the present disclosure comprises: a) a first polypeptide comprising, in order from N-terminus to C-terminus: i) one or more immunomodulatory polypeptides; ii) a T1D peptide; and iii) a first MHC class II polypeptide comprising a substitution of an amino acid (other than a Cys) with a Cys (a “first Cys”), the components of the first polypeptide optionally being joined by one or more linkers that do not comprise a Cys; and b) a second polypeptide comprising, in order from N-terminus to C-terminus: i) a second MHC class II polypeptide comprising a substitution of an amino acid (other than a Cys) with a Cys (a “second Cys”); and ii) optionally an Ig Fc polypeptide, where when the optional Ig Fc is present, the components of the second polypeptide are optionally joined by a linker that does not comprise a Cys; where the first polypeptide and the second polypeptide are linked via a disulfide bond between the first Cys and the second Cys. (See FIG. 5, MOD position 4.) In any of the above embodiments, in some cases the one or more immunomodulatory polypeptides is a PD-L1 polypeptide or variant thereof, in some cases the one or more immunomodulatory polypeptides is a TGF-β polypeptide or variant thereof, in some cases the one or more immunomodulatory polypeptides is an IL-2 polypeptide or variant thereof, and in some cases the one or more immunomodulatory polypeptides is a FasL polypeptide or variant thereof. In some cases, the T1D peptide is a proinsulin peptide. In some cases, the T1D peptide is a GAD peptide. In any of the above embodiments, in some cases, the first MHC class II polypeptide is an MHC class II β chain polypeptide and the second MHC class II polypeptide is an MHC class II α chain polypeptide.

### Position 5 MOD(s)

As another example, in some cases, a TMMP of the present disclosure comprises: a) a first polypeptide comprising, in order from N-terminus to C-terminus: i) a T1D peptide; ii) a first MHC class II polypeptide comprising a substitution of an amino acid (other than a Cys) with a Cys (a “first Cys”); and iii) one or more immunomodulatory polypeptides, the components of the first polypeptide optionally being joined by one or more linkers that do not comprise a Cys; and b) a second polypeptide comprising, in order from N-terminus to C-terminus: i) a second MHC class II polypeptide comprising a substitution of an amino acid (other than a Cys) with a Cys (a “second Cys”); and ii) optionally an Ig Fc polypeptide, where when the optional Ig Fc is present, the components of the second polypeptide are optionally joined by a linker that does not comprise a Cys; where the first polypeptide and the second polypeptide are linked via a disulfide bond between the first Cys and the second Cys. (See FIG. 5, MOD position 5.) In any of the above embodiments, in some cases, the one or more immunomodulatory polypeptides is a PD-L1 polypeptide or variant thereof, in some cases the one or more immunomodulatory polypeptides is a TGF-β polypeptide or variant thereof, in some cases the one or more immunomodulatory polypeptides is an IL-2 polypeptide or variant thereof, and in some cases the one or more immunomodulatory polypeptides is a FasL polypeptide or variant thereof. In some cases, the T1D peptide is a proinsulin peptide. In some cases, the T1D peptide is a GAD peptide. In any of the above embodiments, in some cases, the first MHC class II polypeptide is an MHC class II β chain polypeptide and the second MHC class II polypeptide is an MHC class II α chain polypeptide.

### MHC Class II Polypeptides

As noted above, a TMMP of the present disclosure comprises MHC class II polypeptides.

Naturally occurring MHC class II polypeptides comprise an α chain and a β chain. As used herein, the term “MHC class II polypeptides” include human leukocyte antigen (HLA) α- and β-chains. MHC class II polypeptides include MHC class II DP α and β polypeptides, DM α and β polypeptides, DOA α and β polypeptides, DOB α and β polypeptides, DQ α and β polypeptides, and DR α and β polypeptides. As used herein, a “MHC class II polypeptide” can comprise a MHC class II α chain polypeptide, a MHC class II β chain polypeptide, or only a portion of a MHC class II α or β chain polypeptide. For example, a “MHC class II polypeptide” can be a polypeptide that includes: i) only the α1 domain of a MHC class II α chain polypeptide; ii) only the α2 domain of a MHC class II α chain; iii) only the α1 domain and an α2 domain of a MHC class II α chain; iv) only the β1 domain of a MHC class II β chain; v) only the β2 domain of a MHC class II β chain; vi) only the β1 domain and the β2 domain of a MHC class II β chain; vii) the α1 domain of a MHC class II α chain, the β1 domain of a MHC class II β chain, and the β2 domain of a MHC class II; and the like.

MHC class II polypeptides include allelic forms. The HLA locus is highly polymorphic in nature. As disclosed in the Nomenclature for Factors of the HLA System 2000 (Hum. Immunol.; 62(4):419-68, 2001) there are 221 HLA-DRB 1 alleles, 19 DRB3 alleles, 89 DRB4 alleles, 14 DRB5 alleles, 19 DQA1 alleles and 39 DQB1 alleles, with new alleles being discovered continuously. A 2007 update by the WHO nomenclature Committee for Factors of the HLA System (www.anthonynolan.com/HIG/) showed there are 3 DRA alleles, 494 DRB 1 alleles, 1 DRB2 alleles, 44 DRB3 alleles, 13 DRB4 alleles, 18 DRB5 alleles, 3 DRB6 alleles, 2 DRB7 alleles, 10 DRB8 alleles, 1 DRB9 alleles, 34 DQA1 alleles, 83 DQB1 alleles, 23 DPA1, 126 DPB1 alleles, 4 DMA alleles, 7 DMB alleles, 12 DOA alleles and 9 DOB alleles. As used herein, the term “MHC class II polypeptide” includes allelic forms of any known MHC class II polypeptide.

In some cases, a TMMP of the present disclosure comprises an MHC class II α chain (e.g., a DPA1, DRA1, or DQA1 α chain), without the leader, transmembrane, and intracellular portions (e.g., cytoplasmic tails) that may be present in a naturally-occurring MHC class II α chain. Thus, in some cases, a TMMP of the present disclosure comprises only the α1 and α2 portions of an MHC class II α chain (e.g., only the α1 and α2 portions of a DPA1, DRA1, or DQA1 α chain); and does not include the leader, transmembrane, and intracellular portions (e.g., cytoplasmic tails) that may be present in a naturally-occurring MHC class II α chain.

In some cases, a TMMP of the present disclosure comprises an MHC class II β chain (e.g., a DPB1, DRB1, or DQB1 β chain), without the leader, transmembrane, and intracellular portions (e.g., cytoplasmic tails) that may be present in a naturally-occurring MHC class II β chain. Thus, in some cases, a TMMP of the present disclosure comprises only the β1 and 2 portions of an MHC class II β chain (e.g., only the β1 and β2 portions of a DPB1, DRB1, or DQB1 β chain); and does not include the leader, transmembrane, and intracellular portions (e.g., cytoplasmic tails) that may be present in a naturally-occurring MHC class II β chain.

### MHC Class II Alpha Chains

MHC class II alpha chains comprise an α1 domain and an α2 domain. In some cases, the α1 domain and the α2 domain present in an antigen-presenting cell are from the same MHC class II α chain polypeptide. In some cases, the α1 domain and the α2 domain present in an antigen-presenting cell are from two different MHC class II α chain polypeptides.

MHC class II alpha chains suitable for inclusion in a TMMP of the present disclosure lack a signal peptide. An MHC class II alpha chain suitable for inclusion in a multimeric polypeptide of the present disclosure can have a length of from about 60 amino acids to about 190 amino acids; for example, an MHC class II alpha chain suitable for inclusion in a TMMP of the present disclosure can have a length of from about 60 amino acids to about 80 amino acids, from about 80 amino acids to about 100 amino acids, from about 100 amino acids to about 120 amino acids, from about 120 amino acids to about 140 amino acids, from about 140 amino acids to about 160 amino acids, from about 160 amino acids to about 180 amino acids, or from about 180 amino acids to about 200 amino acids. An MHC class II α1 domain suitable for inclusion in a TMMP of the present disclosure can have a length of from about 30 amino acids to about 95 amino acids; for example, an MHC class II α1 domain suitable for inclusion in a TMMP of the present disclosure can have a length of from about 30 amino acids to about 40 amino acids, from about 40 amino acids to about 50 amino acids, from about 50 amino acids to about 60 amino acids, from about 60 amino acids to about 70 amino acids, from about 70 amino acids to about 80 amino acids, from about 80 amino acids to about 90 amino acids, or from about 90 amino acids to about 95 amino acids. An MHC class II α2 domain suitable for inclusion in a TMMP of the present disclosure can have a length of from about 30 amino acids to about 95 amino acids; for example, an MHC class II α2 domain suitable for inclusion in a TMMP of the present disclosure can have a length of from about 30 amino acids to about 40 amino acids, from about 40 amino acids to about 50 amino acids, from about 50 amino acids to about 60 amino acids, from about 60 amino acids to about 70 amino acids, from about 70 amino acids to about 80 amino acids, from about 80 amino acids to about 90 amino acids, or from about 90 amino acids to about 95 amino acids.

In some cases, an MHC class II α chain polypeptide present in a TMMP of the present disclosure comprises an amino acid substitution, relative to a wild-type MHC class II α chain polypeptide, where the amino acid substitution replaces an amino acid (other than a Cys) with a Cys. Such an amino acid substitution can occur between amino acid 55 and 110 of the MHC class II α chain polypeptide. For example, an amino acid substitution that replaces an amino acid other than a Cys with a Cys can be present between amino acids 55 and 60, between amino acids 60 and 65, between amino acids 65 and 70, between amino acids 70 and 75, between amino acids 75 and 80, between amino acids 80 and 85, between amino acids 85 and 90, between amino acids 90 and 95, between amino acids 95 and 100, between amino acids 100 and 105, or between amino acids 105 and 110.

### DRA Polypeptides

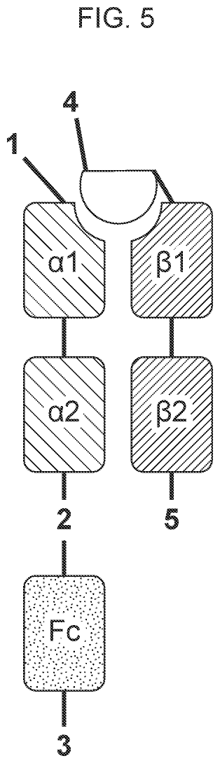

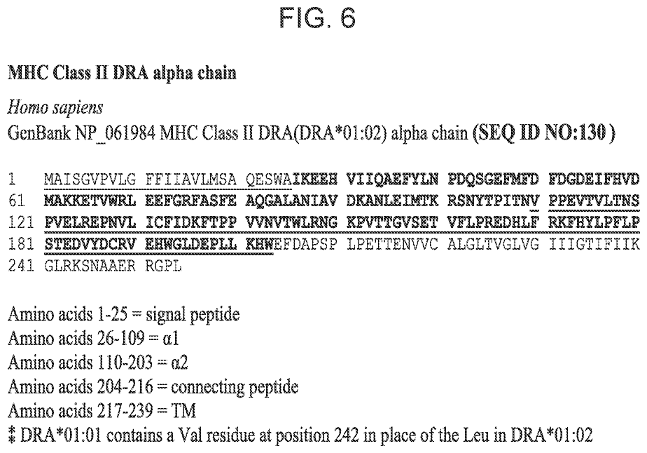

In some cases, a suitable MHC class II α chain polypeptide is a DRA polypeptide, more specifically a DRA1 polypeptide. A DRA polypeptide can have at least 60%, at least 70%, at least 80%, at least 90%, at least 95%, at least 98%, at least 99%, or 100%, amino acid sequence identity with amino acids 26-203 of the DRA amino acid sequence depicted in FIG. 6. In some cases, the DRA polypeptide has a length of about 178 amino acids (e.g., 175, 176, 177, 178, 179, or 180 amino acids).

A “DRA polypeptide” includes allelic variants, e.g., naturally occurring allelic variants. Thus, in some cases, a suitable DRA polypeptide comprises the following amino acid sequence: IKEEH VIIQAEFYLN PDQSGEFMED FDGDEIFHVD MAKKETVWRL EEFGRFASFE AQGALANIAV DKANLEIMTK RSNYTPITNV PPEVTVLTNSPVELREPNVL ICFIDKFTPP VVNVTWLRNG KPVTTGVSET VFLPREDHLF RKFHYLPFLPSTEDVYDCRV EHWGLDEPLL KHW (SEQ ID NO:6, amino acids 26-203 of DRA*01:02, see FIG. 6), or an allelic variant thereof. In some cases, the allelic variant is the DRA*01:01:01:01 allelic variant that differs from DRA*01:02:01 by having a valine in place of the leucine at position 242 of the sequence in FIG. 6. In some cases, a DRA polypeptide suitable for inclusion in a TMMP of the present disclosure comprises an amino acid substitution, relative to a wild-type DRA polypeptide, where the amino acid substitution replaces an amino acid (other than a Cys) with a Cys.

In some cases, a TMMP of the present disclosure comprises a variant DRA polypeptide that comprises a non-naturally occurring Cys residue. For example, in some cases, a TMMP of the present disclosure comprises a variant DRA polypeptide that comprises an amino acid substitution selected from E3C, E4C, F12C, G28C, D29C, I72C, K75C, T80C, P81C, 182C, T93C, N94C, and S95C.

In some cases, a suitable DRA polypeptide comprises the following amino acid sequence: IKEEH VIIQAEFYLN PDQSGEFMFD FDGDEIFHVD MAKKETVWRL EEFGRFASFE AQGALANIAV DKANLECMTK RSNYTPITNV PPEVTVLTNSPVELREPNVL ICFIDKFTPP VVNVTWLRNG KPVTTGVSET VFLPREDHLF RKFHYLPFLPSTEDVYDCRV EHWGLDEPLL KHW (SEQ ID NO:7), where amino acid 172 is substituted with a Cys (shown in bold text). In some cases, a suitable DRA polypeptide comprises the following amino acid sequence: IKEEH VIIQAEFYLN PDQSGEFMFD FDGDEIFHVD MAKKETVWRL EEFGRFASFE AQGALANIAV DKANLEIMTC RSNYTPITNV PPEVTVLTNSPVELREPNVL ICFIDKFTPP VVNVTWLRNG KPVTTGVSET VFLPREDHLF RKFHYLPFLPSTEDVYDCRV EHWGLDEPLL KHW (SEQ ID NO:8), where amino acid K75 is substituted with a Cys (shown in bold text).

In some cases, a suitable DRA polypeptide comprises the following amino acid sequence: IKEEHVIIQAEFYLNPDQSGEFMFDFDGDEIFHVDMAKKETVWRLEEFGRFASFEAQGALANIAV DKANLEIMTKRSNYTPITNVPPEVTVLTNSPVELREPNVLICFIDKFTPPVVNVTWLRNGKPVTT GVSETVFLPREDHLFRKFHYLPFLPSTEDVYDCRVEHWGLDEPLLKHWEFDAPSPLPET (SEQ ID NO:9). In some cases, an amino acid between amino acids 55 and 110 is substituted with a Cys. In some cases, an amino acid between amino acids 70 and 85 is substituted with a Cys. In some cases, a suitable DRA polypeptide comprises the following amino acid sequence: IKEEHVIIQAEFYLNPDQSGEFMFDFDGCEIFHVDMAKKETVWRLEEFGRFASFEAQGALANIAV DKANLEIMTKRSNYTPITNVPPEVTVLTNSPVELREPNVLICFIDKFTPPVVNVTWLRNGKPVTT GVSETVFLPREDHLFRKFHYLPFLPSTEDVYDCRVEHWGLDEPLLKHWEFDAPSPLPET (SEQ ID NO:10), where D29 is substituted with a Cys (shown in bold text). In some cases, a suitable DRA polypeptide comprises the following amino acid sequence: IKEEHVIIQAEFYLNPDQSGEFMFDFDGDEIFHVDMAKKETVWRLEEFGRFASFEAQGALANIAV DKANLECMTKRSNYTPITNVPPEVTVLTNSPVELREPNVLICFIDKFTPPVVNVTWLRNGKPVTT GVSETVFLPREDHLFRKFHYLPFLPSTEDVYDCRVEHWGLDEPLLKHWEFDAPSPLPET (SEQ ID NO:11), where 172 is substituted with a Cys (shown in bold text). In some cases, a suitable DRA polypeptide comprises the following amino acid sequence: IKEEHVIIQAEFYLNPDQSGEFMFDFDGDEIFHVDMAKKETVWRLEEFGRFASFEAQGALANIAV DKANLEIMTCRSNYTPITNVPPEVTVLTNSPVELREPNVLICFIDKFTPPVVNVTWLRNGKPVTT GVSETVFLPREDHLFRKFHYLPFLPSTEDVYDCRVEHWGLDEPLLKHWEFDAPSPLPET (SEQ ID NO:12), where K75 is substituted with a Cys (shown in bold text). In some cases, a suitable DRA polypeptide comprises the following amino acid sequence: IKEEHVIIQAEFYLNPDQSGEFMFDFDGDEIFHVDMAKKETVWRLEEFGRFASFEAQGALANIAV DKANLEIMTKRSNYTCITNVPPEVTVLTNSPVELREPNVLICFIDKFTPPVVNVTWLRNGKPVTT GVSETVFLPREDHLFRKFHYLPFLPSTEDVYDCRVEHWGLDEPLLKHWEFDAPSPLPET (SEQ ID NO:13), where P81 is substituted with a Cys (shown in bold text). In some cases, a suitable DRA polypeptide comprises the following amino acid sequence: IKEEHVIIQAEFYLNPDQSGEFMFDFDGDEIFHVDMAKKETVWRLEEFGRFASFEAQGALANIAV DKANLEIMTKRSNYTPCTNVPPEVTVLTNSPVELREPNVLICFIDKFTPPVVNVTWLRNGKPVTT GVSETVFLPREDHLFRKFHYLPFLPSTEDVYDCRVEHWGLDEPLLKHWEFDAPSPLPET (SEQ ID NO:14), where 182 is substituted with a Cys (shown in bold text).

A suitable DRA α1 domain comprises an amino acid sequence having at least 60%, at least 70%, at least 80%, at least 90%, at least 95%, at least 98%, at least 99%, or 100%, amino acid sequence identity to the following amino acid sequence: VIIQAEFYLN PDQSGEFMED FDGDEIFHVD MAKKETVWRL EEFGRFASFE AQGALANIAV DKANLEIMTK RSNYTPITN (SEQ ID NO:15); and can have a length of about 84 amino acids (e.g., 80, 81, 82, 83, 84, 85, or 86 amino acids). A suitable DRA α1 domain can comprise the following amino acid sequence: VIIQAEFYLN PDQSGEFMED FDGDEIFHVD MAKKETVWRL EEFGRFASFE AQGALANIAV DKANLEIMTK RSNYTPITN (SEQ ID NO:15), or a naturally-occurring allelic variant. A suitable DRA α1 domain can comprise the following amino acid sequence: IKEEH VIIQAEFYLN PDQSGEFMED FDGCEIFHVD MAKKETVWRL EEFGRFASFE AQGALANIAV DKANLEIMTK RSNYTPITN (SEQ ID NO:16), where amino acid D29C is substituted with a Cys (shown in bold text). A suitable DRA α1 domain can comprise the following amino acid sequence: IKEEH VIIQAEFYLN PDQSGEFMFD FDGDEIFHVD MAKKETVWRL EEFGRFASFE AQGALANIAV DKANLECMTK RSNYTPITN (SEQ ID NO:17), where amino acid 172 is substituted with a Cys (shown in bold text). A suitable DRA α1 domain can comprise the following amino acid sequence: IKEEH VIIQAEFYLN PDQSGEFMFD FDGDEIFHVD MAKKETVWRL EEFGRFASFE AQGALANIAV DKANLEIMTC RSNYTPITN (SEQ ID NO:18), where amino acid K75 is substituted with a Cys (shown in bold text). A suitable DRA α1 domain can comprise the following amino acid sequence: IKEEH VIIQAEFYLN PDQSGEFMFD FDGDEIFHVD MAKKETVWRL EEFGRFASFE AQGALANIAV DKANLEIMTK RSNYTCITN (SEQ ID NO:19), where amino acid P81 is substituted with a Cys (shown in bold text). A suitable DRA α1 domain can comprise the following amino acid sequence: IKEEH VIIQAEFYLN PDQSGEFMFD FDGDEIFHVD MAKKETVWRL EEFGRFASFE AQGALANIAV DKANLEIMTK RSNYTPCTN (SEQ ID NO:20), where amino acid 182 is substituted with a Cys (shown in bold text).

A suitable DRA α2 domain comprises an amino acid sequence having at least 60%, at least 70%, at least 80%, at least 90%, at least 95%, at least 98%, at least 99%, or 100%, amino acid sequence identity to the following amino acid sequence: V PPEVTVLTNSPVELREPNVL ICFIDKFTPP VVNVTWLRNG KPVTTGVSET VFLPREDHLF RKFHYLPFLPSTEDVYDCRV EHWGLDEPLL KHW (SEQ ID NO:21); and can have a length of about 94 amino acids (e.g., 90, 91, 92, 93, 94, 95, 96, 97, or 98 amino acids).

### DPA1 Polypeptides

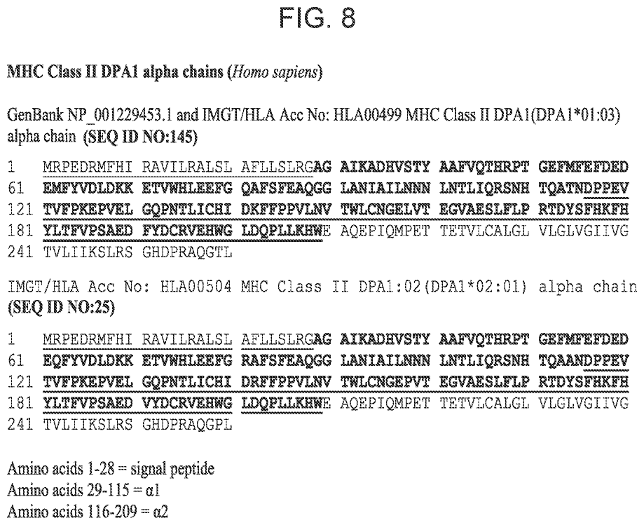

In some cases, a suitable MHC class II α chain polypeptide is a DPA1 polypeptide. A DPA1 polypeptide can have at least 60%, at least 70%, at least 80%, at least 90%, at least 95%, at least 98%, at least 99%, or 100%, amino acid sequence identity with amino acids 29-209 of the DPA1 amino acid sequence depicted in FIG. 8. In some cases, the DPA1 polypeptide has a length of about 181 amino acids (e.g., 178, 179, 180, 181, 182, 183, or 184 amino acids). In some cases, a DPA polypeptide suitable for inclusion in a TMMP of the present disclosure comprises an amino acid substitution, relative to a wild-type DPA polypeptide, where the amino acid substitution replaces an amino acid (other than a Cys) with a Cys.

A “DPA1 polypeptide” includes allelic variants, e.g., naturally occurring allelic variants. Thus, in some cases, a suitable DPA1 polypeptide comprises the following amino acid sequence: AG AIKADHVSTY AAFVQTHRPT GEFMFEFDED EMFYVDLDKK ETVWHLEEFG QAFSFEAQGG LANIAILNNN LNTLIQRSNH TQATNDPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO:22; amino acids 29-209 of DPA1*01:03:01:01, see FIG. 8), or an allelic variant thereof (e.g., DPA1*02:01; see FIG. 8). In some cases, a DPA polypeptide suitable for inclusion in a TMMP of the present disclosure comprises an amino acid substitution, relative to a wild-type DPA polypeptide, where the amino acid substitution replaces an amino acid (other than a Cys) with a Cys.

A suitable DPA1 α1 domain may comprise an amino acid sequence having at least 60%, at least 70%, at least 80%, at least 90%, at least 95%, at least 98%, at least 99%, or 100%, amino acid sequence identity to the following amino acid sequence: AIKADHVSTY AAFVQTHRPT GEFMFEFDED EMFYVDLDKK ETVWHLEEFG QAFSFEAQGG LANIAILNNN LNTLIQRSNH TQATN (SEQ ID NO:23); and can have a length of about 87 amino acids (e.g., 84, 85, 86, 87, 88, or 89 amino acids). A suitable DPA1 α1 domain can comprise the following amino acid sequence: AIKADHVSTY AAFVQTHRPT GEFMFEFDED EMFYVDLDKK ETVWHLEEFG QAFSFEAQGG LANIAILNNN LNTLIQRSNH TQATN (SEQ ID NO:23), or a naturally-occurring allelic variant.

A suitable DPA1 α2 domain may comprise an amino acid sequence having at least 60%, at least 70%, at least 80%, at least 90%, at least 95%, at least 98%, at least 99%, or 100%, amino acid sequence identity to the following amino acid sequence: DPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO:24); and can have a length of about 97 amino acids (e.g., 91, 92, 93, 94, 95, 96, or 97 amino acids). A suitable DPA1 α2 domain can comprise the following amino acid sequence: DPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO:24), or a naturally-occurring allelic variant thereof.

Other DPA1 polypeptides comprise the sequence: MRPEDRMFHIRAVILRALSLAFLLSLRGAGAIKADHVSTYAAFVQTHRPTGEFMFEFDEDEQFY VDLDKKETVWHLEEFGRAFSFEAQGGLANIAILNNNLNTLIQRSNHTQAANDPPEVTVFPKEPV ELGQPNTLICHIDRFFPPVLNVTWLCNGEPVTEGVAESLFLPRTDYSFHKFHYLTFVPSAEDVYD CRVEHWGLDQPLLKHWEAQEPIQMPETTETVLCALGLVLGLVGIIVGTVLIIKSLRSGHDPRAQ GPL (SEQ ID NO:25; amino acids 29-209 of DPA1*02:01:01:01, see FIG. 8), or variant thereof having at least 60%, at least 70%, at least 80%, at least 90%, at least 95%, at least 98%, at least 99%, or 100%, amino acid sequence identity.

A suitable DPA1 α1 domain may comprise an amino acid sequence having at least 60%, at least 70%, at least 80%, at least 90%, at least 95%, at least 98%, at least 99%, or 100%, amino acid sequence identity to the amino acids 29-115 of DPA1*02:01:01:01, SEQ ID NO:25; and can have a length of about 87 amino acids (e.g., 84, 85, 86, 87, 88, or 89 amino acids. A suitable DPA1 α2 domain may comprise an amino acid sequence having at least 60%, at least 70%, at least 80%, at least 90%, at least 95%, at least 98%, at least 99%, or 100%, amino acid sequence identity to amino acids 116 to 209 of DPA1*02:01:01:01, SEQ ID NO:25; and can have a length of about 97 amino acids (e.g., 91, 92, 93, 94, 95, 96, or 97 amino acids).

### DQA1 Polypeptides

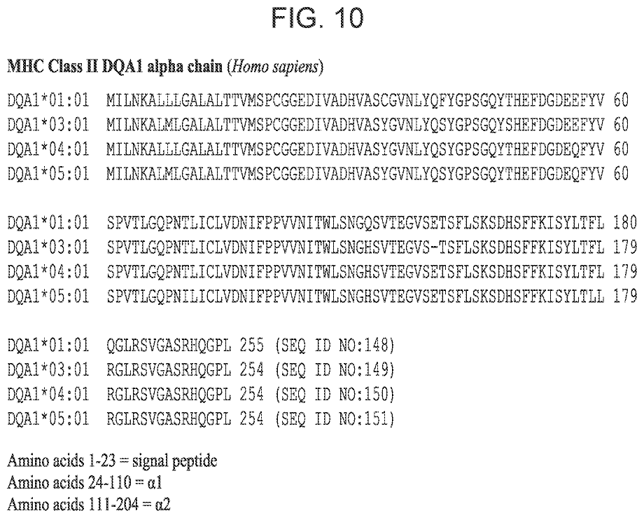

In some cases, a suitable MHC class II α chain polypeptide is a DQA1 polypeptide. A DQA1 polypeptide can have at least 60%, at least 70%, at least 80%, at least 90%, at least 95%, at least 98%, at least 99%, or 100%, amino acid sequence identity with amino acids 24-204 of any of the DQA1 amino acid sequences depicted in FIG. 10. In some cases, the DQA1 polypeptide has a length of about 181 amino acids (e.g., 177, 178, 179, 180, 181, 182, or 183 amino acids). In an embodiment, a DQA1 α chain polypeptide can have at least 60%, at least 70%, at least 80%, at least 90%, at least 95%, at least 98%, at least 99%, or 100%, amino acid sequence identity with amino acids 30-227 of the DQA1*01:01 α chain amino acid in FIG. 10, ImMunoGeneTics (“IMGT”)/HLA Acc No:HLA00601. In an embodiment, a DQA1 α chain polypeptide can have at least 60%, at least 70%, at least 80%, at least 90%, at least 95%, at least 98%, at least 99%, or 100%, amino acid sequence identity with amino acids 30-227 of the DQA1*03:01: α chain amino acid in FIG. 10, IMGT/HLA Acc No:HLA00609. In an embodiment, a DQA1 α chain polypeptide can have at least 60%, at least 70%, at least 80%, at least 90%, at least 95%, at least 98%, at least 99%, or 100%, amino acid sequence identity with amino acids 30-227 of the DQA1*04:01 α chain amino acid in FIG. 10, IMGT/HLA Acc No:HLA00612. In an embodiment, a DQA1 α chain polypeptide can have at least 60%, at least 70%, at least 80%, at least 90%, at least 95%, at least 98%, at least 99%, or 100%, amino acid sequence identity with amino acids 30-227 of the DQA1*05:01 α chain amino acid in FIG. 10, IMGT/HLA Acc No:HLA00613. In some cases, a DQA polypeptide suitable for inclusion in a TMMP of the present disclosure comprises an amino acid substitution, relative to a wild-type DQA polypeptide, where the amino acid substitution replaces an amino acid (other than a Cys) with a Cys.

A “DQA1 polypeptide” includes allelic variants, e.g., naturally occurring allelic variants. Thus, in some cases, a suitable DQA1 polypeptide comprises the following amino acid sequence: EDIVADH VASCGVNLYQ FYGPSGQYTH EFDGDEQFYV DLERKETAWR WPEFSKFGGF DPQGALRNMA VAKHNLNIMI KRYNSTAATN EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO:26), or an allelic variant thereof. In some cases, a DQA polypeptide suitable for inclusion in a TMMP of the present disclosure comprises an amino acid substitution, relative to a wild-type DQA polypeptide, where the amino acid substitution replaces an amino acid (other than a Cys) with a Cys.

A suitable DQA1 α1 domain comprises an amino acid sequence having at least 60%, at least 70%, at least 80%, at least 90%, at least 95%, at least 98%, at least 99%, or 100%, amino acid sequence identity to the following amino acid sequence: EDIVADH VASCGVNLYQ FYGPSGQYTH EFDGDEQFYV DLERKETAWR WPEFSKFGGF DPQGALRNMA VAKHNLNIMI KRYNSTAATN (SEQ ID NO:27); and can have a length of about 87 amino acids (e.g., 84, 85, 86, 87, 88, or 89 amino acids). A suitable DQA1 α1 domain can comprise the following amino acid sequence: EDIVADH VASCGVNLYQ FYGPSGQYTH EFDGDEQFYV DLERKETAWR WPEFSKFGGF DPQGALRNMA VAKHNLNIMI KRYNSTAATN (SEQ ID NO:28), or a naturally-occurring allelic variant.

A suitable DQA1 α2 domain comprises an amino acid sequence having at least 60%, at least 70%, at least 80%, at least 90%, at least 95%, at least 98%, at least 99%, or 100%, amino acid sequence identity to the following amino acid sequence: EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO:29); and can have a length of about 94 amino acids (e.g., 91, 92, 93, 94, 95, 96, or 97 amino acids). A suitable DQA1 α2 domain can comprise the following amino acid sequence: EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO:29), or a naturally-occurring allelic variant thereof.

### MHC Class II Beta Chains

MHC class II beta chains comprise a β1 domain and a β2 domain. In some cases, the β1 domain and the β2 domain present in an antigen-presenting cell are from the same MHC class II β chain polypeptide. In some cases, the β1 domain and the β2 domain present in an antigen-presenting cell are from two different MHC class II β chain polypeptides. In some cases, MHC class II β chain polypeptide suitable for inclusion in a TMMP of the present disclosure comprises an amino acid substitution, relative to a wild-type MHC class II β chain polypeptide, where the amino acid substitution replaces an amino acid (other than a Cys) with a Cys.

MHC class II beta chains suitable for inclusion in a TMMP of the present disclosure lack a signal peptide. An MHC class II beta chain suitable for inclusion in a TMMP of the present disclosure can have a length of from about 60 amino acids to about 210 amino acids; for example, an MHC class II beta chain suitable for inclusion in a TMMP of the present disclosure can have a length of from about 60 amino acids to about 80 amino acids, from about 80 amino acids to about 100 amino acids, from about 100 amino acids to about 120 amino acids, from about 120 amino acids to about 140 amino acids, from about 140 amino acids to about 160 amino acids, from about 160 amino acids to about 180 amino acids, from about 180 amino acids to about 200 amino acids, or from about 200 amino acids to about 210 amino acids. An MHC class II β1 domain suitable for inclusion in a TMMP of the present disclosure can have a length of from about 30 amino acids to about 105 amino acids; for example, an MHC class II β1 domain suitable for inclusion in a TMMP of the present disclosure can have a length of from about 30 amino acids to about 40 amino acids, from about 40 amino acids to about 50 amino acids, from about 50 amino acids to about 60 amino acids, from about 60 amino acids to about 70 amino acids, from about 70 amino acids to about 80 amino acids, from about 80 amino acids to about 90 amino acids, from about 90 amino acids to about 95 amino acids, from about 95 amino acids to about 100 amino acids, or from about 100 amino acids to about 105 amino acids. An MHC class II β2 domain suitable for inclusion in a TMMP of the present disclosure can have a length of from about 30 amino acids to about 105 amino acids; for example, an MHC class II β2 domain suitable for inclusion in a TMMP of the present disclosure can have a length of from about 30 amino acids to about 40 amino acids, from about 40 amino acids to about 50 amino acids, from about 50 amino acids to about 60 amino acids, from about 60 amino acids to about 70 amino acids, from about 70 amino acids to about 80 amino acids, from about 80 amino acids to about 90 amino acids, from about 90 amino acids to about 95 amino acids, from about 95 amino acids to about 100 amino acids, or from about 100 amino acids to about 105 amino acids.

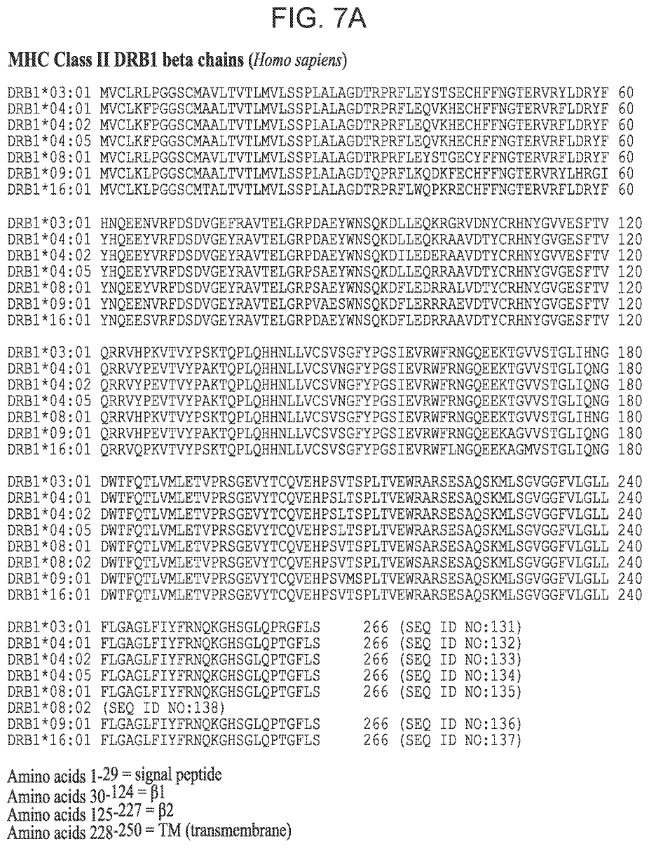

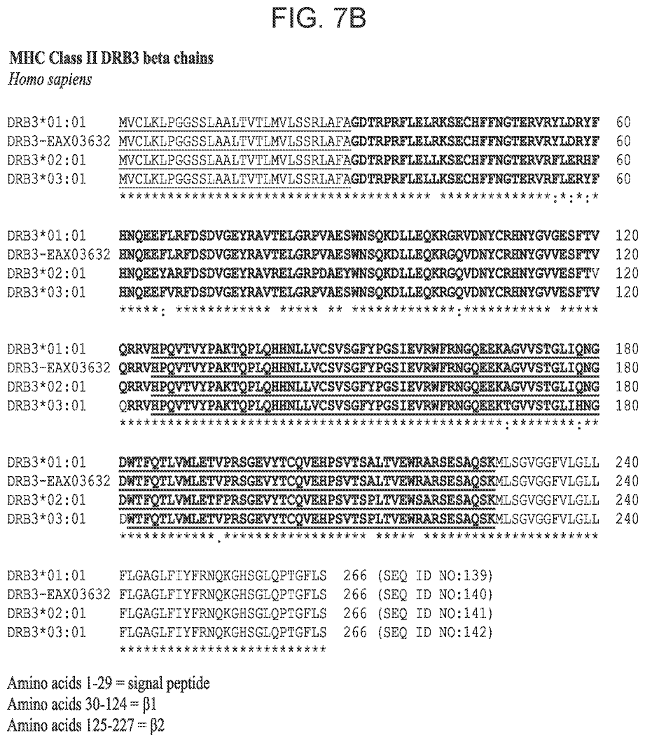

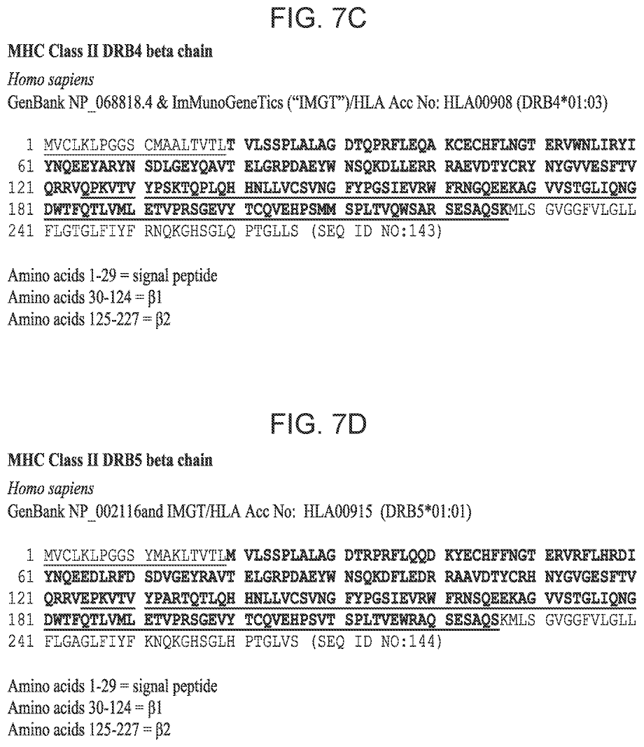

In some cases, the MHC class II β chain polypeptide is a variant DRB MHC class II polypeptide that comprises a non-naturally occurring Cys residue. For example, in some cases, the MHC class II β chain polypeptide is a variant DRB1 MHC class II polypeptide that comprises an amino acid substitution selected from the group consisting of P5C. F7C, Q10C, N19C, G20C, H33C, G151C. D152C, and W153C. In some cases, the MHC class II β chain polypeptide is a variant DRB1 polypeptide comprising an amino acid sequence having at least 60%, at least 70%, at least 80%, at least 90%, at least 95%, at least 98%, or at least 99%, amino acid sequence identity to the following DRB1 amino acid sequence: GDTRPRFLEQVKHECHFENGTERVRFLDRYFYHQEEYVRFDSDVGEYRAVTELGRPDAEYWNS QKDLLEQKRAAVDTYCRHNYGVGESFTVQRRVYPEVTVYPAKTQPLQHHNLLVCSVNGFYPA SIEVRWFRNGQEEKTGVVSTGLIQNGDWTFQTLVMLETVPRSGEVYTCQVEHPSLTSPLTVEWR ARSESAQSKM (SEQ ID NO:30), and comprising an amino acid substitution selected from the group consisting of P5C. F7C. Q10C. N19C. G20C. H33C. G151C. D152C. and W153C. In some cases, the MHC class II β chain polypeptide is a variant DRB3 polypeptide, a variant DRB4 polypeptide, or a variant DRB5 polypeptide comprising an amino acid substitution corresponding to any of the foregoing amino acid substitutions in a variant DRB1 polypeptide. For example, as depicted in FIG. 7B: i) an amino acid corresponding to P5 of DRB1 would be P5 of a mature DRB3 polypeptide (lacking the N-terminal signal peptide MVCLKLPGGSSLAALTVTLMVLSSRLAFA (SEQ ID NO:31)); ii) an amino acid corresponding to F7 of DRB1 would be F7 of a mature DRB3 polypeptide; iii) an amino acid corresponding to Q10 of DRB1 would be L10 of a mature DRB3 polypeptide; iv) an amino acid corresponding to N19 of DRB1 would be N19 of a mature DRB3 polypeptide; v) an amino acid corresponding to G20 of DRB1 would be G20 of a mature DRB3 polypeptide; vi) an amino acid corresponding to H33 of DRB1 would be N33 of a mature DRB3 polypeptide; vii) an amino acid corresponding to G151 of DRB1 would be G151 of a mature DRB3 polypeptide; viii) an amino acid corresponding to D152 of DRB1 would be D152 of a mature DRB3 polypeptide; and ix) an amino acid corresponding to W153 of DRB1 would be W153 of a mature DRB3 polypeptide. As another example, as depicted in FIG. 7C, i) an amino acid corresponding to P5 of DRB1 would be P15 of a mature DRB4 polypeptide (lacking the N-terminal signal peptide MVCLKLPGGSCMAALTVTL (SEQ ID NO:32)); ii) an amino acid corresponding to F7 of DRB1 would be F17 of a mature DRB4 polypeptide; iii) an amino acid corresponding to Q10 of DRB1 would be Q20 of a mature DRB4 polypeptide; iv) an amino acid corresponding to N19 of DRB1 would be N29 of a mature DRB4 polypeptide; v) an amino acid corresponding to G20 of DRB1 would be G30 of a mature DRB4 polypeptide; vi) an amino acid corresponding to H33 of DRB1 would be N43 of a mature DRB4 polypeptide; vii) an amino acid corresponding to G151 of DRB1 would be G161 of a mature DRB4 polypeptide; viii) an amino acid corresponding to D152 of DRB1 would be D162 of a mature DRB4 polypeptide; and ix) an amino acid corresponding to W153 of DRB1 would be W153 of a mature DRB4 polypeptide. As another example, as depicted in FIG. 7D, i) an amino acid corresponding to P5 of DRB1 would be P15 of a mature DRB5 polypeptide (lacking the N-terminal signal peptide MVCLKLPGGSYMAKLTVTL (SEQ ID NO:33)); ii) an amino acid corresponding to F7 of DRB1 would be F17 of a mature DRB5 polypeptide; iii) an amino acid corresponding to Q10 of DRB1 would be Q20 of a mature DRB5 polypeptide; iv) an amino acid corresponding to N19 of DRB1 would be N29 of a mature DRB5 polypeptide; v) an amino acid corresponding to G20 of DRB1 would be G30 of a mature DRB5 polypeptide; vi) an amino acid corresponding to H33 of DRB1 would be N43 of a mature DRB5 polypeptide; vii) an amino acid corresponding to G151 of DRB1 would be G161 of a mature DRB5 polypeptide; viii) an amino acid corresponding to D152 of DRB1 would be D162 of a mature DRB5 polypeptide; and ix) an amino acid corresponding to W153 of DRB1 would be W163 of a mature DRB5 polypeptide.

### DRB1 Polypeptides

In some cases, a suitable MHC class II β chain polypeptide is a DRB1 polypeptide. In an embodiment, a DRB1 polypeptide can have at least 60%, at least 70%, at least 80%, at least 90%, at least 95%, at least 98%, at least 99%, or 100%, amino acid sequence identity with amino acids 30-227 of any DRB1 amino acid sequence depicted in FIG. 7, which displays the DRB1 precursor proteins in which amino acids 1-29 are the signal sequence, 30-124 form the β1 region, 125-227 form the β2 region, and 228-250 form the transmembrane region. In some cases, a DRB1 polypeptide suitable for inclusion in a TMMP of the present disclosure comprises an amino acid substitution, relative to a wild-type DRB1 polypeptide, where the amino acid substitution replaces an amino acid (other than a Cys) with a Cys.

In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-3 (DRB1*03:01) beta chain amino acid sequence sp P01912.2 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-4 (DRB1*04:01) beta chain amino acid sequence sp P13760.1 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-8 (DRB1*08:01) beta chain amino acid sequence sp Q30134.2 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-9 (DRB1*09:01) beta chain amino acid sequence sp Q9TQE0.1 in FIG. 7. In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DRB1-16 (DRB1*16:01) beta chain amino acid sequence sp Q29974.1 (also HLA00876) in FIG. 7. In some cases, the DRB1 β chain polypeptide has a length of about 198 amino acids (e.g., 195, 196, 197, 198, 199, 200, 201, or 202 amino acids).

A “DRB1 polypeptide” includes allelic variants, e.g., naturally occurring allelic variants. Thus, in some cases, a suitable DRB1 polypeptide comprises the following amino acid sequence: DTRPRFLEQVKHECHFFNGTERVRFLDRYFYHQEEYVRFDSDVGEYRAVTELGRPDAEYWNSQ KDLLEQKRAAVDTYCRHNYGVGESFTVQRRVYPEVTVYPAKTQPLQHHNLLVCSVNGFYPGSI EVRWFRNGQEEKTGVVSTGLIQNGDWTFQTLVMLETVPRSGEVYTCQVEHPSLTSPLTVEWRA RSESAQSK (SEQ ID NO:34) (amino acids 31-227 of DRB1-4, see FIG. 7), or an allelic variant thereof.

A suitable DRB1 polypeptide can comprise an amino acid sequence having at least 60%, at least 70%, at least 80%, at least 90%, at least 95%, at least 98%, at least 99%, or 100%, amino acid sequence identity to the following amino acid sequence: GDTRPRFLEQVKHECHFFNGTERVRFLDRYFYHQEEYVRFDSDVGEYRAVTELGRPDAEYWNS QKDLLEQKRAAVDTYCRHNYGVGESFTVQRRVYPEVTVYPAKTQPLQHHNLLVCSVNGFYPA SIEVRWFRNGQEEKTGVVSTGLIQNGDWTFQTLVMLETVPRSGEVYTCQVEHPSLTSPLTVEWR ARSESAQSKM (SEQ ID NO:30); and can have a length of from 195 to 205 amino acids, e.g . . . 199 amino acids.

A suitable DRB1 polypeptide can comprise the following amino acid sequence: GDTRCRFLEQVKHECHFFNGTERVRFLDRYFYHQEEYVRFDSDVGEYRAVTELGRPDAEYWNS QKDLLEQKRAAVDTYCRHNYGVGESFTVQRRVYPEVTVYPAKTQPLQHHNLLVCSVNGFYPA SIEVRWFRNGQEEKTGVVSTGLIQNGDWTFQTLVMLETVPRSGEVYTCQVEHPSLTSPLTVEWR ARSESAQSKM (SEQ ID NO:35), where P5 is substituted with a Cys (shown in bold text).

A suitable DRB1 polypeptide can comprise the following amino acid sequence: GDTRPRFLEQVKHECHFFNGTERVRFLDRYFYHQEEYVREDSDVGEYRAVTELGRPDAEYWNS QKDLLEQKRAAVDTYCRHNYGVGESFTVQRRVYPEVTVYPAKTQPLQHHNLLVCSVNGFYPA SIEVRWFRNGQEEKTGVVSTGLIQNCDWTFQTLVMLETVPRSGEVYTCQVEHPSLTSPLTVEWR ARSESAQSKM (SEQ ID NO:36), where G151 is substituted with a Cys (shown in bold text).

A suitable DRB1 polypeptide can comprise the following amino acid sequence: GDTRPRFLEQVKHECHFFNGTERVRFLDRYFYHQEEYVREDSDVGEYRAVTELGRPDAEYWNS QKDLLEQKRAAVDTYCRHNYGVGESFTVQRRVYPEVTVYPAKTQPLQHHNLLVCSVNGFYPA SIEVRWFRNGQEEKTGVVSTGLIQNGDCTFQTLVMLETVPRSGEVYTCQVEHPSLTSPLTVEWR ARSESAQSKM (SEQ ID NO:37), where W153 is substituted with a Cys (shown in bold text).

A suitable DRB1 β1 domain comprises an amino acid sequence having at least 60%, at least 70%, at least 80%, at least 90%, at least 95%, at least 98%, at least 99%, or 100%, amino acid sequence identity to the following amino acid sequence: DTRPRFLEQVKHECHFFNGTERVRFLDRYFYHQEEYVREDSDVGEYRAVTELGRPDAEYWNSQ KDLLEQKRAAVDTYCRHNYGVGESFTVQRRV (SEQ ID NO:38); and can have a length of about 95 amino acids (e.g., 92, 93, 94, 95, 96, 97, or 98 amino acids). A suitable DRB1 β1 domain can comprise the following amino acid sequence: DTRPRFLEQVKHECHFFNGTERVRFLDRYFYHQEEYVRFDSDVGEYRAVTELGRPDAEYWNSQ KDLLEQKRAAVDTYCRHNYGVGESFTVQRRV (SEQ ID NO:38), or a naturally-occurring allelic variant. A suitable DRB1 β1 domain can comprise the following amino acid sequence: GDTRCRFLEQVKHECHFFNGTERVRFLDRYFYHQEEYVRFDSDVGEYRAVTELGRPDAEYWNS QKDLLEQKRAAVDTYCRHNYGVGESFTVQRRV (SEQ ID NO:39), where P5 is substituted with a Cys (shown in bold text).

A suitable DRB1 β2 domain comprises an amino acid sequence having at least 60%, at least 70%, at least 80%, at least 90%, at least 95%, at least 98%, at least 99%, or 100%, amino acid sequence identity to the following amino acid sequence: YPEVTVYPAKTQPLQHHNLLVCSVNGFYPGSIEVRWFRNGQEEKTGVVSTGLIQNGDWTFQTL VMLETVPRSGEVYTCQVEHPSLTSPLTVEWRARSESAQSK (SEQ ID NO:40); and can have a length of about 103 amino acids (e.g., 100, 101, 102, 103, 104, 105, or 106 amino acids). A suitable DRB1 β2 domain can comprise the following amino acid sequence: YPEVTVYPAKTQPLQHHNLLVCSVNGFYPGSIEVRWFRNGQEEKTGVVSTGLIQNGDWTFQTL VMLETVPRSGEVYTCQVEHPSLTSPLTVEWRARSESAQSK (SEQ ID NO:40), or a naturally-occurring allelic variant thereof. A suitable DRB1 β2 domain can comprise the following amino acid sequence: YPEVTVYPAKTQPLQHHNLLVCSVNGFYPASIEVRWFRNGQEEKTGVVSTGLIQNCDWTFQTL VMLETVPRSGEVYTCQVEHPSLTSPLTVEWRARSESAQSKM (SEQ ID NO:41), where G151 is substituted with a Cys (shown in bold text). A suitable DRB1 β2 domain can comprise the following amino acid sequence: YPEVTVYPAKTQPLQHHNLLVCSVNGFYPASIEVRWFRNGQEEKTGVVSTGLIQNGDCTFQTLV MLETVPRSGEVYTCQVEHPSLTSPLTVEWRARSESAQSKM (SEQ ID NO:42), where W153 is substituted with a Cys (shown in bold text).

### DRB3 Polypeptides

In some cases, a suitable MHC class II β chain polypeptide is a DRB3 polypeptide. In an embodiment, a DRB3 polypeptide can have at least 60%, at least 70%, at least 80%, at least 90%, at least 95%, at least 98%, at least 99%, or 100%, amino acid sequence identity with amino acids 30-227 of any DRB3 amino acid sequence depicted in FIG. 7B, which displays the DRB3 precursor proteins in which amino acids 1-29 are the signal sequence (underlined), 30-124 form the β1 region (shown bolded), 125-227 for the β2 region, and 228-250 the transmembrane region. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DRB1-3 (DRB3*01:01) beta chain amino acid sequence GenBank NP_072049.1 in FIG. 7B. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DRB1-3 beta chain amino acid sequence in GenBank accession EAX03632.1 in FIG. 7B. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DRB1-3 (DRB3*02:01) beta chain amino acid sequence GenBank CAA23781.1 in FIG. 7B. In an embodiment, a DRB3 β chain polypeptide can have at least 60%, at least 70%, at least 80%, at least 90%, at least 95%, at least 98%, at least 99%, or 100%, amino acid sequence identity with amino acids 30-227 of the DRB1-3 (DRB3*03:01) beta chain amino acid sequence GenBank AAN15205.1 in FIG. 7B. In some cases, a DRB3 polypeptide suitable for inclusion in a TMMP of the present disclosure comprises an amino acid substitution, relative to a wild-type DRB3 polypeptide, where the amino acid substitution replaces an amino acid (other than a Cys) with a Cys.

A “DRB3 polypeptide” includes allelic variants, e.g., naturally occurring allelic variants. Thus, in some cases, a suitable DRB3 polypeptide comprises the following amino acid sequence: DTRPRFLELR KSECHFENGT ERVRYLDRYF HNQEEFLRED SDVGEYRAVT ELGRPVAESW NSQKDLLEQK RGRVDNYCRH NYGVGESFTV QRRVHPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:43), or an allelic variant thereof. In some cases, a DRB3 polypeptide suitable for inclusion in a TMMP of the present disclosure comprises an amino acid substitution, relative to a wild-type DRB3 polypeptide, where the amino acid substitution replaces an amino acid (other than a Cys) with a Cys. Thus, e.g., in some cases, the MHC class II β chain polypeptide is a variant DRB3 MHC class II polypeptide that comprises a non-naturally occurring Cys residue; e.g., where the variant DRB3 MHC class II polypeptide comprises an amino acid substitution selected from the group consisting of P5C, F7C, L10C, N19C, G20C, N33C, G151C, D152C, and W153C (of a mature DRB3 polypeptide (lacking the N-terminal signal peptide MVCLKLPGGSSLAALTVTLMVLSSRLAFA (SEQ ID NO:31) depicted in FIG. 7B).

A suitable DRB3 β1 domain comprises an amino acid sequence having at least 60%, at least 70%, at least 80%, at least 90%, at least 95%, at least 98%, at least 99%, or 100%, amino acid sequence identity to the following amino acid sequence: DTRPRFLELR KSECHFENGT ERVRYLDRYF HNQEEFLRFD SDVGEYRAVT ELGRPVAESW NSQKDLLEQK RGRVDNYCRH NYGVGESFTV QRRV (SEQ ID NO:44); and can have a length of about 95 amino acids (e.g., 93, 94, 95, 96, 97, or 98 amino acids). A suitable DRB3 β1 domain can comprise the following amino acid sequence: DTRPRFLELR KSECHFENGT ERVRYLDRYF HNQEEFLRFD SDVGEYRAVT ELGRPVAESW NSQKDLLEQK RGRVDNYCRH NYGVGESFTV QRRV (SEQ ID NO:44), or a naturally-occurring allelic variant.

A suitable DRB3 β2 domain comprises an amino acid sequence having at least 60%, at least 70%, at least 80%, at least 90%, at least 95%, at least 98%, at least 99%, or 100%, amino acid sequence identity to the following amino acid sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:45); and can have a length of about 103 amino acids (e.g., 100, 101, 102, 103, 104, or 105 amino acids). A suitable DRB3 β2 domain can comprise the following amino acid sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:45), or a naturally-occurring allelic variant thereof.

### DRB4 Polypeptides

In some cases, a suitable MHC class II β chain polypeptide is a DRB4 polypeptide. A DRB4 polypeptide can have at least 60%, at least 70%, at least 80%, at least 90%, at least 95%, at least 98%, at least 99%, or 100%, amino acid sequence identity with amino acids 30-227 of the DRB4 amino acid sequence depicted in FIG. 7C. In some cases, the DRB4 polypeptide has a length of about 198 amino acids (e.g., 195, 196, 197, 198, 199, 200, 201, or 202 amino acids). In some cases, a DRB4 polypeptide suitable for inclusion in a TMMP of the present disclosure comprises an amino acid substitution, relative to a wild-type DRB4 polypeptide, where the amino acid substitution replaces an amino acid (other than a Cys) with a Cys.

A “DRB4 polypeptide” includes allelic variants, e.g., naturally occurring allelic variants. Thus, in some cases, a suitable DRB4 polypeptide comprises the following amino acid sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRVQPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO:46), or an allelic variant thereof. In some cases, a DRB4 polypeptide suitable for inclusion in a TMMP of the present disclosure comprises an amino acid substitution, relative to a wild-type DRB4 polypeptide, where the amino acid substitution replaces an amino acid (other than a Cys) with a Cys. Thus, e.g., in some cases, the MHC class II β chain polypeptide is a variant DRB4 MHC class II polypeptide that comprises a non-naturally occurring Cys residue; e.g., where the variant DRB4 MHC class II polypeptide comprises an amino acid substitution selected from the group consisting of P15C. F17C, Q20C. N29C, G30C, N43C, G161C, D162C, and W163C (of a mature DRB4 polypeptide (lacking the N-terminal signal peptide MVCLKLPGGSCMAALTVTL (SEQ ID NO:32) depicted in FIG. 7C).

A suitable DRB4 β1 domain comprises an amino acid sequence having at least 60%, at least 70%, at least 80%, at least 90%, at least 95%, at least 98%, at least 99%, or 100%, amino acid sequence identity to the following amino acid sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRV (SEQ ID NO:47); and can have a length of about 95 amino acids (e.g., 93, 94, 95, 96, 97, or 98 amino acids). A suitable DRB4 β1 domain can comprise the following amino acid sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRV (SEQ ID NO:47), or a naturally-occurring allelic variant.

A suitable DRB4 β2 domain comprises an amino acid sequence having at least 60%, at least 70%, at least 80%, at least 90%, at least 95%, at least 98%, at least 99%, or 100%, amino acid sequence identity to the following amino acid sequence: QPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO:48); and can have a length of about 103 amino acids (e.g., 100, 101, 102, 103, 104, or 105 amino acids). A suitable DRB4 β2 domain can comprise the following amino acid sequence: QPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO:48), or a naturally-occurring allelic variant thereof.

### DRB5 Polypeptides

In some cases, a suitable MHC class II β chain polypeptide is a DRB5 polypeptide. A DRB5 polypeptide can have at least 60%, at least 70%, at least 80%, at least 90%, at least 95%, at least 98%, at least 99%, or 100%, amino acid sequence identity with amino acids 30-227 of the DRB5 amino acid sequence depicted in FIG. 7D. In some cases, the DRB5 polypeptide has a length of about 198 amino acids (e.g., 195, 196, 197, 198, 199, 200, 201, or 202 amino acids). In some cases, a DRB5 polypeptide suitable for inclusion in a TMMP of the present disclosure comprises an amino acid substitution, relative to a wild-type DRB5 polypeptide, where the amino acid substitution replaces an amino acid (other than a Cys) with a Cys.

A “DRB5 polypeptide” includes allelic variants, e.g., naturally occurring allelic variants. Thus, in some cases, a suitable DRB5 polypeptide comprises the following amino acid sequence: M VLSSPLALAG DTRPRFLQQD KYECHFENGT ERVRFLHRDI YNQEEDLRFD SDVGEYRAVT ELGRPDAEYW NSQKDFLEDR RAAVDTYCRH NYGVGESFTV QRRVEPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO:49), or an allelic variant thereof. In some cases, a DRB5 polypeptide suitable for inclusion in a TMMP of the present disclosure comprises an amino acid substitution, relative to a wild-type DRB5 polypeptide, where the amino acid substitution replaces an amino acid (other than a Cys) with a Cys.

Thus, e.g., in some cases, the MHC class II β chain polypeptide is a variant DRB5 MHC class II polypeptide that comprises a non-naturally occurring Cys residue; e.g., where the variant DRB5 MHC class II polypeptide comprises an amino acid substitution selected from the group consisting of P15C. F17C, Q20C, N29C, G30C. N43C, G161C, D162C, and W163C (of a mature DRB5 polypeptide (lacking the N-terminal signal peptide MVCLKLPGGSYMAKLTVTL (SEQ ID NO:33) depicted in FIG. 7D).

A suitable DRB5 β1 domain comprises an amino acid sequence having at least 60%, at least 70%, at least 80%, at least 90%, at least 95%, at least 98%, at least 99%, or 100%, amino acid sequence identity to the following amino acid sequence: M VLSSPLALAG DTRPRFLQQD KYECHFFNGT ERVRFLHRDI YNQEEDLRFD SDVGEYRAVT ELGRPDAEYW NSQKDFLEDR RAAVDTYCRH NYGVGESFTV QRRV (SEQ ID NO:50); and can have a length of about 95 amino acids (e.g., 93, 94, 95, 96, 97, or 98 amino acids). A suitable DRB5 β1 domain can comprise the following amino acid sequence: M VLSSPLALAG DTRPRFLQQD KYECHFENGT ERVRFLHRDI YNQEEDLRFD SDVGEYRAVT ELGRPDAEYW NSQKDFLEDR RAAVDTYCRH NYGVGESFTV QRRV (SEQ ID NO:50), or a naturally-occurring allelic variant.

A suitable DRB5 β2 domain comprises an amino acid sequence having at least 60%, at least 70%, at least 80%, at least 90%, at least 95%, at least 98%, at least 99%, or 100%, amino acid sequence identity to the following amino acid sequence: EPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO:51); and can have a length of about 103 amino acids (e.g., 100, 101, 102, 103, 104, or 105 amino acids). A suitable DRB5 β2 domain can comprise the following amino acid sequence: EPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO:51), or a naturally-occurring allelic variant thereof.

### DPB1 Polypeptides

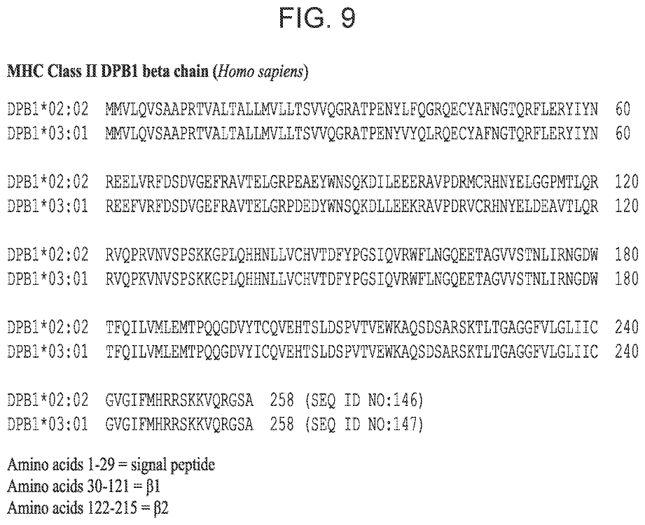

In some cases, a suitable MHC class II β chain polypeptide is a DPB1 polypeptide. A DPB1 polypeptide can have at least 60%, at least 70%, at least 80%, at least 90%, at least 95%, at least 98%, at least 99%, or 100%, amino acid sequence identity with amino acids 30-215 of any of the DPB1 amino acid sequences depicted in FIG. 9. In some cases, the DPB1 polypeptide has a length of about 186 amino acids (e.g., 184, 185, 186, 187, or 188 amino acids). In an embodiment, a DRB1 β chain polypeptide can have at least 60%, at least 70%, at least 80%, at least 90%, at least 95%, at least 98%, at least 99%, or 100%, amino acid sequence identity with amino acids 30-227 of the DPB1*02:02 β chain amino acid sequence in FIG. 9, IMGT/HLA Acc No. HLA00519. In an embodiment, a DPB1 DRB3 β chain polypeptide can have at least 60%, at least 70%, at least 80%, at least 90%, at least 95%, at least 98%, at least 99%, or 100%, amino acid sequence identity with amino acids 30-227 of the DPB1*03:01 β chain amino acid sequence in FIG. 9, IMGT/HLA Acc No.: HLA00520.

A “DPB1 polypeptide” includes allelic variants, e.g., naturally occurring allelic variants. Thus, in some cases, a suitable DPB1 polypeptide comprises the following amino acid sequence: R ATPENYLFQG RQECYAFNGT QRFLERYIYN REEFARFDSD VGEFRAVTEL GRPAAEYWNS QKDILEEKRA VPDRMCRHNY ELGGPMTLQR RVQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO:52), or an allelic variant thereof. In some cases, a DPB1 polypeptide suitable for inclusion in a TMMP of the present disclosure comprises an amino acid substitution, relative to a wild-type DPB1 polypeptide, where the amino acid substitution replaces an amino acid (other than a Cys) with a Cys.

A suitable DPB1 β1 domain comprises an amino acid sequence having at least 60%, at least 70%, at least 80%, at least 90%, at least 95%, at least 98%, at least 99%, or 100%, amino acid sequence identity to the following amino acid sequence: R ATPENYLFQG RQECYAFNGT QRFLERYIYN REEFARFDSD VGEFRAVTEL GRPAAEYWNS QKDILEEKRA VPDRMCRHNY ELGGPMTLQR R (SEQ ID NO:53); and can have a length of about 92 amino acids (e.g., 90, 91, 92, 93, or 94 amino acids). A suitable DPB1 β1 domain can comprise the following amino acid sequence: R ATPENYLFQG RQECYAFNGT QRFLERYIYN REEFARFDSD VGEFRAVTEL GRPAAEYWNS QKDILEEKRA VPDRMCRHNY ELGGPMTLQR R (SEQ ID NO:53), or a naturally-occurring allelic variant.

A suitable DPB1 β2 domain comprises an amino acid sequence having at least 60%, at least 70%, at least 80%, at least 90%, at least 95%, at least 98%, at least 99%, or 100%, amino acid sequence identity to the following amino acid sequence: VQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO:54); and can have a length of about 94 amino acids (e.g., 92, 93, 94, 95, 96, or 97 amino acids). A suitable DPB1 β2 domain can comprise the following amino acid sequence: VQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO:54), or a naturally-occurring allelic variant thereof.

### DQB1 Polypeptides

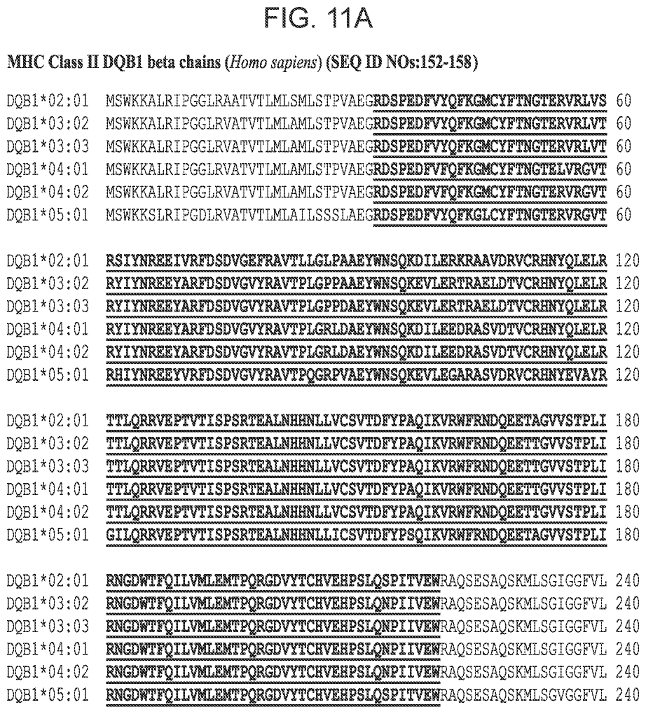

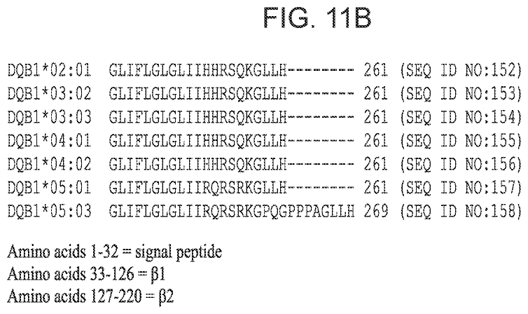

In some cases, a suitable MHC class II β chain polypeptide is a DQB1 polypeptide. A DQB1 polypeptide can have at least 60%, at least 70%, at least 80%, at least 90%, at least 95%, at least 98%, at least 99%, or 100%, amino acid sequence identity with amino acids 33-220 of any one of the DQB1 amino acid sequences depicted in FIG. 11A-11B. In some cases, the DQB1 polypeptide has a length of about 188 amino acids (e.g., 186, 187, 188, 190, 191, or 192 amino acids). In some cases, a DQB1 polypeptide suitable for inclusion in a TMMP of the present disclosure comprises an amino acid substitution, relative to a wild-type DQB1 polypeptide, where the amino acid substitution replaces an amino acid (other than a Cys) with a Cys.

A “DQB1 polypeptide” includes allelic variants, e.g., naturally occurring allelic variants. Thus, in some cases, a suitable DQB1 polypeptide comprises the following amino acid sequence: RDSPEDFV FQFKGMCYFT NGTERVRLVT RYIYNREEYA RFDSDVGVYR AVTPQGRPDA EYWNSQKEVL EGTRAELDTV CRHNYEVAFR GILQRRVEPT VTISPSRTEA LNHHNLLVCS VTDFYPGQIK VRWFRNDQEE TAGVVSTPLI RNGDWTFQIL VMLEMTPQRG DVYTCHVEHP SLQSPITVEW (SEQ ID NO:55), or an allelic variant thereof. In some cases, a DQB1 polypeptide suitable for inclusion in a TMMP of the present disclosure comprises an amino acid substitution, relative to a wild-type DQB1 polypeptide, where the amino acid substitution replaces an amino acid (other than a Cys) with a Cys.

A suitable DQB1 β1 domain comprises an amino acid sequence having at least 60%, at least 70%, at least 80%, at least 90%, at least 95%, at least 98%, at least 99%, or 100%, amino acid sequence identity to the following amino acid sequence: RDSPEDFV FQFKGMCYFT NGTERVRLVT RYIYNREEYA RFDSDVGVYR AVTPQGRPDA EYWNSQKEVL EGTRAELDTV CRHNYEVAFR GILQRR (SEQ ID NO:56); and can have a length of about 94 amino acids (e.g., 92, 93, 94, 95, or 96 amino acids). A suitable DQB1 β1 domain can comprise the following amino acid sequence: RDSPEDFV FQFKGMCYFT NGTERVRLVT RYIYNREEYA RFDSDVGVYR AVTPQGRPDA EYWNSQKEVL EGTRAELDTV CRHNYEVAFR GILQRR (SEQ ID NO:56), or a naturally-occurring allelic variant.

A suitable DQB1 β2 domain comprises an amino acid sequence having at least 60%, at least 70%, at least 80%, at least 90%, at least 95%, at least 98%, at least 99%, or 100%, amino acid sequence identity to the following amino acid sequence: VEPT VTISPSRTEA LNHHNLLVCS VTDFYPGQIK VRWFRNDQEE TAGVVSTPLI RNGDWTFQIL VMLEMTPQRG DVYTCHVEHP SLQSPITVEW (SEQ ID NO:57); and can have a length of about 94 amino acids (e.g., 92, 93, 94, 95, or 96 amino acids). A suitable DQB1 β2 domain can comprise the following amino acid sequence: VEPT VTISPSRTEA LNHHNLLVCS VTDFYPGQIK VRWFRNDQEE TAGVVSTPLI RNGDWTFQIL VMLEMTPQRG DVYTCHVEHP SLQSPITVEW (SEQ ID NO:57), or a naturally-occurring allelic variant thereof.

In an embodiment, a DQB β chain polypeptide can have at least 60%, at least 70%, at least 80%, at least 90%, at least 95%, at least 98%, at least 99%, or 100%, amino acid sequence identity with amino acids 30-227 of the DQB1*02:01 beta chain amino acid sequence in FIG. 11A-11B, IMGT/HLA Acc No: HLA00622.

In an embodiment, a DQB β chain polypeptide can have at least 60%, at least 70%, at least 80%, at least 90%, at least 95%, at least 98%, at least 99%, or 100%, amino acid sequence identity with amino acids 30-227 of the DQB1*03:02 beta chain amino acid sequence in FIG. 11A-11B, IMGT/HLA Acc No: HLA00627.

In an embodiment, a DQB β chain polypeptide can have at least 60%, at least 70%, at least 80%, at least 90%, at least 95%, at least 98%, at least 99%, or 100%, amino acid sequence identity with amino acids 30-227 of the DQB1*03:03 beta chain amino acid sequence in FIG. 11A-11B, IMGT/HLA Acc No: HLA00629.

In an embodiment, a DQB β chain polypeptide can have at least 60%, at least 70%, at least 80%, at least 90%, at least 95%, at least 98%, at least 99%, or 100%, amino acid sequence identity with amino acids 30-227 of the DQB1*05:01 beta chain amino acid sequence in FIG. 11A-11B, IMGT/HLA Acc No: HLA00638.

### Disease Risk-Associated Alleles and Haplotypes

Certain alleles and haplotypes of MHC class II have been associated with disease, e.g., increased risk of developing a particular disease. See, e.g., Erlich et al. (2008) Diabetes 57:1084; Gough and Simmonds (2007) Curr. Genomics 8:453; Mitchell et al. (2007) Robbins Basic Pathology Philadelphia: Saunders, 8^{th} ed.; Margaritte-Jeannin et al. (2004) Tissue Antigens 63:562; and Kurko et al. (2013) Clin. Rev. Allergy Immunol. 45:170.

### MHC Class II Polypeptides in Type 1 Diabetes Mellitus (T1D)

Alleles/isoforms showing increased association with T1D represent suitable sources of MHC II α1, α2, β1, and β2 polypeptide sequences for incorporation into TMMPs directed to the treatment of T1D. T1D is associated with alleles belonging to the HLA-DR3 and HLA-DR4 haplotypes/serotypes, with the strongest risk associated with the HLA-DQ8, (e.g., HLA-DQB1*03:02) and alleles of the HLA-DQ2 serotype. Some high and moderate risk haplotypes and their association with various DR serotypes are shown in Table 1, below, which is adapted from Kantárová and Buc, Physiol. Res. 56: 255-266 (2007).

TABLE 1

| | | | | |
|---|---|---|---|---|
| | DRB | DQ | DQA | DQB |
| Haplotype | allele | serotype | allele | allele |

High risk T1D haplotypes

| | | | | |
|---|---|---|---|---|
| DR3 | DRB1*0301 | DQ 2.5 | DQA1*0501 | DQB1*0201 |
| DR4 | DRB1*0401 | DQ 8.1 | DQA1*0301 | DQB1*0302 |
| DR4 | DRB1*0402 | DQ 8.1 | DQA1*0301 | DQB1*0302 |
| DR4 | DRB1*0405 | DQ 8.1 | DQA1*0301 | DQB1*0302 |

Moderate risk T1D haplotypes

| | | | | |
|---|---|---|---|---|
| DR1 | DRB1*01 | DQ 5 | DQA1*0101 | DQB1*0501 |
| DR8 | DRB1*0801 | | DQA1*0401 | DQB1*0402 |
| DR9 | DRB1*0901 | | DQA1*0301 | DQB1*0303 |

Recognized associations

DPB allele
DPB1*02:02
DPB1*03:01

The stereotypically defined DR3 and DR4 protein isoforms/haplotypes of the DRB1 gene are associated with increased risk that an individual expressing such alleles will develop T1D. The DR3 serotype includes the alleles encoding the DRB1*03:01, *03:02, *03:03, and *03:04 proteins, with the HLA-DRB1*0301 allele often found associated with a predisposition to T1D. The DR4 serotype includes the alleles encoding the DRB1*04:01, *04:02, *04:03, *04:04, *04:05, *04:06, *04:07, *04:08, *04:09, *04:10, *04:11, *04:12, and *04:13 proteins. Certain HLA-DR4 alleles (e.g., HLA-DRB1*0401, HLA-DRB1*0402, and HLA-DRB1*0405) predispose individuals to T1D, whereas HLA-DRB1*04:03 allele/isoform may afford protection. DRB1*16:01 also show an increased frequency in diabetic children relative to healthy controls (Deja, et al., Mediators of Inflammation 2006:1-7 (2006)).

While T1D is associated with DR3 and DR4 alleles as discussed above, among the strongest associated risk factors for T1D, the presence of alleles belonging to the HLA-DQ8 serotype (e.g., the HLA-DQB1*03:02 isoform) and the HLA DQ2 serotype (e.g., HLA-DQB1*0201). HLA-DQ8 T1D susceptible serotypes include the HLA-DQ8.1 serotype (HLA-DQA1*03:01/DQB1* 03:02), The HLA-DQ2 serotype (HLA-DQB1*02) associated with T1D include DQB1*02:01). Jones, et al., Nat. Rev. Immunol. 2006, 6: 271-282. By contrast, individuals that carry the HLADQB1* 0602 allele appear to be protected against type 1 diabetes. Id. DQ2 is most common in Western Europe, North Africa and East Africa, with the highest frequencies observed in parts of Spain and Ireland.

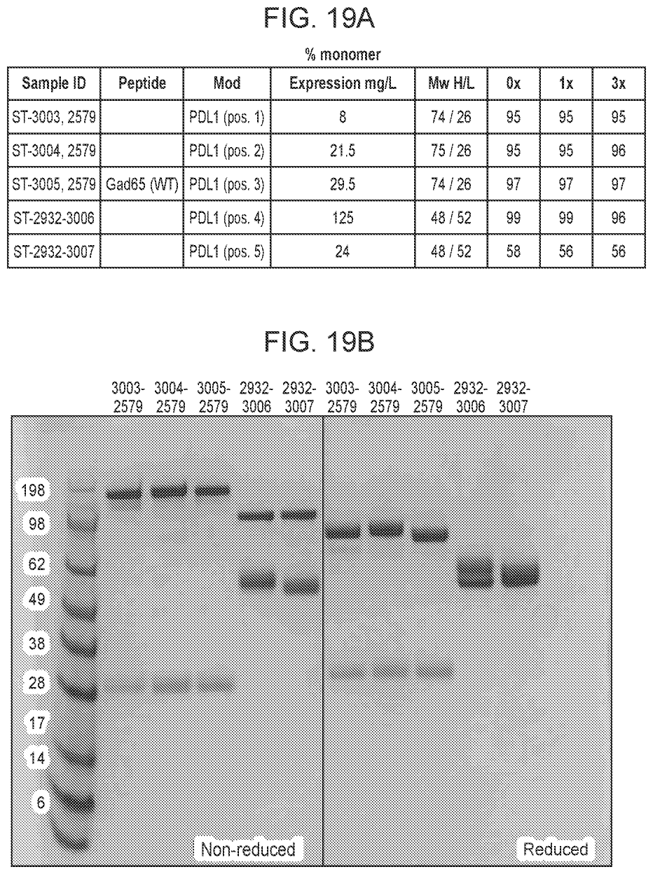

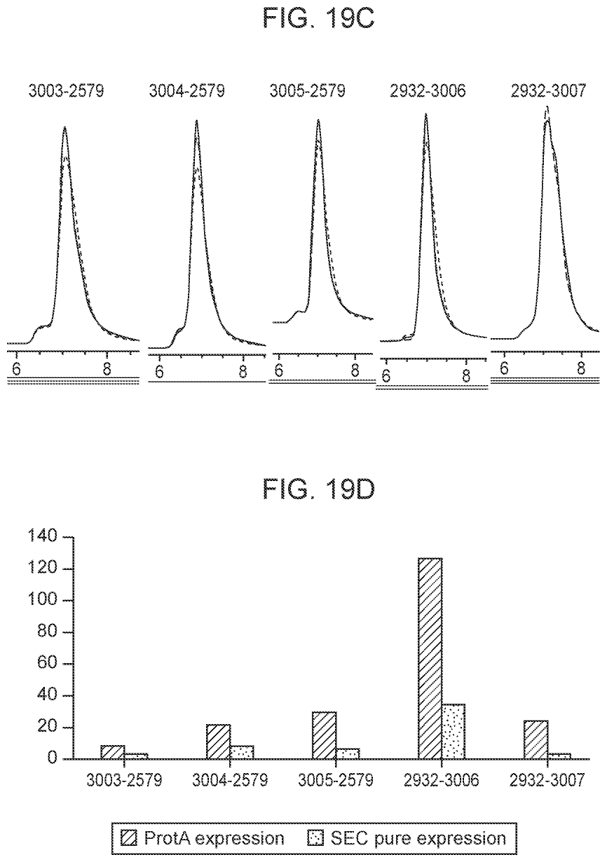

The DQB1 locus alone has also been reported to be associated with T1D when position β57 is a neutral residue such as Ala or Ser. Both the DQ2 and DQ8 serotypes, which are associated with T1D, lack an Asp at the 57β position, and instead have an Ala in its place (see e.g., Ala 89 in FIG. 19B HLA-DQB1*02:01 and FIG. 19C, HLA-DQB1*03:02 respectively) conferred T1D susceptibility. In contrast, DQB1*06:02, which has an Asp) at position β57 of DQB1 (position 89 in FIG. 11A-11B) was found to be associated with resistance to T1D. Jones et al, Nat. Rev. Immunol. 2006, 6: 271-282. Position β57 of the molecule forms a critical residue in peptide binding pocket nine (P9) of the DQB1, which is involved in antigen presentation and T cell receptor (TCR) interaction.

Individuals with the HLA haplotype DQA1*03:01-DRB1*03:02, especially when combined with DQA1*05:01-DRB1*02:01, are highly susceptible (10-20-fold increase) to T1D, see Notkins, A. L., J. Biol. Chem., 2002, 277(46): 43545-48. Among the stereotypically defined groups showing susceptibility to T1D are HLA-DR4.1 (HLA-DRA1*01:01/DRB1*04:01), HLA-DR4.5 (HLA-DRA1*01:01/DRB1*04:05), HLA-DQ2.5 (HLA-DQA1*05:01/DQB1*02:01), and HLA-DQ8.1 (HLA-DQA1*03:01/DQB1*03:02). (see e.g., Jones et al., Nat. Rev. Immunol. 2006, 6: 271-282). The DRβ1*04:05-DQB1*04:01/DRβ1*08:02-DQB1*03:02 genotype has shown to be associated with acute-onset and slow progressive T1D. Fulminant diabetes has been associated with DRβ1*04:05-DQB1*04:01/DRβ1*04:05-DQB1*04:01 genotype, in a Japanese population study Kawabata, et al., Diabetologia 2009, 52:2513-21.

Although the HLA-DR associations with T1D are not as strong as those of HLA-DQ, insulin-reactive T cells derived from lymph nodes draining the pancreas of patients with T1D appear to be HLA-DR4.1 restricted rather than HLA-DQ8 or HLA-DQ2 restricted (Kent et al., Nature 2005 435: 224-228).

The above-mentioned alleles associated with an increased risk of T1D represent suitable candidates from which the α1, α2, β1, and/or β2 polypeptide sequences present in a TMMP of the present disclosure may be taken. In an embodiment, the TMMP is DQ2.5-like with the α1 and α2 polypeptides from DQA1*0501, and the β1 and β2 polypeptides taken from DQB1*0201. In an embodiment, the TMMP is DQ8.1-like with the α1 and α2 polypeptides from DQA1*0301, and the β1 and β2 polypeptides taken from DQB1*0302.

### Scaffold Polypeptides

A TMMP of the present disclosure, whether multimeric or monomeric, can comprise an immunoglobulin or non-immunoglobulin scaffold. A TMMP polypeptide of the present disclosure, whether multimeric or monomeric, can comprise an Fc polypeptide, or can comprise another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, an Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly; SEQ ID NO:236), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

Suitable scaffold polypeptides will in some cases be half-life extending polypeptides. Thus, in some cases, a suitable scaffold polypeptide increases the in vivo half-life (e.g., the serum half-life) of the multimeric polypeptide, compared to a control multimeric polypeptide lacking the scaffold polypeptide. For example, in some cases, a scaffold polypeptide increases the in vivo half-life (e.g., the serum half-life) of the multimeric polypeptide, compared to a control multimeric polypeptide lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, an Fc polypeptide increases the in vivo half-life (e.g., the serum half-life) of the multimeric polypeptide, compared to a control multimeric polypeptide lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

### Fc Polypeptides

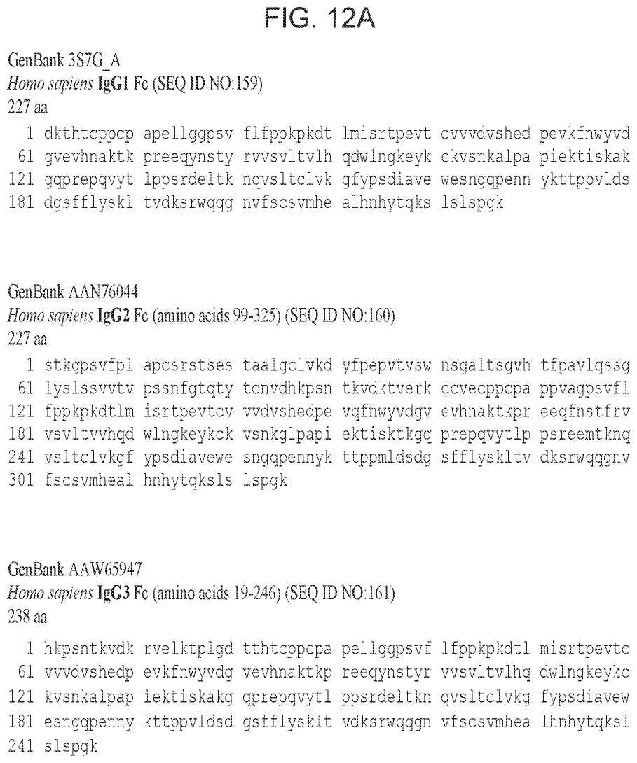

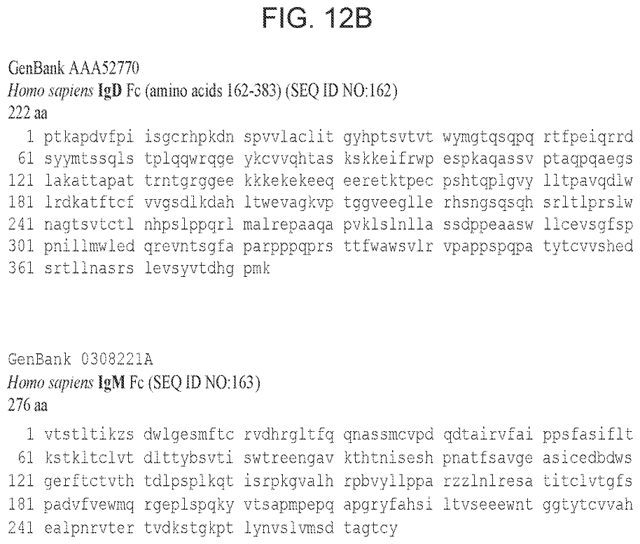

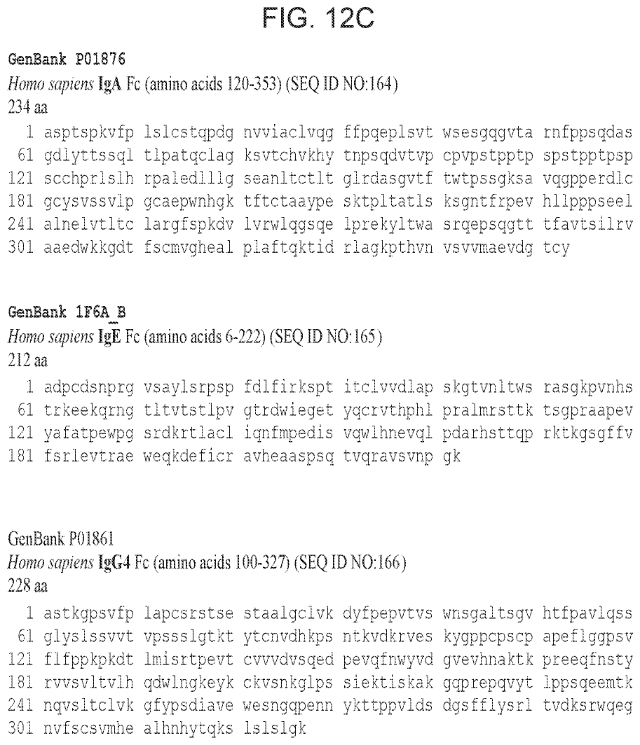

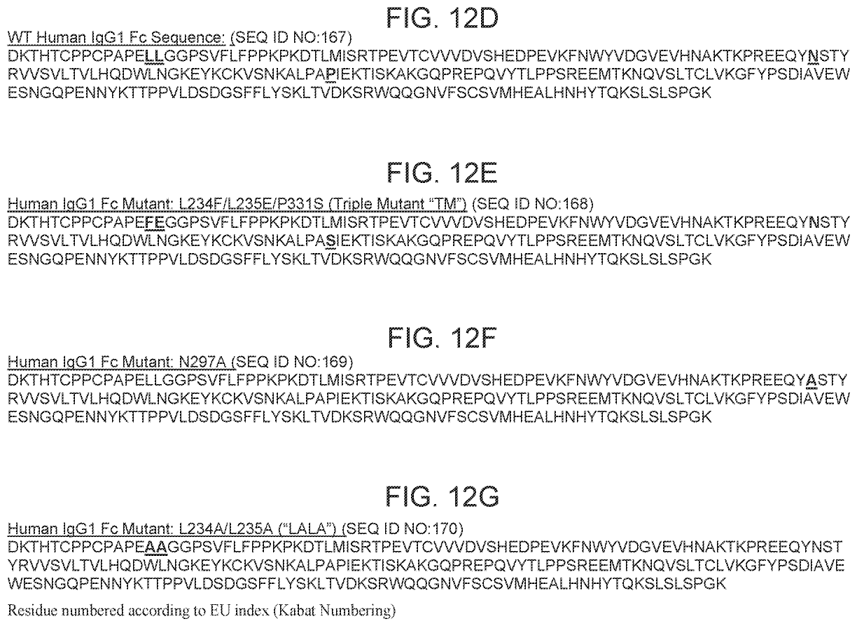

In some cases, the first or the second polypeptide chain of a TMMP of the present disclosure comprises an Fc polypeptide. The Fc polypeptide of a TMMP of the present disclosure can be a human IgG1 Fc, a human IgG2 Fc, a human IgG3 Fc, a human IgG4 Fc, etc. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to an amino acid sequence of an Fc region depicted in FIG. 12A-12G. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 12A. In some cases, the Fc region comprises an amino acid sequence having at least about 70%, at least about 75%, at least about 80%, at least about 85%, at least about 90%, at least about 95%, at least about 98%, or at least about 99%, amino acid sequence identity to the human IgG1 Fc polypeptide depicted in FIG. 12A, and comprises a substitution of N77; e.g., the Fc polypeptide comprises an N77A substitution.

In some cases, the Ig Fc polypeptide induces cell lysis through activation of complement-dependent cytotoxicity (CDC). In some cases, the Ig Fc polypeptide is a variant that substantially does not induce cell lysis through activation of CDC, e.g., an IgG1 Fc polypeptide comprising L234A and L235A substitutions (L14A and L15A substitution of the amino acid sequence depicted in FIG. 12A). Typically, the TMMPs of this disclosure that employ an Ig Fc polypeptide are meant to engage a target T cell through the MHC-epitope complex and then modulate the activity of a T cell through the immunomodulatory polypeptides, and thus will employ a variant that substantially does not induce cell lysis through activation of CDC.

In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG2 Fc polypeptide depicted in FIG. 12A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 99-325 of the human IgG2 Fc polypeptide depicted in FIG. 12A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG3 Fc polypeptide depicted in FIG. 12A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 19-246 of the human IgG3 Fc polypeptide depicted in FIG. 12A.

In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgM Fc polypeptide depicted in FIG. 12B; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-276 to the human IgM Fc polypeptide depicted in FIG. 2B.

In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgA Fc polypeptide depicted in FIG. 12C; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-234 to the human IgA Fc polypeptide depicted in FIG. 12C.

In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG4 Fc polypeptide depicted in FIG. 12C. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 100 to 327 of the human IgG4 Fc polypeptide depicted in FIG. 12C.

In some cases, the IgG4 Fc polypeptide comprises the following amino acid sequence:

(SEQ ID NO: 58)
PPCPSCPAPEFLGGPSVFLFPPKPKDTLMISRTPEVTCVVVDVSQEDPEV
QFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVF
SCSVMHEALHNHYTQKSLSLSPG.

In some cases, the Fc polypeptide present in a TMMP comprises the amino acid sequence depicted in FIG. 12A (human IgG1 Fc). In some cases, the Fc polypeptide present in a TMMP comprises the amino acid sequence depicted in FIG. 12A (human IgG1 Fc), except for a substitution of N297 (N77 of the amino acid sequence depicted in FIG. 12A) with an amino acid other than asparagine. In some cases, the Fc polypeptide present in a TMMP comprises the amino acid sequence depicted in FIG. 12C (human IgG1 Fc comprising an N297A substitution, which is N77 of the amino acid sequence depicted in FIG. 12A). In some cases, the Fc polypeptide present in a TMMP comprises the amino acid sequence depicted in FIG. 12A (human IgG1 Fc), except for a substitution of L234 (L14 of the amino acid sequence depicted in FIG. 12A) with an amino acid other than leucine. In some cases, the Fc polypeptide present in a TMMP comprises the amino acid sequence depicted in FIG. 12A (human IgG1 Fc), except for a substitution of L235 (L15 of the amino acid sequence depicted in FIG. 2A) with an amino acid other than leucine.

In some cases, the Fc polypeptide present in a TMMP comprises the amino acid sequence depicted in FIG. 12E. In some cases, the Fc polypeptide present in a TMMP comprises the amino acid sequence depicted in FIG. 12F. In some cases, the Fc polypeptide present in a TMMP comprises the amino acid sequence depicted in FIG. 12G (human IgG1 Fc comprising an L234A substitution and an L235A substitution, corresponding to positions 14 and 15 of the amino acid sequence depicted in FIG. 12G). In some cases, the Fc polypeptide present in a TMMP comprises the amino acid sequence depicted in FIG. 12A (human IgG1 Fc), except for a substitution of P331 (P111 of the amino acid sequence depicted in FIG. 12A) with an amino acid other than proline; in some cases, the substitution is a P331S substitution. In some cases, the Fc polypeptide present in a TMMP comprises the amino acid sequence depicted in FIG. 12A (human IgG1 Fc), except for substitutions at L234 and L235 (L14 and L15 of the amino acid sequence depicted in FIG. 12A) with amino acids other than leucine. In some cases, the Fc polypeptide present in a TMMP comprises the amino acid sequence depicted in FIG. 12A (human IgG1 Fc), except for substitutions at L234 and L235 (L14 and L15 of the amino acid sequence depicted in FIG. 12A) with amino acids other than leucine, and a substitution of P331 (P111 of the amino acid sequence depicted in FIG. 12A) with an amino acid other than proline. In some cases, the Fc polypeptide present in a TMMP comprises the amino acid sequence depicted in FIG. 12E (human IgG1 Fc comprising L234F, L235E, and P331S substitutions (corresponding to amino acid positions 14, 15, and 111 of the amino acid sequence depicted in FIG. 12E). In some cases, the Fc polypeptide present in a TMMP is an IgG1 Fc polypeptide that comprises L234A and L235A substitutions (substitutions of L14 and L15 of the amino acid sequence depicted in FIG. 12A with Ala), as depicted in FIG. 12G.

In some cases, a TMMP of the present disclosure comprises an Ig Fc polypeptide comprising the following amino acid sequence:

(SEQ ID NO:59)
DKTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFS
CSVMHEALHNHYTQKSLSLSPG,

which is an IgG1 Fc polypeptide comprising L234A and L235A substitutions (L14A and L15A in this sequence; shown in bold text).

### Linkers

As discussed above, a TMMP of the present disclosure can include one or more linker peptides between components of the first and second polypeptides of the TMMP, e.g., between a T1D peptide and an MHC polypeptide; between an MHC polypeptide and an Ig Fc polypeptide; between a first MHC polypeptide and a second MHC polypeptide; between an immunomodulatory polypeptide and an MHC polypeptide; etc. As also noted above, in some cases, a TMMP of the present disclosure can include a Cys-containing peptide linker between the T1D peptide and an MHC class II polypeptide, e.g., between the T1D peptide and an MHC class II β chain polypeptide. Generally, a Cys-containing peptide linker will be used in either the first or second polypeptide of a TMMP in order to intentionally facilitate formation of a disulfide bond between the linker and a desired site on the other polypeptide. Where a Cys-containing linker is inserted in one polypeptide of a TMMP, the remaining linkers in the TMMP will not include a Cys in order to prevent formation of a disulfide bond at an unwanted site in the TMMP, with the exception that a Cys-containing linker could be used in each of the first and second polypeptides when it is desired to link the first and second polypeptides through a disulfide bond formed between the linkers. A TMMP of the present disclosure thus can include: a) a Cys-containing peptide linker between the T1D peptide and an MHC class II polypeptide, e.g., between the T1D peptide and an MHC class II β chain polypeptide; and b) at least one additional peptide linker, where the at least one additional peptide linker does not include a Cys.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. A suitable linker can be from 25 to 35 amino acids in length. A suitable linker can be 25, 26, 27, 28, 29, 30, 31, 32, 33, 34, or 35 amino acids in length. A suitable linker can be from 35 to 45 amino acids in length. A suitable linker can be 35, 36, 37, 38, 39, 40, 41, 42, 43, 44, or 45 amino acids in length. A suitable linker can be from 45 to 50 amino acids in length. A suitable linker can be 45, 46, 47, 48, 49, or 50 amino acids in length.

### Linkers Containing a Cys

A peptide linker comprising a Cys can comprise an amino acid sequence selected from (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where n is 1, 2, 3, 4, 5, 6, 7, 8, 9, or 10). In some cases, a TMMP of the present disclosure comprises a heterodimer comprising a first and a second polypeptide, where the first polypeptide comprises a T1D peptide and an MHC class II polypeptide (e.g., an MHC class II β chain polypeptide), and comprises a linker between the T1D peptide and the MHC class II polypeptide (e.g., the MHC class II β chain polypeptide), where the linker comprises an amino acid sequence selected from (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where n is 1, 2, 3, 4, 5, 6, 7, 8, 9, or 10).

### Linkers not Containing a Cys

Exemplary linkers include glycine polymers (G)n, glycine-serine polymers (including, for example, (GS)ₙ, (GSGGS)ₙ (SEQ ID NO:60) and (GGGS)ₙ (SEQ ID NO:61), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:62), GGSGG (SEQ ID NO:63), GSGSG (SEQ ID NO:64), GSGGG (SEQ ID NO:65), GGGSG (SEQ ID NO:66), GSSSG (SEQ ID NO:67), and the like. Exemplary linkers can include, e.g., Gly(Sera)n. (SEQ ID NO:68) where n is 1, 2, 3, 4, 5, 6, 7, 8, 9, or 10. In some cases, a linker comprises the amino acid sequence (GSSSS)n (SEQ ID NO:69), where n is 4. In some cases, a linker comprises the amino acid sequence (GSSSS)n (SEQ ID NO:70), where n is 5. Exemplary linkers can include, e.g., (GlyGlyGlyGlySer)n (SEQ ID NO:71) (also referred to as “G4S” linkers), where n is 1, 2, 3, 4, 5, 6, 7, 8, 9, or 10. In some cases, a linker comprises the amino acid sequence (GGGGS)n (SEQ ID NO:72), where n is 1. In some cases, a linker comprises the amino acid sequence (GGGGS)n (SEQ ID NO:73), where n is 2. In some cases, a linker comprises the amino acid sequence (GGGGS)n (SEQ ID NO:74), where n is 3. In some cases, a linker comprises the amino acid sequence (GGGGS)n (SEQ ID NO:75), where n is 4. In some cases, a linker comprises the amino acid sequence (GGGGS)n (SEQ ID NO:76), where n is 5. In some cases, a linker comprises the amino acid sequence (GGGGS)n (SEQ ID NO:77), where n is 6. In some cases, a linker comprises the amino acid sequence (GGGGS)n (SEQ ID NO:78), where n is 7. In some cases, a linker comprises the amino acid sequence (GGGGS)n (SEQ ID NO:79), where n is 8. In some cases, a linker comprises the amino acid sequence (GGGGS)n (SEQ ID NO:80), where n is 9. In some cases, a linker comprises the amino acid sequence (GGGGS)n (SEQ ID NO:81), where n is 10. In some cases, a linker comprises the amino acid sequence AAAGG (SEQ ID NO:82). In some cases, a linker comprises the amino acid sequence GGSAAAGG (SEQ ID NO:83). The AAAGG (SEQ ID NO:82) and GGSAAAGG (SEQ ID NO:83) linkers have been found to be useful for linking an MHC class II alpha chain polypeptide (e.g., a DRA class II polypeptide) to an Ig Fc polypeptide (e.g., a human IgG1 Fc polypeptide sequence depicted in FIG. 12G comprising an L234A substitution and an L235A substitution, corresponding to positions 14 and 15 of the amino acid sequence depicted in FIG. 12G).

### Peptides Presenting T1D-Associated Epitopes

As used herein, a “T1D peptide” is a peptide that, when present in a TMMP of this disclosure, presents a T1D-associated epitope capable of being bound by a TCR on the surface of a T cell. A T1D peptide can have a length of from about 4 amino acids to about 25 amino acids, e.g., the T1D peptide can have a length of from 4 amino acids (aa) to 10 aa, from 8 aa to 12 aa, from 10 aa to 15 aa, from 12 aa to 20 aa, from 15 aa to 20 aa, from 15 aa to 25 aa, or from 20 aa to 25 aa. For example, a T1D peptide present in a TMPP of the present disclosure can have a length of 4 amino acids (aa), 5 aa, 6 aa. 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa. 24 aa, or 25 aa. In some cases, a T1D peptide present in a TMPP of the present disclosure has a length of from 5 amino acids to 10 amino acids, e.g., 5 aa, 6 aa, 7 aa, 8 aa, 9 aa, or 10 aa.

Antigens associated with type 1 diabetes (T1D) include, e.g., preproinsulin, proinsulin, insulin, insulin β chain, insulin A chain, 65 kDa isoform of glutamic acid decarboxylase (GAD65), 67 kDa isoform of glutamic acid decarboxylase (GAD67), tyrosine phosphatase (IA-2), heat-shock protein HSP65, islet-specific glucose6-phosphatase catalytic subunit related protein (IGRP), islet antigen 2 (IA2), and zinc transporter (ZnT8). See, e.g., Mallone et al. (2011) Clin. Dev. Immunol. 2011:513210; and U.S. Patent Publication No. 2017/0045529. An antigen “associated with” a particular autoimmune disorder is an antigen that is a target of autoantibodies and/or autoreactive T cells present in individuals with that autoimmune disorder, where such autoantibodies and/or autoreactive T cells mediate a pathological state associated with the autoimmune disorder. A suitable T1D peptide for inclusion in a TMMP of the present disclosure can be an epitope-presenting T1D peptide of from 4 amino acids to about 25 amino acids in length of any one of the aforementioned T1D-associated antigens.

As one non-limiting example, a T1D peptide is proinsulin 73-90 (GAGSLQPLALEGSLQKR; SEQ ID NO:84). As another non-limiting example, a T1D peptide is the following insulin (InsA (1-15) peptide: GIVDQCCTSICSLYQ (SEQ ID NO:85). As another non-limiting example, a T1D peptide is the following insulin (InsA(1-15; D4E) peptide: GIVEQCCTSICSLYQ (SEQ ID NO:86). As another non-limiting example, a T1D peptide is the following GAD65 (555-567) peptide; NFFRMVISNPAAT (SEQ ID NO:87). As another non-limiting example, a T1D peptide is the following GAD65 (555-567; F557I) peptide; NFIRMVISNPAAT (SEQ ID NO:88). As another non-limiting example, a T1D peptide is the following islet antigen 2 (IA2) peptide: SFYLKNVQTQETRTLTQFHF (SEQ ID NO:89). As another non-limiting example, a T1D peptide is the following proinsulin peptide: SLQPLALEGSLQSRG (SEQ ID NO:90). As another non-limiting example, a T1D peptide is the following proinsulin peptide GSLQPLALEGSLQSRGIV (SEQ ID NO:91; proIns 75-92(K88S)).

In some cases, a suitable T1D peptide comprises from 4 to 25 contiguous amino acids of an amino acid sequence having at least 90%, at least 95%, at least 98%, at least 99%, or 100%, amino acid sequence identity to amino acids 25-110 of the following human preproinsulin amino acid sequence (wherein amino acids 1-24 (underlined) is a signal peptide): MALWMRLLPL LALLALWGPD PAAA FVNQHL CGSHLVEALY LVCGERGFFY TPKTRREAED LQVGQVELGG GPGAGSLQPL ALEGSLQKRG IVEQCCTSIC SLYQLENYCN (SEQ ID NO:92); where the T1D peptide has a length of 4 amino acids (aa), 5 aa, 6 aa, 7, aa, 8 aa, 9 aa, 10 aa, 11 aa, 12 aa, 13 aa, 14 aa, 15 aa, 16 aa, 17 aa, 18 aa. 19 aa, 20 aa, 21 aa, 22 aa, 23 aa, 24 aa, or 25 aa. In some cases, the T1D peptide has the amino acid sequence: GAGSLQPLALEGSLQKRG (SEQ ID NO:93) (proIns 73-90). In some cases, the T1D peptide has the amino acid sequence: SLQPLALEGSLQKRG (SEQ ID NO:94) (proIns 76-90). In some cases, the T1D peptide has the amino acid sequence: SLQPLALEGSLQSRG (SEQ ID NO:90) (proIns 76-90; K88S). In some cases, the T1D peptide has the amino acid sequence: QPLALEGSLQKRG (SEQ ID NO:95). In some cases, the T1D peptide has the amino acid sequence: QPLALEGSLQSRG (SEQ ID NO:96).

### Immunomodulatory Polypeptides (“MODs”)

As noted above, immunomodulatory polypeptides (“MODs”) that are suitable for inclusion in a TMPP of the present disclosure include, but are not limited to, IL-2, TGFβ, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, and HVEM. In some cases, the immunomodulatory polypeptide is selected from an IL-2 polypeptide, a 4-1BBL polypeptide, an ICOS-L polypeptide, an OX-40L polypeptide, a CD80 polypeptide, a CD86 polypeptide, a PD-L1 polypeptide, a FasL polypeptide, a TGFβ polypeptide, and a PD-L2 polypeptide.

As also noted above, the immunomodulatory polypeptide can comprise a wild-type amino acid sequence, or can comprise one or more amino acid substitutions relative to a wild-type amino acid sequence. The immunomodulatory polypeptide can comprise only the extracellular portion of a full-length immunomodulatory polypeptide. Thus, for example, the immunomodulatory polypeptide can in some cases exclude one or more of a signal peptide, a transmembrane domain, and an intracellular domain normally found in a naturally-occurring immunomodulatory polypeptide.

In some cases, an immunomodulatory polypeptide suitable for inclusion in a TMPP of the present disclosure comprises all or a portion of (e.g., an extracellular portion of) the amino acid sequence of a naturally-occurring immunomodulatory polypeptide. In other instances, an immunomodulatory polypeptide suitable for inclusion in a TMPP of the present disclosure is a variant immunomodulatory polypeptide that comprises at least one amino acid substitution compared to the amino acid sequence of a naturally-occurring immunomodulatory polypeptide. In some instances, a variant immunomodulatory polypeptide exhibits a binding affinity for a co-immunomodulatory polypeptide that is lower than the affinity of a corresponding naturally-occurring immunomodulatory polypeptide (e.g., an immunomodulatory polypeptide not comprising the amino acid substitution(s) present in the variant) for the co-immunomodulatory polypeptide.

Suitable immunomodulatory domains that exhibit reduced affinity for a co-immunomodulatory domain can have from 1 amino acid (aa) to 20 aa differences from a wild-type immunomodulatory domain. For example, in some cases, a variant immunomodulatory polypeptide present in a TMPP of the present disclosure differs in amino acid sequence by 1 aa. 2 aa, 3 aa, 4 aa, 5 aa, 6 aa. 7 aa, 8 aa, 9 aa, or 10 aa, from a corresponding wild-type immunomodulatory polypeptide. As another example, in some cases, a variant immunomodulatory polypeptide present in a TMPP of the present disclosure differs in amino acid sequence by 11 aa, 12 aa, 13 aa, 14 aa, 15 aa, 16 aa, 17 aa, 18 aa, 19 aa, or 20 aa, from a corresponding wild-type immunomodulatory polypeptide. As an example, in some cases, a variant immunomodulatory polypeptide present in a TMPP of the present disclosure includes 1, 2, 3, 4, 5, 6, 7, 8, 9, or 10 amino acid substitutions, compared to a corresponding reference (e.g., wild-type) immunomodulatory polypeptide.

As discussed above, a variant immunomodulatory polypeptide suitable for inclusion in a TMPP of the present disclosure exhibits reduced affinity for a cognate co-immunomodulatory polypeptide, compared to the affinity of a corresponding wild-type immunomodulatory polypeptide for the cognate co-immunomodulatory polypeptide.

Exemplary pairs of immunomodulatory polypeptide and cognate co-immunomodulatory polypeptide include, but are not limited to those set out in Table 2, below:

TABLE 2

| Immunomodulatory | Cognate Co-Immunomodulatory |
|---|---|
| Polypeptide | Polypeptide |
| 4-1BBL | 4-1BB |
| PD-L1 | PD-1 |
| IL-2 | IL-2 receptor |
| CD80 | CD28 |
| CD86 | CD28 |
| OX40L (CD252) | OX40 (CD134) |
| Fas ligand | Fas |
| ICOS-L | ICOS |
| ICAM | LFA-1 |
| CD30L | CD30 |
| CD40 | CD40L |
| CD83 | CD83L |
| HVEM (CD270) | CD160 |
| JAG1 (CD339) | Notch |
| JAG1 | CD46 |
| CD80 | CTLA4 |
| CD86 | CTLA4 |
| CD70 | CD27 |
| TGFβ | TGFβ receptor |

In some cases, a variant immunomodulatory polypeptide present in a TMPP of the present disclosure has a binding affinity for a cognate co-immunomodulatory polypeptide that is from 100 nM to 100 μM. For example, in some cases, a variant immunomodulatory polypeptide present in a TMPP of the present disclosure has a binding affinity for a cognate co-immunomodulatory polypeptide that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

Binding affinity between an immunomodulatory polypeptide and its cognate co-immunomodulatory polypeptide can be determined by bio-layer interferometry (BLI) using purified immunomodulatory polypeptide and purified cognate co-immunomodulatory polypeptide, following the procedure set forth in published PCT Application WO 2020/132138 A1.

### Wild-Type PD-L1 and PD-L1 Variants

In some cases, at least one of the one or more immunomodulatory polypeptides present in a TMMP of the present disclosure comprises the amino acid sequence of a wild-type PD-L1 polypeptide. In other instances, at least one of the one or more immunomodulatory polypeptides present in a TMMP of the present disclosure is a variant PD-L1 polypeptide. Wild-type PD-L1 and variant PD-L1 polypeptides bind to PD1.

A wild-type human PD-L1 polypeptide can comprise the following amino acid sequence: MRIFAVFIFM TYWHLLNAFT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAEL VIPGNI LNVSIKICLT LSPST (SEQ ID NO:97).

A wild-type PD-L1 polypeptide suitable for inclusion in a TMMP of the present disclosure can comprise the following amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR IL VVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:98).

A wild-type PD-L1 polypeptide suitable for inclusion in a TMMP of the present disclosure can comprise the following amino acid sequence:

(SEQ ID NO: 99)
FTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISY
GGADYKRITVKVNAPYNKINQRILVVDPVTSEHELTCQAEGYPKAEVIWT
SSDHQVLSGKTTTTNSKREEKLFNVTSTLRINTTTNEIFYCTFRRLDPEE
NHTAELVIPELPLAHPPNER.

A wild-type PD-1 polypeptide can comprise the following amino acid sequence:

(SEQ ID NO: 100)
PGWFLDSPDR PWNPPTFSPA LLVVTEGDNA TFTCSFSNTS
ESFVLNWYRM SPSNQTDKLA AFPEDRSQPG QDCRFRVTQL
PNGRDFHMSV VRARRNDSGT YLCGAISLAP KAQIKESLRA
ELRVTERRAE VPTAHPSPSP RPAGQFQTLV VGVVGGLLGS
LVLLVWVLAV ICSRAARGTI GARRTGQPLK EDPSAVPVFS
VDYGELDFQW REKTPEPPVP CVPEQTEYAT IVFPSGMGTS
SPARRGSADG PRSAQPLRPE DGHCSWPL.

In some cases, a variant PD-L1 polypeptide exhibits reduced binding affinity to PD-1 (e.g., a PD-1 polypeptide comprising the amino acid sequence depicted above), compared to the binding affinity of a PD-L1 polypeptide comprising the amino acid sequence: FTVTVPKDLYVVEYGSNMTIECKFPVEKQLDLAALIVYWEMEDKNIIQFVHGEEDLKVQHSSYR QRARLLKDQLSLGNAALQITDVKLQDAGVYRCMISYGGADYKRITVKVNAPYNKINQRILVVD PVTSEHELTCQAEGYPKAEVIWTSSDHQVLSGKTTTTNSKREEKLFNVTSTLRINTTTNEIFYCTF RRLDPEENHTAELVIPELPLAHPPNER (SEQ ID NO:99) or the amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:98). For example, in some cases, a variant PD-L1 polypeptide of the present disclosure binds PD-1 (e.g., a PD-1 polypeptide comprising the PD-1 amino acid sequence depicted above) with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a PD-L1 polypeptide comprising the amino acid sequence: FTVTVPKDLYVVEYGSNMTIECKFPVEKQLDLAALIVYWEMEDKNIIQFVHGEEDLKVQHSSYR QRARLLKDQLSLGNAALQITDVKLQDAGVYRCMISYGGADYKRITVKVNAPYNKINQRILVVD PVTSEHELTCQAEGYPKAEVIWTSSDHQVLSGKTTTTNSKREEKLFNVTSTLRINTTTNEIFYCTF RRLDPEENHTAEL VIPELPLAHPPNER (SEQ ID NO:99) or the amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:98).

In some cases, a variant PD-L1 polypeptide has a binding affinity to PD-1 that is from 1 nM to 1 mM. In some cases, a variant PD-L1 polypeptide of the present disclosure has a binding affinity to PD-1 that is from 100 nM to 100 μM. As another example, in some cases, a variant PD-L1 polypeptide has a binding affinity for PD1 (e.g., a PD1 polypeptide comprising the PD-1 amino acid sequence depicted above) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant PD-L1 polypeptide has a single amino acid substitution compared to one of the PD-L1 amino acid sequences depicted above. In some cases, a variant PD-L1 polypeptide has from 2 to 10 amino acid substitutions compared to one of the PD-L1 amino acid sequences depicted above.

### Wild-Type IL-2 and IL-2 Variants

In some cases, at least one of the one or more immunomodulatory polypeptides present in a TMMP of the present disclosure comprises the amino acid sequence of a wild-type IL-2 polypeptide. In other instances, at least one of the one or more immunomodulatory polypeptides present in a TMMP of the present disclosure is a variant IL-2 polypeptide. Wild-type IL-2 and variant IL-2 polypeptides bind to IL-2 receptor (IL-2R) (e.g., bind to one or more polypeptides of an IL-2R).

A wild-type IL-2 amino acid sequence can be as follows: APTSSSTKKT QLQLEHLLLD LQMILNGINN YKNPKLTRML TFKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:101).

Wild-type IL-2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122; and a gamma chain (IL-2Rγ; also referred to as CD132). Amino acid sequences of human IL-2Rα, IL2Rβ, and IL-2Rγ can be as follows.

Human IL-2Rα:
(SEQ ID NO: 102)
ELCDDDPPE IPHATFKAMA YKEGTMLNCE CKRGFRRIKS
GSLYMLCTGN SSHSSWDNQC QCTSSATRNT TKQVTPQPEE
QKERKTTEMQ SPMQPVDQAS LPGHCREPPP WENEATERIY
HFVVGQMVYY QCVQGYRALH RGPAESVCKM THGKTRWTQP
QLICTGEMET SQFPGEEKPQ ASPEGRPESE TSCLVTTTDF
QIQTEMAATM ETSIFTTEYQ VAVAGCVFLL ISVLLLSGLT
WQRRQRKSRR TI.
Human IL-2Rβ:
(SEQ ID NO: 103)
VNG TSQFTCFYNS RANISCVWSQ DGALQDTSCQ
VHAWPDRRRW NQTCELLPVS QASWACNLIL GAPDSQKLTT
VDIVTLRVLC REGVRWRVMA IQDFKPFENL RLMAPISLQV
VHVETHRCNI SWEISQASHY FERHLEFEAR TLSPGHTWEE
APLLTLKQKQ EWICLETLTP DTQYEFQVRV KPLQGEFTTW
SPWSQPLAFR TKPAALGKDT IPWLGHLLVG LSGAFGFIIL
VYLLINCRNT GPWLKKVLKC NTPDPSKFFS QLSSEHGGDV
QKWLSSPFPS SSFSPGGLAP EISPLEVLER DKVTQLLLQQ
DKVPEPASLS SNHSLTSCFT NQGYFFFHLP DALEIEACQV
YFTYDPYSEE DPDEGVAGAP TGSSPQPLQP LSGEDDAYCT
FPSRDDLLLF SPSLLGGPSP PSTAPGGSGA GEERMPPSLQ
ERVPRDWDPQ PLGPPTPGVP DLVDFQPPPE LVLREAGEEV
PDAGPREGVS FPWSRPPGQG EFRALNARLP LNTDAYLSLQ
ELQGQDPTHL V.
Human IL-2Rγ:
(SEQ ID NO: 104)
LNTTILTP NGNEDTTADF FLTTMPTDSL SVSTLPLPEV
QCFVFNVEYM NCTWNSSSEP QPTNLTLHYW YKNSDNDKVQ
KCSHYLFSEE ITSGCQLQKK EIHLYQTFVV QLQDPREPRR
QATQMLKLQN LVIPWAPENL TLHKLSESQL ELNWNNRFLN
HCLEHLVQYR TDWDHSWTEQ SVDYRHKFSL PSVDGQKRYT
FRVRSRFNPL CGSAQHWSEW SHPIHWGSNT SKENPFLFAL
EAVVISVGSM GLIISLLCVY FWLERTMPRI PTLKNLEDLV
TEYHGNFSAW SGVSKGLAES LQPDYSERLC LVSEIPPKGG
ALGEGPGASP CNQHSPYWAP PCYTLKPET.

In some cases, where a TMPP of the present disclosure comprises a variant IL-2 polypeptide, a “cognate co-immunomodulatory polypeptide” is an IL-2R comprising polypeptides comprising the amino acid sequences of human IL-2Rα, human IL-2Rβ, and IL-2Rγ shown above.

In some cases, a variant IL-2 polypeptide exhibits reduced binding affinity to IL-2R, compared to the binding affinity of an IL-2 polypeptide comprising a wild-type IL-2 polypeptide (e.g., a wild-type IL-2 polypeptide having the amino acid sequence shown above). For example, in some cases, a variant IL-2 polypeptide binds IL-2R with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of an IL-2 polypeptide comprising the amino acid sequence APTSSSTKKT QLQLEHLLLD LQMILNGINN YKNPKLTRML TFKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:101) for an IL-2R (e.g., an IL-2R comprising polypeptides comprising human IL-2Rα, human IL-2Rβ, and IL-2Rγ having the amino acid sequences shown above), when assayed under the same conditions.

In some cases, a variant IL-2 polypeptide has a binding affinity to IL-2R that is from 100 nM to 100 μM. As another example, in some cases, a variant IL-2 polypeptide has a binding affinity for IL-2R (e.g., an IL-2R comprising human IL-2Rα, human IL-2Rβ, and IL-2Rγ having the amino acid sequences shown above) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant IL-2 polypeptide has a single amino acid substitution, or from 2-10 amino acid substitutions compared to the IL-2 amino acid sequence APTSSSTKKT QLQLEHLLLD LQMILNGINN YKNPKLTRML TFKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:101).

Suitable IL-2 variants include a polypeptide that comprises an amino acid sequence having at least 60%, at least 70%, at least 80%, at least 90%, at least 95%, at least 98%, at least 99%, or 100%, amino acid sequence identity to any one of the following amino acid sequences:
APTSSSTKKT QLQLEHLLLD LQMILNGINN YKNPKLTRML TXKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 105), where X is any amino acid other than Phe. In some cases, X is Ala;
APTSSSTKKT QLQLEHLLLX LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 106), where X is any amino acid other than Asp. In some cases, X is Ala;
APTSSSTKKT QLQLXHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 107), where X is any amino acid other than Glu. In some cases, X is Ala;
APTSSSTKKT QLQLEXLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 108), where X is any amino acid other than His. In some cases, X is Ala. In some cases, X is Arg. In some cases, X is Asn. In some cases, X is Asp. In some cases, X is Cys. In some cases, X is Glu. In some cases, X is Gln. In some cases, X is Gly. In some cases, X is Ile. In some cases, X is Lys. In some cases, X is Leu. In some cases, X is Met. In some cases, X is Phe. In some cases, X is Pro. In some cases, X is Ser. In some cases, X is Thr. In some cases, X is Tyr. In some cases, X is Trp. In some cases, X is Val;
APTSSSTKKT QLQLEHLLLD LQMILNGINN YKNPKLTRML TFKFXMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 109), where X is any amino acid other than Tyr. In some cases, X is Ala;
APTSSSTKKT QLQLEHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCXSIIS TLT (SEQ ID NO: 110), where X is any amino acid other than Gln. In some cases, X is Ala;
APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:111), where X₁ is any amino acid other than His, and where X₂ is any amino acid other than Phe. In some cases, X₁ is Ala. In some cases, X₂ is Ala. In some cases, X₁ is Ala; and X₂ is Ala. In some cases, X₁ is Thr; and X₂ is Ala;
APTSSSTKKT QLQLEHLLLX₁ LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 112), where X₁ is any amino acid other than Asp; and where X₂ is any amino acid other than Phe. In some cases, X₁ is Ala. In some cases, X₂ is Ala. In some cases, X₁ is Ala; and X₂ is Ala;
APTSSSTKKT QLQLX₁HLLLX₂ LQMILNGINN YKNPKLTRML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:113), where X₁ is any amino acid other than Glu; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala;
APTSSSTKKT QLQLEX₁LLLX₂ LQMILNGINN YKNPKLTRML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:114), where X₁ is any amino acid other than His; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala;
APTSSSTKKT QLQLEHLLLX₁ LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCX₃SIIS TLT (SEQ ID NO: 115), where X₁ is any amino acid other than Asp; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala;
APTSSSTKKT QLQLEHLLLX₁ LQMILNGINN YKNPKLTRML TX₂KFX₃MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:116), where X₁ is any amino acid other than Asp; where X₂ is any amino acid other than Phe; and where X₃ is any amino acid other than Tyr. In some cases, X₁ is Ala. In some cases, X₂ is Ala. In some cases, X₃ is Ala. In some cases, X₁ is Ala; X₂ is Ala; and X₃ is Ala;
APTSSSTKKT QLQLEX₁LLLX₂ LQMILNGINN YKNPKLTRML TX₃KFX₄MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:117), where X₁ is any amino acid other than His; where X₂ is any amino acid other than Asp; where X₃ is any amino acid other than Phe; and where X₄ is any amino acid other than Tyr. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala;
APTSSSTKKT QLQLEHLLLX₁ LQMILNGINN YKNPKLTRML TX₂KFX₃MPKKA TELKHLQCLE EELKPLEEVL NLAQSKNFHL RPRDLISNIN VIVLELKGSE TTFMCEYADE TATIVEFLNR WITFCX₄SIIS TLT (SEQ ID NO: 118), where X₁ is any amino acid other than Asp; where X₂ is any amino acid other than Phe; where X₃ is any amino acid other than Tyr; and where X₄ is any amino acid other than Gln. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala;
APTSSSTKKT QLQLEX₁LLLX₂ LQMILNGINN YKNPKLTRML TX₃KFX₄MPKKA TELKHLQCLE EELKPLEEVL NLAQSKNFHL RPRDLISNIN VIVLELKGSE TTFMCEYADE TATIVEFLNR WITFCX₅SIIS TLT (SEQ ID NO:119), where X₁ is any amino acid other than His; where X₂ is any amino acid other than Asp; where X₃ is any amino acid other than Phe; where X₄ is any amino acid other than Tyr; and where X₅ is any amino acid other than Gln. In some cases, X₁ is Ala. In some cases, X₂ is Ala. In some cases, X₃ is Ala. In some cases, X₄ is Ala. In some cases, X₅ is Ala. In some cases, X₁ is Ala; X₂ is Ala; X₃ is Ala; X₄ is Ala; X₅ is Ala; and
APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCX₃SIIS TLT (SEQ ID NO:120), where X₁ is any amino acid other than His; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala.

In some cases, a suitable variant IL-2 polypeptide comprises the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:121), i.e., the variant IL-2 polypeptide has the amino acid sequence of wild-type IL-2 but with H16A and F42A substitutions (shown in bold).

### Wild-Type TGF-β and Variant TGF-β Polypeptides

In some cases, at least one of the one or more immunomodulatory polypeptides present in a TMMP of the present disclosure comprises the amino acid sequence of a wild-type TGF-β polypeptide. In other instances, at least one of the one or more immunomodulatory polypeptides present in a TMMP of the present disclosure is a variant TGF-β polypeptide. Wild-type TGF-β and variant TGF-polypeptides bind to TGF receptor.

As noted above, in some cases, the immunomodulatory polypeptide present in a TMMP of the present disclosure is a TGF-β polypeptide. Amino acid sequences of TGF-β polypeptides are known in the art. In some cases, the immunomodulatory polypeptide present in a TMMP of the present disclosure is a TGF-β1 polypeptide, immunomodulatory polypeptide present in a TMMP of the present disclosure is a TGF-β2 polypeptide, immunomodulatory polypeptide present in a TMMP of the present disclosure is a TGF-β3 polypeptide. A suitable TGF-β polypeptide can comprise an amino acid sequence having at least 60%, at least 70%, at least 80%, at least 90%, at least 95%, at least 98%, at least 99%, or 100%, amino acid sequence identity to the mature form of a human TGF-β1 polypeptide, a human TGF-β2 polypeptide, or a human TGF-β3 polypeptide. A suitable TGF-β polypeptide can have a length of from about 100 amino acids to about 125 amino acids; for example, a suitable TGF-β polypeptide can have a length of from about 100 amino acids to about 105 amino acids, from about 105 amino acids to about 110 amino acids, from about 110 amino acids to about 115 amino acids, from about 115 amino acids to about 120 amino acids, or from about 120 amino acids to about 125 amino acids.

A suitable TGF-β1 polypeptide can comprise an amino acid sequence having at least 60%, at least 70%, at least 80%, at least 90%, at least 95%, at least 98%, at least 99%, or 100%, amino acid sequence identity to the following TGF-β1 amino acid sequence: AL DTNYCFSSTE KNCCVRQLYI DFRKDLGWKW IHEPKGYHAN FCLGPCPYIW SLDTQYSKVL ALYNQHNPGA SAAPCCVPQA LEPLPIVYYV GRKPKVEQLS NMIVRSCKCS (SEQ ID NO:122); or the foregoing sequence comprising a C77S substitution; where the TGF-β1 polypeptide has a length of about 112 amino acids.

A suitable TGF-β2 polypeptide can comprise an amino acid sequence having at least 60%, at least 70%, at least 80%, at least 90%, at least 95%, at least 98%, at least 99%, or 100%, amino acid sequence identity to the following TGF-β2 amino acid sequence: ALDAAYCF RNVQDNCCLR PLYIDFKRDL GWKWIHEPKG YNANFCAGAC PYLWSSDTQH SRVLSLYNTI NPEASASPCC VSQDLEPLTI LYYIGKTPKI EQLSNMIVKS CKCS (SEQ ID NO:123); or the foregoing sequence comprising a C79S substitution, where the TGF-β2 polypeptide has a length of about 112 amino acids.

A suitable TGF-β3 polypeptide can comprise an amino acid sequence having at least 60%, at least 70%, at least 80%, at least 90%, at least 95%, at least 98%, at least 99%, or 100% amino acid sequence identity to the following TGF-β3 amino acid sequence: ALDTNYCFRN LEENCCVRPL YIDFRQDLGW KWVHEPKGYY ANFCSGPCPY LRSADTTHST VLGLYNTLNP EASASPCCVP QDLEPLTILY YVGRTPKVEQ LSNMVVKSCK CS (SEQ ID NO:124); or the foregoing sequence comprising a C77S substitution, where the TGF-β3 polypeptide has a length of about 112 amino acids.

### FasL and FasL Variants

In some cases, at least one of the one or more immunomodulatory polypeptides present in a TMMP of the present disclosure comprises the amino acid sequence of a wild-type Fas ligand (FasL) polypeptide. In other instances, at least one of the one or more immunomodulatory polypeptides present in a TMMP of the present disclosure is a variant FasL polypeptide. Wild-type FasL and variant FasL polypeptides bind to Fas receptor (FasR). FasL is also known as CD95L or CD178.

Human FasL polypeptide can have the following amino acid sequence: MQQPFNYPYP QIYWVDSSAS SPWAPPGTVL PCPTSVPRRP GQRRPPPPPP PPPLPPPPPP PPLPPLPLPP LKKRGNHSTG LCLLVMFFMV LVALVGLGLG MFQLFHLQKE LAELRESTSQ MHTASSLEKQ IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVEN LTSADHLYVN VSELSLVNFE ESQTFFGLYK L (SEQ ID NO:125). In some cases, a FasL polypeptide suitable for inclusion in a TMMP of the present disclosure comprises an amino acid sequence having at least 60%, at least 70%, at least 80%, at least 90%, at least 95%, at least 98%, at least 99%, or 100%, amino acid sequence identity to a contiguous stretch of from about 150 amino acids to about 175, from about 175 amino acids to about 200 amino acids, from about 200 amino acids to about 250 amino acids, or from about 250 amino acids to about 281 amino acids, of the following amino acid sequence: MQQPFNYPYP QIYWVDSSAS SPWAPPGTVL PCPTSVPRRP GQRRPPPPPP PPPLPPPPPP PPLPPLPLPP LKKRGNHSTG LCLLVMFFMV LVALVGLGLG MFQLFHLQKE LAELRESTSQ MHTASSLEKQ IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVEN LTSADHLYVN VSELSLVNFE ESQTFFGLYK L (SEQ ID NO:125).

In some cases, a FasL polypeptide suitable for inclusion in a TMMP of the present disclosure comprises an amino acid sequence having at least 60%, at least 70%, at least 80%, at least 90%, at least 95%, at least 98%, at least 99%, or 100%, amino acid sequence identity to the following amino acid sequence: IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVEN LTSADHLYVN VSELSLVNFE ESQTFFGLYK (SEQ ID NO:237); and has a length of about 150, 151, or 152 amino acids.

In some cases, a FasL polypeptide suitable for inclusion in a TMMP of the present disclosure comprises an amino acid sequence having at least 60%, at least 70%, at least 80%, at least 90%, at least 95%, at least 98%, at least 99%, or 100%, amino acid sequence identity to the following amino acid sequence: QLFHLQKELAELRESTSQMHTASSLEKQIGHPSPPPEKKELRKVAHLTGKSNSRSMPLEWEDTY GIVLLSGVKYKKGGLVINETGLYFVYSKVYFRGQSCNNLPLSHKVYMRNSKYPQDLVMMEGK MMSYCTTGQMWARSSYLGAVENLTSADHLYVNVSELSLVNFEESQTFFGLYKL (SEQ ID NO:126); and can have a length of about 179 amino acids.

A FasR polypeptide can comprise an amino acid sequence having at least 60%, at least 70%, at least 80%, at least 90%, at least 95%, at least 98%, at least 99%, or 100%, amino acid sequence identity to the following amino acid sequence: MLGIWTLLPL VLTSVARLSS KSVNAQVTDI NSKGLELRKT VTTVETQNLE GLHHDGQFCH KPCPPGERKA RDCTVNGDEP DCVPCQEGKE YTDKAHFSSK CRRCRLCDEG HGLEVEINCT RTQNTKCRCK PNFFCNSTVC EHCDPCTKCE HGIIKECTLT SNTKCKEEGS RSNLGWLCLL LLPIPLIVWV KRKEVQKTCR KHRKENQGSH ESPTLNPETV AINLSDVDLS KYITTIAGVM TLSQVKGFVR KNGVNEAKID EIKNDNVQDT AEQKVOLLRN WHQLHGKKEA YDTLIKDLKK ANLCTLAEKI QTIILKDITS DSENSNFRNE IQSLV (SEQ ID NO:127).

### Exemplary TMMPs

As noted above, a TMMP of the present disclosure comprises a heterodimer (e.g., comprises two heterodimers), where the heterodimer comprises a first polypeptide chain and a second polypeptide chain that are disulfide bonded to one another.

TMMPs that are Disulfide Bonded Via a Cys in the MHC Class II Alpha Chain and a Cys in the MHC Class II Beta Chain

As noted above, in some cases, a TMMP of the present disclosure comprises a heterodimer comprising a first polypeptide chain comprising: i) a T1D peptide; and ii) a first MHC class II polypeptide; and b) a second polypeptide comprising a second MHC class II polypeptide, where the first and/or the second polypeptides comprise one or more immunomodulatory polypeptides, optionally wherein the first and/or the second polypeptide comprises an Ig Fc polypeptide; where the first MHC class II polypeptide comprises a substitution of an amino acid (other than a Cys) with a Cys (a “first Cys”), and the second MHC class II polypeptide comprises a substitution of an amino acid (other than a Cys) with a Cys (a “second” Cys), where the first polypeptide and the second polypeptide are joined by a disulfide bond formed between the first Cys and the second Cys.

The first MHC class II polypeptide can be a DRB MHC class II polypeptide comprising an amino acid sequence having at least 80%, at least 85%, at least 90%, at least 95%, at least 98%, or at least 99% amino acid sequence identity to the amino acid sequence depicted in FIG. 14A; and having an amino acid substitution selected from P5C, F7C, Q10C, N19C, G20C, H33C, G151C, D152C, and W153C. The second MHC class II polypeptide can be a DRA MHC class II polypeptide comprising an amino acid sequence having at least 80%, at least 85%, at least 90%, at least 95%, at least 98%, or at least 99%, amino acid sequence identity to the amino acid sequence depicted in FIG. 13A; and having an amino acid substitution selected from E3C, E4C, F12C, G28C, D29C, I72C, K75C, T80C, P81C, 182C, T93C, N94C, and S95C.

As one example, in some cases, the first MHC class II polypeptide is a DRB MHC class II polypeptide comprising a substitution at a residue selected from the group consisting of P5C, H33C, G151C, and W153, and the second MHC class II polypeptide is a DRA MHC class II polypeptide comprising a substitution selected from the group consisting of P81C, 182C, and D29C. For example, the MHC class II β chain polypeptide can comprise an amino acid sequence having at least 80%, at least 85%, at least 90%, at least 95%, at least 98%, or at least 99% amino acid sequence identity to the amino acid sequence depicted in FIG. 14A, where the polypeptide comprises a Cys at a residue position selected from the group consisting of 5, 33, 151, and 153; and the MHC class II α chain polypeptide can comprise an amino acid sequence having at least 80%, at least 85%, at least 90%, at least 95%, at least 98%, or at least 99% amino acid sequence identity to the amino acid sequence depicted in FIG. 13A, where the polypeptide comprises a Cys at a residue selected from the group consisting of 81, 82, and 29.

As another example, the disulfide can be formed between one of the specific pairs of Cys residues in Table 3, below:

TABLE 3

| DRB MHC Class II | DRA MHC Class II |
|---|---|
| Polypeptide Substitution | Polypeptide Substitution |
| N19C | E3C |
| G20C | E3C |
| N19C | E4C |
| G20C | E4C |
| F7C | F12C |
| Q10C | F12C |
| G151C | G28C |
| D152C | G28C |
| W153C | G28C |
| G151C | D29C |
| D152C | D29C |
| W153C | D29C |
| F7C | T80C |
| P5C | T80C |
| H33C | T80C |
| P5C | P81C |
| H33C | P81C |
| F7C | P81C |
| H33C | I82C |
| F7C | I82C |
| P5C | I82C |
| Q156C | T93C |
| W153C | T93C |
| Q156C | N94C |
| N120C | N94C |
| Q156C | S95C |
| N120C | S95C |

For example, the DRB MHC class II polypeptide can comprise an amino acid sequence having at least 80%, at least 85%, at least 90%, at least 95%, at least 98%, or at least 99% amino acid sequence identity to the amino acid sequence depicted in FIG. 14A, and the DRA MHC class II polypeptide can comprise an amino acid sequence having at least 80%, at least 85%, at least 90%, at least 95%, at least 98%, or at least 99% amino acid sequence identity to the amino acid sequence depicted in FIG. 13A, wherein the β chain polypeptide and α chain polypeptides are joined by a disulfide formed between a pair of Cys residues selected from the group consisting of: β chain polypeptide residue 5 and α chain polypeptide residue 81; β chain polypeptide residue 33 and α chain polypeptide residue 81; β chain polypeptide residue 33 and α chain polypeptide residue 82; β chain polypeptide residue 151 and α chain polypeptide residue 29; and β chain polypeptide residue 153 and α chain polypeptide residue 29.

### TMMP Bonded Through a Disulfide Bond Between β PC and α P81C

As one example, a TMMP of the present disclosure comprises a heterodimer comprising: a) a first polypeptide comprising, in order from N-terminus to C-terminus: i) a T1D peptide; and ii) a DRB MHC class II polypeptide comprising a P5C substitution; and b) a second polypeptide comprising, in order form N-terminus to C-terminus: i) one or more immunomodulatory polypeptides; ii) a DRA MHC class II polypeptide comprising a P81C substitution; and (optionally) iii) an Ig Fc polypeptide. (See FIG. 5, MOD Position 1.) As another example, a TMMP of the present disclosure comprises a heterodimer comprising: a) a first polypeptide comprising, in order from N-terminus to C-terminus: i) a T1D peptide; and ii) a DRB MHC class II polypeptide comprising a P5C substitution; and b) a second polypeptide comprising, in order form N-terminus to C-terminus: i) a DRA MHC class II polypeptide comprising a P81C substitution; ii) an Ig Fc polypeptide; and iii) one or more immunomodulatory polypeptides. (See FIG. 5, MOD Position 3.) Alternatively, the one or more immunomodulatory polypeptides can be at Position 2, 4 or 5 as those positions are described above and depicted schematically in FIG. 5. In the above embodiments, the first polypeptide and the second polypeptide are linked via a disulfide bond between the Cys at residue 5 in the DRB MHC class II polypeptide and the Cys at residue 81 in the DRA MHC class II polypeptide. In any of the above embodiments, linkers that do not comprise a Cys optionally can be used to connect the components of the first or second polypeptide. In any of the above embodiments, in some cases, the one or more immunomodulatory polypeptides is a PD-L1 polypeptide or variant thereof, a TGF-β polypeptide or variant thereof, an IL-2 polypeptide or variant thereof, or a FasL polypeptide or variant thereof. In some cases, the Ig Fc polypeptide is present and is a human IgG1 Fc polypeptide, optionally comprising L234A and L235A substitutions (L14A and L15A of the amino acid sequence depicted in FIG. 12A). In some cases, the DRB MHC class II polypeptide comprises an amino acid sequence having at least 80%, at least 85%, at least 90%, at least 95%, at least 98%, or at least 99% amino acid sequence identity to the amino acid sequence depicted in FIG. 14B, and comprises a Cys at position 5. In some cases, the DRA MHC class II polypeptide comprises an amino acid sequence having at least 80%, at least 85%, at least 90%, at least 95%, at least 98%, or at least 99% amino acid sequence identity to the amino acid sequence depicted in FIG. 13J, and comprises a Cys at position 81. In some cases, the T1D peptide is a proinsulin peptide. In some cases, the T1D peptide is a proinsulin peptide selected from SLQPLALEGSLQKRG (SEQ ID NO:94; proIns 76-90), SLQPLALEGSLQSRG (SEQ ID NO:90; proIns 76-90; K88S), and GAGSLQPLALEGSLQKRG (SEQ ID NO:93; proIns 73-90). In some cases, the T1D peptide is a GAD peptide. In some cases, the T1D peptide is a GAD peptide selected from NFFRMVISNPAAT (SEQ ID NO:87; GAD65 555-567) and NFIRMVISNPAAT (SEQ ID NO:88; GAD65 555-567; F557I).

### TMMP Bonded Through a Disulfide Bond Between β H33C and a P81C

As one example, a TMMP of the present disclosure comprises a heterodimer comprising: a) a first polypeptide comprising, in order from N-terminus to C-terminus: i) a T1D peptide; and ii) a DRB MHC class II polypeptide comprising an H33C substitution; and b) a second polypeptide comprising, in order form N-terminus to C-terminus: i) one or more immunomodulatory polypeptides; ii) a DRA MHC class II polypeptide comprising a P81C substitution; and (optionally) iii) an Ig Fc polypeptide. (See FIG. 5, MOD Position 1.) As another example, a TMMP of the present disclosure comprises a heterodimer comprising: a) a first polypeptide comprising, in order from N-terminus to C-terminus: i) a T1D peptide; and ii) a DRB MHC class II polypeptide comprising an H33C substitution; and b) a second polypeptide comprising, in order form N-terminus to C-terminus: i) a DRA MHC class II polypeptide comprising a P81C substitution; ii) an Ig Fc polypeptide; and iii) one or more immunomodulatory polypeptides. (See FIG. 5, MOD Position 3). Alternatively, the one or more immunomodulatory polypeptides can be at Position 2, 4 or 5 as those positions are described above and depicted schematically in FIG. 5. In the above embodiments, the first polypeptide and the second polypeptide are linked via a disulfide bond between the Cys at residue 33 in the DRB MHC class II polypeptide and the Cys at residue 81 in the DRA MHC class II polypeptide. In any of the above embodiments, linkers that do not comprise a Cys can be used to connect the components of the first or second polypeptide. In any of the above embodiments, in some cases, the one or more immunomodulatory polypeptides is a PD-L1 polypeptide or variant thereof, a TGF-β polypeptide or variant thereof, an IL-2 polypeptide or variant thereof, or a FasL polypeptide or variant thereof. In some cases, the Ig Fc polypeptide is present and is a human IgG1 Fc polypeptide, optionally comprising L234A and L235A substitutions (L14A and L15A of the amino acid sequence depicted in FIG. 12A). In some cases, the DRB MHC class II polypeptide comprises an amino acid sequence having at least 80%, at least 85%, at least 90%, at least 95%, at least 98%, at least 99%, or 100%, amino acid sequence identity to the amino acid sequence depicted in FIG. 14G, and comprises a Cys at position 33. In some cases, the DRA MHC class II polypeptide comprises an amino acid sequence having at least 80%, at least 85%, at least 90%, at least 95%, at least 98%, at least 99%, or 100%, amino acid sequence identity to the amino acid sequence depicted in FIG. 13J, and comprises a Cys at position 81. In some cases, the T1D peptide is a proinsulin peptide. In some cases, the T1D peptide is a proinsulin peptide selected from SLQPLALEGSLQKRG (SEQ ID NO:94; proIns 76-90), SLQPLALEGSLQSRG (SEQ ID NO:90; proIns 76-90; K88S), and GAGSLQPLALEGSLQKRG (SEQ ID NO:93; proIns 73-90). In some cases, the T1D peptide is a GAD peptide. In some cases, the T1D peptide is a GAD peptide selected from NFFRMVISNPAAT (SEQ ID NO:87; GAD65 555-567) and NFIRMVISNPAAT (SEQ ID NO:88; GAD65 555-567; F557I).

### TMMP Bonded Through a Disulfide Bond Between β H33C and a 182C

As one example, a TMMP of the present disclosure comprises a heterodimer comprising: a) a first polypeptide comprising, in order from N-terminus to C-terminus: i) a T1D peptide; and ii) a DRB MHC class II polypeptide comprising an H33C substitution; and b) a second polypeptide comprising, in order form N-terminus to C-terminus: i) one or more immunomodulatory polypeptides; ii) a DRA MHC class II polypeptide comprising an 182C substitution; and (optionally) iii) an Ig Fc polypeptide. (See FIG. 5, MOD Position 1.) As another example, a TMMP of the present disclosure comprises a heterodimer comprising: a) a first polypeptide comprising, in order from N-terminus to C-terminus: i) a T1D peptide; and ii) a DRB MHC class II polypeptide comprising an H33C substitution; and b) a second polypeptide comprising, in order form N-terminus to C-terminus: i) a DRA MHC class II polypeptide comprising an 182C substitution; ii) an Ig Fc polypeptide; and iii) one or more immunomodulatory polypeptides. (See FIG. 5, MOD Position 3.) Alternatively, the one or more immunomodulatory polypeptides can be at Position 2, 4 or 5 as those positions are described above and depicted schematically in FIG. 5. In the above embodiments, the first polypeptide and the second polypeptide are linked via a disulfide bond between the Cys at residue 33 in the DRB MHC class II polypeptide and the Cys at residue 82 in the DRA MHC class II polypeptide. In any of the above embodiments, linkers that do not comprise a Cys optionally can be used to connect the components of the first or second polypeptide. In any of the above embodiments, in some cases, the one or more immunomodulatory polypeptides is a PD-L1 polypeptide or variant thereof, a TGF-β polypeptide or variant thereof, an IL-2 polypeptide or variant thereof, or a FasL polypeptide or variant thereof. In some cases, the Ig Fc polypeptide is present and is a human IgG1 Fc polypeptide, optionally comprising L234A and L235A substitutions (L14A and L15A of the amino acid sequence depicted in FIG. 12A). In some cases, the DRB MHC class II polypeptide comprises an amino acid sequence having at least 80%, at least 85%, at least 90%, at least 95%, at least 98%, at least 99%, or 100%, amino acid sequence identity to the amino acid sequence depicted in FIG. 14G, and comprises a Cys at position 33. In some cases, the DRA MHC class II polypeptide comprises an amino acid sequence having at least 80%, at least 85%, at least 90%, at least 95%, at least 98%, at least 99%, or 100%, amino acid sequence identity to the amino acid sequence depicted in FIG. 13K, and comprises a Cys at position 82. In some cases, the T1D peptide is a proinsulin peptide. In some cases, the T1D peptide is a proinsulin peptide selected from SLQPLALEGSLQKRG (SEQ ID NO:94; proIns 76-90), SLQPLALEGSLQSRG (SEQ ID NO:90; proIns 76-90; K88S), and GAGSLQPLALEGSLQKRG (SEQ ID NO:93; proIns 73-90). In some cases, the T1D peptide is a GAD peptide. In some cases, the T1D peptide is a GAD peptide selected from NFFRMVISNPAAT (SEQ ID NO:87; GAD65 555-567) and NFIRMVISNPAAT (SEQ ID NO: 88; GAD65 555-567; F557I).

### TMMP Bonded Through a Disulfide Bond Between β G151C and a D29C

As one example, a TMMP of the present disclosure comprises a heterodimer comprising: a) a first polypeptide comprising, in order from N-terminus to C-terminus: i) a T1D peptide; and ii) a DRB MHC class II polypeptide comprising a G151C substitution; and b) a second polypeptide comprising, in order form N-terminus to C-terminus: i) one or more immunomodulatory polypeptides; ii) a DRA MHC class II polypeptide comprising a D29C substitution; and (optionally) iii) an Ig Fc polypeptide. (See FIG. 5, MOD Position 1.) As another example, a TMMP of the present disclosure comprises a heterodimer comprising: a) a first polypeptide comprising, in order from N-terminus to C-terminus: i) a T1D peptide; and ii) a DRB MHC class II polypeptide comprising a G151C substitution; and b) a second polypeptide comprising, in order form N-terminus to C-terminus: i) a DRA MHC class II polypeptide comprising a D29C substitution; ii) an Ig Fc polypeptide; and iii) one or more immunomodulatory polypeptides. (See FIG. 5, MOD Position 3.) [Alternatively, the one or more immunomodulatory polypeptides can be at Position 2, 4 or 5 as those positions are described above and depicted schematically in FIG. 5. In the above embodiments, the first polypeptide and the second polypeptide are linked via a disulfide bond between the Cys at residue 151 in the DRB MHC class II polypeptide and the Cys at residue 29 in the DRA MHC class II polypeptide. In any of the above embodiments, linkers that do not comprise a Cys optionally can be used to connect the components of the first or second polypeptide. In any of the above embodiments, in some cases, the one or more immunomodulatory polypeptides is a PD-L1 polypeptide or variant thereof, a TGF-β polypeptide or variant thereof, an IL-2 polypeptide or variant thereof, or a FasL polypeptide or variant thereof. In some cases, the Ig Fc polypeptide is present and is a human IgG1 Fc polypeptide, optionally comprising L234A and L235A substitutions (L14A and L15A of the amino acid sequence depicted in FIG. 12A). In some cases, the DRB MHC class II polypeptide comprises an amino acid sequence having at least 80%, at least 85%, at least 90%, at least 95%, at least 98%, or at least 99%, amino acid sequence identity to the amino acid sequence depicted in FIG. 14H, and comprises a Cys at position 151. In some cases, the DRA MHC class II polypeptide comprises an amino acid sequence having at least 80%, at least 85%, at least 90%, at least 95%, at least 98%, at least 99%, or 100%, amino acid sequence identity to the amino acid sequence depicted in FIG. 13F, and comprises a Cys at position 29. In some cases, the T1D peptide is a proinsulin peptide. In some cases, the T1D peptide is a proinsulin peptide selected from SLQPLALEGSLQKRG (SEQ ID NO:94; proIns 76-90), SLQPLALEGSLQSRG (SEQ ID NO:90; proIns 76-90; K88S), and GAGSLQPLALEGSLQKRG (SEQ ID NO:93; proIns 73-90). In some cases, the T1D peptide is a GAD peptide. In some cases, the T1D peptide is a GAD peptide selected from NFFRMVISNPAAT (SEQ ID NO:87; GAD65 555-567) and NFIRMVISNPAAT (SEQ ID NO:88; GAD65 555-567; F557I).

### TMMP Bonded Through a Disulfide Bond Between B W153C and a D29C

As one example, a TMMP of the present disclosure comprises a heterodimer comprising: a) a first polypeptide comprising, in order from N-terminus to C-terminus: i) a T1D peptide; and ii) a DRB MHC class II polypeptide comprising a W513C substitution; and b) a second polypeptide comprising, in order form N-terminus to C-terminus: i) one or more immunomodulatory polypeptides; ii) a DRA MHC class II polypeptide comprising a D29C substitution; and (optionally) iii) an Ig Fc polypeptide. (See FIG. 5, MOD Position 1.) As another example, a TMMP of the present disclosure comprises a heterodimer comprising: a) a first polypeptide comprising, in order from N-terminus to C-terminus: i) a T1D peptide; and ii) a DRB MHC class II polypeptide comprising a W513C substitution; and b) a second polypeptide comprising, in order form N-terminus to C-terminus: i) a DRA MHC class II α chain polypeptide comprising a D29C substitution; ii) an Ig Fc polypeptide; and iii) one or more immunomodulatory polypeptides. (See FIG. 5, MOD Position 3) Alternatively, the one or more immunomodulatory polypeptides can be at Position 2, 4 or 5 as those positions are described above and depicted schematically in FIG. 5. In the above embodiments, the first polypeptide and the second polypeptide are linked via a disulfide bond between the Cys at residue 153 in the DRB MHC class II polypeptide and the Cys at residue 29 in the DRA MHC class II polypeptide. In any of the above embodiments, linkers that do not comprise a Cys optionally can be used to connect the components of the first or second polypeptide. In any of the above embodiments, in some cases, the one or more immunomodulatory polypeptides is a PD-L1 polypeptide or variant thereof, a TGF-β polypeptide or variant thereof, an IL-2 polypeptide or variant thereof, or a FasL polypeptide or variant thereof. In some cases, the Ig Fc polypeptide is present and is a human IgG1 Fc polypeptide, optionally comprising L234A and L235A substitutions (L14A and L15A of the amino acid sequence depicted in FIG. 12A). In some cases, the DRB MHC class II polypeptide comprises an amino acid sequence having at least 80%, at least 85%, at least 90%, at least 95%, at least 98%, at least 99%, or 100%, amino acid sequence identity to the amino acid sequence depicted in FIG. 14J, and comprises a Cys at position 153. In some cases, the DRA MHC class II polypeptide comprises an amino acid sequence having at least 80%, at least 85%, at least 90%, at least 95%, at least 98%, or at least 99% amino acid sequence identity to the amino acid sequence depicted in FIG. 13F, and comprises a Cys at position 29. In some cases, the T1D peptide is a proinsulin peptide. In some cases, the T1D peptide is a proinsulin peptide selected from SLQPLALEGSLQKRG (SEQ ID NO:94; proIns 76-90), SLQPLALEGSLQSRG (SEQ ID NO:90; proIns 76-90; K88S), and GAGSLQPLALEGSLQKRG (SEQ ID NO:93; proIns 73-90). In some cases, the T1D peptide is a GAD peptide. In some cases, the T1D peptide is a GAD peptide selected from NFFRMVISNPAAT (SEQ ID NO:87; GAD65 555-567) and NFIRMVISNPAAT (SEQ ID NO:88; GAD65 555-567; F557I).

TMMPs that are Disulfide Bonded Via a Cys in a Linker Between the T1D Peptide and the First MHC Class II Polypeptide and a Cys in the Second MHC Class II Polypeptide

As noted above, in some cases, a TMMP of the present disclosure comprises a heterodimer comprising a first polypeptide chain comprising: i) a T1D peptide; ii) a peptide linker comprising a Cys; and iii) a first MHC class II polypeptide; and b) a second polypeptide comprising a second MHC class II polypeptide, where the first and/or the second polypeptides comprise one or more immunomodulatory polypeptides, optionally wherein the first or the second polypeptide comprises an Ig Fc polypeptide, where the second MHC class II polypeptide comprises a substitution of an amino acid (other than a Cys) with a Cys; and where the first and the second polypeptides are linked via a disulfide bond between the Cys in the peptide linker and the Cys provided by the substitution in the second MHC class II polypeptide.

For example, in some cases, the peptide linker can comprise an amino acid sequence selected from: (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where n is 1, 2, 3, 4, 5, 6, 7, 8, 9, or 10, with n=2 or 3 generally being useful); the first MHC class II polypeptide is a DRB MHC class II polypeptide; and the second MHC class II polypeptide is a DRA MHC class II polypeptide that comprises a substitution of an amino acid (other than a Cys) with a Cys.

Specific examples of the Cys-containing linkers between the T1D peptide and a DRB MHC class II polypeptide and Cys residues in the DRA MHC class II polypeptide that can form a disulfide bond are provided in Table 4, below:

TABLE 4

| Cys-Containing Linker | |
|---|---|
| Between the T1D pep- | DRA MHC |
| tide and the DRB MHC | class II |
| Class II | polypeptide |
| Polypeptide | substitution |
| (CGGGS)(GGGGS)n | I72C |
| (SEQ ID NO: 1) | |
| (GCGGS)(GGGGS)n | I72C |
| (SEQ ID NO: 2) | |
| (GGCGS)(GGGGS)n | I72C |
| (SEQ ID NO: 3) | |
| (GGGCS)(GGGGS)n | I72C |
| (SEQ ID NO: 4) | |
| (GGGGC)(GGGGS)n | I72C |
| (SEQ ID NO: 5) | |
| (CGGGS)(GGGGS)n | K75C |
| (SEQ ID NO: 1) | |
| (GCGGS)(GGGGS)n | K75C |
| (SEQ ID NO: 2) | |
| (GGCGS)(GGGGS)n | K75C |
| (SEQ ID NO: 3) | |
| (GGGCS)(GGGGS)n | K75C |
| (SEQ ID NO: 4) | |
| (GGGGC)(GGGGS)n | K75C |
| (SEQ ID NO: 5) | |

In some cases, where the first and second MHC class II polypeptides are DRB and DRA polypeptides, respectively, the DRB MHC class II polypeptide can comprise an amino acid sequence having at least 80%, at least 85%, at least 90%, at least 95%, at least 98%, at least 99%, or 100%, amino acid sequence identity to the amino acid sequence depicted in FIG. 14A. The DRA MHC class II polypeptide can comprise an amino acid sequence having at least 80%, at least 85%, at least 90%, at least 95%, at least 98%, or at least 99%, amino acid sequence identity to the amino acid sequence depicted in FIG. 13A, and can comprise a Cys at, e.g., position 72 or position 75. In any of the above embodiments, n=0, 1, 2, 3, 4 or more, with 2 or 3 typically being used to create a total linker length of 15 or 20 amino acids, although longer lengths may be possible. In any of the above embodiments, linkers that do not comprise a Cys optionally can be used to connect the other components of the first or second polypeptide. In some cases, the Ig Fc polypeptide is present and is a human IgG1 Fc polypeptide, optionally comprising L234A and L235A substitutions (L14A and L15A of the amino acid sequence depicted in FIG. 12A). In some cases, the T1D peptide is a proinsulin peptide. In some cases, the T1D peptide is a proinsulin peptide selected from SLQPLALEGSLQKRG (SEQ ID NO:94; proIns 76-90), SLQPLALEGSLQSRG (SEQ ID NO:90; proIns 76-90; K88S), and GAGSLQPLALEGSLQKRG (SEQ ID NO:93; proIns 73-90). In some cases, the T1D peptide is a GAD peptide. In some cases, the T1D peptide is a GAD peptide selected from NFFRMVISNPAAT (SEQ ID NO:87; GAD65 555-567) and NFIRMVISNPAAT (SEQ ID NO:88; GAD65 555-567; F557I). In any of the above embodiments, in some cases, the one or more immunomodulatory polypeptides is a PD-L1 polypeptide or variant thereof, a TGF-β polypeptide or variant thereof, an IL-2 polypeptide or variant thereof, or a FasL polypeptide or variant thereof. The one or more immunomodulatory polypeptides can be at Position 1, Position 2. Position 3, Position 4, or Position 5; where the immunomodulatory polypeptide positions are described above and depicted schematically in FIG. 5.

The following are examples of the TMMPs described above in which the first and the second polypeptides are linked via a disulfide bond between the Cys in the peptide linker and the Cys provided by the substitution in the second MHC class II polypeptide, and wherein the first polypeptide is a DRB MHC class II polypeptide (i.e., a DR β chain polypeptide), and the second polypeptide is a DRA MHC class II polypeptide (i.e., a DR α chain polypeptide).

As one example, a TMMP of the present disclosure comprises a heterodimer comprising one of the following five first polypeptides and one of the following two second polypeptides to create a heterodimer having one or more immunomodulatory polypeptides at MOD position 1 or 3:
a1) a first polypeptide comprising, in order from N-terminus to C-terminus: i) a T1D peptide; ii) a linker comprising the amino acid sequence (CGGGS)(GGGGS)n (SEQ ID NO:1), where n is an integer from 1 to 10; and iii) an MHC class II β chain polypeptide (e.g., a DRB MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 14A); or alternatively
a2) a first polypeptide comprising, in order from N-terminus to C-terminus: i) a T1D peptide; ii) a linker comprising the amino acid sequence (GCGGS)(GGGGS)n (SEQ ID NO:2), where n is an integer from 1 to 10; and iii) an MHC class II β chain polypeptide (e.g., a DRB MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 14A); or alternatively
a3) a first polypeptide comprising, in order from N-terminus to C-terminus: i) a T1D peptide; ii) a linker comprising the amino acid sequence (GGCGS)(GGGGS)n (SEQ ID NO:3), where n is an integer from 1 to 10; and iii) an MHC class II β chain polypeptide (e.g., a DRB MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 14A); or alternatively
a4) a first polypeptide comprising, in order from N-terminus to C-terminus: i) a T1D peptide; ii) a linker comprising the amino acid sequence (GGGCS)(GGGGS)n (SEQ ID NO:4), where n is an integer from 1 to 10; and iii) an MHC class II β chain polypeptide (e.g., a DRB MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 14A); or alternatively
a5) a first polypeptide comprising, in order from N-terminus to C-terminus: i) a T1D peptide; ii) a linker comprising the amino acid sequence (GGGGC)(GGGGS)n (SEQ ID NO:5), where n is an integer from 1 to 10; and iii) an MHC class II β chain polypeptide (e.g., a DRB MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 14A); and
b1) a second polypeptide comprising, in order from N-terminus to C-terminus: i) an MHC class II α chain comprising a Cys at position 72 (e.g., a DRA MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 13G, and comprising a Cys at position 72); an ii) an Ig Fc polypeptide; and iii) one or more immunomodulatory polypeptides. (See FIG. 5, MOD Position 1.); or, alternatively
b2) a second polypeptide comprising, in order from N-terminus to C-terminus: i) an MHC class II α chain comprising a Cys at position 75 (e.g., a DRA MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 13G, and comprising a Cys at position 75); an ii) an Ig Fc polypeptide; and iii) one or more immunomodulatory polypeptides. (See FIG. 5, MOD Position 1); or, alternatively
b3) a second polypeptide comprising, in order from N-terminus to C-terminus: i) an MHC class II α chain comprising a Cys at position 72 (e.g., a DRA MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 3G, and comprising a Cys at position 72); an ii) an Ig Fc polypeptide; and iii) one or more immunomodulatory polypeptides. (See FIG. 5, MOD Position 3.); or, alternatively
b4) a second polypeptide comprising, in order from N-terminus to C-terminus: i) an MHC class II α chain comprising a Cys at position 75 (e.g., a DRA MHC class II polypeptide comprising an amino acid sequence having at least 80%, at least 85%, at least 90%, at least 95%, at least 98%, at least 99%, or 100% amino acid sequence identity to the amino acid sequence depicted in FIG. 13G, and comprising a Cys at position 75); an ii) an Ig Fc polypeptide; and iii) one or more immunomodulatory polypeptides. (See FIG. 5, MOD Position 3.)

Each of the above combinations of first and second polypeptides for making a TMMP is thus expressly disclosed, i.e., a1-b1, a2-b1, a3-b1, a4-b1, a5-b1, a1-b2, a2-b2, a3-b2, a4-b2 and a5-b2, a1-b3, a2-b3, a3-b3, a4-b3, a5-b3, and a1-b4, a2-b4, a3-b4, a4-b4, and a5-b4.

Alternatively, the one or more immunomodulatory polypeptides in the second polypeptide can be at Position 2, Position 4, or Position 5, which are described above and depicted schematically in FIG. 5.

As noted above, in any of the above embodiments, n=0, 1, 2, 3, 4 or more, with 2 or 3 typically being used to create a total linker length of 15 or 20 amino acids, although longer lengths may be used. In any of the above embodiments, linkers that do not comprise a Cys optionally can be used to connect the other components of the second polypeptide (e.g., a (G4S)n (SEQ ID NO:71) linker can be used between the PD-L1 polypeptide and the DRA MHC class II polypeptide, where n=1, 2, 3, 4, 5, 6, 7, 8, 9, or 10, and a GGSAAAGG (SEQ ID NO:83) linker or AAAGG (SEQ ID NO:82) linker can be used between the DRA MHC class II polypeptide and the Ig Fc polypeptide). In the above embodiments, in some cases, the Ig Fc polypeptide is a human IgG1 Fc polypeptide comprising L234A and L235A substitutions (L14A and L15A of the amino acid sequence depicted in FIG. 12A). In some cases, the T1D peptide is a proinsulin peptide. In some cases, the T1D peptide is a proinsulin peptide selected from SLQPLALEGSLQKRG (SEQ ID NO:94; proIns 76-90), SLQPLALEGSLQSRG (SEQ ID NO:90; proIns 76-90; K88S), and GAGSLQPLALEGSLQKRG (SEQ ID NO:93; proIns 73-90). In some cases, the T1D peptide is a GAD peptide. In some cases, the T1D peptide is a GAD peptide selected from NFFRMVISNPAAT (SEQ ID NO:87; GAD65 555-567) and NFIRMVISNPAAT (SEQ ID NO:88; GAD65 555-567; F557I).

In any of the above embodiments, in some cases, the one or more immunomodulatory polypeptides is a PD-L1 polypeptide or variant thereof, a TGF-β polypeptide or variant thereof, an IL-2 polypeptide or variant thereof, or a FasL polypeptide or variant thereof. In some cases, when the one or more immunomodulatory polypeptides is a PD-L1 polypeptide or variant thereof, the PD-L1 polypeptide comprises the amino acid sequence

(SEQ ID NO: 99)
FTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISY
GGADYKRITVKVNAPYNKINQRILVVDPVTSEHELTCQAEGYPKAEVIWT
SSDHQVLSGKTTTTNSKREEKLFNVTSTLRINTTTNEIFYCTFRRLDPEE
NHTAELVIPELPLAHPPNER.

In some cases, when the one or more immunomodulatory polypeptides comprises an IL-2 polypeptide or variant thereof, the IL-2 polypeptide comprises the following amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:121), where the IL-2 polypeptide is a variant comprising H16A and F42A substitutions relative to wild-type IL-2.

Hence, for example, a TMMP in accordance with the above embodiments can comprise the combination of first polypeptide chain a3 and second polypeptide chain b2, wherein
i) the MHC class II β chain polypeptide is a DRB MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 14A,
ii) the MHC class II α chain polypeptide is a DRA MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 13G, and comprising a Cys at position 75,
iii) the T1D peptide is SLQPLALEGSLQSRG (SEQ ID NO:90; proIns 76-90; K88S),
iv) the linker between the T1D peptide and the DRB MHC class II polypeptide is (GGCGS)(GGGGS)n (SEQ ID NO:128), where n=2 (for a total of 15 amino acids in the linker between the T1D peptide and the DRB MHC class II polypeptide),
v) the Ig Fc polypeptide is a human IgG1 Fc polypeptide comprising L234A and L235A substitutions (L14A and L15A of the amino acid sequence depicted in FIG. 12A),
v) the one or more immunomodulatory polypeptides comprise a PD-L1 polypeptide comprising the sequence:

(SEQ ID NO: 99)
FTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISY
GGADYKRITVKVNAPYNKINQRILVVDPVTSEHELTCQAEGYPKAEVIWT
SSDHQVLSGKTTTTNSKREEKLFNVTSTLRINTTTNEIFYCTFRRLDPEE
NHTAELVIPELPLAHPPNER,

vi) there is a (G4S)₄ (SEQ ID NO:75) linker between the PD-L1 polypeptide and the DRA MHC class II polypeptide,
vii) there is a GGSAAAGG (SEQ ID NO:83) linker between the DRA MHC class II polypeptide and the Ig Fc polypeptide, and
viii) there is a disulfide bond joining the Cys in the (GGCGS)(GGGGS), (SEQ ID NO:128) linker and the Cys at the K75C substitution in the DRA MHC class II polypeptide.

As another example, a TMMP of the present disclosure can comprise: a) a first polypeptide comprising the amino acid sequence of construct “2938” depicted in FIG. 15A; and b) a second polypeptide comprising the amino acid sequence of construct “3893” depicted in FIG. 15B.

### Antigen-Presenting, Disulfide-Linked Heterodimers (i.e., Mod-Less TMMPs)

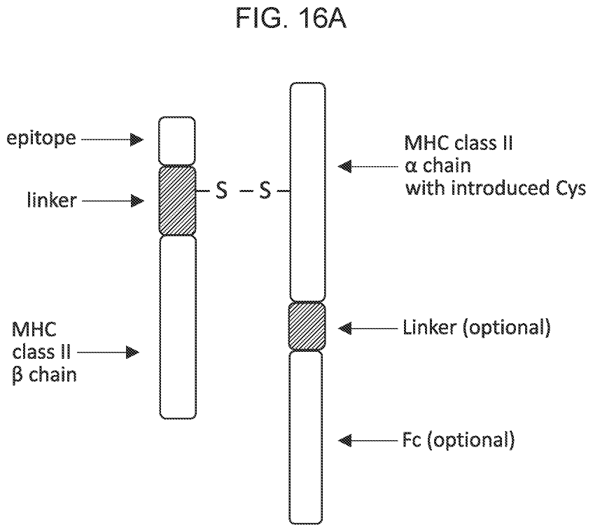

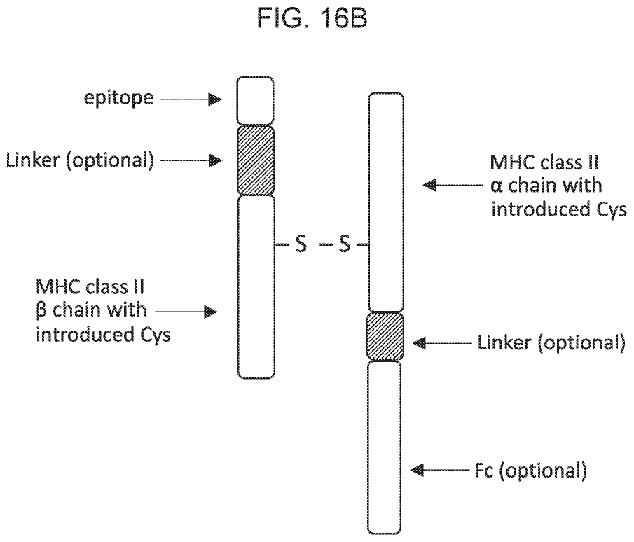

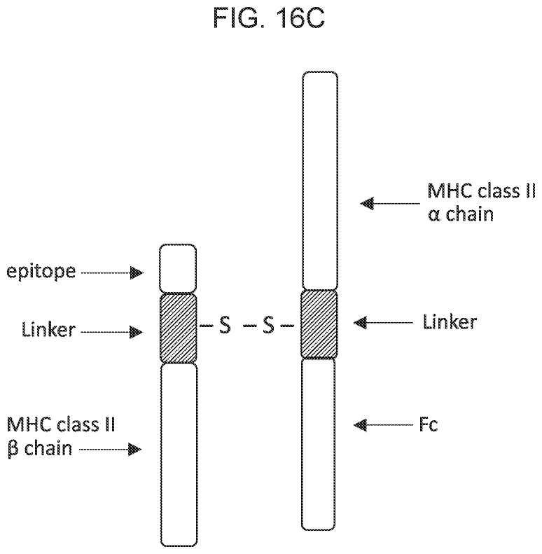

The present disclosure provides an antigen-presenting polypeptide (APP) that comprises a heterodimer (e.g., comprises a heterodimer or a homodimer of two heterodimers), where the heterodimer comprises: a) a first polypeptide comprising a T1D peptide; and ii) a first MHC class II polypeptide; and b) a second polypeptide comprising a second MHC class II polypeptide, where the heterodimer does not include an immunomodulatory polypeptide, where the first polypeptide and the second polypeptide are disulfide linked to one another, optionally where the first or the second polypeptide comprises an Ig Fc polypeptide. An APP of the present disclosure is depicted schematically in FIG. 16A-16C. An APP of the present disclosure is useful for diagnostic applications and therapeutic applications. As discussed below, when used for diagnostic applications, the APP also can comprise a detectable label so that binding of the APP to a target T cell is detected by detecting the detectable label.

Any of the TMMPs described above may be used without the immunomodulatory polypeptide(s) component as an APP. Further, because the APP comprises an Ig Fc polypeptide, an APP typically will be a homodimer comprising two heterodimers that are joined by disulfide bonds that spontaneously form between the two Ig Fc polypeptides. The APP comprises a suitable T1D peptides as described above. In some cases, the first MHC class II polypeptide is an MHC class II β chain polypeptide such as a DRB MHC class II polypeptide. In some cases, the second MHC class II polypeptide is an MHC class II α chain polypeptide such as a DRA MHC class II polypeptide. Suitable Ig Fc polypeptides are as described above. In some cases, the Ig Fc polypeptide is one that induces cell lysis through activation of complement-dependent cytotoxicity (CDC). In some cases, the Ig Fc polypeptide is one that can elicit antibody-dependent cellular cytotoxicity (ADCC). In some cases, the Ig Fc polypeptide binds complement or a complement component. In some cases, the Ig Fc polypeptide is one that substantially does not induce cell lysis through activation of CDC. When the Ig Fc polypeptide is one that that induces cell lysis through activation of CDC, and/or elicits ADCC, the APP can bind to and destroy the target T cell. An APP used for diagnostic purposes typically will have an Ig Fc polypeptide is one that substantially does not induce cell lysis through activation of CDC or elicit ADCC.

In some cases, therefore, an APP of the present disclosure comprises a heterodimer comprising: a) a first polypeptide comprising, in order from N-terminus to C-terminus: i) a T1D peptide; and ii) a first MHC class II polypeptide comprising a substitution of an amino acid (other than a Cys) with a Cys (a “first Cys”); and b) a second polypeptide comprising, in order form N-terminus to C-terminus: i) a second MHC class II polypeptide comprising a substitution of an amino acid (other than a Cys) with a Cys (a “second Cys”); and ii) an Ig Fc polypeptide, where the first polypeptide and the second polypeptide are disulfide bonded via the first Cys and the second Cys. The first MHC class II polypeptide can be a DRB MHC class II polypeptide comprising an amino acid sequence having at least 80%, at least 85%, at least 90%, at least 95%, at least 98%, or at least 99%, amino acid sequence identity to the amino acid sequence depicted in FIG. 14A; and having an amino acid substitution selected from P5C, F7C, Q10C, N19C, G20C. H33C, G151C, D152C, and W153C. The second MHC class II polypeptide can be a DRA MHC class II polypeptide comprising an amino acid sequence having at least 80%, at least 85%, at least 90%, at least 95%, at least 98%, or at least 99%, amino acid sequence identity to the amino acid sequence depicted in FIG. 13A; and having an amino acid substitution selected from E3C, E4C. F12C, G28C, D29C, 172C, K75C. T80C, P81C, 182C, T93C, N94C, and $95C. In some cases, the Ig Fc polypeptide induces cell lysis through activation of complement-dependent cytotoxicity (CDC) and/or elicits antibody-dependent cellular cytotoxicity (ADCC). In some cases, the Ig Fc polypeptide is a variant that substantially does not induce cell lysis through activation of CDC, e.g., an IgG1 Fc polypeptide comprising L234A and L235A substitutions (L14A and L15A of the amino acid sequence depicted in FIG. 12A). In some cases, the T1D peptide is a proinsulin peptide. In some cases, the T1D peptide is a proinsulin peptide selected from SLQPLALEGSLQKRG (SEQ ID NO:94; proIns 76-90), SLQPLALEGSLQSRG (SEQ ID NO:90; proIns 76-90; K88S), and GAGSLQPLALEGSLQKRG (SEQ ID NO:93; proIns 73-90). In some cases, the T1D peptide is a GAD peptide. In some cases, the T1D peptide is a GAD peptide selected from NFFRMVISNPAAT (SEQ ID NO:87; GAD65 555-567) and NFIRMVISNPAAT (SEQ ID NO:88; GAD65 555-567; F557I).

In some cases, an APP of the present disclosure comprises a heterodimer comprising: a) a first polypeptide comprising, in order from N-terminus to C-terminus: i) a T1D peptide; ii) a linker comprising a Cys (a “linker Cys”), and iii) a first MHC class II polypeptide; and b) a second polypeptide comprising, in order form N-terminus to C-terminus: i) a second MHC class II polypeptide comprising a substitution of an amino acid (other than a Cys) with a Cys (a “second Cys”); and ii) an Ig Fc polypeptide, where the first polypeptide and the second polypeptide are disulfide bonded via the linker Cys and the second Cys. The first MHC class II polypeptide can be a DRB MHC class II polypeptide comprising an amino acid sequence having at least 80%, at least 85%, at least 90%, at least 95%, at least 98%, at least 99%, or at least 100% amino acid sequence identity to the amino acid sequence depicted in FIG. 14A. The second MHC class II polypeptide can be a DRA MHC class II polypeptide comprising an amino acid sequence having at least 80%, at least 85%, at least 90%, at least 95%, at least 98%, or at least 99%, amino acid sequence identity to the amino acid sequence depicted in FIG. 13A; and having an amino acid substitution selected from 172C or K75C. In some cases, the Ig Fe polypeptide induces cell lysis through activation of complement-dependent cytotoxicity (CDC) and/or elicits antibody-dependent cellular cytotoxicity (ADCC). In some cases, the Ig Fc polypeptide is a variant that substantially does not induce cell lysis through activation of CDC, e.g., an IgG1 Fc polypeptide comprising L234A and L235A substitutions (L14A and L15A of the amino acid sequence depicted in FIG. 12A). In some cases, the T1D peptide is a proinsulin peptide. In some cases, the T1D peptide is a proinsulin peptide selected from SLQPLALEGSLQKRG (SEQ ID NO:94; proIns 76-90), SLQPLALEGSLQSRG (SEQ ID NO:90; proIns 76-90; K88S), and GAGSLQPLALEGSLQKRG (SEQ ID NO:93; proIns 73-90). In some cases, the T1D peptide is a GAD peptide. In some cases, the T1D peptide is a GAD peptide selected from NFFRMVISNPAAT (SEQ ID NO:87; GAD65 555-567) and NFIRMVISNPAAT (SEQ ID NO:88; GAD65 555-567; F557I).

For example, in some cases, the peptide linker can comprise an amino acid sequence selected from: (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where n is 1, 2, 3, 4, 5, 6, 7, 8, 9, or 10, with n=2 or 3 generally being useful); the first MHC class II polypeptide is a DRB MHC class II polypeptide; and the second MHC class II polypeptide is a DRA MHC class II polypeptide that comprises a substitution of an amino acid (other than a Cys) with a Cys.

Specific examples of the Cys-containing linkers between the T1D peptide and a DRB MHC class II polypeptide and Cys residues in the DRA MHC class II polypeptide that can form a disulfide bond are provided in the Table 5, below:

TABLE 5

| Cys-Containing Linker | DRA MHC |
|---|---|
| Between the T1D pep- | class II |
| tide and the DRB MHC | polypeptide |
| Class II Polypeptide | substitution |
| (CGGGS)(GGGGS)n | I72C |
| (SEQ ID NO: 1) | |
| (GCGGS)(GGGGS)n | I72C |
| (SEQ ID NO: 2) | |
| (GGCGS)(GGGGS)n | I72C |
| (SEQ ID NO: 3) | |
| (GGGCS)(GGGGS)n | I72C |
| (SEQ ID NO: 4) | |
| (GGGGC)(GGGGS)n | I72C |
| (SEQ ID NO: 5) | |
| (CGGGS)(GGGGS)n | K75C |
| (SEQ ID NO: 1) | |
| (GCGGS)(GGGGS)n | K75C |
| (SEQ ID NO: 2) | |
| (GGCGS)(GGGGS)n | K75C |
| (SEQ ID NO: 3) | |
| (GGGCS)(GGGGS)n | K75C |
| (SEQ ID NO: 4) | |
| (GGGGC)(GGGGS)n | K75C |
| (SEQ ID NO: 5) | |

As noted above, in any of the above APP embodiments, n=0, 1, 2, 3, 4 or more, with 2 or 3 typically being used to create a total linker length of 15 or 20 amino acids, although longer lengths may be possible. In any of the above embodiments, linkers that do not comprise a Cys optionally can be used to connect the other components of second polypeptide. In some cases, the Ig Fc polypeptide induces cell lysis through activation of complement-dependent cytotoxicity (CDC) and/or elicits antibody-dependent cellular cytotoxicity (ADCC). In some cases, the Ig Fc polypeptide is present and is a human IgG1 Fc polypeptide, optionally comprising L234A and L235A substitutions (L14A and L15A of the amino acid sequence depicted in FIG. 12A). In some cases, the T1D peptide is a proinsulin peptide. In some cases, the T1D peptide is a proinsulin peptide selected from SLQPLALEGSLQKRG (SEQ ID NO:94; proIns 76-90), SLQPLALEGSLQSRG (SEQ ID NO:94; proIns 76-90; K88S), and GAGSLQPLALEGSLQKRG (SEQ ID NO:93; proIns 73-90). In some cases, the T1D peptide is a GAD peptide. In some cases, the T1D peptide is a GAD peptide selected from NFFRMVISNPAAT (SEQ ID NO:87; GAD65 555-567) and NFIRMVISNPAAT (SEQ ID NO:88; GAD65 555-567; F557I).

The following are examples of the APPs described above in which the first and the second polypeptides are linked via a disulfide bond between the Cys in the peptide linker and the Cys provided by the substitution in the second MHC class II polypeptide, and wherein the first polypeptide is a DRB MHC class II polypeptide (i.e., a DR β chain polypeptide), and the second polypeptide is a DRA MHC class II polypeptide (i.e., a DR α chain polypeptide).

As one example, an APP of the present disclosure comprises a heterodimer comprising one of the following five first polypeptides and one of the following two second polypeptides:
a1) a first polypeptide comprising, in order from N-terminus to C-terminus: i) a T1D peptide; ii) a linker comprising the amino acid sequence (CGGGS)(GGGGS)n (SEQ ID NO:1), where n is an integer from 1 to 10; and iii) an MHC class II β chain polypeptide (e.g., a DRB MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 14A); or alternatively
a2) a first polypeptide comprising, in order from N-terminus to C-terminus: i) a T1D peptide; ii) a linker comprising the amino acid sequence (GCGGS)(GGGGS)n (SEQ ID NO:2), where n is an integer from 1 to 10; and iii) an MHC class II β chain polypeptide (e.g., a DRB MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 14A); or alternatively
a3) a first polypeptide comprising, in order from N-terminus to C-terminus: i) a T1D peptide; ii) a linker comprising the amino acid sequence (GGCGS)(GGGGS)n (SEQ ID NO:3), where n is an integer from 1 to 10; and iii) an MHC class II β chain polypeptide (e.g., a DRB MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 4A); or alternatively
a4) a first polypeptide comprising, in order from N-terminus to C-terminus: i) a T1D peptide; ii) a linker comprising the amino acid sequence (GGGCS)(GGGGS)n (SEQ ID NO:4), where n is an integer from 1 to 10; and iii) an MHC class II β chain polypeptide (e.g., a DRB MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 14A); or alternatively
a5) a first polypeptide comprising, in order from N-terminus to C-terminus: i) a T1D peptide; ii) a linker comprising the amino acid sequence (GGGGC)(GGGGS)n (SEQ ID NO:5), where n is an integer from 1 to 10; and iii) an MHC class II β chain polypeptide (e.g., a DRB MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 14A); and
b1) a second polypeptide comprising, in order from N-terminus to C-terminus: i) an MHC class II α chain comprising a Cys at position 72 (e.g., a DRA MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 13G, and comprising a Cys at position 72); and an ii) an Ig Fc polypeptide; or, alternatively
b2) a second polypeptide comprising, in order from N-terminus to C-terminus: i) an MHC class II α chain comprising a Cys at position 75 (e.g., a DRA MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 13G, and comprising a Cys at position 75); and ii) an Ig Fc polypeptide.

Each of the above combinations of first and second polypeptides for making an APP is thus expressly disclosed, i.e., a1-b1, a2-b1, a3-b1, a4-b1, a5-b1, and a1-b2, a2-b2, a3-b2, a4-b2 and a5-b2.

As noted above, in any of the above embodiments, n=0, 1, 2, 3, 4 or more, with 2 or 3 typically being used to create a total linker length of 15 or 20 amino acids, although longer lengths may be usable. In any of the above embodiments, linkers that do not comprise a Cys optionally can be used to connect the components of the second polypeptide (e.g., a GGSAAAGG (SEQ ID NO:83) linker or AAAGG (SEQ ID NO:82) linker can be used between the DRA MHC class II polypeptide and the Ig Fc polypeptide). In the above embodiments, in some cases, the Ig Fc polypeptide is a human IgG1 Fc polypeptide comprising L234A and L235A substitutions (L14A and L15A of the amino acid sequence depicted in FIG. 12A). In some cases, the Ig Fc polypeptide induces cell lysis through activation of complement-dependent cytotoxicity (CDC) and/or elicits antibody-dependent cellular cytotoxicity (ADCC). In some cases, the T1D peptide is a proinsulin peptide. In some cases, the T1D peptide is a proinsulin peptide selected from SLQPLALEGSLQKRG (SEQ ID NO:94; proIns 76-90), SLQPLALEGSLQSRG (SEQ ID NO:90; proIns 76-90; K88S), and GAGSLQPLALEGSLQKRG (SEQ ID NO:93; proIns 73-90). In some cases, the T1D peptide is a GAD peptide. In some cases, the T1D peptide is a GAD peptide selected from NFFRMVISNPAAT (SEQ ID NO:87; GAD65 555-567) and NFIRMVISNPAAT (SEQ ID NO:88; GAD65 555-567; F557I).

Hence, for example, a TMMP in accordance with the above embodiments can comprise the combination of first polypeptide chain α3 and second polypeptide chain b2, wherein
i) the MHC class II β chain polypeptide is a DRB MHC class II polypeptide comprising an amino acid sequence having at least 80%, at least 85%, at least 90%, at least 95%, at least 98%, at least 99%, or 100% amino acid sequence identity to the amino acid sequence depicted in FIG. 14A,
ii) the MHC class II α chain polypeptide is a DRA MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 13G, and comprising a Cys at position 75,
iii) the T1D peptide is SLQPLALEGSLQSRG (SEQ ID NO:90; proIns 76-90; K88S),
iv) the linker between the T1D peptide and the DRB MHC class II polypeptide is (GGCGS)(GGGGS)n (SEQ ID NO:128), where n=2 (for a total of 15 amino acids in the linker between the T1D peptide and the DRB MHC class II polypeptide),
v) the Ig Fc polypeptide is a human IgG1 Fc polypeptide comprising the amino acid sequence depicted in FIG. 12A, or a variant thereof such as the amino acid sequence depicted in FIG. 12A comprising L234A and L235A substitutions (L14A and L15A of the amino acid sequence depicted in FIG. 12A),
vi) there is a GGSAAAGG (SEQ ID NO:83) linker between the DRA MHC class II polypeptide and the Ig Fc polypeptide, and
viii) there is a disulfide bond joining the Cys in the (GGCGS)(GGGGS): (SEQ ID NO:128) linker and the Cys at the K75C substitution in the DRA MHC class II polypeptide.

A disulfide-bonded APP of the present disclosure will in some cases exhibit greater stability than a control non-disulfide-bonded APP. An example of a control APP is an APP comprising first and second polypeptide chains 2744 and 2932 (depicted in FIG. 15AA and FIG. 15Z, respectively), or 2639 and 2932 (depicted in FIG. 15C and FIG. 15Z, respectively). A disulfide-bonded APP of the present disclosure will in some cases exhibit at least 10%, at least 15%, at least 20%, at least 25%, at least 30%, at least 50%, at least 75%, at least 2-fold, at least 5-fold, at least 10-fold, at least 50-fold, at least 100-fold, or greater than 100-fold, greater stability in the PBS buffer solution containing 500 mM NaCl (described above) in vitro over a specified period of time and at a specified temperature (e.g., in a solution at a temperature of from 37° C. to 42° C. for a period of time of from 1 hour to 28 days; e.g., for 1 hour at 37° C.; 1 day at 37° C.; 5 days at 37° C.; 1 hour at 42° C.; 1 day at 42° C.; 5 days at 42° C.; 5 days at 37° C.; 10 days at 37° C.; 14 days at 37° C.; 28 days at 37° C.; and the like) than a control non-disulfide-bonded APP. A disulfide-bonded APP of the present disclosure will in some cases exhibit greater expression than a control non-disulfide-bonded APP. A disulfide-bonded APP of the present disclosure will in some cases exhibit both greater stability and greater expression than a control non-disulfide-bonded APP. For example, in some cases, an APP of the present disclosure is expressed (produced) in a Chinese hamster ovary (CHO) cell in vitro at a level that is at least 10%, at least 20%, at least 30%, at least 40%, at least 50%, at least 60%, at least 70%, at least 80%, at least 90%, at least 2-fold, at least 2.5-fold, at least 3-fold, at least 4-fold, at least 5-fold, at least 10-fold, at least 20-fold, at least 50-fold, at least 100-fold, or more than 100-fold, higher than the expression level of a control APP lacking the at least one disulfide bond when expressed under the same conditions and in the same CHO cells.

Expression levels can be determined by: i) producing the APP in a CHO cell in vitro; and ii) determining the amount of APP produced by the mammalian cell. For example, the APP can be isolated from the CHO cells and/or from culture medium in which the CHO cells are cultured, where isolation of the APP can be carried out by affinity chromatography, e.g., on a Protein A column, a Protein G column, or the like. An example of a suitable mammalian cell is a CHO cell; e.g., an Expi-CHO-S™ cell (e.g., ThermoFisher Scientific, Catalog #A29127).

As one example, an APP of the present disclosure comprises: a) a first polypeptide comprising the amino acid sequence of construct 2986, depicted in FIG. 15I, comprising an F7C substitution (Cys-7) in the MHC class II β chain relative to the MHC class II β chain sequence depicted in FIG. 14A; and b) a second polypeptide comprising the amino acid sequence of construct 2983, depicted in FIG. 15H, comprising a P81C substitution (Cys-81) in the MHC class II α chain relative to the MHC class II α chain sequence depicted in FIG. 13A, where the first polypeptide and the second polypeptide are covalently linked to one another via a disulfide bond between Cys-7 and Cys-81.

As another example, an APP of the present disclosure comprises: a) a first polypeptide comprising the amino acid sequence of construct 2987, depicted in FIG. 15J, comprising an P5C substitution (Cys-5) in the MHC class II β chain relative to the MHC class II β chain sequence depicted in FIG. 14A; and b) a second polypeptide comprising the amino acid sequence of construct 2983, depicted in FIG. 15H, comprising a P81C substitution (Cys-81) in the MHC class II α chain relative to the MHC class II α chain sequence depicted in FIG. 13A, where the first polypeptide and the second polypeptide are covalently linked to one another via a disulfide bond between Cys-5 and Cys-81.

As another example, an APP of the present disclosure comprises: a) a first polypeptide comprising the amino acid sequence of construct 3027, depicted in FIG. 15K, comprising an H33C substitution (Cys-33) in the MHC class II β chain relative to the MHC class II β chain sequence depicted in FIG. 14A; and b) a second polypeptide comprising the amino acid sequence of construct 2983, depicted in FIG. 15H, comprising a P81C substitution (Cys-81) in the MHC class II α chain relative to the MHC class II α chain sequence depicted in FIG. 13A, where the first polypeptide and the second polypeptide are covalently linked to one another via a disulfide bond between Cys-33 and Cys-81.

As another example, an APP of the present disclosure comprises: a) a first polypeptide comprising the amino acid sequence of construct 2988, depicted in FIG. 15M, comprising an N19C substitution (N19C) in the MHC class II β chain relative to the MHC class II β chain sequence depicted in FIG. 14A; and b) a second polypeptide comprising the amino acid sequence of construct 2984, depicted in FIG. 15L, comprising an E4C substitution (Cys-4) in the MHC class II α chain relative to the MHC class II α chain sequence depicted in FIG. 13A, where the first polypeptide and the second polypeptide are covalently linked to one another via a disulfide bond between Cys-19 and Cys-4.

As another example, an APP of the present disclosure comprises: a) a first polypeptide comprising the amino acid sequence of construct 3032, depicted in FIG. 15N, comprising a G20C substitution (Cys-20) in the MHC class II β chain relative to the MHC class II β chain sequence depicted in FIG. 14A; and b) a second polypeptide comprising the amino acid sequence of construct 2984, depicted in FIG. 15L, comprising an E4C substitution (Cys-4) in the MHC class II α chain relative to the MHC class II α chain sequence depicted in FIG. 13A, where the first polypeptide and the second polypeptide are covalently linked to one another via a disulfide bond between Cys-20 and Cys-4.

As another example, an APP of the present disclosure comprises: a) a first polypeptide comprising the amino acid sequence of construct 2989, depicted in FIG. 15P, comprising a Q56C substitution (Cys-156) in the MHC class II β chain relative to the MHC class II β chain sequence depicted in FIG. 14A; and b) a second polypeptide comprising the amino acid sequence of construct 2985, depicted in FIG. 15O, comprising a T93C substitution (Cys-93) in the MHC class II α chain relative to the MHC class II α chain sequence depicted in FIG. 13A, where the first polypeptide and the second polypeptide are covalently linked to one another via a disulfide bond between Cys-156 and Cys-93.

As another example, an APP of the present disclosure comprises: a) a first polypeptide comprising the amino acid sequence of construct 3030, depicted in FIG. 15Q, comprising a W153C substitution (Cys-153) in the MHC class II β chain relative to the MHC class II β chain sequence depicted in FIG. 14A; and b) a second polypeptide comprising the amino acid sequence of construct 2985, depicted in FIG. 15O, comprising a T93C substitution (Cys-93) in the MHC class II α chain relative to the MHC class II α chain sequence depicted in FIG. 13A, where the first polypeptide and the second polypeptide are covalently linked to one another via a disulfide bond between Cys-153 and Cys-93.

As another example, an APP of the present disclosure comprises: a) a first polypeptide comprising the amino acid sequence of construct 2986, depicted in FIG. 15I, comprising an F7C substitution (Cys-7) in the MHC class II β chain relative to the MHC class II β chain sequence depicted in FIG. 14A; and b) a second polypeptide comprising the amino acid sequence of construct 3018, depicted in FIG. 15R, comprising an F12C substitution (Cys-12) in the MHC class II α chain relative to the MHC class II α chain sequence depicted in FIG. 13A, where the first polypeptide and the second polypeptide are covalently linked to one another via a disulfide bond between Cys-7 and Cys-12.

As another example, an APP of the present disclosure comprises: a) a first polypeptide comprising the amino acid sequence of construct 3026, depicted in FIG. 15S, comprising a Q10C substitution (Cys-10) in the MHC class II β chain relative to the MHC class II β chain sequence depicted in FIG. 14A; and b) a second polypeptide comprising the amino acid sequence of construct 3018, depicted in FIG. 15R, comprising an F12C substitution (Cys-12) in the MHC class II α chain relative to the MHC class II α chain sequence depicted in FIG. 13A, where the first polypeptide and the second polypeptide are covalently linked to one another via a disulfide bond between Cys-10 and Cys-12.

As another example, an APP of the present disclosure comprises: a) a first polypeptide comprising the amino acid sequence of construct 2986, depicted in FIG. 15I, comprising an F7C substitution (Cys-7) in the MHC class II β chain relative to the MHC class II β chain sequence depicted in FIG. 14A; and b) a second polypeptide comprising the amino acid sequence of construct 3019, depicted in FIG. 15T, comprising a T80C substitution (Cys-80) in the MHC class II α chain relative to the MHC class II α chain sequence depicted in FIG. 13A, where the first polypeptide and the second polypeptide are covalently linked to one another via a disulfide bond between Cys-7 and Cys-80.

As another example, an APP of the present disclosure comprises: a) a first polypeptide comprising the amino acid sequence of construct 2987, depicted in FIG. 15J, comprising a P5C substitution (Cys-5) in the MHC class II β chain relative to the MHC class II β chain sequence depicted in FIG. 14A; and b) a second polypeptide comprising the amino acid sequence of construct 3019, depicted in FIG. 15T, comprising a T80C substitution (Cys-80) in the MHC class II α chain relative to the MHC class II α chain sequence depicted in FIG. 13A, where the first polypeptide and the second polypeptide are covalently linked to one another via a disulfide bond between Cys-5 and Cys-80.

As another example, an APP of the present disclosure comprises: a) a first polypeptide comprising the amino acid sequence of construct 3027, depicted in FIG. 15K, comprising an 182C substitution (Cys-82) in the MHC class II β chain relative to the MHC class II β chain sequence depicted in FIG. 14A; and b) a second polypeptide comprising the amino acid sequence of construct 3019, depicted in FIG. 15T, comprising a T80C substitution (Cys-80) in the MHC class II α chain relative to the MHC class II α chain sequence depicted in FIG. 13A, where the first polypeptide and the second polypeptide are covalently linked to one another via a disulfide bond between Cys-82 and Cys-80.

As another example, an APP of the present disclosure comprises: a) a first polypeptide comprising the amino acid sequence of construct 2986, depicted in FIG. 15I, comprising an F7C substitution (Cys-7) in the MHC class II β chain relative to the MHC class II β chain sequence depicted in FIG. 14A; and b) a second polypeptide comprising the amino acid sequence of construct 3018, depicted in FIG. 15R, comprising an F12C substitution (Cys-12) in the MHC class II α chain relative to the MHC class II α chain sequence depicted in FIG. 13A, where the first polypeptide and the second polypeptide are covalently linked to one another via a disulfide bond between Cys-7 and Cys-12.

As another example, an APP of the present disclosure comprises: a) a first polypeptide comprising the amino acid sequence of construct 3026, depicted in FIG. 15S, comprising a Q10C substitution (Cys-10) in the MHC class II β chain relative to the MHC class II β chain sequence depicted in FIG. 14A; and b) a second polypeptide comprising the amino acid sequence of construct 3018, depicted in FIG. 15R, comprising an F12C substitution (Cys-12) in the MHC class II α chain relative to the MHC class II α chain sequence depicted in FIG. 13A, where the first polypeptide and the second polypeptide are covalently linked to one another via a disulfide bond between Cys-10 and Cys-12.

As another example, an APP of the present disclosure comprises: a) a first polypeptide comprising the amino acid sequence of construct 2986, depicted in FIG. 15I, comprising an F7C substitution (Cys-7) in the MHC class II β chain relative to the MHC class II β chain sequence depicted in FIG. 14A; and b) a second polypeptide comprising the amino acid sequence of construct 3019, depicted in FIG. 15T, comprising a T80C substitution (Cys-80) in the MHC class II α chain relative to the MHC class II α chain sequence depicted in FIG. 13A, where the first polypeptide and the second polypeptide are covalently linked to one another via a disulfide bond between Cys-7 and Cys-80.

As another example, an APP of the present disclosure comprises: a) a first polypeptide comprising the amino acid sequence of construct 2987, depicted in FIG. 15J, comprising a P5C substitution (Cys-5) in the MHC class II β chain relative to the MHC class II β chain sequence depicted in FIG. 14A; and b) a second polypeptide comprising the amino acid sequence of construct 3019, depicted in FIG. 15T, comprising a T80C substitution (Cys-80) in the MHC class II α chain relative to the MHC class II α chain sequence depicted in FIG. 13A, where the first polypeptide and the second polypeptide are covalently linked to one another via a disulfide bond between Cys-5 and Cys-80.

As another example, an APP of the present disclosure comprises: a) a first polypeptide comprising the amino acid sequence of construct 3027, depicted in FIG. 15K, comprising an 182C substitution (Cys-82) in the MHC class II β chain relative to the MHC class II β chain sequence depicted in FIG. 14A; and b) a second polypeptide comprising the amino acid sequence of construct 3019, depicted in FIG. 15T, comprising a T80C substitution (Cys-80) in the MHC class II α chain relative to the MHC class II α chain sequence depicted in FIG. 13A, where the first polypeptide and the second polypeptide are covalently linked to one another via a disulfide bond between Cys-82 and Cys-80.

As another example, an APP of the present disclosure comprises: a) a first polypeptide comprising the amino acid sequence of construct 2986, depicted in FIG. 15I, comprising an F7C substitution (Cys-7) in the MHC class II β chain relative to the MHC class II β chain sequence depicted in FIG. 14A; and b) a second polypeptide comprising the amino acid sequence of construct 3020, depicted in FIG. 15U, comprising an 182C substitution (Cys-82) in the MHC class II α chain relative to the MHC class II α chain sequence depicted in FIG. 13A, where the first polypeptide and the second polypeptide are covalently linked to one another via a disulfide bond between Cys-7 and Cys-82.

As another example, an APP of the present disclosure comprises: a) a first polypeptide comprising the amino acid sequence of construct 2987, depicted in FIG. 15J, comprising a P5C substitution (Cys-5) in the MHC class II β chain relative to the MHC class II β chain sequence depicted in FIG. 14A; and b) a second polypeptide comprising the amino acid sequence of construct 3020, depicted in FIG. 15U, comprising an 182C substitution (Cys-82) in the MHC class II α chain relative to the MHC class II α chain sequence depicted in FIG. 13A, where the first polypeptide and the second polypeptide are covalently linked to one another via a disulfide bond between Cys-5 and Cys-82.

As another example, an APP of the present disclosure comprises: a) a first polypeptide comprising the amino acid sequence of construct 3027, depicted in FIG. 15V, comprising an H33C substitution (Cys-33) in the MHC class II β chain relative to the MHC class II β chain sequence depicted in FIG. 14A; and b) a second polypeptide comprising the amino acid sequence of construct 3020, depicted in FIG. 15U, comprising an 182C substitution (Cys-82) in the MHC class II α chain relative to the MHC class II α chain sequence depicted in FIG. 13A, where the first polypeptide and the second polypeptide are covalently linked to one another via a disulfide bond between Cys-33 and Cys-82.

As another example, an APP of the present disclosure comprises: a) a first polypeptide comprising the amino acid sequence of construct 3028, depicted in FIG. 15X, comprising a G151C substitution (Cys-151) in the MHC class II β chain relative to the MHC class II β chain sequence depicted in FIG. 14A; and b) a second polypeptide comprising the amino acid sequence of construct 3021, depicted in FIG. 15W, comprising a G28C substitution (Cys-28) in the MHC class II α chain relative to the MHC class II α chain sequence depicted in FIG. 13A, where the first polypeptide and the second polypeptide are covalently linked to one another via a disulfide bond between Cys-151 and Cys-28.

As another example, an APP of the present disclosure comprises: a) a first polypeptide comprising the amino acid sequence of construct 3029, depicted in FIG. 15Y, comprising a D152C substitution (Cys-152) in the MHC class II β chain relative to the MHC class II β chain sequence depicted in FIG. 14A; and b) a second polypeptide comprising the amino acid sequence of construct 3021, depicted in FIG. 15W, comprising a G28C substitution (Cys-28) in the MHC class II α chain relative to the MHC class II α chain sequence depicted in FIG. 13A, where the first polypeptide and the second polypeptide are covalently linked to one another via a disulfide bond between Cys-152 and Cys-28.

As another example, an APP of the present disclosure comprises: a) a first polypeptide comprising the amino acid sequence of construct 3030, depicted in FIG. 15Q, comprising a W153C substitution (Cys-153) in the MHC class II β chain relative to the MHC class II β chain sequence depicted in FIG. 14A; and b) a second polypeptide comprising the amino acid sequence of construct 3021, depicted in FIG. 15W, comprising a G28C substitution (Cys-28) in the MHC class II α chain relative to the MHC class II α chain sequence depicted in FIG. 13A, where the first polypeptide and the second polypeptide are covalently linked to one another via a disulfide bond between Cys-153 and Cys-28.

As another example, an APP of the present disclosure comprises: a) a first polypeptide comprising the amino acid sequence of construct 3028, depicted in FIG. 15X, comprising a G151C substitution (Cys-151) in the MHC class II β chain relative to the MHC class II β chain sequence depicted in FIG. 14A; and b) a second polypeptide comprising the amino acid sequence of construct 3022, depicted in FIG. 15BB, comprising a D29C substitution (Cys-29) in the MHC class II α chain relative to the MHC class II α chain sequence depicted in FIG. 13A, where the first polypeptide and the second polypeptide are covalently linked to one another via a disulfide bond between Cys-151 and Cys-29.

As another example, an APP of the present disclosure comprises: a) a first polypeptide comprising the amino acid sequence of construct 3029, depicted in FIG. 15Y, comprising a D152C substitution (Cys-152) in the MHC class II β chain relative to the MHC class II β chain sequence depicted in FIG. 14A; and b) a second polypeptide comprising the amino acid sequence of construct 3022, depicted in FIG. 15BB, comprising a D29C substitution (Cys-29) in the MHC class II α chain relative to the MHC class II α chain sequence depicted in FIG. 13A, where the first polypeptide and the second polypeptide are covalently linked to one another via a disulfide bond between Cys-152 and Cys-29.

As another example, an APP of the present disclosure comprises: a) a first polypeptide comprising the amino acid sequence of construct 3030, depicted in FIG. 15Q, comprising a W153C substitution (Cys-153) in the MHC class II β chain relative to the MHC class II β chain sequence depicted in FIG. 14A; and b) a second polypeptide comprising the amino acid sequence of construct 3022, depicted in FIG. 15BB, comprising a D29C substitution (Cys-29) in the MHC class II α chain relative to the MHC class II α chain sequence depicted in FIG. 13A, where the first polypeptide and the second polypeptide are covalently linked to one another via a disulfide bond between Cys-153 and Cys-29.

As another example, an APP of the present disclosure comprises: a) a first polypeptide comprising the amino acid sequence of construct 2989, depicted in FIG. 15P, comprising a Q156C substitution (Cys-156) in the MHC class II β chain relative to the MHC class II β chain sequence depicted in FIG. 14A; and b) a second polypeptide comprising the amino acid sequence of construct 3023, depicted in FIG. 15CC, comprising an N94C substitution (Cys-94) in the MHC class II α chain relative to the MHC class II α chain sequence depicted in FIG. 13A, where the first polypeptide and the second polypeptide are covalently linked to one another via a disulfide bond between Cys-156 and Cys-94.

As another example, an APP of the present disclosure comprises: a) a first polypeptide comprising the amino acid sequence of construct 3031, depicted in FIG. 15DD, comprising an N120C substitution (Cys-120) in the MHC class II β chain relative to the MHC class II β chain sequence depicted in FIG. 14A; and b) a second polypeptide comprising the amino acid sequence of construct 3023, depicted in FIG. 15CC, comprising an N94C substitution (Cys-94) in the MHC class II α chain relative to the MHC class II α chain sequence depicted in FIG. 13A, where the first polypeptide and the second polypeptide are covalently linked to one another via a disulfide bond between Cys-120 and Cys-94.

As another example, an APP of the present disclosure comprises: a) a first polypeptide comprising the amino acid sequence of construct 2989, depicted in FIG. 15P, comprising a Q156C substitution (Cys-156) in the MHC class II β chain relative to the MHC class II β chain sequence depicted in FIG. 14A; and b) a second polypeptide comprising the amino acid sequence of construct 3024, depicted in FIG. 15EE, comprising an S95C substitution (Cys-95) in the MHC class II α chain relative to the MHC class II α chain sequence depicted in FIG. 13A, where the first polypeptide and the second polypeptide are covalently linked to one another via a disulfide bond between Cys-156 and Cys-95.

As another example, an APP of the present disclosure comprises: a) a first polypeptide comprising the amino acid sequence of construct 3031, depicted in FIG. 15DD, comprising an N120C substitution (Cys-120) in the MHC class II β chain relative to the MHC class II β chain sequence depicted in FIG. 14A; and b) a second polypeptide comprising the amino acid sequence of construct 3024, depicted in FIG. 15EE, comprising an S95C substitution (Cys-95) in the MHC class II α chain relative to the MHC class II α chain sequence depicted in FIG. 13A, where the first polypeptide and the second polypeptide are covalently linked to one another via a disulfide bond between Cys-120 and Cys-95.

As another example, an APP of the present disclosure comprises: a) a first polypeptide comprising the amino acid sequence of construct 2988, depicted in FIG. 15M, comprising an N19C substitution (Cys-19) in the MHC class II β chain relative to the MHC class II β chain sequence depicted in FIG. 14A; and b) a second polypeptide comprising the amino acid sequence of construct 3025, depicted in FIG. 15FF, comprising an E3C substitution (Cys-3) in the MHC class II α chain relative to the MHC class II α chain sequence depicted in FIG. 13A, where the first polypeptide and the second polypeptide are covalently linked to one another via a disulfide bond between Cys-19 and Cys-3.

As another example, an APP of the present disclosure comprises: a) a first polypeptide comprising the amino acid sequence of construct 3032, depicted in FIG. 15N, comprising a G20C substitution (Cys-20) in the MHC class II β chain relative to the MHC class II β chain sequence depicted in FIG. 14A; and b) a second polypeptide comprising the amino acid sequence of construct 3025, depicted in FIG. 15FF, comprising an E3C substitution (Cys-3) in the MHC class II α chain relative to the MHC class II α chain sequence depicted in FIG. 13A, where the first polypeptide and the second polypeptide are covalently linked to one another via a disulfide bond between Cys-20 and Cys-3.

### Nucleic Acids

The present disclosure provides a nucleic acid comprising a nucleotide sequence encoding a TMPP or an APP of the present disclosure. For example, the present disclosure provides a single nucleic acid comprising nucleotide sequences encoding both the first polypeptide and the second polypeptide of a TMPP or APP of the present disclosure. As another example, the present disclosure provides: a) a first nucleic acid comprising a nucleotide sequence encoding the first polypeptide of a TMMP or APP of the present disclosure; and b) a second nucleic acid comprising a nucleotide sequence encoding the second polypeptide of a TMMP or APP of the present disclosure. In some cases, the nucleic acid is a recombinant expression vector; thus, the present disclosure provides a recombinant expression vector comprising a nucleotide sequence encoding a TMPP or APP of the present disclosure. The discussion, below, of nucleic acids refers to nucleic acids encoding TMPPs of the present disclosure; however, the discussion applies as well to nucleic acids encoding APPs of the present disclosure.

### Separate Nucleic Acids Encoding Individual Polypeptide Chains of a TMPP or APP

The present disclosure provides nucleic acids comprising nucleotide sequences encoding a TMMP or APP of the present disclosure. As noted above, in some cases, the individual polypeptide chains of a TMMP of the present disclosure are encoded in separate nucleic acids. In some cases, nucleotide sequences encoding the separate polypeptide chains of a TMMP or APP of the present disclosure are operably linked to transcriptional control elements, e.g., promoters, such as promoters that are functional in a eukaryotic cell, where the promoter can be a constitutive promoter or an inducible promoter.

For example, the present disclosure provides a first nucleic acid and a second nucleic acid, where the first nucleic acid comprises a nucleotide sequence encoding the first polypeptide of a TMMP or APP of the present disclosure, and where the second nucleic acid comprises a nucleotide sequence encoding the second polypeptide of the TMMP or APP. In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

### Nucleic Acid Encoding Two or More Polypeptides Present in a TMMP or APP

The present disclosure provides a nucleic acid comprising nucleotide sequences encoding the first polypeptide and the second polypeptide of a TMMP or APP of the present disclosure. In some cases, the nucleotide sequences encoding the first polypeptide and the second polypeptide of a TMMP or APP of the present disclosure includes a proteolytically cleavable linker interposed between the nucleotide sequence encoding the first polypeptide and the nucleotide sequence encoding the second polypeptide. In some cases, the nucleotide sequences encoding the first polypeptide and the second polypeptide of a TMMP or APP of the present disclosure includes an internal ribosome entry site (IRES) interposed between the nucleotide sequence encoding the first polypeptide and the nucleotide sequence encoding the second polypeptide. In some cases, the nucleotide sequences encoding the first polypeptide and the second polypeptide of a TMMP or APP of the present disclosure includes a ribosome skipping signal (or cis-acting hydrolase element, CHYSEL) interposed between the nucleotide sequence encoding the first polypeptide and the nucleotide sequence encoding the second polypeptide. Examples of nucleic acids are described below, where a proteolytically cleavable linker is provided between nucleotide sequences encoding the first polypeptide and the second polypeptide of a TMMP or APP of the present disclosure; in any of these embodiments, an IRES or a ribosome skipping signal can be used in place of the nucleotide sequence encoding the proteolytically cleavable linker.

In some cases, a first nucleic acid (e.g., a recombinant expression vector, an mRNA, a viral RNA, etc.) comprises a nucleotide sequence encoding a first polypeptide chain of a TMMP or APP of the present disclosure; and a second nucleic acid (e.g., a recombinant expression vector, an mRNA, a viral RNA, etc.) comprises a nucleotide sequence encoding a second polypeptide chain of a TMMP or APP of the present disclosure. In some cases, the nucleotide sequence encoding the first polypeptide, and the second nucleotide sequence encoding the second polypeptide, are each operably linked to transcriptional control elements, e.g., promoters, such as promoters that are functional in a eukaryotic cell, where the promoter can be a constitutive promoter or an inducible promoter.

### Recombinant Expression Vectors

The present disclosure provides recombinant expression vectors comprising nucleic acids of the present disclosure. In some cases, the recombinant expression vector is a non-viral vector. In some cases, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some cases, a nucleotide sequence encoding a TMMP or APP of the present disclosure is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some cases, a nucleotide sequence encoding a DNA-targeting RNA and/or a site-directed modifying polypeptide is operably linked to multiple control elements that allow expression of the nucleotide sequence encoding a DNA-targeting RNA and/or a site-directed modifying polypeptide in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### Genetically Modified Host Cells

The present disclosure provides a genetically modified host cell, where the host cell is genetically modified with a nucleic acid(s) of the present disclosure.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, Hela cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), human embryonic kidney (HEK) cells (ATCC No. CRL1573), HLHepG2 cells, and the like.

Genetically modified host cells can be used to produce a TMMP or an APP of the present disclosure. For example, a genetically modified host cell can be used to produce a TMMP of the present disclosure, or an APP of the present disclosure. An expression vector(s) comprising nucleotide sequences encoding the polypeptide(s) is/are introduced into a host cell, generating a genetically modified host cell, which genetically modified host cell produces the polypeptide(s).

### Methods of Producing a TMMP or an APP

The present disclosure provides methods of producing TMMP or an APP of the present disclosure. The methods generally involve culturing, in a culture medium, a host cell (e.g., a genetically modified host cell of the present disclosure) that is genetically modified with a recombinant expression vector(s) comprising a nucleotide sequence(s) encoding the TMMP or the APP; and isolating the TMMP or the APP from the genetically modified host cell and/or the culture medium. As noted above, in some cases, the individual polypeptide chains of a TMMP or APP of the present disclosure are encoded in separate recombinant expression vectors. In some cases, all polypeptide chains of a TMMP or APP of the present disclosure are encoded in a single recombinant expression vector.

Isolation of the TMMP or the APP from the expression host cell (e.g., from a lysate of the expression host cell) and/or the culture medium in which the host cell is cultured, can be carried out using standard methods of protein purification.

For example, a lysate may be prepared of the expression host and the lysate purified using high performance liquid chromatography (HPLC), exclusion chromatography (e.g., size exclusion chromatography), gel electrophoresis, affinity chromatography, or other purification technique. Alternatively, where the TMMP or APP is secreted from the expression host cell into the culture medium, the TMMP or APP can be purified from the culture medium using HPLC, exclusion chromatography, gel electrophoresis, affinity chromatography, or other purification technique. In some cases, the TMMP or APP is purified, e.g., a composition is generated that comprises at least 80% by weight, at least about 85% by weight, at least about 95% by weight, or at least about 99.5% by weight, of the TMMP or APP in relation to contaminants related to the method of preparation of the product and its purification. The percentages can be based upon total protein.

In some cases, e.g., where the TMMP or APP comprises an affinity tag, the TMMP or APP can be purified using an immobilized binding partner of the affinity tag. For example, where a TMMP or an APP comprises an Ig Fc polypeptide, the TMMP or the APP can be isolated from genetically modified mammalian host cell and/or from culture medium in which the mammalian cells are cultured, where isolation of the TMMP or APP can be carried out by affinity chromatography, e.g., on a Protein A column, a Protein G column, or the like. An example of a suitable mammalian cell is a CHO cell; e.g., an Expi-CHO-S™ cell (e.g., ThermoFisher Scientific, Catalog #A29127).

The first and second polypeptides will self-assemble into heterodimers by spontaneously forming disulfide bonds between the above-discussed Cys residues in the first and second polypeptides. As also noted above, when both heterodimers include Ig Fc polypeptides, disulfide bonds will spontaneously form between the respective Ig Fc polypeptides to covalently link the two heterodimers to one another (depicted schematically in FIG. 1E).

### Compositions

The present disclosure provides compositions, including pharmaceutical compositions, comprising a TMMP or an APP of the present disclosure. The present disclosure provides compositions, including pharmaceutical compositions, comprising a nucleic acid or a recombinant expression vector of the present disclosure. The discussion, below, of compositions refers to compositions comprising a TMMP of the present disclosure; however, the discussion applies equally to an APP of the present disclosure.

### Compositions Comprising a TMMP or an APP

A composition of the present disclosure can comprise, in addition to a TMMP of the present disclosure, or an APP of the present disclosure, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

The composition may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A pharmaceutical composition can comprise: i) a TMMP of the present disclosure; and ii) a pharmaceutically acceptable excipient. A pharmaceutical composition can comprise: i) an APP of the present disclosure; and ii) a pharmaceutically acceptable excipient. In some cases, a subject pharmaceutical composition will be suitable for administration to a subject, e.g., will be sterile. For example, in some embodiments, a subject pharmaceutical composition will be suitable for administration to a human subject, e.g., where the composition is sterile and is free of detectable pyrogens and/or other toxins.

The protein compositions may comprise other components, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

For example, compositions may include aqueous solution, powder form, granules, tablets, pills, suppositories, capsules, suspensions, sprays, and the like. The composition may be formulated according to the various routes of administration described below.

Where a TMMP of the present disclosure is administered as an injectable (e.g. subcutaneously, intraperitoneally, intramuscularly, intralymphatically, and/or intravenously) directly into a tissue, a formulation can be provided as a ready-to-use dosage form, or as non-aqueous form (e.g. a reconstitutable storage-stable powder) or aqueous form, such as liquid composed of pharmaceutically acceptable carriers and excipients. The protein-containing formulations may also be provided so as to enhance serum half-life of the subject protein following administration. For example, the protein may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

In some cases, a composition of the present disclosure comprises: a) a TMMP of the present disclosure; and b) saline (e.g., 0.9% NaCl). In some cases, the composition is sterile. In some cases, the composition is suitable for administration to a human subject, e.g., where the composition is sterile and is free of detectable pyrogens and/or other toxins. Thus, the present disclosure provides a composition comprising: a) a TMMP of the present disclosure; and b) saline (e.g., 0.9% NaCl), where the composition is sterile and is free of detectable pyrogens and/or other toxins.

Other examples of formulations suitable for parenteral administration include isotonic sterile injection solutions, anti-oxidants, bacteriostats, and solutes that render the formulation isotonic with the blood of the intended recipient, suspending agents, solubilizers, thickening agents, stabilizers, and preservatives. For example, a subject pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of a TMMP of the present disclosure in a formulation can vary widely (e.g., from less than about 0.1%, usually at or at least about 2% to as much as 20% to 50% or more by weight) and will usually be selected primarily based on fluid volumes, viscosities, and patient-based factors in accordance with the particular mode of administration selected and the patient's needs.

The present disclosure provides a container comprising a composition of the present disclosure, e.g., a liquid composition. The container can be, e.g., a syringe, an ampoule, and the like. In some cases, the container is sterile. In some cases, both the container and the composition are sterile.

### Compositions Comprising a Nucleic Acid or a Recombinant Expression Vector

The present disclosure provides compositions, e.g., pharmaceutical compositions, comprising a nucleic acid or a recombinant expression vector of the present disclosure. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A composition of the present disclosure can include: a) one or more nucleic acids or one or more recombinant expression vectors comprising nucleotide sequences encoding a TMMP or an APP of the present disclosure; and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative. Suitable buffers include, but are not limited to, (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl₂, KCl, MgSO₄, etc.

A pharmaceutical formulation of the present disclosure can include a nucleic acid or recombinant expression vector of the present disclosure in an amount of from about 0.001% to about 90% (w/w). In the description of formulations, below, “subject nucleic acid or recombinant expression vector” will be understood to include a nucleic acid or recombinant expression vector of the present disclosure. For example, in some cases, a subject formulation comprises a nucleic acid or recombinant expression vector of the present disclosure.

A subject nucleic acid or recombinant expression vector can be admixed, encapsulated, conjugated or otherwise associated with other compounds or mixtures of compounds; such compounds can include, e.g., liposomes or receptor-targeted molecules. A subject nucleic acid or recombinant expression vector can be combined in a formulation with one or more components that assist in uptake, distribution and/or absorption.

A subject nucleic acid or recombinant expression vector composition can be formulated into any of many possible dosage forms such as, but not limited to, tablets, capsules, gel capsules, liquid syrups, soft gels, suppositories, and enemas. A subject nucleic acid or recombinant expression vector composition can also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

A formulation comprising a subject nucleic acid or recombinant expression vector can be a liposomal formulation. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes that can interact with negatively charged DNA molecules to form a stable complex. Liposomes that are pH sensitive or negatively charged are believed to entrap DNA rather than complex with it. Both cationic and noncationic liposomes can be used to deliver a subject nucleic acid or recombinant expression vector.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein by reference in its entirety.

The formulations and compositions of the present disclosure may also include surfactants. The use of surfactants in drug products, formulations and in emulsions is well known in the art. Surfactants and their uses are further described in U.S. Pat. No. 6,287,860.

In one embodiment, various penetration enhancers are included, to effect the efficient delivery of nucleic acids. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein by reference in its entirety.

Compositions and formulations for oral administration include powders or granules, microparticulates, nanoparticulates, suspensions or solutions in water or non-aqueous media, capsules, gel capsules, sachets, tablets, or minitablets. Thickeners, flavoring agents, diluents, emulsifiers, dispersing aids or binders may be desirable. Suitable oral formulations include those in which a subject antisense nucleic acid is administered in conjunction with one or more penetration enhancers surfactants and chelators. Suitable surfactants include, but are not limited to, fatty acids and/or esters or salts thereof, bile acids and/or salts thereof. Suitable bile acids/salts and fatty acids and their uses are further described in U.S. Pat. No. 6,287,860. Also suitable are combinations of penetration enhancers, for example, fatty acids/salts in combination with bile acids/salts. An exemplary suitable combination is the sodium salt of lauric acid, capric acid, and UDCA. Further penetration enhancers include, but are not limited to, polyoxyethylene-9-lauryl ether, and polyoxyethylene-20-cetyl ether. Suitable penetration enhancers also include propylene glycol, dimethylsulfoxide, triethanolamine, N,N-dimethylacetamide, N,N-dimethylformamide, 2-pyrrolidone and derivatives thereof, tetrahydrofurfuryl alcohol, and AZONE™.

### Methods

A TMMP of the present disclosure is useful for modulating an activity of a T cell. Thus, the present disclosure provides methods of modulating an activity of a T cell, the methods generally involving contacting a target T cell with a TMMP of the present disclosure.

An APP of the present disclosure is useful for various research, therapeutic, and diagnostic purposes. For example, an APP of the present disclosure can be used to label, directly or indirectly, an antigen-specific T cell. An APP of the present disclosure can provide effector functions in an antigen-specific manner.

### Methods of Modulating T Cell Activity

The present disclosure provides a method of selectively modulating the activity of an epitope-specific T cell, the method comprising contacting the T cell with a TMMP of the present disclosure, where contacting the T cell with a TMMP of the present disclosure selectively modulates the activity of the epitope-specific T cell. In some cases, the contacting occurs in vivo. In some cases, the contacting occurs in vitro.

In some cases, a TMMP of the present disclosure reduces activity of an autoreactive T cell and/or an autoreactive B cell. In some cases, a TMMP of the present disclosure increases the number and/or activity of a regulatory T cell (Treg), resulting in reduced activity of an autoreactive T cell and/or an autoreactive B cell.

In some cases, the T cell being contacted with a TMMP of the present disclosure is a regulatory T cell (Treg). Tregs are CD4⁺, FOXP3⁺, and CD25⁺. Tregs can suppress autoreactive T cells. In some cases, a method of the present disclosure activates Tregs, thereby reducing autoreactive T cell activity.

The present disclosure provides a method of increasing proliferation of Tregs, the method comprising contacting Tregs with a TMMP of the present disclosure, where the contacting increases proliferation of Tregs. The present disclosure provides a method of increasing the number of Tregs in an individual, the method comprising administering to the individual a TMMP of the present disclosure, where the administering results in an increase in the number of Tregs in the individual. For example, the number of Tregs can be increased by at least 5%, at least 10%, at least 15%, at least 20%, at least 25%, at least 30%, at least 40%, at least 50%, at least 60%, at least 70%, at least 80%, at least 90%, at least 2-fold, at least 2.5-fold, at least 5-fold, at least 10-fold, or more than 10-fold.

### Methods of Detecting an Antigen-Specific T Cell

The present disclosure provides a method of detecting an antigen-specific T-cell. The methods comprise contacting a T cell with an APP of the present disclosure; and detecting binding of the APP to the T cell.

The present disclosure provides a method of detecting an antigen-specific T cell, the method comprising contacting a T cell with an APP of the present disclosure, wherein binding of the APP to the T cell indicates that the T cell is specific for the epitope present in the APP.

In some cases, the APP comprises a detectable label. Suitable detectable labels include, but are not limited to, a radioisotope, a fluorescent polypeptide, or an enzyme that generates a fluorescent product, and an enzyme that generates a colored product. Where the APP comprises a detectable label, binding of the APP to the T cell is detected by detecting the detectable label.

In some cases, an APP of the present disclosure comprises a detectable label suitable for use in in vivo imaging, e.g., suitable for use in positron emission tomography (PET), single photon emission tomography (SPECT), near infrared (NIR) optical imaging, x-ray imaging, computer-assisted tomography (CAT), or magnetic resonance imaging (MRI), or other in vivo imaging method. Examples of suitable labels for in vivo imaging include gadolinium chelates (e.g., gadolinium chelates with DTPA (diethylenetriamine penta-acetic acid), DTPA-bismethylamide (BMA), DOTA (dodecane tetraacetic acid), or HP-DO3A (1,4,7-tris(carboxymethyl)-10-(2′-hydroxypropyl)-1,4,7,10-tetraazacycl ododecane)), iron chelates, magnesium chelates, manganese chelates, copper chelates, chromium chelates, iodine-based materials, and radionuclides. Suitable radionuclides include, but are not limited to, ¹²³I, ¹²⁵I, ¹³⁰I, ¹³¹I, ¹³³I, ¹³⁵I, ⁴⁷Sc, ⁷²As, ⁷²Se, ⁹⁰Y, ⁸⁸Y, ⁹⁷Ru, ¹⁰⁰Pd, ¹⁰¹mRh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga, ⁶⁸Ga, ⁶⁴Cu, ⁶⁷Cu, ⁷⁵Br, ⁷⁷Br, ⁹⁹mTc, ¹⁴C, ¹³N, ¹⁵O, ³²P, ³³P, and ¹⁸F. In some cases, the detectable label is a positron-emitting isotope such as ¹¹C, ¹³N, ¹⁵O, ¹⁸F, ⁶⁴Cu, ⁶⁸Ga, ⁷⁸Br, ⁸²Rb, ⁸⁶Y, ⁹⁰Y, ²²Na, ²⁶Al, ⁴⁰K, ⁸³Sr, ⁸⁹Zr, or ¹²⁴I. In some cases, the detectable label is ⁶⁴Cu.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilized ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

In some cases, binding of the APP to the T cell is detected using a detectably labeled antibody specific for the APP. An antibody specific for the APP can comprise a detectable label such as a radioisotope, a fluorescent polypeptide, or an enzyme that generates a fluorescent product, or an enzyme that generates a colored product.

In some cases, the T cell being detected is present in a sample comprising a plurality of T cells. For example, a T cell being detected can be present in a sample comprising from 10 to 10⁹ T cells, e.g., from 10 to 10², from 10² to 10⁴, from 10⁴ to 10⁶, from 10⁶ to 10⁷, from 10⁷ to 10⁸, or from 10⁸ to 10⁹, or more than 10⁹, T cells.

### Treatment Methods

The present disclosure provides treatment methods, the methods comprising administering to the individual an amount of a TMMP of the present disclosure, or one or more nucleic acids or expression vectors encoding the TMMP effective to selectively modulate the activity of an epitope-specific T cell in an individual and to treat the individual. In some cases, a treatment method of the present disclosure comprises administering to an individual in need thereof one or more recombinant expression vectors comprising nucleotide sequences encoding a TMMP of the present disclosure. In some cases, a treatment method of the present disclosure comprises administering to an individual in need thereof one or more mRNA molecules comprising nucleotide sequences encoding a TMMP of the present disclosure. In some cases, a treatment method of the present disclosure comprises administering to an individual in need thereof a TMMP of the present disclosure. A TMMP of the present disclosure is useful for treating type 1 diabetes (T1D).

The present disclosure provides a method of selectively modulating the activity of an epitope-specific T cell in an individual, the method comprising administering to the individual an effective amount of a TMMP of the present disclosure, or one or more nucleic acids (e.g., expression vectors; mRNA; etc.) comprising nucleotide sequences encoding the TMMP, where the TMMP selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a TMMP of the present disclosure. A treatment method of the present disclosure, comprising administering an effective amount of a TMMP of the present disclosure, is suitable for treating T1D.

In some cases, the immunomodulatory polypeptide is an inhibitory polypeptide, and a T TMMP of the present disclosure inhibits activity of the epitope-specific T cell. In some cases, the epitope is a self-epitope, and a TMMP of the present disclosure selectively inhibits the activity of a T cell specific for the self-epitope.

The present disclosure provides a method of treating T1D in an individual, the method comprising administering to the individual an effective amount of a TMMP of the present disclosure, or one or more nucleic acids comprising nucleotide sequences encoding the TMMP, where the TMMP comprises a T1D peptide (as described above), and where the TMMP comprises an inhibitory immunomodulatory polypeptide. In some cases, an “effective amount” of a TMMP of the present disclosure is an amount that, when administered in one or more doses to an individual in need thereof, reduces the number of self-reactive (i.e., reactive with aTID-associated antigen) CD4⁺ and/or CD8⁺ T cells by at least 10%, at least 15%, at least 20%, at least 25%, at least 30%, at least 40%, at least 50%, at least 60%, at least 70%, at least 80%, at least 90%, or at least 95%, compared to number of self-reactive T cells in the individual before administration of the TMMP, or in the absence of administration with the TMMP. In some cases, an “effective amount” of a TMMP of the present disclosure is an amount that, when administered in one or more doses to an individual in need thereof, reduces production of Th2 cytokines in the individual. In some cases, an “effective amount” of a TMMP of the present disclosure is an amount that, when administered in one or more doses to an individual in need thereof, ameliorates one or more symptoms associated with T1D in the individual. In some instances, the TMMP reduces the number of CD4⁺ self-reactive T cells (i.e., the number of CD4⁺ T cells reactive with a T1D-associated antigen), which in turn leads to a reduction in CD8⁺ self-reactive T cells. In some instances, the TMMP increases the number of CD4⁺ Tregs, which in turn reduces the number of CD4⁺ self-reactive T cells and/or CD8⁺ T self-reactive T cells.

As noted above, in some cases, in carrying out a subject treatment method, a TMMP of the present disclosure is administered to an individual in need thereof, as the polypeptide per se. In other instances, in carrying out a subject treatment method, one or more nucleic acids comprising nucleotide sequences encoding a TMMP is/are administering to an individual in need thereof. Thus, in other instances, one or more nucleic acids of the present disclosure, e.g., one or more recombinant expression vectors of the present disclosure, is/are administered to an individual in need thereof.

The present disclosure also provides treatment methods, the methods comprising administering to the individual an amount of an APP of the present disclosure, or one or more nucleic acids or expression vectors encoding the APP effective to selectively engage with an epitope-specific T cell in an individual in order to treat the individual, e.g., by depleting epitope-specific T cells when the Ig Fc polypeptide is one that that induces cell lysis through activation of CDC, and/or elicits ADCC. In some cases, a treatment method of the present disclosure comprises administering to an individual in need thereof one or more recombinant expression vectors comprising nucleotide sequences encoding an APP of the present disclosure. In some cases, a treatment method of the present disclosure comprises administering to an individual in need thereof one or more mRNA molecules comprising nucleotide sequences encoding an APP of the present disclosure. In some cases, a treatment method of the present disclosure comprises administering to an individual in need thereof an APP of the present disclosure. An APP of the present disclosure is useful for treating type 1 diabetes (T1D) by selectively engaging with an epitope-specific T cell in an individual, including by, e.g., depleting epitope-specific T cells when the Ig Fc polypeptide is one that that induces cell lysis through activation of CDC, and/or elicits ADCC.

The present disclosure provides a method of treating T1D in an individual, the method comprising administering to the individual an effective amount of an APP of the present disclosure, or one or more nucleic acids comprising nucleotide sequences encoding the APP, where the APP comprises a T1D peptide (as described above). In some cases, an “effective amount” of an APP of the present disclosure is an amount that, when administered in one or more doses to an individual in need thereof, reduces the number of self-reactive (i.e., reactive with aTID-associated antigen) CD4⁺ and/or CD8⁺ T cells by at least 10%, at least 15%, at least 20%, at least 25%, at least 30%, at least 40%, at least 50%, at least 60%, at least 70%, at least 80%, at least 90%, or at least 95%, compared to number of self-reactive T cells in the individual before administration of the APP, or in the absence of administration with the APP. In some cases, an “effective amount” of an APP of the present disclosure is an amount that, when administered in one or more doses to an individual in need thereof, reduces production of Th2 cytokines in the individual. In some cases, an “effective amount” of an APP of the present disclosure is an amount that, when administered in one or more doses to an individual in need thereof, ameliorates one or more symptoms associated with T1D in the individual. In some instances, the APP reduces the number of CD4⁺ self-reactive T cells (i.e., the number of CD4⁺ T cells reactive with a T1D-associated antigen), which in turn leads to a reduction in CD8⁺ self-reactive T cells. In some instances (e.g., when the Ig Fc polypeptide of the APP substantially does not induce cell lysis through activation of CDC, and/or elicits ADCC), the APP increases the number of CD4⁺ Tregs, which in turn reduces the number of CD4⁺ self-reactive T cells and/or CD8⁺ T self-reactive T cells.

As noted above, in some cases, in carrying out a subject treatment method, an APP of the present disclosure is administered to an individual in need thereof, as the polypeptide per se. In other instances, in carrying out a subject treatment method, one or more nucleic acids comprising nucleotide sequences encoding an APP is/are administering to an individual in need thereof. Thus, in other instances, one or more nucleic acids of the present disclosure, e.g., one or more recombinant expression vectors of the present disclosure, is/are administered to an individual in need thereof.

### Formulations

Suitable formulations are described above, where suitable formulations include a pharmaceutically acceptable excipient. In some cases, a suitable formulation comprises: a) a TMMP or APP of the present disclosure; and b) a pharmaceutically acceptable excipient. In some cases, a suitable formulation comprises: a) a nucleic acid comprising a nucleotide sequence encoding a TMMP or APP of the present disclosure; and b) a pharmaceutically acceptable excipient; in some instances, the nucleic acid is an mRNA. In some cases, a suitable formulation comprises: a) a first nucleic acid comprising a nucleotide sequence encoding the first polypeptide of a TMMP or APP of the present disclosure; b) a second nucleic acid comprising a nucleotide sequence encoding the second polypeptide of a TMMP or APP of the present disclosure; and c) a pharmaceutically acceptable excipient. In some cases, a suitable formulation comprises: a) a recombinant expression vector comprising a nucleotide sequence encoding a TMMP or APP of the present disclosure; and b) a pharmaceutically acceptable excipient. In some cases, a suitable formulation comprises: a) a first recombinant expression vector comprising a nucleotide sequence encoding the first polypeptide of a TMMP or APP of the present disclosure; b) a second recombinant expression vector comprising a nucleotide sequence encoding the second polypeptide of a TMMP or APP of the present disclosure; and c) a pharmaceutically acceptable excipient.

Suitable pharmaceutically acceptable excipients are described above.

### Dosages

A suitable dosage can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A TMMP or APP (whether as a single heterodimer or, as described above, as a homodimer comprising two heterodimers linked together) of the present disclosure may be administered in amounts between 1 ng/kg body weight and 20 mg/kg body weight per dose, e.g. between 0.1 mg/kg body weight to 10 mg/kg body weight, e.g. from 0.1 mg/kg body weight to 0.5 mg/kg body weight, from 0.5 mg/kg body weight to 1 mg/kg body weight, from 1.0 mg/kg body weight to 5 mg/kg body weight, from 5 mg/kg body weight to 10 mg/kg body weight, from 10 mg/kg body weight to 15 mg/kg body weight, and from 15 mg/kg body weight to 20 mg/kg body weight; however, doses below 0.1 mg/kg body weight or above 20 mg/kg are envisioned, especially considering the aforementioned factors. Amounts thus include from about 0.1 mg/kg body weight to about 0.5 mg/kg body weight, from about 0.5 mg/kg body weight to about 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight. If the regimen is a continuous infusion, it can also be in the range of 1 μg to 10 mg per kilogram of body weight per minute.

Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the T1D disease state, wherein a TMMP or APP of the present disclosure is administered in maintenance doses, ranging from those recited above, i.e., 0.1 mg/kg body weight to about 0.5 mg/kg body weight, from about 0.5 mg/kg body weight to about 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

Those of skill will readily appreciate that dose levels can vary as a function of the specific TMMP or APP, the severity of the symptoms and the susceptibility of the subject to side effects. Preferred dosages for a given compound are readily determinable by those of skill in the art by a variety of means.

In some cases, multiple doses of a TMMP or APP of the present disclosure, a nucleic acid of the present disclosure, or a recombinant expression vector of the present disclosure are administered. The frequency of administration of a TMMP or APP of the present disclosure, a nucleic acid of the present disclosure, or a recombinant expression vector of the present disclosure can vary depending on any of a variety of factors, e.g., severity of the symptoms, patient response, etc. For example, in some cases, a TMMP or APP of the present disclosure, a nucleic acid of the present disclosure, or a recombinant expression vector of the present disclosure is administered once per month, twice per month, three times per month, every other week (qow), one every three weeks, once every four weeks, once per week (qw), twice per week (biw), three times per week (tiw), four times per week, five times per week, six times per week, every other day (qod), daily (qd), twice a day (qid), or three times a day (tid).

The duration of administration of a TMMP or APP of the present disclosure, a nucleic acid of the present disclosure, or a recombinant expression vector of the present disclosure, e.g., the period of time over which a TMMP or APP of the present disclosure, a nucleic acid of the present disclosure, or a recombinant expression vector of the present disclosure is administered, can vary, depending on any of a variety of factors, e.g., patient response, etc. For example, a TMMP or APP of the present disclosure, a nucleic acid of the present disclosure, or a recombinant expression vector of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more, including continued administration for the patient's life.

Where treatment is of a finite duration, following successful treatment, it may be desirable to have the patient undergo maintenance therapy to prevent the recurrence of the T1D disease state, wherein a TMMP or APP of the present disclosure is administered in maintenance doses, ranging from those recited above, i.e., 0.1 mg/kg body weight to about 0.5 mg/kg body weight, from about 0.5 mg/kg body weight to about 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

### Routes of Administration

An active agent (a TMMP or APP of the present disclosure, a nucleic acid of the present disclosure, or a recombinant expression vector of the present disclosure) is administered to an individual using any available method and route suitable for drug delivery, including in vivo and in vitro methods, as well as systemic and localized routes of administration. A TMMP or APP of the present disclosure, a nucleic acid of the present disclosure, or a recombinant expression vector of the present disclosure can be administered to a host using any available conventional methods and routes suitable for delivery of conventional drugs, including systemic or localized routes. In general, routes of administration contemplated for use in a method of the present disclosure include, but are not necessarily limited to, enteral, parenteral, and inhalational routes.

Conventional and pharmaceutically acceptable routes of administration include intramuscular, intratracheal, intralymphatic, subcutaneous, intradermal, topical application, intravenous, intraarterial, rectal, nasal, oral, and other enteral and parenteral routes of administration. Of these, intravenous, intramuscular and subcutaneous may be more commonly employed. Routes of administration may be combined, if desired, or adjusted depending upon the TMMP or APP and/or the desired effect. A TMMP or APP of the present disclosure, or a nucleic acid or recombinant expression vector of the present disclosure, can be administered in a single dose or in multiple doses.

In some cases, a TMMP or APP of the present disclosure, a nucleic acid of the present disclosure, or a recombinant expression vector of the present disclosure is administered intravenously. In some cases, a TMMP or APP of the present disclosure, a nucleic acid of the present disclosure, or a recombinant expression vector of the present disclosure is administered intramuscularly. In some cases, a TMMP or APP of the present disclosure, a nucleic acid of the present disclosure, or a recombinant expression vector of the present disclosure is administered intralymphatically. In some cases, a TMMP of the present disclosure, a nucleic acid of the present disclosure, or a recombinant expression vector of the present disclosure is administered subcutaneously.

### Subjects Suitable for Treatment

Subjects suitable for treatment with a method of the present disclosure include individuals who have T1D, including individuals who have been diagnosed as having T1D, and individuals who have been treated for T1D but who failed to respond to the treatment. Suitable subjects also may include individuals who have been diagnosed as being likely to develop T1D or who have symptoms indicating the imminent onset of T1D.

### Methods of Selectively Delivering an Immunomodulatory Polypeptide

The present disclosure provides a method of delivering an immunomodulatory polypeptide (such as a wild-type (wt) or variant of immunomodulatory polypeptide described herein, e.g., PD-L1, IL-2, TGFβ, or FasL) to a selected T cell or a selected T cell population, e.g., in a manner such that a TCR specific for a given T1D epitope is targeted. The present disclosure provides a method of delivering a wt or variant immunomodulatory polypeptide disclosed herein selectively to a target T cell bearing a TCR specific for the epitope present in a TMMP of the present disclosure. In this context, the term “selective delivery” means that the immunomodulatory polypeptide is not delivered to all T cells, but rather that the majority of T cells to which the immunomodulatory polypeptide is delivered are T cells specific for the T1D peptide, thereby minimizing the modulation of non-target T cells. Thus, e.g., less than 50%, less than 40%, less than 30%, less than 25%, less than 20%, less than 15%, less than 10%, less than 5%, or less than 4%, 3%, 2% or 1%, of the non-target T cells bind the TMMP and, as a result, the immunomodulatory polypeptide is not delivered to the non-target T cells that do not bind the TMMP.

The method comprises contacting a population of T cells, in vivo or in vitro, with a TMMP of the present disclosure. The population of T cells can be a mixed population that comprises: i) the target T cell; and ii) non-target T cells that are not specific for the epitope (e.g., T cells that are specific for an epitope(s) other than the epitope to which the epitope-specific T cell binds). The epitope-specific T cell is specific for the T1D peptide epitope present in the TMMP binds to the TMMP. Contacting the population of T cells with the TMMP thus delivers the immunomodulatory polypeptide present in the TMMP selectively to the T cell(s) that are specific for the epitope present in the TMMP. Thus, e.g., less than 50%, less than 40%, less than 30%, less than 25%, less than 20%, less than 15%, less than 10%, less than 5%, or less than 4%, 3%, 2% or 1%, of the non-target T cells bind the TMMP and, as a result, the immunomodulatory polypeptide is not delivered to the non-target T cells that are not bound to the TMMP.

In some cases, the population of T cells is in vivo. In some cases, the population of T cells is in vitro, and a biological response (e.g., T cell activation and/or expansion and/or phenotypic differentiation) of the target T cell population to the TMMP of the present disclosure is elicited in the context of an in vitro culture. For example, a mixed population of T cells can be obtained from an individual, and can be contacted with the TMMP in vitro. Such contacting can comprise single or multiple exposures of the population of T cells to a defined dose(s) and/or exposure schedule(s). In some cases, said contacting results in selectively modulating target T cells within the population of T cells and results, e.g., in generation of a population of activated and/or expanded target T cells. As an example, a mixed population of T cells can be peripheral blood mononuclear cells (PBMC). For example, PBMC from a patient can be obtained by standard blood drawing and PBMC enrichment techniques before being exposed to 0.1-1000 nM of a TMMP of the present disclosure under standard lymphocyte culture conditions. At time points before, during, and after exposure of the mixed T cell population at a defined dose and schedule, the abundance of target T cells in the in vitro culture can be monitored by specific peptide-MHC multimers and/or phenotypic markers and/or functional activity (e.g. cytokine ELISpot assays). In some cases, upon achieving an optimal abundance and/or phenotype of antigen specific cells in vitro, all or a portion of the population of activated and/or expanded target T cells is administered to the individual (the individual from whom the mixed population of T cells was obtained).

In some cases, the population of T cells is in vitro. For example, a mixed population of T cells is obtained from an individual, and is contacted with a TMMP of the present disclosure in vitro. Such contacting, which can comprise single or multiple exposures of the T cells to a defined dose(s) and/or exposure schedule(s) in the context of in vitro cell culture, can be used to determine whether the mixed population of T cells includes T cells that are specific for the epitope presented by the TMMP. The presence of T cells that are specific for the T1D peptide of the TMMP can be determined by assaying a sample comprising a mixed population of T cells, which population of T cells comprises T cells that are not specific for the epitope (non-target T cells) and may comprise T cells that are specific for the epitope (target T cells). Known assays can be used to detect activation and/or proliferation of the target T cells, thereby providing an in vitro assay that can determine whether a particular TMMP possesses an epitope that binds to T cells present in the individual and thus whether the TMMP has potential use as a therapeutic composition for that individual. Suitable known assays for detection of activation and/or proliferation of target T cells include, e.g., flow cytometric characterization of T cell phenotype and/or antigen specificity and/or proliferation. Such an assay to detect the presence of epitope-specific T cells, e.g., a companion diagnostic, can further include additional assays (e.g. effector cytokine ELISpot assays) and/or appropriate controls (e.g. antigen-specific and antigen-nonspecific multimeric peptide-HLA staining reagents) to determine whether the TMMP is selectively binding/activating and/or expanding the target T cell. Thus, for example, the present disclosure provides a method of detecting, in a mixed population of T cells obtained from an individual, the presence of a target T cell that binds an epitope of interest, the method comprising: a) contacting in vitro the mixed population of T cells with a TMMP of the present disclosure, wherein the multimeric polypeptide comprises the epitope of interest; and b) detecting activation and/or proliferation of T cells in response to said contacting, wherein activated and/or proliferated T cells indicates the presence of the target T cell. Alternatively, and/or in addition, if activation and/or expansion (proliferation) of the desired T cell population is obtained using the TMMP, then all or a portion of the population of T cells comprising the activated/expanded T cells can be administered back to the individual as a therapy.

In some instances, the population of T cells is in vivo in an individual. In such instances, a method of the present disclosure for selectively delivering an immunomodulatory polypeptide as described herein to an epitope-specific T cell comprises administering the TMMP to the individual.

The epitope-specific T cell to which an immunomodulatory polypeptide is being selectively delivered is also referred to herein as a “target T cell.” In some cases, the target T cell is a regulatory T cell (Treg). In some cases, the Treg inhibits or suppresses activity of an autoreactive T cell.

The present disclosure provides a method of delivering an inhibitory immunomodulatory polypeptide (such as wt or variant of PD-L1 or FasL) to a selected T cell or a selected T cell population, e.g., in a manner such that a TCR specific for a given epitope is targeted. The present disclosure provides a method of delivering such an inhibitory immunomodulatory polypeptide selectively to a target T cell bearing a TCR specific for the epitope present in a TMMP of the present disclosure. The method comprises contacting a population of T cells with a TMMP of the present disclosure. The population of T cells can be a mixed population that comprises: i) the target T cell; and ii) non-target T cells that are not specific for the epitope (e.g., T cells that are specific for an epitope(s) other than the epitope to which the epitope-specific T cell binds). The epitope-specific T cell is specific for the epitope-presenting peptide present in the TMMP, and binds to the TMMP. Contacting the population of T cells with the TMMP thus delivers the inhibitory immunomodulatory polypeptide present in the TMMP selectively to the T cell(s) that are specific for the T1D peptide present in the TMMP.

For example, a TMMP of the present disclosure is contacted with a population of T cells comprising: i) a target T cell(s) that is specific for the epitope present in the TMMP; and ii) a non-target T cell(s), e.g., a T cell(s) that is specific for a second epitope(s) that is not the epitope present in the TMMP. Contacting the population results in selective delivery of an inhibitory immunomodulatory polypeptide(s) an inhibitory immunomodulatory polypeptide (such as wt or variant of PD-L1 or FasL) which is present in the TMMP, to the target T cell. Thus, e.g., less than 50%, less than 40%, less than 30%, less than 25%, less than 20%, less than 15%, less than 10%, less than 5%, or less than 4%, 3%, 2% or 1%, of the non-target T cells bind the TMMP and, as a result, the immunomodulatory polypeptide is not delivered to the non-target T cells.

In some cases, the population of T cells is in vitro. In some cases, the population of T cells is in vitro, and a biological response (e.g., T cell activation and/or expansion and/or phenotypic differentiation) of the target T cell population to the TMMP of the present disclosure is elicited in the context of an in vitro culture. For example, a mixed population of T cells can be obtained from an individual, and can be contacted with the TMMP in vitro. Such contacting can comprise single or multiple exposures of the population of T cells to a defined dose(s) and/or exposure schedule(s). In some cases, said contacting results in selectively binding/activating and/or expanding target T cells within the population of T cells, and results in generation of a population of activated and/or expanded target T cells. As an example, a mixed population of T cells can be peripheral blood mononuclear cells (PBMC). For example, PBMC from a patient can be obtained by standard blood drawing and PBMC enrichment techniques before being exposed to 0.1-1000 nM of a TMMP of the present disclosure under standard lymphocyte culture conditions. At time points before, during, and after exposure of the mixed T cell population at a defined dose and schedule, the abundance of target T cells in the in vitro culture can be monitored by specific peptide-MHC multimers and/or phenotypic markers and/or functional activity (e.g. cytokine ELISpot assays). In some cases, upon achieving an optimal abundance and/or phenotype of antigen specific cells in vitro, all or a portion of the population of activated and/or expanded target T cells is administered to the individual (the individual from whom the mixed population of T cells was obtained).

In some cases, the population of T cells is in vitro. For example, a mixed population of T cells is obtained from an individual, and is contacted with a TMMP of the present disclosure in vitro. Such contacting, which can comprise single or multiple exposures of the T cells to a defined dose(s) and/or exposure schedule(s) in the context of in vitro cell culture, can be used to determine whether the mixed population of T cells includes T cells that are specific for the epitope presented by the TMMP. The presence of T cells that are specific for the T1D peptide epitope of the TMMP can be determined by assaying a sample comprising a mixed population of T cells, which population of T cells comprises T cells that are not specific for the epitope (non-target T cells) and may comprise T cells that are specific for the epitope (target T cells). Known assays can be used to detect activation and/or proliferation of the target T cells, thereby providing an in vitro assay that can determine whether a particular TMMP possesses an epitope that binds to T cells present in the individual and thus whether the TMMP has potential use as a therapeutic composition for that individual. Suitable known assays for detection of activation and/or proliferation of target T cells include, e.g., flow cytometric characterization of T cell phenotype and/or antigen specificity and/or proliferation. Such an assay to detect the presence of epitope-specific T cells, e.g., a companion diagnostic, can further include additional assays (e.g. effector cytokine ELISpot assays) and/or appropriate controls (e.g. antigen-specific and antigen-nonspecific multimeric peptide-HLA staining reagents) to determine whether the TMMP is selectively binding/activating and/or expanding the target T cell. Thus, for example, the present disclosure provides a method of detecting, in a mixed population of T cells obtained from an individual, the presence of a target T cell that binds an epitope of interest, the method comprising: a) contacting in vitro the mixed population of T cells with a TMMP of the present disclosure, wherein the multimeric polypeptide comprises the epitope of interest; and b) detecting activation and/or proliferation of T cells in response to said contacting, wherein activated and/or proliferated T cells indicates the presence of the target T cell. Alternatively, and/or in addition, if activation and/or expansion (proliferation) of the desired T cell population is obtained using the TMMP, then all or a portion of the population of T cells comprising the activated/expanded T cells can be administered back to the individual as a therapy.

In some instances, the population of T cells is in vivo in an individual. In such instances, a method of the present disclosure for selectively delivering an inhibitory immunomodulatory polypeptide (such as wt or variant of PD-L1 or FasL) to an epitope-specific T cell comprises administering the TMMP to the individual.

The epitope-specific T cell to which an inhibitory immunomodulatory polypeptide is being selectively delivered is also referred to herein as a “target T cell.” In some cases, the target T cell is a regulatory T cell (Treg). In some cases, the Treg inhibits or suppresses activity of an autoreactive T cell. In some cases, the target T cell is a CD4⁺ T cell. In some cases, the target T cell is a CD4⁺ T cell that is specific for an autoantigen.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A T-cell modulatory multimeric polypeptide (TMMP) comprising at least one heterodimer, wherein each heterodimer comprises:
a) a first polypeptide comprising:
   i) a peptide that displays a Type 1 Diabetes-associated epitope capable of being bound by a T-cell receptor (a “T1D peptide”); and
   ii) a first major histocompatibility complex (MHC) class II polypeptide; and
   iii) optionally a linker that links the T1D peptide to the first MHC class II polypeptide; and
b) a second polypeptide comprising a second MHC class II polypeptide,
   wherein one or both polypeptides of the heterodimer comprises one or more immunomodulatory polypeptides,
   wherein the first and the second polypeptide of the heterodimer are covalently linked to one another via at least one disulfide bond, and
   wherein one polypeptide of the heterodimer optionally comprises an immunoglobulin (Ig) Fc polypeptide or a non-Ig scaffold, and wherein when the optional Ig Fc is present, it is optionally joined to the first or second polypeptide via a linker.
2. A TMMP of aspect 1, wherein
a1) the first polypeptide comprises, in order from N-terminus to C-terminus:
   i) the T1D peptide; and
   ii) an MHC class II β polypeptide; and
b1) the second polypeptide comprises, in order from N-terminus to C-terminus:
   i) the one or more immunomodulatory polypeptides;
   ii) an MHC class II α polypeptide; and
   iii) an Ig Fc polypeptide; or
a2) the first polypeptide comprises, in order from N-terminus to C-terminus:
   i) the T1D peptide; and
   ii) an MHC class II β polypeptide; and
b2) the second polypeptide comprises, in order from N-terminus to C-terminus:
   i) an MHC class II α polypeptide;
   ii) an Ig Fc polypeptide; and
   iii) the one or more immunomodulatory polypeptides; or
a3) the first polypeptide comprises, in order from N-terminus to C-terminus:
   i) the T1D peptide;
   ii) an MHC class II β polypeptide; and
b3) the second polypeptide comprises, in order from N-terminus to C-terminus:
   i) an MHC class II α polypeptide;
   ii) the one or more immunomodulatory polypeptides; and
   iii) an Ig Fc polypeptide; or
a4) the first polypeptide comprises, in order from N-terminus to C-terminus:
   i) the one or more immunomodulatory polypeptides;
   ii) the T1D peptide; and
   iii) an MHC class II β polypeptide; and
b4) the second polypeptide comprises, in order from N-terminus to C-terminus:
   i) an MHC class II α polypeptide; and
   ii) an Ig Fc polypeptide; or
a5) the first polypeptide comprises, in order from N-terminus to C-terminus:
   i) the T1D peptide;
   ii) an MHC class II β polypeptide; and
   iii) the one or more immunomodulatory polypeptides; and
b5) the second polypeptide comprises, in order from N-terminus to C-terminus:
   i) an MHC class II α polypeptide; and
   ii) an Ig Fc polypeptide;
   wherein, in any of the above TMMPs, the components of the first polypeptide optionally may be joined by one or more linkers, and the components of the second polypeptide optionally may be joined by one or more linkers.
3. A TMMP of aspect 2, wherein the MHC class II α polypeptide comprises an amino acid sequence having at least 95%, at least 98% or at least 99%, amino acid sequence identity to a DRA1*01:01 polypeptide; and the MHC class II β polypeptide comprises an amino acid sequence having at least 95%, at least 98%, or at least 99% amino acid sequence identity to a DRB1*04:01 polypeptide.
4. A TMMP of any one of aspects 1-3, wherein the one or more immunomodulatory polypeptides are selected from the group consisting of an IL-2 polypeptide, PD-L1 polypeptide, a FasL polypeptide, a TGF-β polypeptide, and combinations thereof.
5. A TMMP of any one of aspects 1-3, wherein at least one of the one or more immunomodulatory polypeptides is a PD-L1 polypeptide.
6. A TMMP of aspect 5, wherein the immunomodulatory polypeptide comprises a PD-L1 polypeptide extracellular domain.
7. A TMMP of aspect 6, wherein the immunomodulatory polypeptide comprises an amino acid sequence having at least 95%, at least 98%, at least 99%, or 100% amino acid sequence identity to the PD-L1 amino acid sequence depicted in FIG. 130, and has a length of 200 amino acids or about 220 amino acids.
8. A TMMP of any one of aspects 1-7, wherein the T1D peptide has a length of from about 4 amino acids to about 25 amino acids.
9. A TMMP of any one of aspects 1-8, wherein the T1D peptide comprises is a proinsulin peptide or a glutamic acid decarboxylase (GAD) peptide.
10. A TMMP of aspect 9, wherein: (a) the T1D peptide is a proinsulin peptide selected from SLQPLALEGSLQKRG (SEQ ID NO:94; proIns 76-90), SLQPLALEGSLQSRG (SEQ ID NO:90; proIns 76-90; K88S), and GAGSLQPLALEGSLQKRG (SEQ ID NO:93; proIns 73-90); or (b) wherein the T1D peptide is a GAD peptide selected from NFFRMVISNPAAT (SEQ ID NO:87; GAD65 555-567) and NFIRMVISNPAAT (SEQ ID NO:88; GAD65 555-567; F557I).
11. A TMMP of any one of aspects 1-8, wherein the T1D peptide comprises the amino acid sequence SLQPLALEGSLQSRG (SEQ ID NO:90).
12. A TMMP of any one of aspects 1-11, wherein the first MHC class II polypeptide is an MHC class II β chain polypeptide (comprising an amino acid sequence having at least 95%, at least 98%, or at least 99% amino acid sequence identity to a DRB1*04:01 polypeptide), and wherein the second MHC class II polypeptide is an MHC class II α chain polypeptide (e.g., having at least 95%, at least 98% or at least 99%, amino acid sequence identity to a DRA1*01:01 polypeptide).
13. A TMMP of any one of aspects 1-12, wherein the first MHC class II polypeptide comprises a Cys-containing linker between the T1D peptide and the MHC Class II β chain polypeptide, and wherein a disulfide bond is formed between the Cys in the linker and a Cys in the second MHC class II polypeptide.
14. A TMMP of aspect 13, wherein the linker comprises an amino acid sequence selected from (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where the linker is from about 15 amino acids to about 20 amino acids, such as (GGCGS)(GGGGS)n where n=2 (SEQ ID NO:128) or n=3 (SEQ ID NO:129)).
15. A TMMP of aspect 13, wherein the second MHC class II polypeptide is a variant MHC class II polypeptide that comprises a non-naturally occurring Cys residue.
16. A TMMP of aspect 15, wherein the second MHC class II polypeptide is a DRA MHC class II polypeptide (e.g., having at least 95%, at least 98% or at least 99%, amino acid sequence identity to a DRA1*01:01 polypeptide).
17. A TMMP of aspect 15 or aspect 16, wherein the non-naturally occurring Cys residue is located at an amino acid residue from amino acids 55 to 110 of the second MHC Class II polypeptide.
18. A TMMP of any one of aspects 15-17, wherein the second MHC class II polypeptide is a variant DRA MHC class II polypeptide comprising a Cys at position 72 or 75 based on the amino acid numbering depicted in FIG. 13A.
19. A TMMP of aspect 18, wherein the variant DRA MHC class II polypeptide comprises a K75C substitution or an I72C substitution (e.g., a DRA MHC class II polypeptide comprising an amino acid sequence having at least 80%, at least 85%, at least 90%, at least 95%, at least 98%, at least 99%, or 100% amino acid sequence identity to the amino acid sequence depicted in FIG. 13G or 13H).
20. A TMMP of any one of aspects 14-19, wherein the TMMP comprises:
a1) a first polypeptide comprising, in order from N-terminus to C-terminus: i) a T1D peptide; ii) a linker comprising an amino acid sequence selected from the group consisting of (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where the linker is from about 15 amino acids to about 20 amino acids, such as (GGCGS)(GGGGS)n where n=2 (SEQ ID NO:128) or n=3 (SEQ ID NO:129)); and iii) a DRB MHC class II polypeptide comprising an amino acid sequence having at least 80%, at least 85%, at least 90%, at least 95%, at least 98%, at least 99%, or 100% amino acid sequence identity to the amino acid sequence depicted in FIG. 14A; and
b1) a second polypeptide comprising, in order from N-terminus to C-terminus: i) a DRA MHC class II polypeptide comprising an amino acid sequence having at least 80%, at least 85%, at least 90%, at least 95%, at least 98% or at least 99%, amino acid sequence identity to the amino acid sequence depicted in FIG. 13G, and comprising a Cys at position 72 or 75; an ii) a variant human IgG1 Fc polypeptide that substantially does not induce cell lysis through activation of CDC (e.g., a human IgG1 Fc polypeptide comprising L234A and L235A substitutions shown as L14A and L15A of the amino acid sequence depicted in FIG. 12A); and iii) one or more immunomodulatory polypeptides, wherein the one or more immunomodulatory polypeptides is a wt or variant PD-L1 polypeptide (e.g., comprising an amino acid sequence having at least 80%, at least 85%, at least 90%, at least 95%, at least 98%, at least 99%, or 100% sequence identity to the PD-L1 amino acid sequence depicted in FIG. 130, and having a length of about 220 amino acids); or, alternatively
b2) a second polypeptide comprising, in order from N-terminus to C-terminus: i) a DRA MHC class II polypeptide comprising an amino acid sequence having at least 80%, at least 85%, at least 90%, at least 95%, at least 98%, at least 99%, or 100% amino acid sequence identity to the amino acid sequence depicted in FIG. 13G or 13H, and comprising a Cys at position 72 or 75; an ii) a variant human IgG1 Fc polypeptide that substantially does not induce cell lysis through activation of CDC (e.g., a human IgG1 Fc polypeptide comprising L234A and L235A substitutions, shown as L14A and L15A of the amino acid sequence depicted in FIG. 12A); and iii) one or more immunomodulatory polypeptides, wherein the one or more immunomodulatory polypeptides is a wt or variant PD-L1 polypeptide (e.g., comprising an amino acid sequence having at least 80%, at least 85%, at least 90%, at least 95%, at least 98%, at least 99%, or 100% sequence identity to the PD-L1 amino acid sequence depicted in FIG. 130, and having a length of about 220 amino acids).
21. A TMMP of aspect 1, wherein:
i) the first polypeptide comprises the amino acid sequence of the 3893 polypeptide depicted in FIG. 15B; and
ii) the first polypeptide comprises the amino acid sequence of the 2938 polypeptide depicted in FIG. 15A.
22. A TMMP of any one of aspects 1-12, wherein the disulfide bond is formed between:
i) a Cys in the first MHC class II polypeptide; and
ii) a Cys in the second MHC class II polypeptide.
23. A TMMP of aspect 22, wherein: i) the first MHC class II polypeptide is a variant MHC class II polypeptide (e.g., an MHC class II β chain polypeptide such as a DRB MHC class II polypeptide) that comprises a non-naturally occurring Cys residue; and ii) the second MHC class II polypeptide is a variant MHC class II polypeptide (e.g., an MHC class II α chain polypeptide such as a DRA MHC class II polypeptide) that comprises a non-naturally occurring Cys residue.
24. A TMMP of aspect 23, wherein the MHC class II polypeptide is a variant DRB MHC class II polypeptide (e.g., comprising an amino acid sequence having at least 95%, at least 98%, or at least 99% amino acid sequence identity to a DRB1*04:01 polypeptide) that comprises an amino acid substitution selected from the group consisting of P5C, F7C, Q10C, N19C, G20C, H33C, G151C, D152C, and W153C.
25. A TMMP of aspect 23 or aspect 24, wherein the MHC class II polypeptide is a variant DRA MHC class II polypeptide (e.g., having at least 95%, at least 98% or at least 99%, amino acid sequence identity to a DRA1*01:01 polypeptide) that comprises an amino acid substitution selected from E3C, E4C, F12C, G28C, D29C, I72C, K75C, T80C, P81C, 182C, T93C, N94C, and S95C.
26. A TMMP of aspect 24 or 25, wherein:
a) the amino acid substitution in the DRA MHC class II polypeptide is P81C; and the amino acid substitution in the DRB MHC class II polypeptide is F7C;
b) the amino acid substitution in the DRA MHC class II polypeptide is P81C; and the amino acid substitution in the DRB MHC class II polypeptide is P5C;
c) the amino acid substitution in the DRA MHC class II polypeptide is P81C; and the amino acid substitution in the DRB MHC class II polypeptide is H33C;
d) the amino acid substitution in the DRA MHC class II polypeptide is E4C; and the amino acid substitution in the DRB MHC class II polypeptide is N19C;
e) the amino acid substitution in the DRA MHC class II polypeptide is E4C; and the amino acid substitution in the DRB MHC class II polypeptide is G20C;
f) the amino acid substitution in the DRA MHC class II polypeptide is T93C; and the amino acid substitution in the DRB MHC class II polypeptide is Q156C;
g) the amino acid substitution in the DRA MHC class II polypeptide is T93C; and the amino acid substitution in the DRB MHC class II polypeptide is W153C;
h) the amino acid substitution in the DRA MHC class II polypeptide is F12C; and the amino acid substitution in the DRB MHC class II polypeptide is F7C;
i) the amino acid substitution in the DRA MHC class II polypeptide is F12C; and the amino acid substitution in the DRB MHC class II polypeptide is Q10C;
j) the amino acid substitution in the DRA MHC class II polypeptide is T80C; and the amino acid substitution in the DRB MHC class II polypeptide is F7C;
k) the amino acid substitution in the DRA MHC class II polypeptide is T80C; and the amino acid substitution in the DRB MHC class II polypeptide is P5C;
l) the amino acid substitution in the DRA MHC class II polypeptide is T80C; and the amino acid substitution in the DRB MHC class II polypeptide is I82C;
m) the amino acid substitution in the DRA MHC class II polypeptide is I82C; and the amino acid substitution in the DRB MHC class II polypeptide is F7C;
n) the amino acid substitution in the DRA MHC class II polypeptide is I82C; and the amino acid substitution in the DRB MHC class II polypeptide is P5C;
o) the amino acid substitution in the DRA MHC class II polypeptide is 182C; and the amino acid substitution in the DRB MHC class II polypeptide is H33C;
p) the amino acid substitution in the DRA MHC class II polypeptide is G28C; and the amino acid substitution in the DRB MHC class II polypeptide is G151C;
q) the amino acid substitution in the DRA MHC class II polypeptide is G28C; and the amino acid substitution in the DRB MHC class II polypeptide is D152C;
r) the amino acid substitution in the DRA MHC class II polypeptide is G28C; and the amino acid substitution in the DRB MHC class II polypeptide is W153C;
s) the amino acid substitution in the DRA MHC class II polypeptide is D29C; and the amino acid substitution in the DRB MHC class II polypeptide is G151C;
t) the amino acid substitution in the DRA MHC class II polypeptide is D29C; and the amino acid substitution in the DRB MHC class II polypeptide is D152C;
u) the amino acid substitution in the DRA MHC class II polypeptide is D29C; and the amino acid substitution in the DRB MHC class II polypeptide is W153C;
v) the amino acid substitution in the DRA MHC class II polypeptide is N94C; and the amino acid substitution in the DRB MHC class II polypeptide is Q156C;
w) the amino acid substitution in the DRA MHC class II polypeptide is N94C; and the amino acid substitution in the DRB MHC class II polypeptide is N120C;
y) the amino acid substitution in the DRA MHC class II polypeptide is S95C; and the amino acid substitution in the DRB MHC class II polypeptide is Q156C;
z) the amino acid substitution in the DRA MHC class II polypeptide is S95C; and the amino acid substitution in the DRB MHC class II polypeptide is N120C;
aa) the amino acid substitution in the DRA MHC class II polypeptide is E3C; and the amino acid substitution in the DRB MHC class II polypeptide is N19C; or
bb) the amino acid substitution in the DRA MHC class II polypeptide is E3C; and the amino acid substitution in the DRB MHC class II polypeptide is G20C.
27. A TMMP comprising two heterodimers according to any one of aspects 1-26, wherein each heterodimer comprises an Ig Fc polypeptide (e.g., a variant human IgG1 Fc polypeptide that substantially does not induce cell lysis through activation of CDC such as a human IgG1 Fc polypeptide comprising L234A and L235A substitutions, shown as L14A and L15A of the amino acid sequence depicted in FIG. 12A), and wherein the heterodimers are disulfide bonded to each other though their respective Ig Fc components.
28. A composition comprising:
a) a TMMP of any one of aspects 1-27; and
b) a pharmaceutically acceptable excipient.
29. One or more nucleic acids comprising nucleotide sequences encoding a T-cell modulatory multimeric polypeptide of any one of aspects 1-27.
30. One or more expression vectors comprising the one or more nucleic acids of aspect 29.
31. A host cell genetically modified with the one or more nucleic acids of aspect 29 or the one or more expression vectors of aspect 30.
32. A method of selectively modulating the activity of CD4⁺ T cells specific for a type 1 diabetes-associated epitope in an individual, the method comprising contacting the CD4⁺ T cells with a TMMP of any one of aspects 1-27.
33. A method of reducing the number and/or activity of CD4⁺ and/or CD8⁺ self-reactive T cells specific for a type 1 diabetes-associated epitope in an individual, the method comprising contacting the CD4⁺ T cells with a TMMP of any one of aspects 1-27.
34. A method of treating type 1 diabetes (T1D) in an individual, the method comprising administering to an individual in need thereof an effective amount of a TMMP of any one of aspects 1-27, or an effective amount of the composition of aspect 28.
35. An antigen-presenting polypeptide (APP) comprising:
a) a first polypeptide comprising:
   i) a peptide that displays a Type 1 Diabetes-associated epitope capable of being bound by a T-cell receptor (a “T1D peptide”); and
   ii) a first major histocompatibility complex (MHC) class II polypeptide; and
   iii) optionally a linker that links the T1D peptide to the first MHC class II polypeptide; and
b) a second polypeptide comprising a second MHC class II polypeptide,
   wherein the first and the second polypeptide of the heterodimer are covalently linked to one another via at least one disulfide bond,
   wherein one of the polypeptides of the heterodimer comprises an immunoglobulin (Ig) Fc polypeptide, optionally joined to the first or second polypeptide via a linker, and
wherein the APP does not include an immunomodulatory polypeptide.
36. An APP of aspect 35, wherein the first MHC class II polypeptide is an MHC class II β chain polypeptide and wherein the second MHC class II polypeptide is an MHC class II α chain polypeptide.
37. An APP of aspect 36, wherein the MHC class II α polypeptide comprises an amino acid sequence having at least 95%, at least 98% or at least 99%, amino acid sequence identity to a DRA1*01:01 polypeptide; and the MHC class II β polypeptide comprises an amino acid sequence having at least 95%, at least 98%, or at least 99% amino acid sequence identity to a DRB1*04:01 polypeptide.
38. An APP of any one of aspects 35-37, wherein the T1D peptide has a length of from about 4 amino acids to about 25 amino acids.
39. An APP of any one of aspects 35-38, wherein the T1D peptide comprises is a proinsulin peptide or a glutamic acid decarboxylase (GAD) peptide.
40. An APP of aspect 39, wherein the T1D peptide is a proinsulin peptide selected from SLQPLALEGSLQKRG (SEQ ID NO:94; proIns 76-90), SLQPLALEGSLQSRG (SEQ ID NO:90; proIns 76-90; K88S), and GAGSLQPLALEGSLQKRG (SEQ ID NO:93; proIns 73-90).
41. An APP of aspect 39, wherein the T1D peptide is a GAD peptide selected from NFFRMVISNPAAT (SEQ ID NO:87; GAD65 555-567) and NFIRMVISNPAAT (SEQ ID NO:88; GAD65 555-567; F557I).
42. An APP of any one of aspects 36-41, wherein the first polypeptide comprises a Cys-containing linker between the T1D peptide and the MHC Class II β chain polypeptide, and wherein a disulfide bond is formed between the Cys in the linker and a Cys in the second MHC class II polypeptide.
43. An APP of aspect 42, wherein the linker comprises an amino acid sequence selected from (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where the linker is from about 15 amino acids to about 20 amino acids, such as (GGCGS)(GGGGS)n where n=2 (SEQ ID NO:128) or n=3 (SEQ ID NO:129)).
44. An APP of aspect 42 or aspect 43, wherein the second MHC class II polypeptide is a variant MHC class II polypeptide that comprises a non-naturally occurring Cys residue.
45. An APP of aspect 44, wherein the second MHC class II polypeptide is a DRA MHC class II polypeptide (e.g., having at least 95%, at least 98% or at least 99% amino acid sequence identity to a DRA1*01:01 polypeptide).
46. An APP of aspect 44 or 45, wherein the non-naturally occurring Cys residue is located at an amino acid residue from amino acids 55 to 110 of the second MHC Class II polypeptide.
47. An APP of any one of aspects 42-46, wherein the second MHC class II polypeptide is a variant DRA MHC class II polypeptide comprising a Cys at position 72 or 75 based on the amino acid numbering depicted in FIG. 13A.
48. An APP of any one of aspects 42-47, wherein the variant DRA MHC class II polypeptide comprises a K75C substitution or an I72C substitution (e.g., a DRA MHC class II polypeptide comprising an amino acid sequence having at least 80%, at least 85%, at least 90%, at least 95%, at least 98%, at least 99%, or 100% amino acid sequence identity to the amino acid sequence depicted in FIG. 13G or 13H).
49. An APP of any one of aspects 42-48, comprising:
a1) a first polypeptide comprising, in order from N-terminus to C-terminus: i) a T1D peptide; ii) a linker comprising an amino acid sequence selected from the group consisting of (CGGGS)(GGGGS)n (SEQ ID NO:1), (GCGGS)(GGGGS)n (SEQ ID NO:2), (GGCGS)(GGGGS)n (SEQ ID NO:3), (GGGCS)(GGGGS)n (SEQ ID NO:4), and (GGGGC)(GGGGS)n (SEQ ID NO:5), where n is an integer from 1 to 10 (e.g., where the linker is from about 15 amino acids to about 20 amino acids, such as (GGCGS)(GGGGS)n where n=2 (SEQ ID NO:128) or n=3 (SEQ ID NO:129); and iii) an MHC class II β chain polypeptide (e.g., a DRB MHC class II polypeptide comprising an amino acid sequence having at least 80%, at least 85%, at least 90%, at least 95%, at least 98%, at least 99%, or 100%, amino acid sequence identity to the amino acid sequence depicted in FIG. 14A); and
b1) a second polypeptide comprising, in order from N-terminus to C-terminus: i) an MHC class II α chain comprising Cys at position 72 or 75 (e.g., a DRA MHC class II polypeptide comprising an amino acid sequence having at least 80%, at least 85%, at least 90%, at least 95%, at least 98%, at least 99%, or 100% amino acid sequence identity to the amino acid sequence depicted in FIG. 13G or 13H, and comprising a Cys at position 72 or 75); and an ii) a variant human IgG1 Fc polypeptide that substantially does not induce cell lysis through activation of CDC (e.g., a human IgG1 Fc polypeptide comprising L234A and L235A substitutions, shown as L14A and L15A of the amino acid sequence depicted in FIG. 12A).
50. An APP of any one of aspects 35-41, wherein the disulfide bond is formed between:
i) a Cys in the first MHC class II polypeptide; and
ii) a Cys in the second MHC class II polypeptide.
51. An APP of aspect 50, wherein: i) the first MHC class II polypeptide is a variant MHC class II polypeptide that comprises a non-naturally occurring Cys residue; and ii) the second MHC class II polypeptide is a variant MHC class II polypeptide that comprises a non-naturally occurring Cys residue.
52. An APP of aspect 50 or aspect 51, wherein the first MHC class II polypeptide is a DRB MHC class II polypeptide, and wherein the second MHC class II polypeptide is a DRA MHC class II polypeptide.
53. An APP of aspect 52, wherein the DRB MHC class II polypeptide comprises an amino acid substitution selected from the group consisting of P5C, F7C, Q10C, N19C, G20C, H33C, G151C, D152C, and W153C.
54. An APP of aspect 52 or aspect 53, wherein DRA MHC class II polypeptide comprises an amino acid substitution selected from the group consisting of E3C, E4C, F12C, G28C, D29C, I72C, K75C, T80C, P81C, 182C, T93C, N94C, and S95C.
55. An APP of aspect 53 or 54, wherein:
a) the amino acid substitution in the DRA MHC class II polypeptide is P81C; and the amino acid substitution in the DRB MHC class II polypeptide is F7C;
b) the amino acid substitution in the DRA MHC class II polypeptide is P81C; and the amino acid substitution in the DRB MHC class II polypeptide is P5C;
c) the amino acid substitution in the DRA MHC class II polypeptide is P81C; and the amino acid substitution in the DRB MHC class II polypeptide is H33C;
d) the amino acid substitution in the DRA MHC class II polypeptide is E4C; and the amino acid substitution in the DRB MHC class II polypeptide is N19C;
e) the amino acid substitution in the DRA MHC class II polypeptide is E4C; and the amino acid substitution in the DRB MHC class II polypeptide is G20C;
f) the amino acid substitution in the DRA MHC class II polypeptide is T93C; and the amino acid substitution in the DRB MHC class II polypeptide is Q156C;
g) the amino acid substitution in the DRA MHC class II polypeptide is T93C; and the amino acid substitution in the DRB MHC class II polypeptide is W153C;
h) the amino acid substitution in the DRA MHC class II polypeptide is F12C; and the amino acid substitution in the DRB MHC class II polypeptide is F7C;
i) the amino acid substitution in the DRA MHC class II polypeptide is F12C; and the amino acid substitution in the DRB MHC class II polypeptide is Q10C;
j) the amino acid substitution in the DRA MHC class II polypeptide is T80C; and the amino acid substitution in the DRB MHC class II polypeptide is F7C;
k) the amino acid substitution in the DRA MHC class II polypeptide is T80C; and the amino acid substitution in the DRB MHC class II polypeptide is P5C;
l) the amino acid substitution in the DRA MHC class II polypeptide is T80C; and the amino acid substitution in the DRB MHC class II polypeptide is I82C;
m) the amino acid substitution in the DRA MHC class II polypeptide is 182C; and the amino acid substitution in the DRB MHC class II polypeptide is F7C;
n) the amino acid substitution in the DRA MHC class II polypeptide is 182C; and the amino acid substitution in the DRB MHC class II polypeptide is P5C;
o) the amino acid substitution in the DRA MHC class II polypeptide is 182C; and the amino acid substitution in the DRB MHC class II polypeptide is H33C;
p) the amino acid substitution in the DRA MHC class II polypeptide is G28C; and the amino acid substitution in the DRB MHC class II polypeptide is G151C;
q) the amino acid substitution in the DRA MHC class II polypeptide is G28C; and the amino acid substitution in the DRB MHC class II polypeptide is D152C;
r) the amino acid substitution in the DRA MHC class II polypeptide is G28C; and the amino acid substitution in the DRB MHC class II polypeptide is W153C;
s) the amino acid substitution in the DRA MHC class II polypeptide is D29C; and the amino acid substitution in the DRB MHC class II polypeptide is G151C;
t) the amino acid substitution in the DRA MHC class II polypeptide is D29C; and the amino acid substitution in the DRB MHC class II polypeptide is D152C;
u) the amino acid substitution in the DRA MHC class II polypeptide is D29C; and the amino acid substitution in the DRB MHC class II polypeptide is W153C;
v) the amino acid substitution in the DRA MHC class II polypeptide is N94C; and the amino acid substitution in the DRB MHC class II polypeptide is Q156C;
w) the amino acid substitution in the DRA MHC class II polypeptide is N94C; and the amino acid substitution in the DRB MHC class II polypeptide is N120C;
y) the amino acid substitution in the DRA MHC class II polypeptide is S95C; and the amino acid substitution in the DRB MHC class II polypeptide is Q156C;
z) the amino acid substitution in the DRA MHC class II polypeptide is S95C; and the amino acid substitution in the DRB MHC class II polypeptide is N120C;
aa) the amino acid substitution in the DRA MHC class II polypeptide is E3C; and the amino acid substitution in the DRB MHC class II polypeptide is N19C; or
bb) the amino acid substitution in the DRA MHC class II polypeptide is E3C; and the amino acid substitution in the DRB MHC class II polypeptide is G20C.
56. An APP of any one of aspects 35-55, wherein the second polypeptide comprises the Ig Fc polypeptide.
57. An APP of any one of aspects 35-56, wherein the Ig Fc polypeptide induces cell lysis through activation of complement-dependent cytotoxicity (CDC) and/or elicits antibody-dependent cellular cytotoxicity (ADCC).
58. An APP of any one of aspects 35-56, further comprising a detectable label that permits binding of the APP to a target T cell to be detected.
59. An APP comprising two heterodimers according to any of aspects 35-58, wherein each heterodimer comprises an Ig Fc polypeptide, and wherein the heterodimers are disulfide bonded to each other though their respective Ig Fc components.
60. A composition comprising:
a) an APP of any one of aspects 35-59; and
b) a pharmaceutically acceptable excipient.
61. One or more nucleic acids comprising nucleotide sequences encoding an APP of any one of aspects 35-59.
62. One or more expression vectors comprising the one or more nucleic acids of aspect 61.
63. A host cell genetically modified with the one or more nucleic acids of aspect 61 or the one or more expression vectors of aspect 62.
64. A method of reducing the number and/or activity of CD4⁺ and/or CD8⁺ self-reactive T cells specific for a type 1 diabetes-associated epitope in an individual, the method comprising contacting the CD4⁺ T cells with an APP of aspect 57.
65. A method of treating type 1 diabetes (T1D) in an individual, the method comprising administering to an individual in need thereof an effective amount of an APP of aspect 57.
66. A method of detecting a CD4⁺ T cell specific for a type 1 diabetes-associated epitope in an individual, the method comprising the steps of contacting the CD4⁺ T cell with an APP of aspect 58, and detecting the presence of the label.
67. A TMMP according to aspect 27, wherein at least 50%, at least 60%, at least 70%, at least 80%, or at least 90% of an initial quantity of TMMP monomer remains present as a monomer in a PBS buffer solution after 3 days at 37° C. at a TMMP concentration of 9.5 mg/mL, 1 mg/mL, or 0.1 mg/mL,
wherein the PBS buffer solution, not including the TMMP monomer, comprises sodium chloride (500 mM), sodium phosphate dibasic (10 mM), potassium phosphate monobasic (2 mM), potassium chloride (2.7 mM), and water, and has a pH of 7.5+0.15, and
wherein the amount of monomer is determined using size exclusion chromatography using a Superdex 200 Increase (3.2×300 mm) column (e.g., from GE Healthcare), with a running buffer of the PBS buffer solution, and wherein the flow rate is 0.15 ml/minute.
68. A TMMP according to aspect 27, wherein at least 50%, at least 60%, at least 70%, or at least 80% of an initial quantity of TMMP monomer remains present as a monomer in a PBS buffer solution after 5 days at 37° C. at a TMMP concentration of 9.5 mg/mL, 1 mg/mL, or 0.1 mg/mL,
wherein the PBS buffer solution, not including the TMMP monomer, comprises sodium chloride (500 mM), sodium phosphate dibasic (10 mM), potassium phosphate monobasic (2 mM), potassium chloride (2.7 mM), and water, and has a pH of 7.5+0.15, and
wherein the amount of monomer is determined using size exclusion chromatography using a Superdex 200 Increase (3.2×300 mm) column (e.g., from GE Healthcare), with a running buffer of the PBS buffer solution, and wherein the flow rate is 0.15 ml/minute.
69. A TMMP according to aspect 27, wherein at least 50%, at least 60%, or at least 70% of an initial quantity of TMMP monomer remains present as a monomer in a PBS buffer solution after 3 days at 42° C. at a TMMP concentration of 9.5 mg/mL, 1 mg/mL, or 0.1 mg/mL,
wherein the PBS buffer solution, not including the TMMP monomer, comprises sodium chloride (500 mM), sodium phosphate dibasic (10 mM), potassium phosphate monobasic (2 mM), potassium chloride (2.7 mM), and water, and has a pH of 7.5+0.15, and
wherein the amount of monomer is determined using size exclusion chromatography using a Superdex 200 Increase (3.2×300 mm) column (e.g., from GE Healthcare), with a running buffer of the PBS buffer solution, and wherein the flow rate is 0.15 ml/minute.
70. A TMMP according to aspect 27, wherein at least 50%, at least 60%, at least 70%, at least 80%, or at least 90% of an initial quantity of TMMP monomer remains present as a monomer in a PBS buffer solution after 5 days at 42° C. at a TMMP concentration of 9.5 mg/mL, 1 mg/mL, or 0.1 mg/mL,
wherein the PBS buffer solution, not including the TMMP monomer, comprises sodium chloride (500 mM), sodium phosphate dibasic (10 mM), potassium phosphate monobasic (2 mM), potassium chloride (2.7 mM), and water, and has a pH of 7.5+0.15, and
wherein the amount of monomer is determined using size exclusion chromatography using a Superdex 200 Increase (3.2×300 mm) column (e.g., from GE Healthcare), with a running buffer of the PBS buffer solution, and wherein the flow rate is 0.15 ml/minute.
71. A TMMP according to any one of aspects 67-70, wherein the TMMP is in the PBS buffer solution at a concentration of 9.5 mg/mL.
72. A TMMP according to any one of aspects 67-70, wherein the TMMP is in the PBS buffer solution at a concentration of 1 mg/mL.
73. A TMMP according to any one of aspects 67-70, wherein the TMMP is in the PBS buffer solution at a concentration of 0.1 mg/mL.

### EXAMPLES

The following examples are put forth so as to provide those of ordinary skill in the art with a complete disclosure and description of how to make and use aspects of the present disclosure, and are not intended to limit the scope of what the inventors regard as their disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Disulfide Bond Placement

One set of constructs (“body disulfide” set) was made that included two heterodimers, each heterodimer including: a) a first polypeptide comprising: i) a peptide epitope (e.g., a proinsulin peptide or a GAD65 peptide); and ii) an hDRB1*0401 β chain polypeptide comprising an amino acid substitution providing a Cys (a “first Cys”); and b) a second polypeptide comprising: i) an hDRA1*0101 α chain polypeptide comprising an amino acid substitution providing a Cys (a “second Cys”); and ii) an Ig Fc polypeptide, where the first and second polypeptides are covalently linked to one another via a disulfide between the first Cys and the second Cys. A second set of constructs (“linker disulfide set”) was made that included two heterodimers, each heterodimer including: a) a first polypeptide comprising: i) a peptide epitope (e.g., a proinsulin peptide or a GAD65 peptide); ii) a peptide linker comprising a Cys; and iii) an hDRB1*0401 β chain polypeptide; and b) a second polypeptide comprising: i) an hDRA1*0101 α chain polypeptide comprising an amino acid substitution providing a Cys; and ii) an Ig Fc polypeptide, where the first and second polypeptides are covalently linked to one another via a disulfide between the Cys in the peptide linker and the Cys provided by the amino acid substitution in the α chain.

The ability of the first and the second polypeptides to form disulfide-linked heterodimers was tested by running the constructs on sodium dodecyl sulfate (SDS) polyacrylamide gels (PAGE) containing dithiothreitol (reducing) or not containing dithiothreitol (non-reducing).

A protein consisting of 2 heterodimers, and a total of 4 polypeptide chains, is referred to in this Example as a “monomer.” Whether a protein was present in solution as a monomer was determined using size exclusion chromatography (SEC). SEC was performed using the PBS containing 500 mM NaCl (described above) as the running buffer; and a flow rate of 0.15 ml/minute.

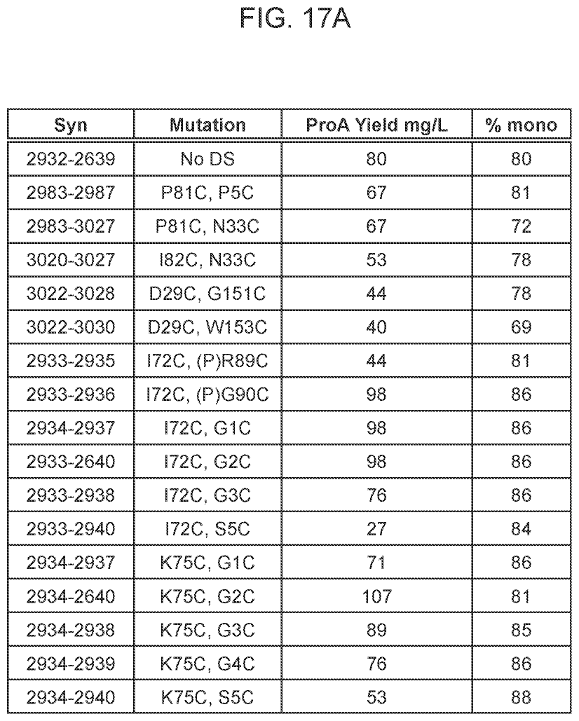

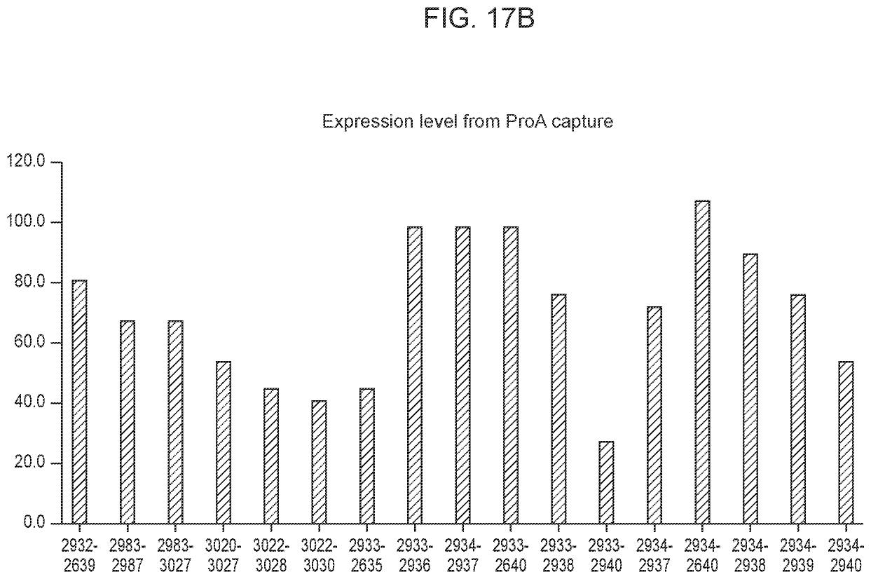

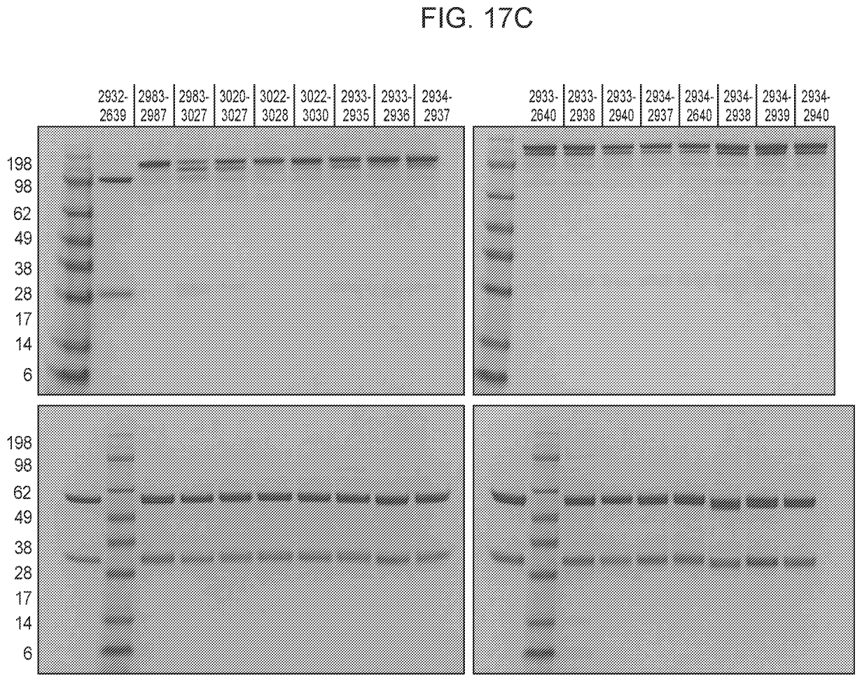

FIG. 17A depicts various “body disulfide” and “linker disulfide” constructs, the position of the Cys residues forming a disulfide bond between the two polypeptides of a heterodimer, the yield from a Protein A column, and the percent monomer. Amino acid sequences of the various polypeptide chains of the constructs are depicted in FIG. 15. FIG. 17B depicts expression level from the Protein A column. FIG. 17C shows reducing (lower panels) and non-reducing (upper panels) SDS-PAGE results for the various proteins.

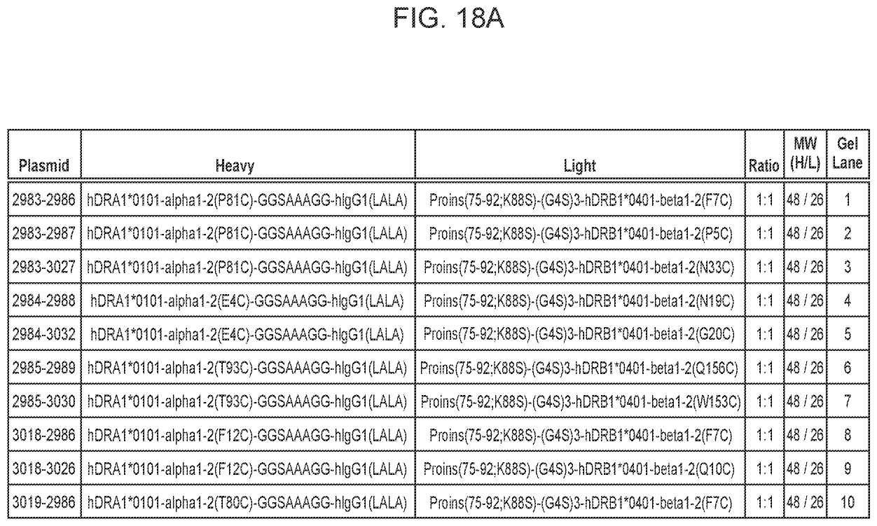

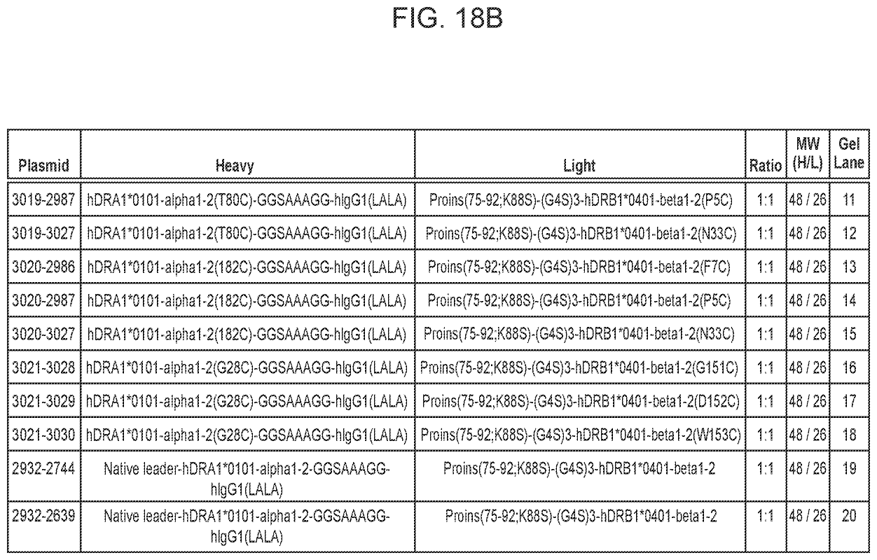

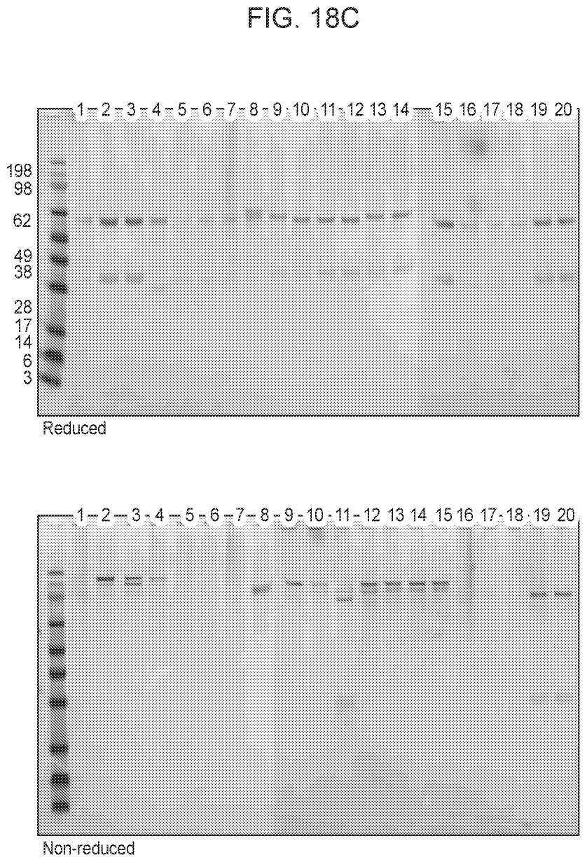

FIG. 18A-18B depict various “body disulfide” constructs and features of the two polypeptides of the heterodimers. Amino acid sequences of the various polypeptide chains of the constructs are depicted in FIG. 15. The constructs were analyzed on reducing and non-reducing SDS-PAGE. The results are shown in FIG. 18C.

### Example 2: MOD Placement

Various constructs were made to test the effect of the position of the immunomodulatory polypeptide (“MOD”) in a TMMP. The various possibilities tested are Positions 1-5, depicted schematically in FIG. 5. TMMPs were generated that comprise: a) PD-L1 as the MOD, at various positions; b) GAD65 (555-567) as the T1D peptide; and c) MHC class II DRA α chain and DRB β chains. The two polypeptide chains of the heterodimers were not covalently linked. Amino acid sequences of the polypeptide chains of the constructs are depicted in FIG. 15.

The results are shown in FIG. 18A-19D. FIG. 19A shows expression levels of the various constructs. FIG. 19B depicts reducing and non-reducing SDS-PAGE results. FIG. 19C depicts SEC data used to determine the % monomer. FIG. 19D shows expression levels determined by either Protein A column yields or SEC data.

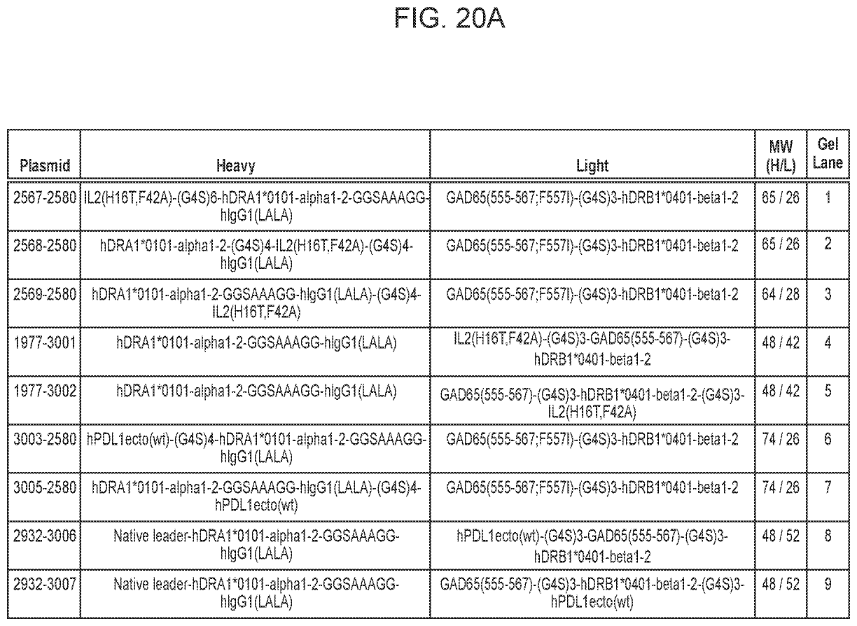

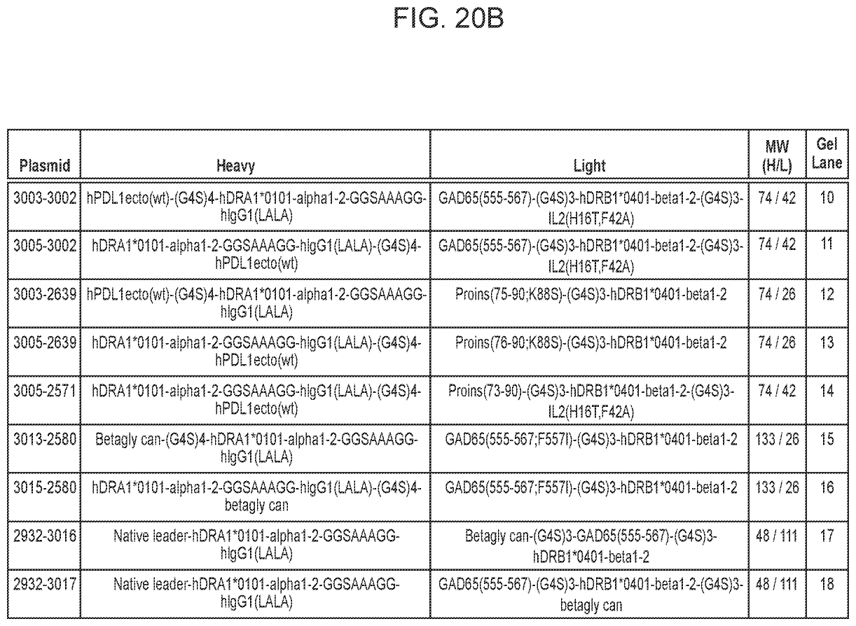

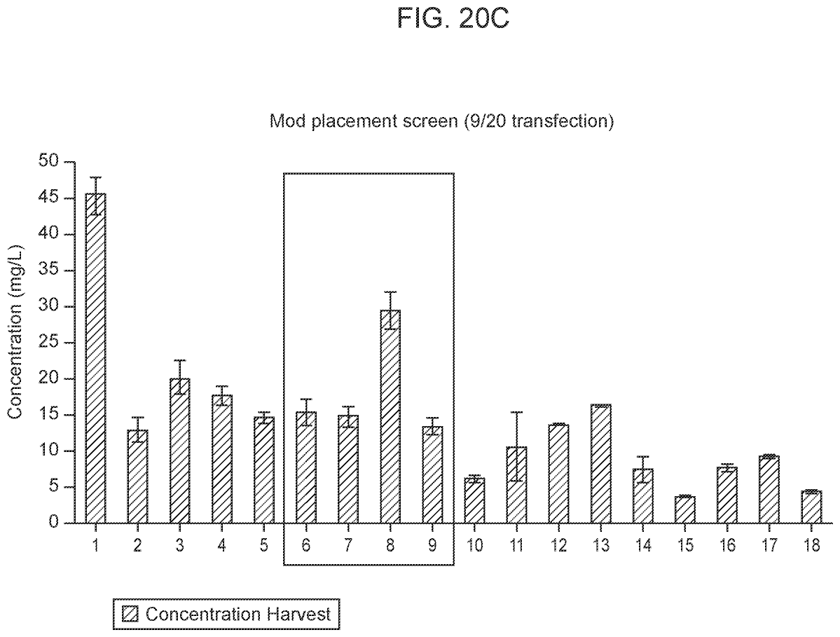

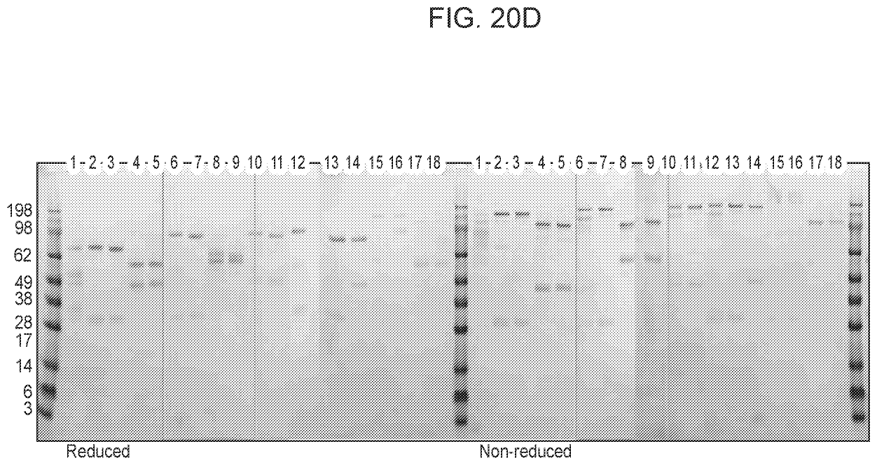

Additional constructs were made and tested. Features of the constructs are shown in FIG. 20A-20B. Production levels of the constructs are shown in FIG. 20C. Results of non-reducing and reducing SDS-PAGE analysis of the constructs are shown in FIG. 20D.

### Example 3: In Vitro Stability

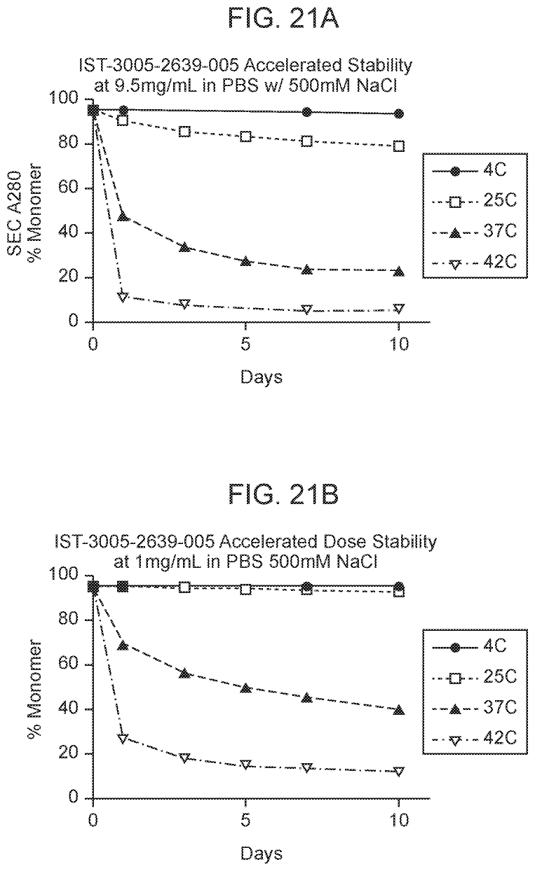

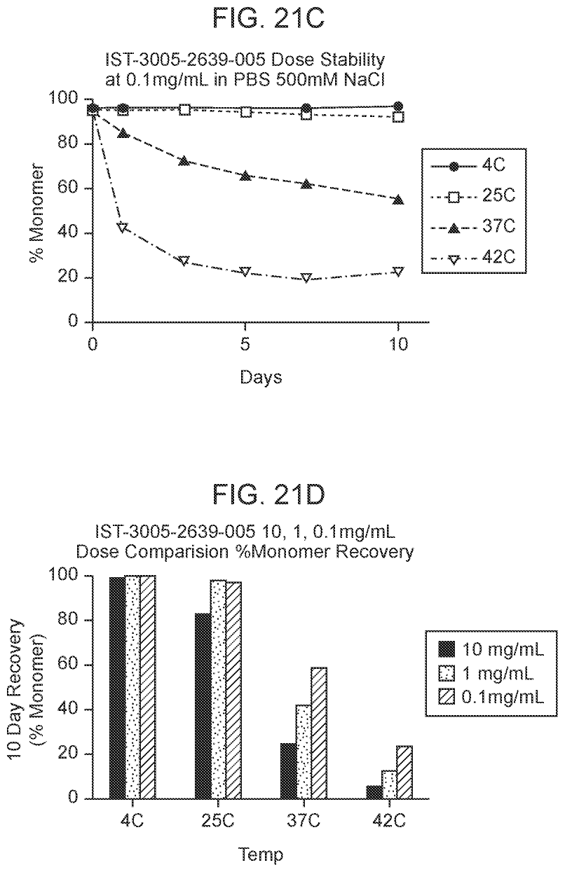

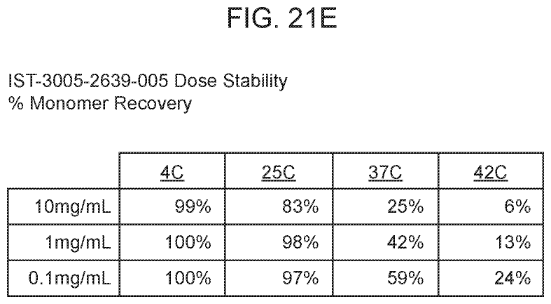

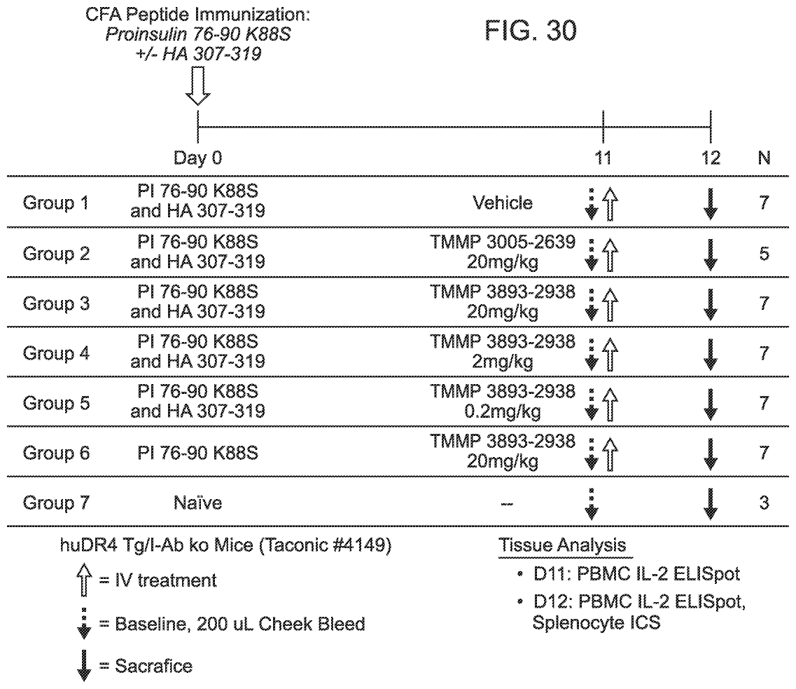

The in vitro stability of TMMP 3005-2639 was tested. TMMP 3005-2639 in a solution of PBS containing 500 mM NaCl (described above), at TMMP concentrations of 9.5 mg/mL, 1 mg/mL, or 0.1 mg/mL, was kept at 4° C., 25° C., 37° C., or 42° C. for up to 10 days. At various time points, the % monomer was determined. The results are shown in FIG. 21A-21E. FIG. 21A-21C show the % monomer at 9.5 mg/mL (FIG. 21A), 1 mg/mL (FIG. 21B), and 0.1 mg/mL (FIG. 21C). FIG. 30D shows % monomer recovery at the various concentrations and temperatures. The % monomer recovery data from FIG. 21D are summarized in FIG. 21E. The data show that increased aggregation upon thermal stress is proportional to increased concentration and temperature.

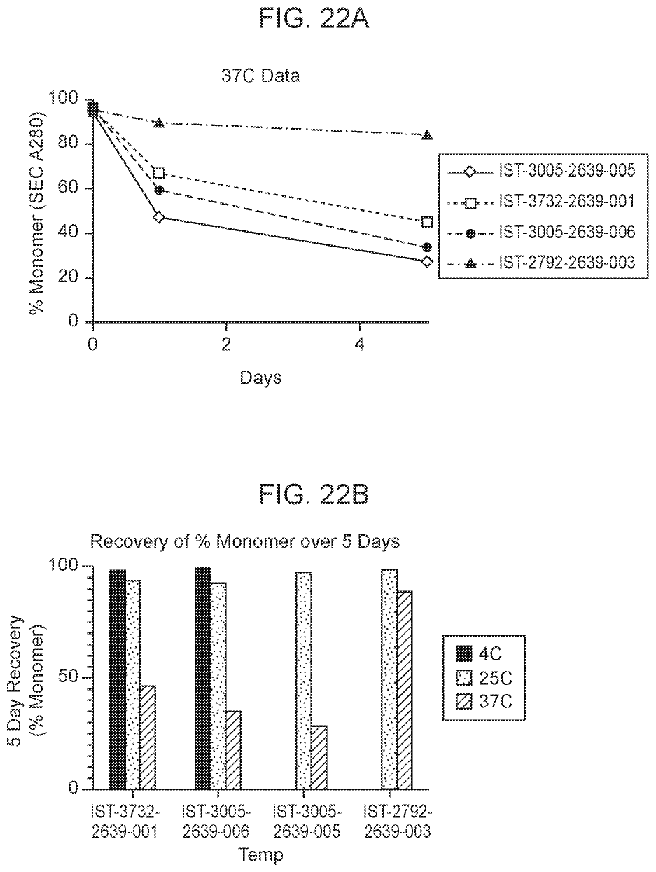

The in vitro stability of disulfide-stabilized TMMP, in which the MOD is at Position 3, was tested. The disulfide-stabilized TMMP was 3732-2639 (FIG. 15NN and FIG. 15C). Control TMMPs, in which the first and second polypeptides were not disulfide bonded, were also tested. The control TMMPs were: i) 2792-2639 (FIG. 15OO and FIG. 15C); and ii) 3005-2639 (FIG. 15D and FIG. 15C). Two different batches of TMMP 3005-2639, where the batches were designated “005” and “006,” were tested. The data are shown in FIG. 22A and FIG. 22B. FIG. 22A depicts the % monomer remaining after 5 days at 37° C. FIG. 22B depict the % monomer after 5 days at 4° C., 25° C., and 37° C. The data indicate that disulfide linkage between the first polypeptide and the second polypeptide of a TMMP increases stability.

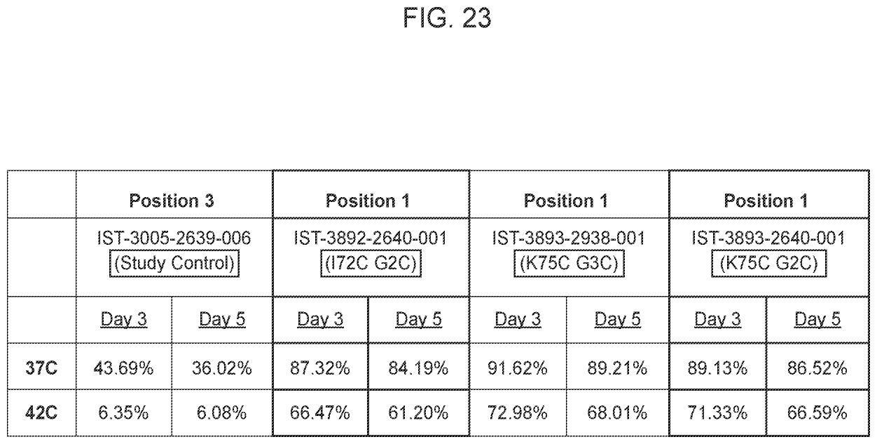

The in vitro stability of various disulfide-stabilized TMMPs, in which the MOD (PD-L1) is at Position 1, and in which the disulfide bond was between i) a Cys in the linker between the proIns epitope (proIns 76-90 (K88S)) and the MHC class II β chain; and ii) a Cys in the MHC class II α chain, was tested. The TMMPs were: i) 3892-2640 (FIG. 15PP and FIG. 15JJ); ii) 3893-2938 (FIG. 15B and FIG. 15A); and iii) 3893-2640 (FIG. 15B and FIG. 15JJ). A control TMMP, 3005-2639 (FIG. 15D and FIG. 15C), in which the first and second polypeptides were not disulfide bonded, was also tested. The in vitro stability of a TMMP concentration of 9.5 mg/mL was tested for the following conditions: i) 37° C. for 3 days; ii) 37° C. for 5 days; iii) 42° C. for 3 days; and iv) 42° C. for 5 days. The data are shown in FIG. 23. The data indicate that disulfide linkage between the first polypeptide and the second polypeptide of a TMMP increases stability.

### Example 4: Expression Levels

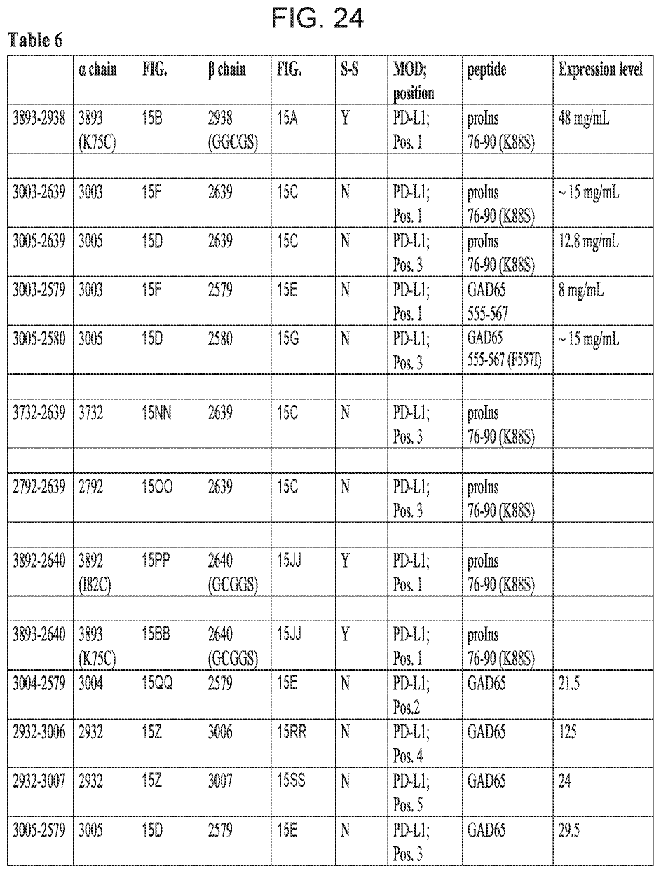

Expression levels of various TMMPs are shown in the table presented in FIG. 24. It is noted that expression levels can vary with the conditions and scale employed. For example, while further expression results in a larger scale showed that 3003-2639 remained low, expression of 3005-2639 was able to achieve 30-40 mg/L expression titer.

### Example 5: Effect of TMMPs on Functional Responses of Epitope-Specific CD4⁺ T Cells In Vitro

The effect of TMMPs on functional responses of proinsulin-specific CD4⁺ T cells was assessed. The ability of Proins-PDL1 Pos3 (3005-2639) and Proins-PDL1 Pos 1 (3893-2938) to suppress Proinsulin-specific CD4 cells was assessed in an overnight suppression assay. TMMP 3005-2639 comprises two heterodimers; each heterodimer comprises: a) a 3005 polypeptide (FIG. 15D); and b) a 2639 polypeptide (FIG. 15C), where the 3005 polypeptide and the 2639 polypeptide are non-covalently associated. TMMP 3892-2938 comprises two heterodimers; each heterodimer comprises: a) a 3893 polypeptide (FIG. 15B); and b) a 2938 polypeptide (FIG. 15A), where the 3893 polypeptide and the 2938 polypeptide are covalently linked via a disulfide bond.

Peripheral blood mononuclear cells (PBMCs) from multiple healthy or T1D patients were expanded with proinsulin (PI) 76-90K88S peptide and recombinant human IL-2 (rhIL-2) in RPMI+10% human serum for 14 days. Expanded PBMCs were washed and incubated (2e6 cells/well, 24 well plate) with increasing concentrations of indicated TMMPs overnight at 37° C., 5% CO₂. The TMMP-treated cells were washed, and functional response of antigen-specific cells was assessed using an IFNγ ELISPOT upon re-stimulation with PI 76-90K88S peptide. Additionally, the TMMP-treated, washed cells were cultured in presence of autologous PBMCs (1:1) and rhIL-2 to assess duration of suppressive effect. IFNγ ELISPOT was performed at the indicated time points.

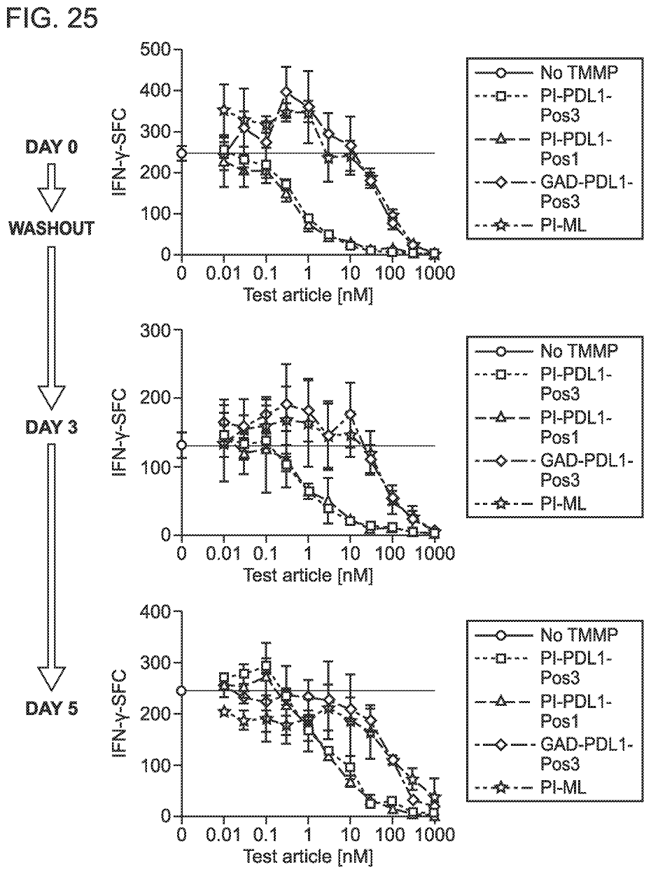

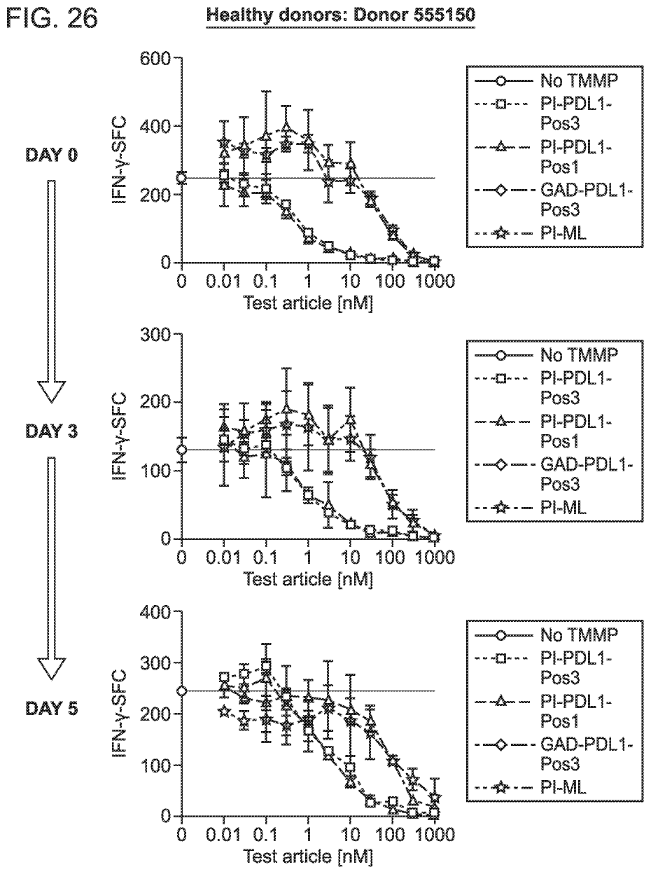

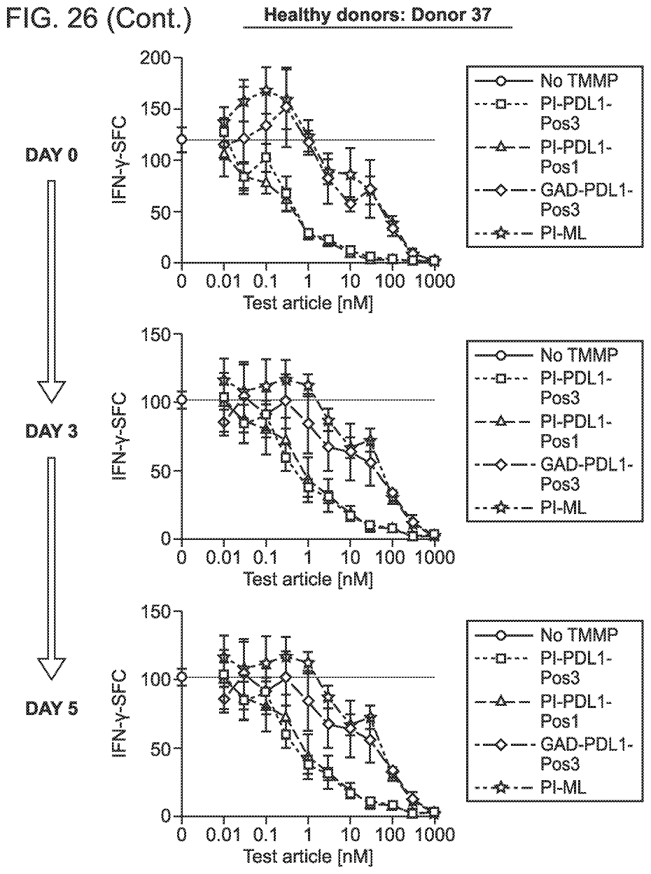

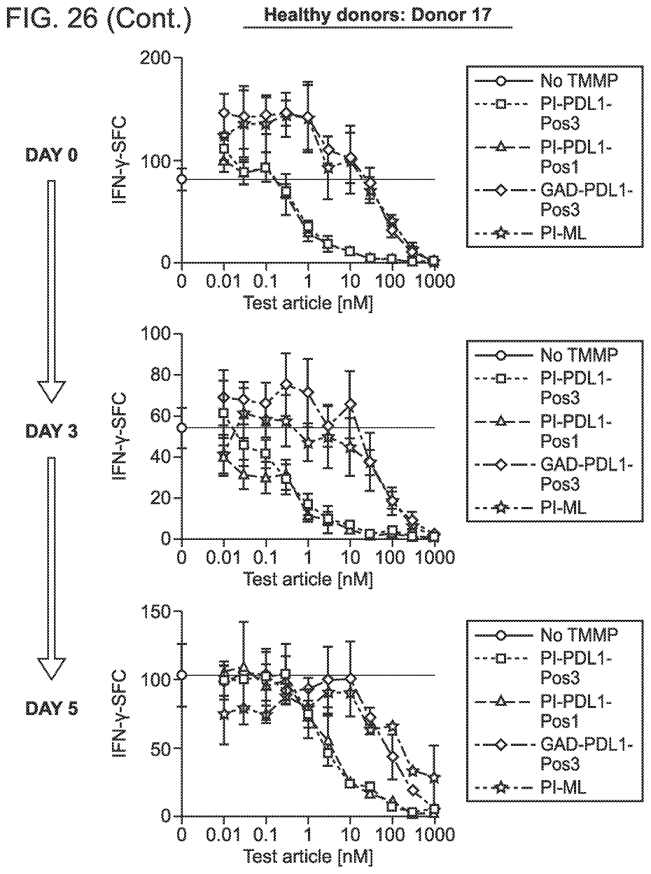

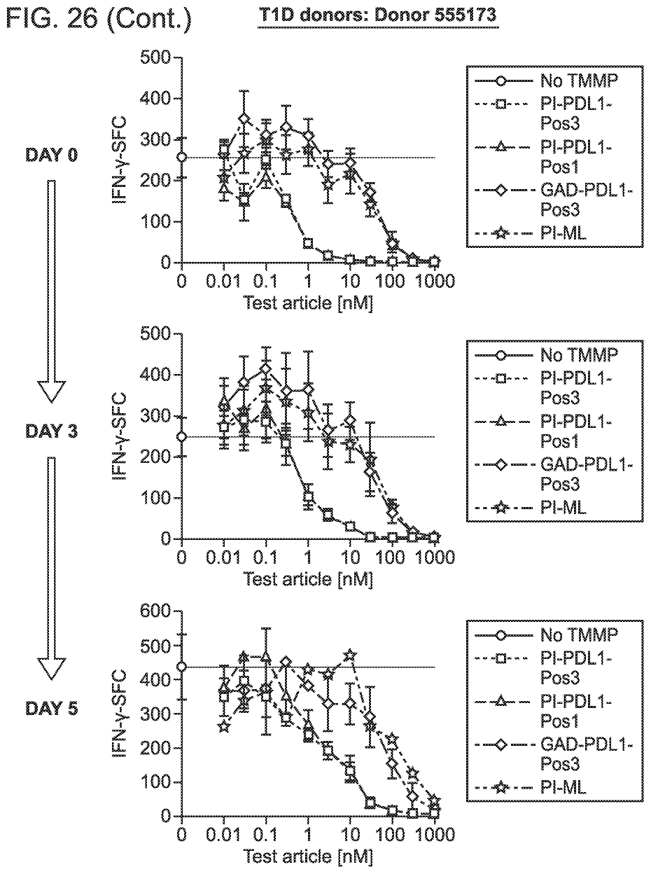

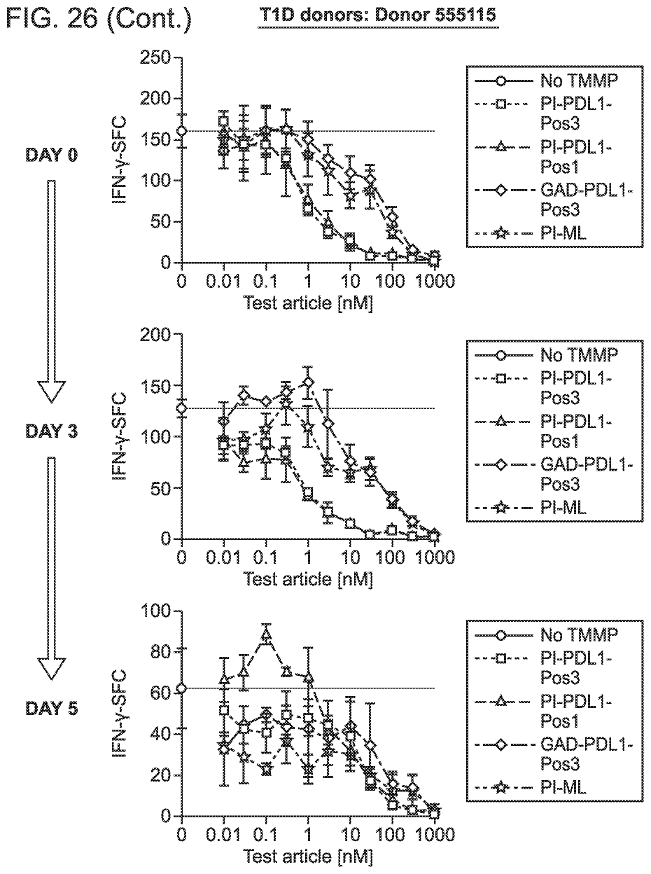

The results are shown in FIG. 25 and FIG. 26. As shown in FIG. 25, functional response of Proinsulin-specific CD4⁺ T cells were strongly suppressed by overnight treatment with 3005-2639 and 3893-2938 as compared to control molecules and remained suppressed 3-5 days post-treatment. As shown in FIG. 26, the antigen-specific, PD-L1-dependent suppression of PI-specific CD4⁺ T cells was observed across multiple donors.

### Example 6: Suppression of Epitope-Specific CD4⁺ T Cells In Vitro

The ability of GAD65-PDL1 Pos3 (3005-2580) to suppress GAD65-specific CD4 cells was assessed in an overnight suppression assay. The TMMP comprises two heterodimers; each heterodimer comprises: a) a 3005 polypeptide (FIG. 15D); and b) a 2580 polypeptide (FIG. 15G). PBMCs from a T1D patient were expanded with GAD65 555-567F557I peptide and recombinant human IL-2 in RPMI+10% human serum for 14 days. Expanded PBMCs were washed and incubated (2e6 cells/well. 24 well plate) with increasing concentrations of indicated TMMPs overnight at 37° C., 5% CO₂. The TMMP-treated cells were washed, and functional response of antigen-specific cells was assessed using an IFNγ ELISPOT upon re-stimulation with GAD65 555-567F557I peptide. Additionally, the TMMP-treated, washed cells were cultured in presence of autologous PBMCs (1:1) and rhIL-2 to assess duration of suppressive effect. IFNγ ELISPOT was performed at the indicated time points.

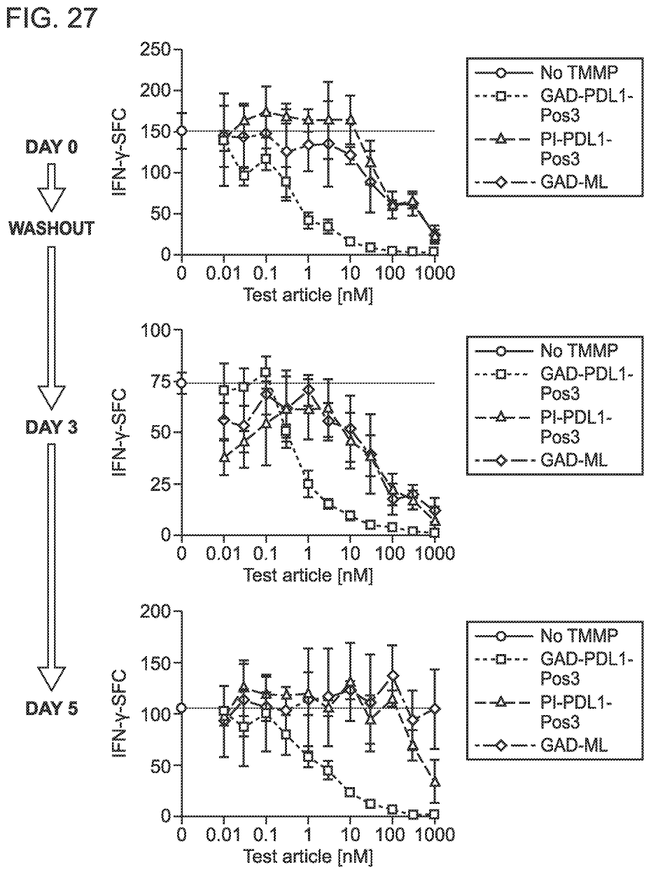

The results are shown in FIG. 27. Functional response of GAD65-specific CD4⁺ T cells were strongly suppressed by overnight treatment with 3005-2580 as compared to control molecules and remained suppressed 3-5 days post-treatment.

### Example 7: Suppression of Epitope-Specific CD4⁺ T Cells In Vitro

The ability of Proins-PDL1 Pos3 (3005-2639) and Proins-PDL1 Pos 1 (3893-2938) to suppress Proinsulin-specific CD4 cells in the presence of peptide stimulation was assessed in a 5-day assay. PBMCs from multiple healthy or T1D patients were expanded with PI 76-90K88S peptide and recombinant human IL-2 in RPMI+10% human serum for 14 days. CD4 cells were purified by negative selection from expanded PBMCs and stimulated with PI 76-90K88S peptide loaded autologous dendritic cells (DCs) in presence of increasing concentrations of indicated TMMPs (DCs were prepared as follows—autologous monocytes were isolated by negative selection and matured with IL4⁺ granulocyte-monocyte colony stimulating factor (GMCSF) for 24 hrs, followed by activation with TNFα, IL1β, IL6, and PGE2 for 24 hrs). After 5 days of culture, TMMP-treated cells were washed, and functional response of antigen-specific cells was assessed using an IFNγ ELISPOT upon re-stimulation with PI 76-90K88S peptide. Additionally, the TMMP-treated, washed cells were cultured in presence of autologous PBMCs (1:1) and rhIL-2 to assess duration of suppressive effect. IFNγ ELISPOT was performed at the indicated time points.

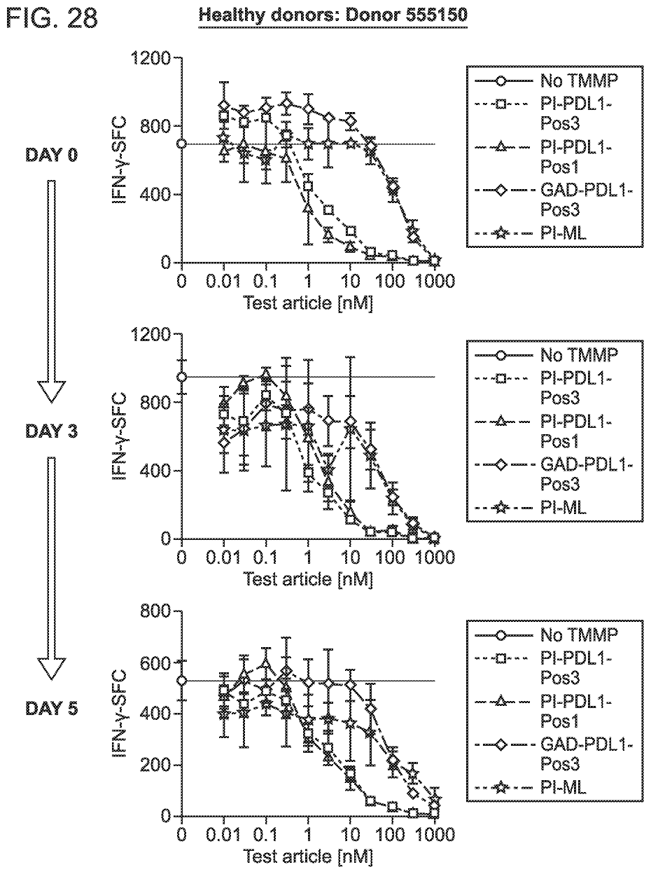

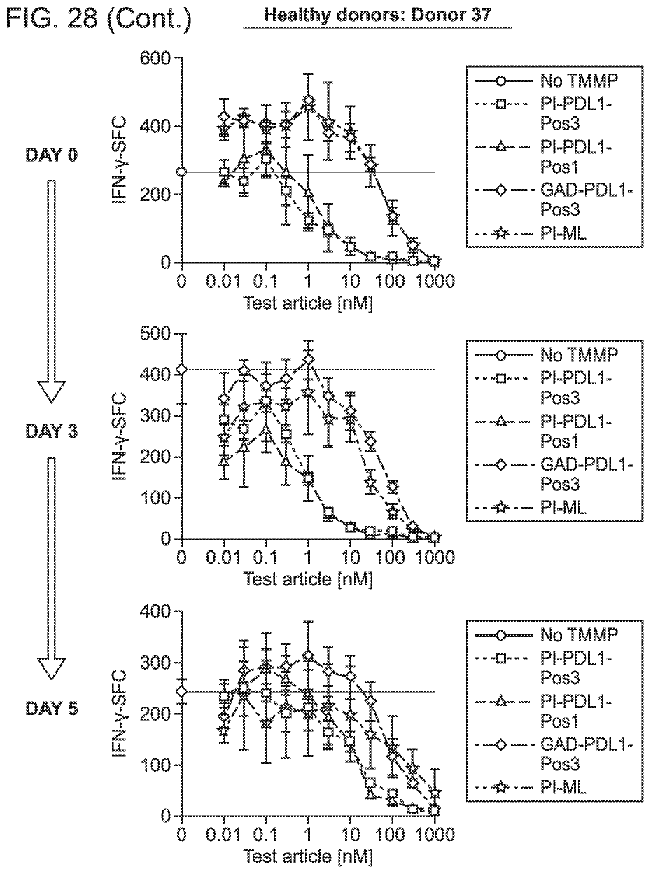

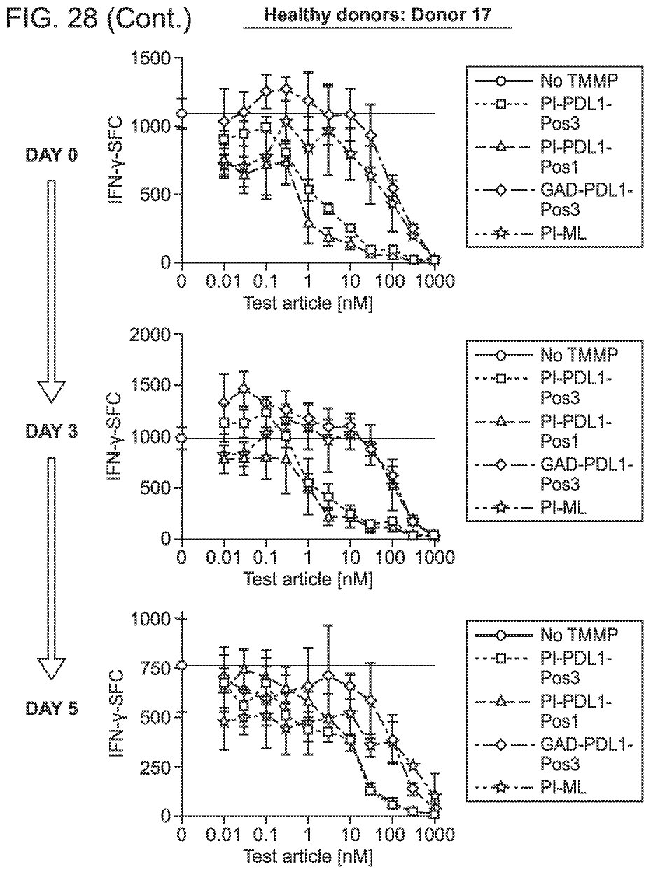

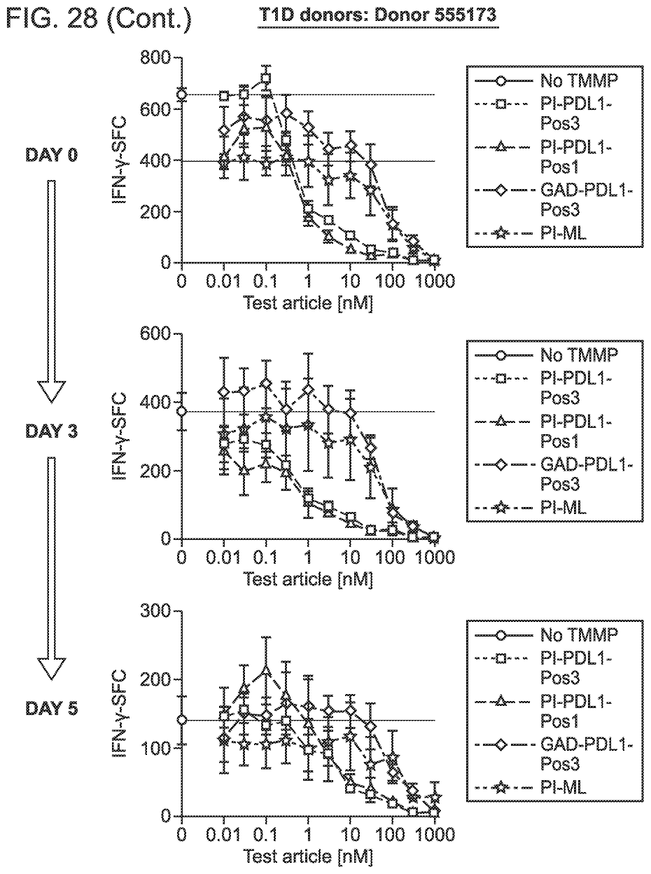

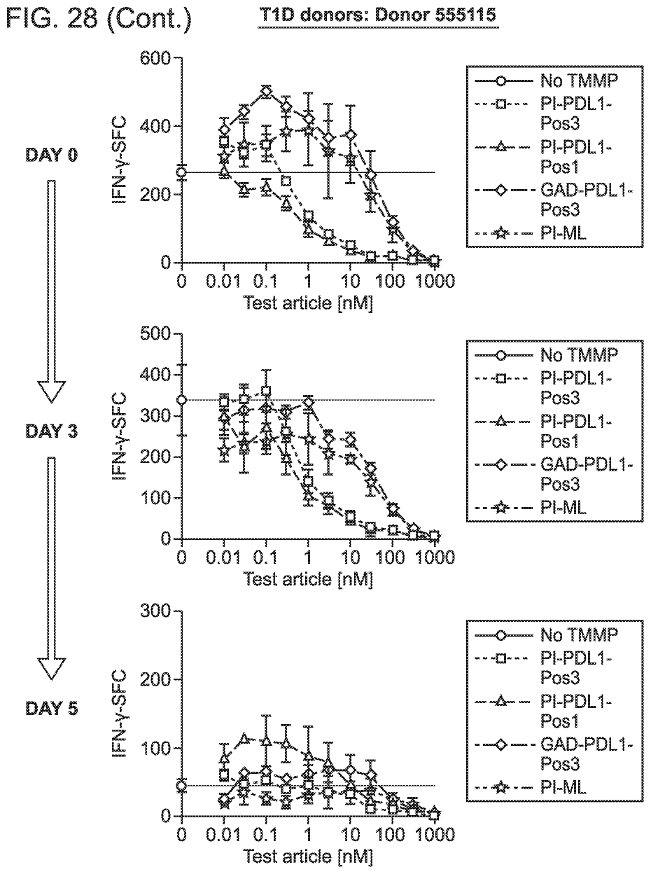

The results are shown in FIG. 28. Functional response of Proinsulin-specific CD4⁺ T cells stimulated for 5 days are strongly suppressed by treatment with 3005-2639 and 3893-2938 as compared to control molecules and remain suppressed 3-5 days post-treatment. The antigen-specific, PD-L1-dependent suppression of PI-specific CD4⁺ T cells was observed across multiple donors.

### Example 8: Pharmacokinetic Analysis and In Vivo Activity

A pharmacokinetic analysis of TMMP 3893-2938 was conducted. The effect of TMMP 3893-2938 on proinsulin-specific responses in vivo was assessed.

### Pharmacokinetics of TMMP 3893-2938 and TMMP 3005-2639

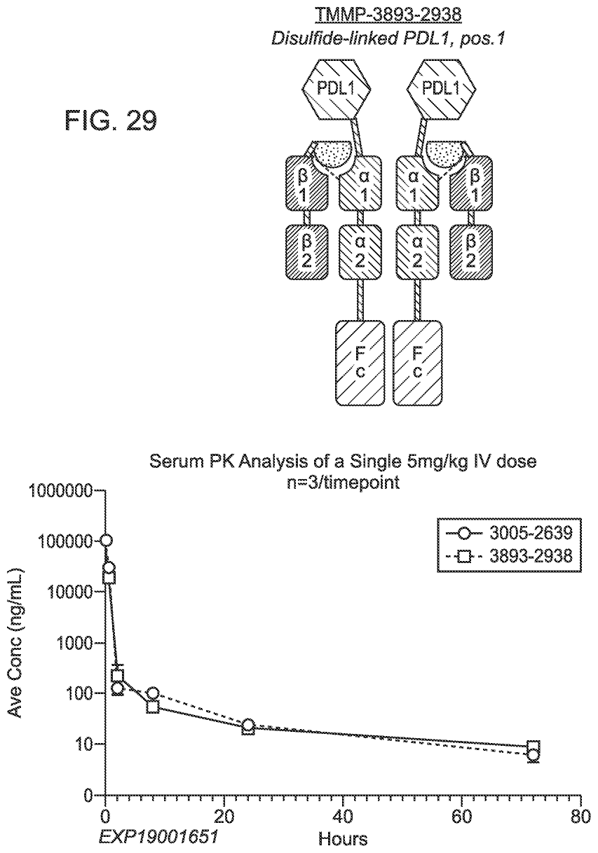

The pharmacokinetics of TMMP 3005-2639 (FIG. 15C-15D) and TMMP 3893-2938 (FIG. 15A-15B); depicted schematically in FIG. 29) were assessed in female albino C57Bl/6 mice. TMMPs were administered intravenously at 5 mg/kg, and serum samples were collected for up to 72 hours post-dose. The concentration of each TMMP in serum was determined relative to a standard curve using a ligand binding assay, in which each TMMP was captured by an anti-PDL1 antibody, and then detected using a sulfotagged anti-DR4 antibody.

The data are shown in FIG. 29. The data indicate that the pharmacokinetics of TMMP 3893-2938 are similar to those of TMMP 3005-2639.

### In Vivo Activity of 3893-2938

The in vivo activity of Proins-DR4-PDL1 TMMP 3893-2938 was tested in human HLA-DRB1*04 transgenic mice. The study design is depicted in FIG. 30. Mice were simultaneously immunized with Proins (PI; 76-90, K88S) and influenza HA (307-319) peptides in Complete Freund's Adjuvant (CFA) on Day 0. On Day 11 (before treatment with TMMPs) blood samples were drawn from mice, PBMCs purified, and seeded in an IL-2 ELISpot assay. PBMCs were stimulated with PMA/ionomycin, an irrelevant HIV peptide, Proins (PI; 76-90, K88S), or HA (307-319) peptides.

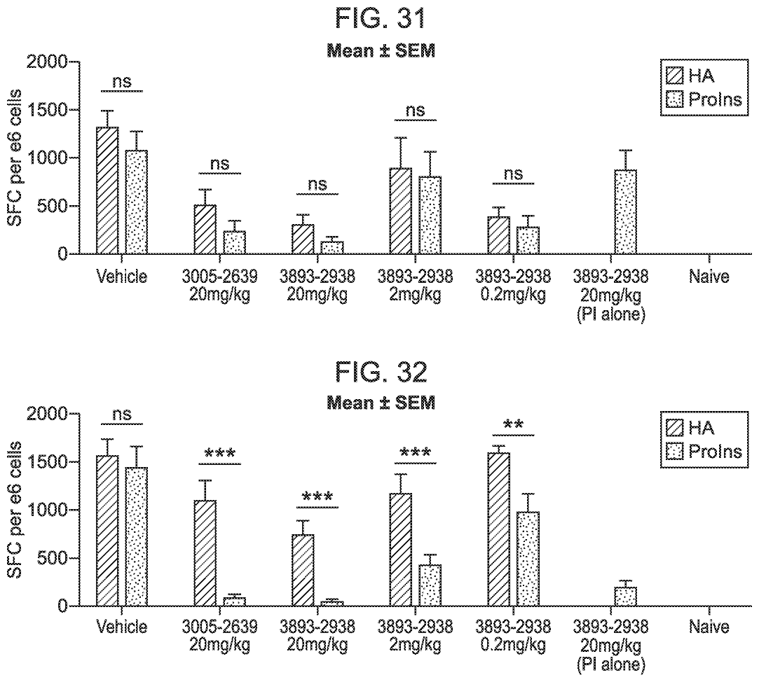

The data are shown in FIG. 31. The frequencies of Proins- and HA-reactive T cells are shown for each animal. All animals demonstrated successful immunization with the relevant immunizing peptides. While variable per group, each animal exhibited similar functional responses to both Proins and HA peptide stimulation 11 days post-immunization.

From the same experiment as above, on Day 11 post-immunization the mice were treated intravenously with (1) vehicle, or (2) 20 mg/kg of Proins-DR4-PDL1 IST-3005-2639, or (3) 20 mg/kg of Proins-DR4-PDL1 IST-3893-2938, or (4) 2 mg/kg of Proins-DR4-PDL1 IST-3893-2938, or (5) 0.2 mg/kg of Proins-DR4-PDL1 IST-3893-2938, or (6) 20 mg/kg of Proins-DR4-PDL1 IST-3893-2938 after being immunized with PI peptide alone. On Day 12 (post-treatment) blood samples were drawn from mice, PBMCs purified, and seeded in an IL-2 ELISpot assay. PBMCs were stimulated as above.

The data are shown in FIG. 32. The frequencies of Proins- and HA-reactive T cells are shown for each animal. A single treatment with Proins-DR4-PDL1 TMMP (TMMP-3005-2639 or TMMP-3893-2938) on Day 11 post-immunization resulted in a dose-dependent reduction in the frequency of Proins-reactive T cells on Day 12 post-immunization. This suppression of Proins-reactive T cells was antigen specific, as HA-reactive T cells were not suppressed. TMMP 3893-2938 (in which the two polypeptides of the heterodimer disulfide linked) demonstrated comparable suppression to TMMP 3005-2639 (in which the two polypeptides of the heterodimer are non-covalently associated). Comparable suppression of proinsulin-specific responses was observed, regardless whether animals were co-immunized against HA. Proins-specific responses were suppressed in a dose-dependent manner by TMMP 3893-2938.

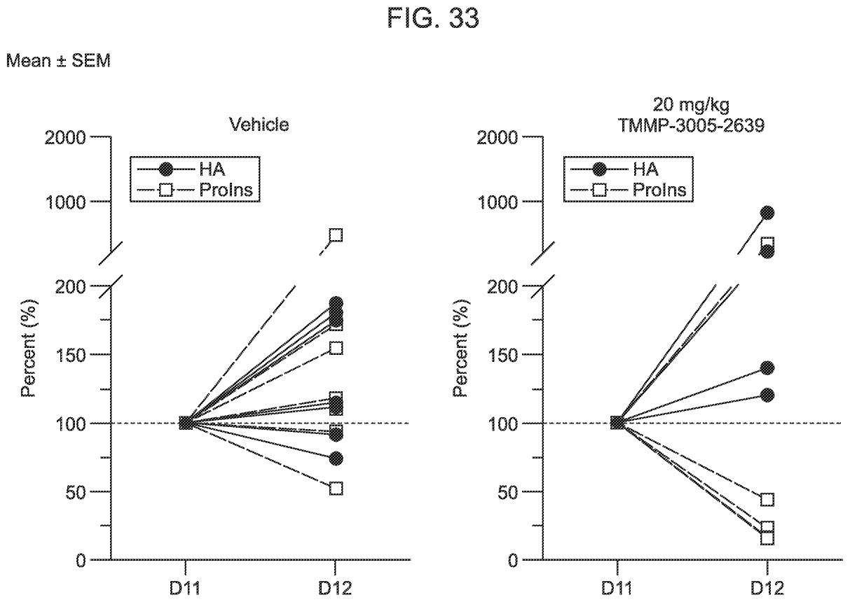

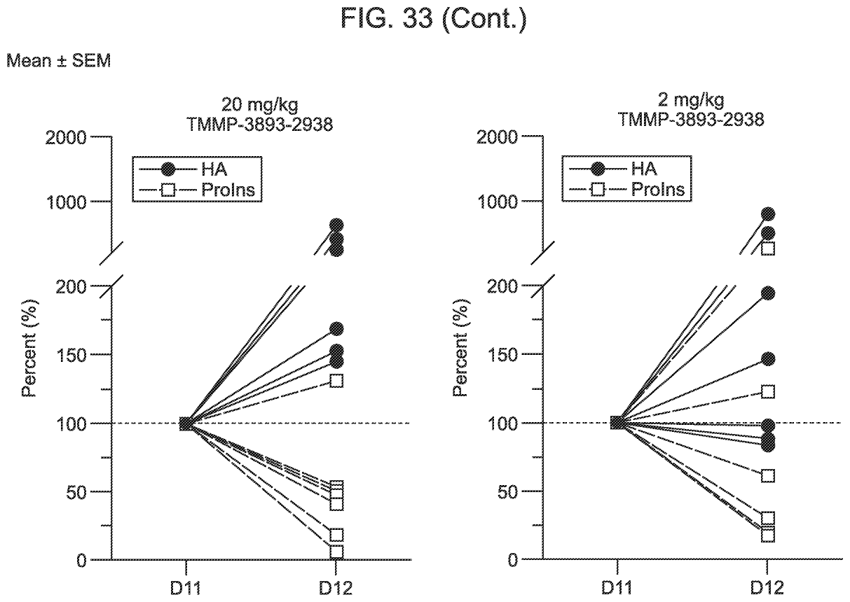

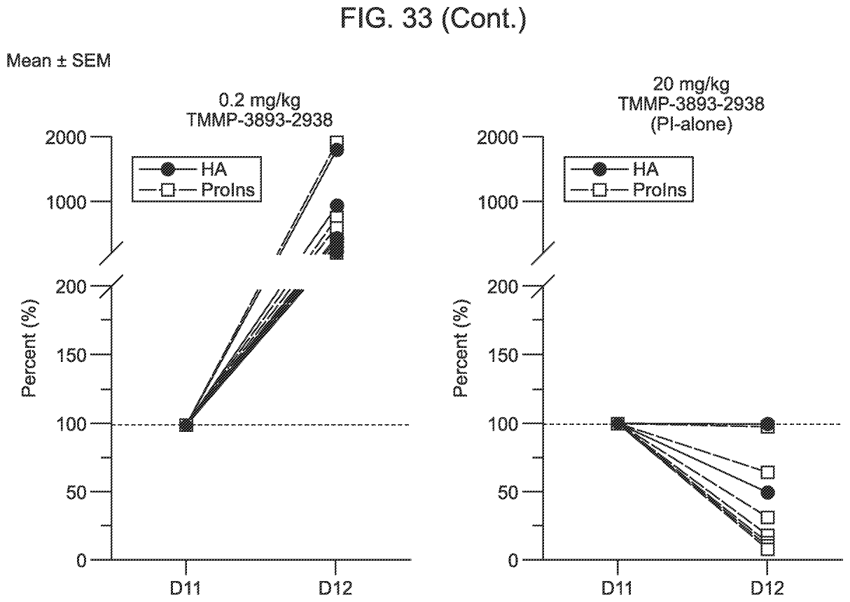

The results depicted in FIG. 32 were quantified by another means. The data are shown in FIG. 33. The percent relative change in frequency of PI and HA reactive cells post-treatment (Day 12) is shown relative to the pre-treatment (Day 11) sample from each individual mouse. This result demonstrates that a 20 mg/kg dose of TMMP 3005-2639 or TMMP 3892-2938 results in reduced frequency of PI-reactive cells while the frequency of HA-reactive cells continues to increase, confirming the antigen-specificity of this treatment effect.

### Suppression of Proins-Specific Cytokine Production Following a Single Dose of TMMP

Inhibition of cytokine-producing T cells by TMMP treatment was further demonstrated using intracellular cytokine staining and flow cytometry. Spleens were harvested from all animals in the above experiment on Day 12 post-immunization (post-treatment). Spleens were then dissociated into single cell suspensions and stimulated with Proins (PI; 76-90, K88S) or HA (307-319) peptides and cytokine production was analyzed by flow cytometry.

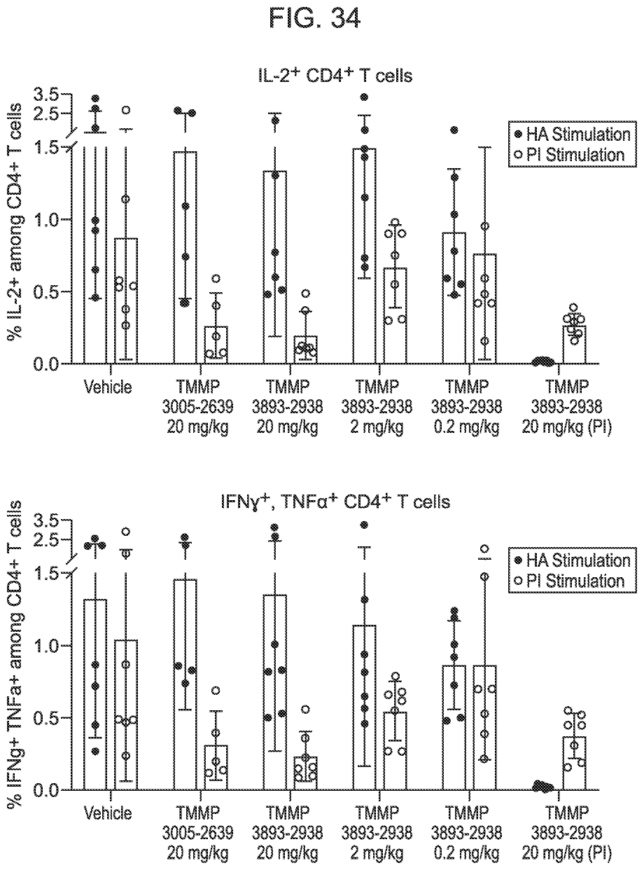

The data are shown in FIG. 34. Splenic CD4+ T cells from immunized animals produced IL-2 and/or IFNγ plus TNFα upon stimulation with Proins or HA peptides. In these animals, treatment with Proins-DR4-PDL1 TMMP 3005-2639 or TMMP 3893-2938 reduced the frequency of splenic CD4+ T cells producing cytokine in response to Proins stimulation. In contrast, the frequency of HA-reactive CD4+ T cells was not altered by treatment with Proins-DR4-PDL1 TMMP. These data confirm the ELISpot data showing suppression of Proins-specific responses by both TMMP 3893-2938 and TMMP 3005-2639, and demonstrate suppression of multiple cytokines by treatment with either TMMP 3893-2938 or TMMP 3005-2639.

ProIns-Specific T Cells are CD4⁺, CD44^{hi}, and PD-1⁺.

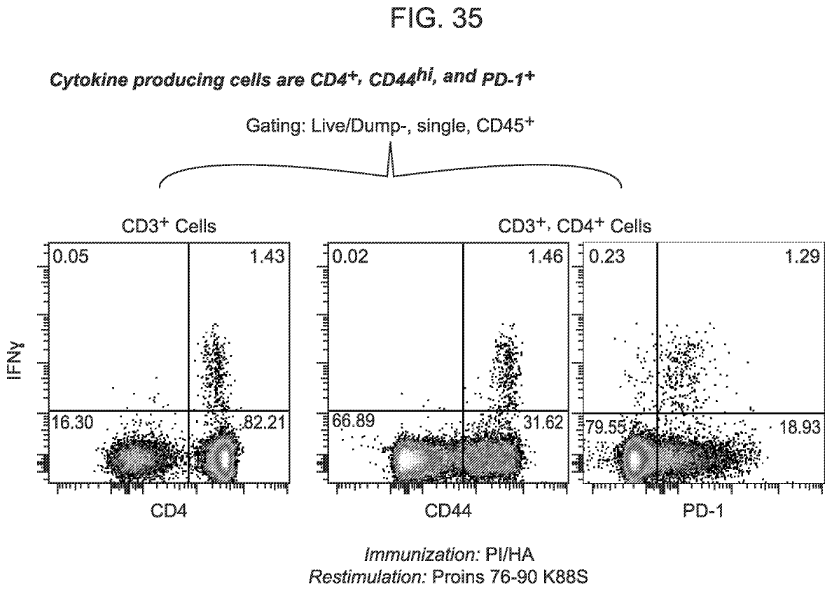

Cell surface marker expression in the Proins-reactive CD4+ T cells in the above experiment was further quantified by measuring the geometric mean fluorescence intensity (gMFI) of staining in cytokine-positive cells. The data are shown in FIG. 35. CD3+CD4+ T cells producing IFNγ in response to PI peptide stimulation were observed to express high levels of CD44 and PD1.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1.-66. (canceled)

67. A method of selectively modulating the activity of CD4⁺ T cells specific for a type 1 diabetes-associated epitope in a human patient, comprising administering to a human in need thereof a therapeutically effective amount of a pharmaceutical composition comprising a T-cell modulatory multimeric polypeptide (TMMP),
wherein the TMMP is a homodimer of two heterodimers, wherein each heterodimer comprises a first polypeptide having the amino acid sequence set forth in SEQ ID NO:185 and a second polypeptide having the amino acid sequence set forth in SEQ ID NO:186, and wherein the heterodimers are covalently linked to one another through disulfide bonds formed between the Ig Fc polypeptides in the second polypeptide of each heterodimer.

68. A method of reducing the number and/or activity of CD4⁺ and/or CD8⁺ self-reactive T cells specific for a type 1 diabetes-associated epitope in a human, the method comprising administering to a human in need thereof a therapeutically effective amount of a pharmaceutical composition comprising a T-cell modulatory multimeric polypeptide (TMMP),
wherein the TMMP is a homodimer of two heterodimers, wherein each heterodimer comprises a first polypeptide having the amino acid sequence set forth in SEQ ID NO:185 and a second polypeptide having the amino acid sequence set forth in SEQ ID NO:186, and wherein the heterodimers are covalently linked to one another through disulfide bonds formed between the Ig Fc polypeptides in the second polypeptide of each heterodimer.

69. A method of treating type 1 diabetes comprising administering to a human in need thereof a therapeutically effective amount of a pharmaceutical composition comprising a T-cell modulatory multimeric polypeptide (TMMP),
wherein the TMMP is a homodimer of two heterodimers, wherein each heterodimer comprises a first polypeptide having the amino acid sequence set forth in SEQ ID NO: 185 and a second polypeptide having the amino acid sequence set forth in SEQ ID NO: 186, and wherein the heterodimers are covalently linked to one another through disulfide bonds formed between the Ig Fc polypeptides in the second polypeptide of each heterodimer.


SEQUENCE LISTING (237 sequences)

SEQ ID NO: 1 - protein, 55 aa, synthetic construct
  VARIANT 11..55: note = Wherein up to 9 copies of GGGGS may be omitted
CGGGSGGGGSGGGGSGGGGSGGGGSGGGGSGGGGSGGGGSGGGGSGGGGSGGGGS

SEQ ID NO: 2 - protein, 55 aa, synthetic construct
  VARIANT 11..55: note = Wherein up to 9 copies of GGGGS may be omitted
GCGGSGGGGSGGGGSGGGGSGGGGSGGGGSGGGGSGGGGSGGGGSGGGGSGGGGS

SEQ ID NO: 3 - protein, 55 aa, synthetic construct
  VARIANT 11..55: note = Wherein up to 9 copies of GGGGS may be omitted
GGCGSGGGGSGGGGSGGGGSGGGGSGGGGSGGGGSGGGGSGGGGSGGGGSGGGGS

SEQ ID NO: 4 - protein, 55 aa, synthetic construct
  VARIANT 11..55: note = Wherein up to 9 copies of GGGGS may be omitted
GGGCSGGGGSGGGGSGGGGSGGGGSGGGGSGGGGSGGGGSGGGGSGGGGSGGGGS

SEQ ID NO: 5 - protein, 55 aa, synthetic construct
  VARIANT 11..55: note = Wherein up to 9 copies of GGGGS may be omitted
GGGGCGGGGSGGGGGSGGGGSGGGGSGGGGSGGGSGGGGSGGGGSGGGGSGGGGS

SEQ ID NO: 6 - protein, 178 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 7 - protein, 178 aa, synthetic construct
IKEEHVIIQAEFYLNPDQSGEFMFDFDGDEIFHVDMAKKETVWRLEEFGRFASFEAQGALANIAVDKANLECMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 8 - protein, 178 aa, synthetic construct
IKEEHVIIQAEFYLNPDQSGEFMFDFDGDEIFHVDMAKKETVWRLEEFGRFASFEAQGALANIAVDKANLEIMTCRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 9 - protein, 189 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 10 - protein, 189 aa, synthetic construct
IKEEHVIIQAEFYLNPDQSGEFMFDFDGCEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 11 - protein, 189 aa, synthetic construct
IKEEHVIIQAEFYLNPDQSGEFMFDFDGDEIFHVDMAKKETVWRLEEFGRFASFEAQGALANIAVDKANLECMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 12 - protein, 189 aa, synthetic construct
IKEEHVIIQAEFYLNPDQSGEFMFDFDGDEIFHVDMAKKETVWRLEEFGRFASFEAQGALANIAVDKANLEIMTCRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 13 - protein, 189 aa, synthetic construct
IKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 14 - protein, 189 aa, synthetic construct
IKEEHVIIQAEFYLNPDQSGEFMFDFDGDEIFHVDMAKKETVWRLEEFGRFASFEAQGALANIAVDKANLEIMTKRSNYTPCTNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 15 - protein, 79 aa, synthetic construct
VIIQAEFYLNPDQSGEFMFDFDGDEIFHVDMAKKETVWRLEEFGRFASFEAQGALANIAVDKANLEIMTKRSNYTPITN

SEQ ID NO: 16 - protein, 84 aa, synthetic construct
IKEEHVIIQAEFYLNPDQSGEFMFDFDGCEIFHVDMAKKETVWRLEEFGRFASFEAQGALANIAVDKANLEIMTKRSNYTPITN

SEQ ID NO: 17 - protein, 84 aa, synthetic construct
IKEEHVIIQAEFYLNPDQSGEFMFDFDGDEIFHVDMAKKETVWRLEEFGRFASFEAQGALANIAVDKANLECMTKRSNYTPITN

SEQ ID NO: 18 - protein, 84 aa, synthetic construct
IKEEHVIIQAEFYLNPDQSGEFMFDFDGDEIFHVDMAKKETVWRLEEFGRFASFEAQGALANIAVDKANLEIMTCRSNYTPITN

SEQ ID NO: 19 - protein, 84 aa, synthetic construct
IKEEHVIIQAEFYLNPDQSGEFMFDFDGDEIFHVDMAKKETVWRLEEFGRFASFEAQGALANIAVDKANLEIMTKRSNYTCITN

SEQ ID NO: 20 - protein, 84 aa, synthetic construct
IKEEHVIIQAEFYLNPDQSGEFMFDFDGDEIFHVDMAKKETVWRLEEFGRFASFEAQGALANIAVDKANLEIMTKRSNYTPCTN

SEQ ID NO: 21 - protein, 94 aa, synthetic construct
VPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 22 - protein, 181 aa, Homo sapiens
AGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHW

SEQ ID NO: 23 - protein, 85 aa, synthetic construct
AIKADHVSTYAAFVQTHRPTGEFMFEFDEDEMFYVDLDKKETVWHLEEFGQAFSFEAQGGLANIAILNNNLNTLIQRSNHTQATN

SEQ ID NO: 24 - protein, 94 aa, synthetic construct
DPPEVTVFPKEPVELGQPNTLICHIDKFFPPVLNVTWLCNGELVTEGVAESLFLPRTDYSFHKFHYLTFVPSAEDFYDCRVEHWGLDQPLLKHW

SEQ ID NO: 25 - protein, 260 aa, Homo sapiens [sequence omitted]

SEQ ID NO: 26 - protein, 181 aa, synthetic construct
EDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHW

SEQ ID NO: 27 - protein, 87 aa, synthetic construct
EDIVADHVASCGVNLYQFYGPSGQYTHEFDGDEQFYVDLERKETAWRWPEFSKFGGFDPQGALRNMAVAKHNLNIMIKRYNSTAATN

SEQ ID NO: 28 - protein, 87 aa, synthetic construct; identical to SEQ ID NO: 27

SEQ ID NO: 29 - protein, 94 aa, synthetic construct
EVPEVTVFSKSPVTLGQPNTLICLVDNIFPPVVNITWLSNGQSVTEGVSETSFLSKSDHSFFKISYLTFLPSADEIYDCKVEHWGLDQPLLKHW

SEQ ID NO: 30 - protein, 199 aa, Homo sapiens
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 31 - protein, 29 aa, synthetic construct
MVCLKLPGGSSLAALTVTLMVLSSRLAFA

SEQ ID NO: 32 - protein, 19 aa, synthetic construct
MVCLKLPGGSCMAALTVTL

SEQ ID NO: 33 - protein, 19 aa, synthetic construct
MVCLKLPGGSYMAKLTVTL

SEQ ID NO: 34 - protein, 197 aa, synthetic construct
DTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 35 - protein, 199 aa, synthetic construct
GDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 36 - protein, 199 aa, synthetic construct
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNCDWTFQTLVMLETVPRSGEVYTCQVEHPSLTSPLTVEWRARSESAQSKM

SEQ ID NO: 37 - protein, 199 aa, synthetic construct
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDCTFQTLVMLETVPRSGEVYTCQVEHPSLTSPLTVEWRARSESAQSKM

SEQ ID NO: 38 - protein, 94 aa, synthetic construct
DTRPRFLEQVKHECHFFNGTERVRFLDRYFYHQEEYVRFDSDVGEYRAVTELGRPDAEYWNSQKDLLEQKRAAVDTYCRHNYGVGESFTVQRRV

SEQ ID NO: 39 - protein, 95 aa, synthetic construct
GDTRCRFLEQVKHECHFFNGTERVRFLDRYFYHQEEYVRFDSDVGEYRAVTELGRPDAEYWNSQKDLLEQKRAAVDTYCRHNYGVGESFTVQRRV

SEQ ID NO: 40 - protein, 103 aa, synthetic construct
YPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 41 - protein, 104 aa, synthetic construct
YPEVTVYPAKTQPLQHHNLLVCSVNGFYPASIEVRWFRNGQEEKTGVVSTGLIQNCDWTFQTLVMLETVPRSGEVYTCQVEHPSLTSPLTVEWRARSESAQSKM

SEQ ID NO: 42 - protein, 104 aa, synthetic construct
YPEVTVYPAKTQPLQHHNLLVCSVNGFYPASIEVRWFRNGQEEKTGVVSTGLIQNGDCTFQTLVMLETVPRSGEVYTCQVEHPSLTSPLTVEWRARSESAQSKM

SEQ ID NO: 43 - protein, 197 aa, synthetic construct
DTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 44 - protein, 94 aa, synthetic construct
DTRPRFLELRKSECHFFNGTERVRYLDRYFHNQEEFLRFDSDVGEYRAVTELGRPVAESWNSQKDLLEQKRGRVDNYCRHNYGVGESFTVQRRV

SEQ ID NO: 45 - protein, 103 aa, synthetic construct
HPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 46 - protein, 208 aa, synthetic construct
TVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSK

SEQ ID NO: 47 - protein, 105 aa, synthetic construct
TVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRV

SEQ ID NO: 48 - protein, 103 aa, synthetic construct
QPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSK

SEQ ID NO: 49 - protein, 207 aa, synthetic construct
MVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQS

SEQ ID NO: 50 - protein, 105 aa, synthetic construct
MVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRV

SEQ ID NO: 51 - protein, 102 aa, synthetic construct
EPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQS

SEQ ID NO: 52 - protein, 186 aa, synthetic construct
RATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEW

SEQ ID NO: 53 - protein, 92 aa, synthetic construct
RATPENYLFQGRQECYAFNGTQRFLERYIYNREEFARFDSDVGEFRAVTELGRPAAEYWNSQKDILEEKRAVPDRMCRHNYELGGPMTLQRR

SEQ ID NO: 54 - protein, 94 aa, synthetic construct
VQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEW

SEQ ID NO: 55 - protein, 188 aa, synthetic construct
RDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEW

SEQ ID NO: 56 - protein, 94 aa, synthetic construct
RDSPEDFVFQFKGMCYFTNGTERVRLVTRYIYNREEYARFDSDVGVYRAVTPQGRPDAEYWNSQKEVLEGTRAELDTVCRHNYEVAFRGILQRR

SEQ ID NO: 57 - protein, 94 aa, synthetic construct
VEPTVTISPSRTEALNHHNLLVCSVTDFYPGQIKVRWFRNDQEETAGVVSTPLIRNGDWTFQILVMLEMTPQRGDVYTCHVEHPSLQSPITVEW

SEQ ID NO: 58 - protein, 223 aa, synthetic construct
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 59 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 5 aa, synthetic construct
GSGGS

SEQ ID NO: 61 - protein, 4 aa, synthetic construct
GGGS

SEQ ID NO: 62 - protein, 4 aa, synthetic construct
GGSG

SEQ ID NO: 63 - protein, 5 aa, synthetic construct
GGSGG

SEQ ID NO: 64 - protein, 5 aa, synthetic construct
GSGSG

SEQ ID NO: 65 - protein, 5 aa, synthetic construct
GSGGG

SEQ ID NO: 66 - protein, 5 aa, synthetic construct
GGGSG

SEQ ID NO: 67 - protein, 5 aa, synthetic construct
GSSSG

SEQ ID NO: 68 - protein, 41 aa, synthetic construct
  VARIANT 6..41: note = Wherein up to 9 copies of SSSS may be omitted
GSSSSSSSSSSSSSSSSSSSSSSSSSSSSSSSSSSSSSSSS

SEQ ID NO: 69 - protein, 20 aa, synthetic construct
GSSSSGSSSSGSSSSGSSSS

SEQ ID NO: 70 - protein, 25 aa, synthetic construct
GSSSSGSSSSGSSSSGSSSSGSSSS

SEQ ID NO: 71 - protein, 50 aa, synthetic construct
  VARIANT 6..50: note = Wherein up to 9 copies of GGGGS may be omitted
GGGGSGGGGSGGGGSGGGGSGGGGSGGGGSGGGGSGGGGSGGGGSGGGGS

SEQ ID NO: 72 - protein, 5 aa, synthetic construct
GGGGS

SEQ ID NO: 73 - protein, 10 aa, synthetic construct
GGGGSGGGGS

SEQ ID NO: 74 - protein, 15 aa, synthetic construct
GGGGSGGGGSGGGGS

SEQ ID NO: 75 - protein, 20 aa, synthetic construct
GGGGSGGGGSGGGGSGGGGS

SEQ ID NO: 76 - protein, 25 aa, synthetic construct
GGGGSGGGGSGGGGSGGGGSGGGGS

SEQ ID NO: 77 - protein, 30 aa, synthetic construct
GGGGSGGGGSGGGGSGGGGSGGGGSGGGGS

SEQ ID NO: 78 - protein, 35 aa, synthetic construct
GGGGSGGGGSGGGGSGGGGSGGGGSGGGGSGGGGS

SEQ ID NO: 79 - protein, 40 aa, synthetic construct
GGGGSGGGGSGGGGSGGGGSGGGGSGGGGSGGGGSGGGGS

SEQ ID NO: 80 - protein, 45 aa, synthetic construct
GGGGSGGGGSGGGGSGGGGSGGGGSGGGGSGGGGSGGGGSGGGGS

SEQ ID NO: 81 - protein, 50 aa, synthetic construct; identical to SEQ ID NO: 71

SEQ ID NO: 82 - protein, 5 aa, synthetic construct
AAAGG

SEQ ID NO: 83 - protein, 8 aa, synthetic construct
GGSAAAGG

SEQ ID NO: 84 - protein, 17 aa, Homo sapiens
GAGSLQPLALEGSLQKR

SEQ ID NO: 85 - protein, 15 aa, Homo sapiens
GIVDQCCTSICSLYQ

SEQ ID NO: 86 - protein, 15 aa, Homo sapiens
GIVEQCCTSICSLYQ

SEQ ID NO: 87 - protein, 13 aa, Homo sapiens
NFFRMVISNPAAT

SEQ ID NO: 88 - protein, 13 aa, Homo sapiens
NFIRMVISNPAAT

SEQ ID NO: 89 - protein, 20 aa, Homo sapiens
SFYLKNVQTQETRTLTQFHF

SEQ ID NO: 90 - protein, 15 aa, Homo sapiens
SLQPLALEGSLQSRG

SEQ ID NO: 91 - protein, 18 aa, Homo sapiens
GSLQPLALEGSLQSRGIV

SEQ ID NO: 92 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 93 - protein, 18 aa, Homo sapiens
GAGSLQPLALEGSLQKRG

SEQ ID NO: 94 - protein, 15 aa, Homo sapiens
SLQPLALEGSLQKRG

SEQ ID NO: 95 - protein, 13 aa, Homo sapiens
QPLALEGSLQKRG

SEQ ID NO: 96 - protein, 13 aa, Homo sapiens
QPLALEGSLQSRG

SEQ ID NO: 97 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 98 - protein, 219 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 99 - protein, 220 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 100 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 101 - protein, 133 aa, Homo sapiens [sequence omitted]

SEQ ID NO: 102 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 103 - protein, 524 aa, Homo sapiens
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 104 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 105 - protein, 133 aa, synthetic construct
  VARIANT 42: note = X is any amino acid other than Phe. In some cases, X is Ala
APTSSSTKKTQLQLEHLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 106 - protein, 133 aa, synthetic construct
  VARIANT 20: note = X is any amino acid other than Asp. In some cases, X is Ala
APTSSSTKKTQLQLEHLLLXLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 107 - protein, 133 aa, synthetic construct
  VARIANT 15: note = X is any amino acid other than Glu. In some cases, X is Ala
APTSSSTKKTQLQLXHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 108 - protein, 133 aa, synthetic construct
  VARIANT 16: note = X is any amino acid other than His. In some cases, X is Ala, Arg, Asn, Asp, Cys, Glu, Gln, Gly, Lys, Leu, Met, Phe, Pro, Ser, Thr, Tyr, Trp or Val
APTSSSTKKTQLQLEXLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 109 - protein, 133 aa, synthetic construct
  VARIANT 45: note = X is any amino acid other than Tyr. In some cases, X is Ala
APTSSSTKKTQLQLEHLLLDLQMILNGINNYKNPKLTRMLTFKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 110 - protein, 133 aa, synthetic construct
  VARIANT 126: note = X is any amino acid other than Gln. In some cases, X is Ala
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 111 - protein, 133 aa, synthetic construct
  VARIANT 16: note = X is any amino acid other than His. In some cases, X is Ala or Thr
  VARIANT 42: note = X is any amino acid other than Phe. In some cases, X is Ala
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 112 - protein, 133 aa, synthetic construct
  VARIANT 20: note = X is any amino acid other than Asp. In some cases, X is Ala
  VARIANT 42: note = X is any amino acid other than Phe. In some cases, X is Ala
APTSSSTKKTQLQLEHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 113 - protein, 133 aa, synthetic construct
  VARIANT 15: note = X is any amino acid other than Glu. In some cases, X is Ala
  VARIANT 20: note = X is any amino acid other than Asp. In some cases, X is Ala
  VARIANT 42: note = X is any amino acid other than Phe. In some cases, X is Ala
APTSSSTKKTQLQLXHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 114 - protein, 133 aa, synthetic construct
  VARIANT 16: note = X is any amino acid other than His. In some cases, X is Ala
  VARIANT 20: note = X is any amino acid other than Asp. In some cases, X is Ala
  VARIANT 42: note = X is any amino acid other than Phe. In some cases, X is Ala
APTSSSTKKTQLQLEXLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 115 - protein, 133 aa, synthetic construct
  VARIANT 20: note = X is any amino acid other than Asp. In some cases, X is Ala
  VARIANT 42: note = X is any amino acid other than Phe. In some cases, X is Ala
  VARIANT 126: note = X is any amino acid other than Gln. In some cases, X is Ala
APTSSSTKKTQLQLEHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 116 - protein, 133 aa, synthetic construct
  VARIANT 20: note = X is any amino acid other than Asp. In some cases, X is Ala
  VARIANT 42: note = X is any amino acid other than Phe. In some cases, X is Ala
  VARIANT 45: note = X is any amino acid other than Tyr. In some cases, X is Ala
APTSSSTKKTQLQLEHLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 117 - protein, 133 aa, synthetic construct
  VARIANT 16: note = X is any amino acid other than His. In some cases, X is Ala
  VARIANT 20: note = X is any amino acid other than Asp. In some cases, X is Ala
  VARIANT 42: note = X is any amino acid other than Phe. In some cases, X is Ala
  VARIANT 45: note = X is any amino acid other than Tyr. In some cases, X is Ala
APTSSSTKKTQLQLEXLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 118 - protein, 133 aa, synthetic construct
  VARIANT 20: note = X is any amino acid other than Asp. In some cases, X is Ala
  VARIANT 42: note = X is any amino acid other than Phe. In some cases, X is Ala
  VARIANT 45: note = X is any amino acid other than Tyr. In some cases, X is Ala
  VARIANT 126: note = X is any amino acid other than Gln. In some cases, X is Ala
APTSSSTKKTQLQLEHLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCXSIISTLT

SEQ ID NO: 119 - protein, 133 aa, synthetic construct
  VARIANT 16: note = X is any amino acid other than His. In some cases, X is Ala
  VARIANT 20: note = X is any amino acid other than Asp. In some cases, X is Ala
  VARIANT 42: note = X is any amino acid other than Phe. In some cases, X is Ala
  VARIANT 45: note = X is any amino acid other than Tyr. In some cases, X is Ala
  VARIANT 126: note = X is any amino acid other than Gln. In some cases, X is Ala
APTSSSTKKTQLQLEXLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCXSIISTLT

SEQ ID NO: 120 - protein, 133 aa, synthetic construct
  VARIANT 16: note = X is any amino acid other than His. In some cases, X is Ala
  VARIANT 42: note = X is any amino acid other than Phe. In some cases, X is Ala
  VARIANT 126: note = X is any amino acid other than Gln. In some cases, X is Ala
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 121 - protein, 133 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 122 - protein, 112 aa, synthetic construct
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 123 - protein, 112 aa, synthetic construct
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 124 - protein, 112 aa, synthetic construct [sequence omitted]

SEQ ID NO: 125 - protein, 281 aa, synthetic construct
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 126 - protein, 179 aa, synthetic construct
QLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 127 - protein, 335 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 128 - protein, 15 aa, synthetic construct
GGCGSGGGGSGGGGS

SEQ ID NO: 129 - protein, 20 aa, synthetic construct
GGCGSGGGGSGGGGSGGGGS

SEQ ID NO: 130 - protein, 254 aa, synthetic construct
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 131 - protein, 266 aa, Homo sapiens
MVCLRLPGGSCMAVLTVTLMVLSSPLALAGDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 132 - protein, 266 aa, Homo sapiens [sequence omitted]

SEQ ID NO: 133 - protein, 266 aa, Homo sapiens [sequence omitted]

SEQ ID NO: 134 - protein, 266 aa, Homo sapiens [sequence omitted]

SEQ ID NO: 135 - protein, 266 aa, Homo sapiens [sequence omitted]

SEQ ID NO: 136 - protein, 266 aa, Homo sapiens [sequence omitted]

SEQ ID NO: 137 - protein, 266 aa, Homo sapiens [sequence omitted]

SEQ ID NO: 138 - protein, 60 aa, Homo sapiens
DWTFQTLVMLETVPRSGEVYTCQVEHPSVTSPLTVEWSARSESAQSKMLSGVGGFVLGLL

SEQ ID NO: 139 - protein, 266 aa, Homo sapiens
MVCLKLPGGSSLAALTVTLMVLSSRLAFAGDTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSKMLSGVGGFVLGLLFLGAGLFIYFRNQKGHSGLQPTGFLS

SEQ ID NO: 140 - protein, 266 aa, Homo sapiens [sequence omitted]

SEQ ID NO: 141 - protein, 266 aa, Homo sapiens
MVCLKLPGGSSLAALTVTLMVLSSRLAFAGDTRPRFLELLKSECHFFNGTERVRFLERHFHNQEEYARFDSDVGEYRAVRELGRPDAEYWNSQKDLLEQKRGQVDNYCRHNYGVVESFTVQRRVHPQVTVYPAKTQPLQHHNLLVCSVSGFYPGSIEVRWFRNGQEEKAGVVSTGLIQNGDWTFQTLVMLETFPRSGEVYTCQVEHPSVTSPLTVEWSARSESAQSKMLSGVGGFVLGLLFLGAGLFIYFRNQKGHSGLQPTGFLS

SEQ ID NO: 142 - protein, 266 aa, Homo sapiens [sequence omitted]

SEQ ID NO: 143 - protein, 266 aa, Homo sapiens
MVCLKLPGGSCMAALTVTLTVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSKMLSGVGGFVLGLLFLGTGLFIYFRNQKGHSGLQPTGLLS

SEQ ID NO: 144 - protein, 266 aa, Homo sapiens
MVCLKLPGGSYMAKLTVTLMVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQSKMLSGVGGFVLGLLFLGAGLFIYFKNQKGHSGLHPTGLVS

SEQ ID NO: 145 - protein, 260 aa, Homo sapiens
MRPEDRMFHIRAVILRALSLAFLLSLRGAGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 146 - protein, 258 aa, Homo sapiens
MMVLQVSAAPRTVALTALLMVLLTSVVQGRATPENYLFQGRQECYAFNGTQRFLERYIYNREELVRFDSDVGEFRAVTELGRPEAEYWNSQKDILEEERAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSKTLTGAGGFVLGLIICGVGIFMHRRSKKVQRGSA

SEQ ID NO: 147 - protein, 258 aa, Homo sapiens
MMVLQVSAAPRTVALTALLMVLLTSVVQGRATPENYVYQLRQECYAFNGTQRFLERYIYNREEFVRFDSDVGEFRAVTELGRPDEDYWNSQKDLLEEKRAVPDRVCRHNYELDEAVTLQRRVQPKVNVSPSKKGPLQHHNLLVCHVTDFYPGSIQVRWFLNGQEETAGVVSTNLIRNGDWTFQILVMLEMTPQQGDVYICQVEHTSLDSPVTVEWKAQSDSARSKTLTGAGGFVLGLIICGVGIFMHRRSKKVQRGSA

SEQ ID NO: 148 - protein, 135 aa, Homo sapiens
MILNKALLLGALALTTVMSPCGGEDIVADHVASCGVNLYQFYGPSGQYTHEFDGDEEFYVSPVTLGQPNTLICLVDNIFPPVVNITWLSNGQSVTEGVSETSFLSKSDHSFFKISYLTFLQGLRSVGASRHQGPL

SEQ ID NO: 149 - protein, 134 aa, Homo sapiens
MILNKALMLGALALTTVMSPCGGEDIVADHVASYGVNLYQSYGPSGQYSHEFDGDEEFYVSPVTLGQPNTLICLVDNIFPPVVNITWLSNGHSVTEGVSTSFLSKSDHSFFKISYLTFLRGLRSVGASRHQGPL

SEQ ID NO: 150 - protein, 135 aa, Homo sapiens
MILNKALLLGALALTTVMSPCGGEDIVADHVASYGVNLYQSYGPSGQYTHEFDGDEQFYVSPVTLGQPNTLICLVDNIFPPVVNITWLSNGHSVTEGVSETSFLSKSDHSFFKISYLTFLRGLRSVGASRHQGPL

SEQ ID NO: 151 - protein, 135 aa, Homo sapiens
MILNKALMLGALALTTVMSPCGGEDIVADHVASYGVNLYQSYGPSGQYTHEFDGDEQFYVSPVTLGQPNILICLVDNIFPPVVNITWLSNGHSVTEGVSETSFLSKSDHSFFKISYLTLLRGLRSVGASRHQGPL

SEQ ID NO: 152 - protein, 261 aa, Homo sapiens
MSWKKALRIPGGLRAATVTLMLSMLSTPVAEGRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKMLSGIGGFVLGLIFLGLGLIIHHRSQKGLLH

SEQ ID NO: 153 - protein, 261 aa, Homo sapiens [sequence omitted]

SEQ ID NO: 154 - protein, 261 aa, Homo sapiens [sequence omitted]

SEQ ID NO: 155 - protein, 261 aa, Homo sapiens [sequence omitted]

SEQ ID NO: 156 - protein, 261 aa, Homo sapiens [sequence omitted]

SEQ ID NO: 157 - protein, 261 aa, Homo sapiens [sequence omitted]

SEQ ID NO: 158 - protein, 29 aa, Homo sapiens
GLIFLGLGLIIRQRSRKGPQGPPPAGLLH

SEQ ID NO: 159 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 161 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 163 - protein, 276 aa, Homo sapiens
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 164 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 165 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 166 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 167 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 227 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 189 aa, synthetic construct
IKCEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 172 - protein, 189 aa, synthetic construct
IKECHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 173 - protein, 189 aa, synthetic construct
IKEEHVIIQAECYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 174 - protein, 189 aa, synthetic construct
IKEEHVIIQAEFYLNPDQSGEFMFDFDCDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 175 - protein, 189 aa, synthetic construct
IKEEHVIIQAEFYLNPDQSGEFMFDFDGDEIFHVDMAKKETVWRLEEFGRFASFEAQGALANIAVDKANLEIMTKRSNYCPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 176 - protein, 189 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLCNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 177 - protein, 189 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTCSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 178 - protein, 189 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTNCPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 179 - protein, 199 aa, synthetic construct
GDTRPRCLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 180 - protein, 199 aa, synthetic construct
GDTRPRFLECVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 181 - protein, 199 aa, synthetic construct
GDTRPRFLEQVKHECHFFCGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 182 - protein, 199 aa, synthetic construct
GDTRPRFLEQVKHECHFFNCTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 183 - protein, 199 aa, synthetic construct
GDTRPRFLEQVKHECHFFNGTERVRFLDRYFYCQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 184 - protein, 199 aa, synthetic construct
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGCWTFQTLVMLETVPRSGEVYTCQVEHPSLTSPLTVEWRARSESAQSKM

SEQ ID NO: 185 - protein, 229 aa, synthetic construct
SLQPLALEGSLQSRGGGCGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 186 - protein, 663 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERGGGGSGGGGSGGGGSGGGGSIKEEHVIIQAEFYLNPDQSGEFMFDFDGDEIFHVDMAKKETVWRLEEFGRFASFEAQGALANIAVDKANLEIMTCRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 187 - protein, 229 aa, synthetic construct
SLQPLALEGSLQSRGGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 188 - protein, 663 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 189 - protein, 227 aa, synthetic construct
NFFRMVISNPAATGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 190 - protein, 663 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 191 - protein, 227 aa, synthetic construct
NFIRMVISNPAATGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 192 - protein, 423 aa, synthetic construct
IKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 193 - protein, 7 aa, synthetic construct
GGSAAGG

SEQ ID NO: 194 - protein, 232 aa, synthetic construct
GSLQPLALEGSLQSRGIVGGGGSGGGGSGGGGSGDTRPRCLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 195 - protein, 232 aa, synthetic construct
GSLQPLALEGSLQSRGIVGGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 196 - protein, 232 aa, synthetic construct
GSLQPLALEGSLQSRGIVGGGGSGGGGSGGGGSGDTRPRFLEQVKHECHFFNGTERVRFLDRYFYCQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 197 - protein, 423 aa, synthetic construct
IKECHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 198 - protein, 232 aa, synthetic construct
GSLQPLALEGSLQSRGIVGGGGSGGGGSGGGGSGDTRPRFLEQVKHECHFFCGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 199 - protein, 232 aa, synthetic construct
GSLQPLALEGSLQSRGIVGGGGSGGGGSGGGGSGDTRPRFLEQVKHECHFFNCTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 200 - protein, 423 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLCNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 201 - protein, 232 aa, synthetic construct
GSLQPLALEGSLQSRGIVGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFCTLVMLETVPRSGEVYTCQVEHPSLTSPLTVEWRARSESAQSKM

SEQ ID NO: 202 - protein, 232 aa, synthetic construct
GSLQPLALEGSLQSRGIVGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDCTFQTLVMLETVPRSGEVYTCQVEHPSLTSPLTVEWRARSESAQSKM

SEQ ID NO: 203 - protein, 423 aa, synthetic construct
IKEEHVIIQAECYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 204 - protein, 232 aa, synthetic construct
GSLQPLALEGSLQSRGIVGGGGSGGGGSGGGGSGDTRPRFLECVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 205 - protein, 423 aa, synthetic construct
IKEEHVIIQAEFYLNPDQSGEFMFDFDGDEIFHVDMAKKETVWRLEEFGRFASFEAQGALANIAVDKANLEIMTKRSNYCPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 206 - protein, 423 aa, synthetic construct
IKEEHVIIQAEFYLNPDQSGEFMFDFDGDEIFHVDMAKKETVWRLEEFGRFASFEAQGALANIAVDKANLEIMTKRSNYTPCTNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 207 - protein, 423 aa, synthetic construct
IKEEHVIIQAEFYLNPDQSGEFMFDFDCDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 208 - protein, 232 aa, synthetic construct
GSLQPLALEGSLQSRGIVGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNCDWTFQTLVMLETVPRSGEVYTCQVEHPSLTSPLTVEWRARSESAQSKM

SEQ ID NO: 209 - protein, 232 aa, synthetic construct
GSLQPLALEGSLQSRGIVGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGCWTFQTLVMLETVPRSGEVYTCQVEHPSLTSPLTVEWRARSESAQSKM

SEQ ID NO: 210 - protein, 423 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 211 - protein, 232 aa, synthetic construct
GSLQPLALEGSLQSRGIVGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 212 - protein, 423 aa, synthetic construct
IKEEHVIIQAEFYLNPDQSGEFMFDFDGCEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 213 - protein, 423 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTCSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 214 - protein, 232 aa, synthetic construct
GSLQPLALEGSLQSRGIVGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVCGFYPASIEVRWFRNGQEEKTGVVSTGLIQNGDWTFQTLVMLETVPRSGEVYTCQVEHPSLTSPLTVEWRARSESAQSKM

SEQ ID NO: 215 - protein, 423 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTNCPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 216 - protein, 423 aa, synthetic construct
IKCEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 217 - protein, 423 aa, synthetic construct
IKEEHVIIQAEFYLNPDQSGEFMFDFDGDEIFHVDMAKKETVWRLEEFGRFASFEAQGALANIAVDKANLECMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 218 - protein, 423 aa, synthetic construct
IKEEHVIIQAEFYLNPDQSGEFMFDFDGDEIFHVDMAKKETVWRLEEFGRFASFEAQGALANIAVDKANLEIMTCRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 219 - protein, 229 aa, synthetic construct
SLQPLALEGSLQSRGCGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 220 - protein, 15 aa, synthetic construct
CGGGSGGGGSGGGGS

SEQ ID NO: 221 - protein, 229 aa, synthetic construct
SLQPLALEGSLQSRGGCGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 222 - protein, 15 aa, synthetic construct
GCGGSGGGGSGGGGS

SEQ ID NO: 223 - protein, 229 aa, synthetic construct
SLQPLALEGSLQSRGGGGGCGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 224 - protein, 15 aa, synthetic construct
GGGGCGGGGSGGGGS

SEQ ID NO: 225 - protein, 229 aa, synthetic construct
SLQPLALEGSLQSRGGGGCSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 226 - protein, 15 aa, synthetic construct
GGGCSGGGGSGGGGS

SEQ ID NO: 227 - protein, 663 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPCNKINQRILVVDPVTSEHELTCQAECYPKAEVIWTSSDHQVLSGKTTTTNSKREEKLFNVTSTLRINTTTNEIFYCTFRRLDPEENHTAELVIPELPLAHPPNER

SEQ ID NO: 228 - protein, 387 aa, synthetic construct
QAVHAAHAEINGGGGSGGGGSGGGGSLEQPNVAISLSRTEALNHHNTLVCSVTDFYPAKIKVRWFRNGQEETVGVSSTQLIRNGDWTFQVLVMLEMTPHQGEVYTCHVEHPSLKSPITVEWRAQSESARSKGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 229 - protein, 11 aa, synthetic construct
QAVHAAHAEIN

SEQ ID NO: 230 - protein, 663 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERGGGGSGGGGSGGGGSGGGGSIKEEHVIIQAEFYLNPDQSGEFMFDFDGDEIFHVDMAKKETVWRLEEFGRFASFEAQGALANIAVDKANLECMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 231 - protein, 675 aa, synthetic construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGGGSGGGGSGGGGSGGGGSFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 232 - protein, 462 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERGGGGSGGGGSGGGGSNFFRMVISNPAATGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 233 - protein, 462 aa, synthetic construct
NFFRMVISNPAATGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 234 - protein, 5 aa, synthetic construct
GGCGS

SEQ ID NO: 235 - protein, 5 aa, synthetic construct
GCGGS

SEQ ID NO: 236 - protein, 5 aa, synthetic construct
  VARIANT 4: note = X is any amino acid other than proline
VPGXG

SEQ ID NO: 237 - protein, 150 aa, synthetic construct
IGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYK